(19) United States
(12) Patent Application Publication
(10) Pub. No.: US 2024/0425478 A1
(43) Pub. Date: Dec. 26, 2024

(54) CYCLOPENTYLPYRAZOLE CDK2 INHIBITORS

(71) Applicant: Genentech, Inc., South San Francisco, CA (US)

(72) Inventors: Jessica Marie GRANDNER, South San Francisco, CA (US); Kevin M. JOHNSON, Wentzville, MO (US); Steven MAGNUSON, South San Francisco, CA (US); Jeremy Mark MURRAY, South San Francisco, CA (US); Brendan T. PARR, South San Francisco, CA (US); Vishal A. VERMA, South San Francisco, CA (US); Yong WANG, Burlingame, CA (US); Mingshuo ZENG, South San Francisco, CA (US); Melissa A. ASHLEY, South San Francisco, CA (US)

(21) Appl. No.: 18/698,664

(22) PCT Filed: Oct. 4, 2022
(86) PCT No.: PCT/US22/77501

§ 371 Date: Apr. 4, 2024

Related U.S. Application Data
(60) Provisional application No. 63/252,394, filed on Oct. 5, 2021.

(51) Int. Cl. C07D 401/12; A61K 31/4439; A61K 31/496; A61K 31/497; A61K 31/501; A61K 31/506; A61K 31/53; A61K 31/5377; C07D 401/14; C07D 403/12; C07D 405/14

(52) U.S. Cl. CPC C07D 401/12; A61K 31/4439; A61K 31/496; A61K 31/497; A61K 31/501; A61K 31/506; A61K 31/53; A61K 31/5377; C07D 401/14; C07D 403/12; C07D 405/14

(57) ABSTRACT

Described here are compounds of Formula (I) and pharmaceutically acceptable salts thereof, pharmaceutical compositions comprising same, and their use and preparation.

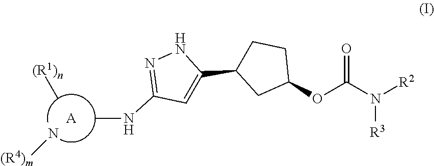

### CROSS-REFERENCES TO RELATED APPLICATIONS

This application claims priority benefit to U.S. provisional 63/252,394 filed Oct. 5 2021, which is incorporated herein in its entirety.

### BACKGROUND

Throughout eukaryotes, the cell cycle is controlled by the family of cyclin-dependent kinases (CDKs). CDKs are activated by binding of different regulatory cyclin proteins to promote cell cycle progression and many cell cycle-dependent events. Distinct cyclin partners are expressed at different times in the cell cycle to promote proliferation: cyclins D1/2/3 are expressed in G1, cyclins E1/2 are expressed at G1/S, cyclin A2 is expressed during S/G2, and cyclin B1/2/3 are expressed during G2/M. The human genome encodes 21 CDKs, but only a few —CDK1, CDK2, CDK4, CDK6, and CDK7—have been shown to play a direct role in the cell cycle with cyclin partners in most mammalian cell types. See, e.g., Lu et al., Toxicological Sciences (2020) 177:226-234, and Asghar et al., Nature Rev. (2015) 14:130-146. CDK family members share high sequence homology, presenting challenges for development of isoform selective small molecule inhibitors. See, e.g., Asghar et al., Nature Rev. (2015) 14:130-146.

In general, the mammalian cell cycle requires the sequential activation of three interphase CDKs 2, 4, and 6 to drive cells through the interphase, followed by mitosis, which is controlled by CDK1. CDK4/6 together with D-type cyclins are activated during the G1 phase, followed by increased expression of E-type cyclins that activate CDK2 to drive the G1/S transition. CDK2 is activated by A-type cyclins to drive the transition from S phase to mitosis. CDK1 is first activated by A-type cyclins and later by B-type cyclins to drive the completion of the cell cycle through mitosis. Increased cell proliferation is a result of direct or indirect deregulation of this cell division cycle. See, e.g., Lu et al., Toxicological Sciences (2020) 177:226-234.

CDK4/6 inhibitors have been found useful in the treatment of cancer, but also have been associated with negative side effects, such as neutropenia. See, e.g., Thill and Schmidt Ther. Adv. Med. Oncol. (2018) 10:1-12. Gastrointestinal toxicity, such as from intestinal cell proliferation, has been associated with CDK1 inhibition, and CDK1 inhibition may have broader effects on all proliferative cells based on the results of mouse knockout studies. See, e.g., Lu et al., Toxicological Sciences (2020) 177:226-234; Santamaria et al., Nature (2007) 448:811-815. Deregulation of CDK2 has been shown to occur frequently in cancers, such as breast cancer (see, e.g., Scaltriti et al., PNAS (2011) 108:3761-3766, Akli et al., Cancer Res. (2011) 71:3377-3386) and ovarian cancer (see, e.g., Yang et al., Oncotarget (2015) 6:20801-20812), as well as many others. There is also accumulating evidence that deficiency or inhibition of CDK2 will not have an adverse effect on normal (non-cancerous tissues); see, e.g., Barbacid et al., Cold Spring Harbor Symposia on Quantitative Biology (2005) 233-240, describing CDK2 (−/−) mice and conditional null mice were viable, with no major cell cycle defects apart from sterility, and Chauhan et al., Biochemical Journal (2016) 473:2783-2798, similarly indicating ‘kinase dead’ mutant mouse models were also sterile, but displayed normal mitotic cell cycle progression. Accordingly there is a need to develop selective inhibitors of CDK2, minimizing CDK4/6 and CDK1 inhibition.

### SUMMARY

In one embodiment, provided is a compound of Formula (I):

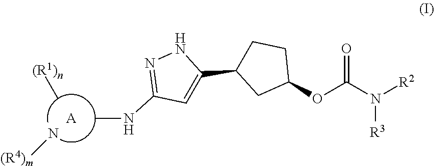

or a pharmaceutically acceptable salt thereof, wherein:
Ring A is a 6-membered heteroaryl having 1 at least N-ring heteroatom, and having 0 to 2 additional N-ring heteroatoms;
each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, —S(O)₂R^{1a}, C₃₋₈ cycloalkyl, a 3 to 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl, a 3- to 6-membered heterocycloalkyl having 1 or 2 heteroatoms each independently N or O, or C₁₋₆ alkylaryl, wherein the cycloalkyl, the heterocycloalkyl, and the aryl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, C₁₋₄ alkyl, C₂₋₄ alkoxyalkyl, or C₁₋₃ haloalkyl;
subscript n is an integer 0, 1, 2, 3, or 4; and
subscript m is an integer 0 or 1.

In another embodiment, provided is a pharmaceutical composition comprising a compound as described herein, or a pharmaceutically acceptable salt thereof, and a pharmaceutically acceptable excipient.

In another embodiment, provided is a method of treating a CDK2-mediated disorder in a human in need thereof, the method comprising administering to the human a therapeutically effective amount of a compound as described herein, or a pharmaceutically acceptable salt thereof, or a pharmaceutical composition as described herein.

In another embodiment, provided is a method for manufacturing a medicament for treating a CDK2-mediated disorder in a human in need thereof, characterized in that the compound as described herein, or a pharmaceutically acceptable salt thereof, or a pharmaceutical composition as described herein, is used.

In another embodiment, provided is use of a compound as described herein, or a pharmaceutically acceptable salt thereof, or a pharmaceutical composition as described herein, for the manufacture of a medicament for the treatment in a human of a CDK2-mediated disorder.

In another embodiment, provided is the compound as described herein, or a pharmaceutically acceptable salt thereof, or a pharmaceutical composition as described herein, for use in the treatment of a CDK2-mediated disorder in a human in need thereof.

### DETAILED DESCRIPTION

### I. General

Provided herein are cyclopentylpyrazole inhibitors of CDK2 of Formula (I), or pharmaceutically acceptable salt thereof, pharmaceutical compositions thereof, and their use, e.g., for treating CDK2-mediated diseases such as cancer, and for their preparation.

### II. Definitions

Unless specifically indicated otherwise, all technical and scientific terms used herein have the same meaning as commonly understood by those of ordinary skill in the art. In addition, any method or material similar or equivalent to a method or material described herein can be used. For purposes as described herein, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. The abbreviation “Me” refers to the alkyl group methyl (—CH₃).

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The alkoxy component is as defined above. Examples of the alkoxyalkyl group include, but are not limited to, 2-ethoxy-ethyl and methoxymethyl.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2-trifluoroethoxy, perfluoroethoxy, etc.

Cycloalkyl, heterocycloalkyl, aryl, and heteroaryl bicyclic (fused, bridged, or spirocyclic) or polycyclic (fused, bridged, or spirocyclic) ring systems are defined based on the nature of the ring system and/or point of attachment. For example, if the entire bicyclic or polycyclic ring system is fully non-aromatic and contains at least one ring heteroatom, then the ring system is a heterocycloalkyl bicyclic or polycyclic ring system. If the entire bicyclic or polycyclic ring system is fully aromatic and contains at least one ring heteroatom, then the ring system is considered a heteroaryl bicyclic or polycyclic ring system. If the bicyclic or polycyclic ring system contains a mix of non-aromatic and aromatic ring systems, then it is the point of attachment that dictates the nature of the ring system: if attached to the non-aromatic cycloalkyl or heterocycloalkyl ring, it is considered a cycloalkyl or heterocycloalkyl bicyclic or polycyclic ring system; if it is attached to the aromatic aryl or heteroaryl ring, it is considered an aryl or heteroaryl bicyclic or polycyclic ring system.

“Cycloalkyl” refers to a non-aromatic, saturated or partially unsaturated, monocyclic, bicyclic, or polycyclic ring system containing from 3 to 12 ring carbon atoms, but no heteroatom ring atoms. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, bicyclo[1.1.1]pentane, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl.

“Heterocycloalkyl” or “heterocyclyl” refers to a non-aromatic, saturated or unsaturated, monocyclic, bicyclic, or polycyclic ring system having from 3 to 12 ring carbon or heteroatoms, wherein the ring system contains from 1 to 4 ring heteroatoms selected from N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring carbon atoms and any suitable number of rings, but no heteroatom ring atoms. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

“Heteroaryl” refers to a monocyclic or fused bicyclic or tricyclic aromatic ring assembly containing 5 to 16 ring carbon and heteroatoms, where from 1 to 5 of the ring atoms are a heteroatom selected from N, O and S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can include groups such as pyridinones, such as pyridin-2-one, pyridin-3-one, or pyridin-4-one, pyridazinones, such as pyridazin-3(2H)-one or pyridazin-4(1H)-one, pyrimidinones, such as pyrimidin-2(1H)-one or pyrimidin-4(3H)-one and pyrazinones. By way of example, the tautomerization of each is illustrated:

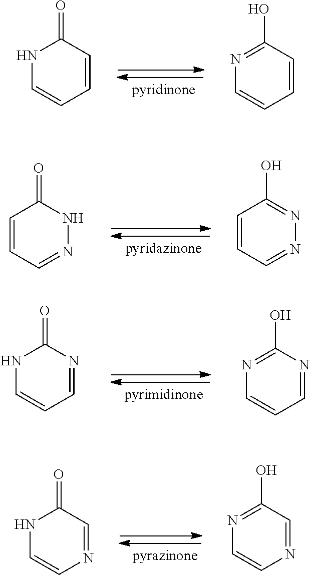

The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Substitution” or “substitute” refers to replacement of a hydrogen atom with a different (i.e., non-hydrogen) group, as described in more detail herein. A subscript n or m which is zero (0), and/or a group which is substituted with zero (0) substituents, means the absence of a R¹ or R⁴ group (in the case of variable n or m) and/or no replacement of hydrogen with the given listing of substituents.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Therapeutically effective amount or dose” or “therapeutically sufficient amount or dose” or “effective amount or dose” or “sufficient amount or dose” are used interchangeably to refer to a dose that produces therapeutic effects for which it is administered. The exact dose will depend on the purpose of the treatment, and will be ascertainable by one skilled in the art using known techniques.

“Subject” refers to animals such as mammals, including, but not limited to, primates (e.g., humans), cows, sheep, goats, horses, dogs, cats, rabbits, rats, mice and the like. In some embodiments, the subject is a human.

### III. Compounds

Provided are compounds and pharmaceutically acceptable salts, of any one of Formula (I).

In some embodiments, provided is a compound of Formula (I):

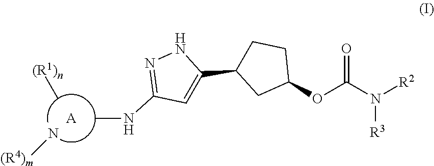

or a pharmaceutically acceptable salt thereof, wherein:
Ring A is a 6-membered heteroaryl having at least 1 N-ring heteroatom, and having 0 to 2 additional N-ring heteroatoms;
each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, —S(O)₂R^{1a}, C₃₋₈ cycloalkyl, a 3, 4, 5, or 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl, a 3- to 6-membered heterocycloalkyl having 1 or 2 heteroatoms each independently N or O, or C₁₋₆ alkylaryl, wherein the cycloalkyl, the heterocycloalkyl, and the aryl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, C₁₋₄ alkyl, C₂₋₄ alkoxyalkyl or C₁₋₃ haloalkyl;
subscript n is an integer 0, 1, 2, 3, or 4; and
subscript m is an integer 0 or 1.

In some embodiments, provided is a compound of Formula (I):

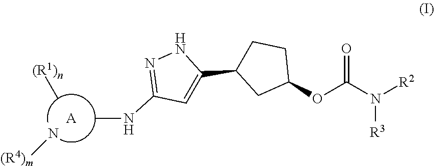

or a pharmaceutically acceptable salt thereof, wherein:
Ring A is

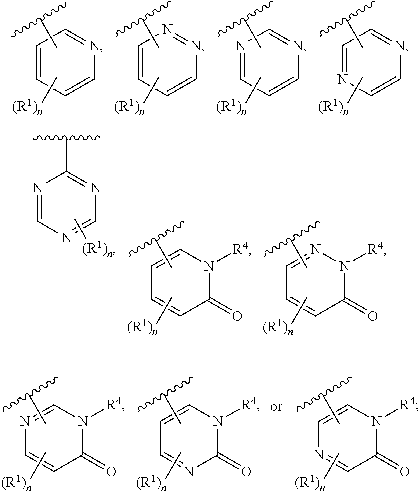

each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R¹, —S(O)₂R^{1a}, C₃₋₈ cycloalkyl, a 3, 4, 5, or 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl, a 3- to 6-membered heterocycloalkyl having 1 or 2 heteroatoms each independently N or O, or C₁₋₆ alkylaryl, wherein the cycloalkyl, the heterocycloalkyl, and the aryl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, C₁₋₄ alkyl, C₂₋₄ alkoxyalkyl or C₁₋₃ haloalkyl;
subscript n is an integer 0, 1, 2, 3, or 4; and
   subscript m is an integer 0 or 1.

In some embodiments, provided is a compound of:

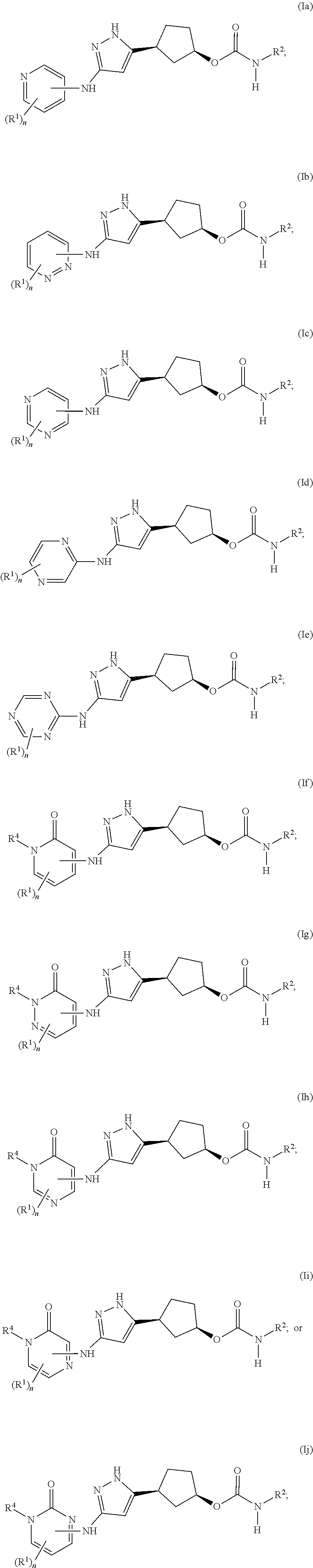

or a pharmaceutically acceptable salt thereof, wherein:
each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, —S(O)₂R^{1a}, C₃₋₈ cycloalkyl, a 3 to 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl, a 3- to 6-membered heterocycloalkyl having 1 or 2 heteroatoms each independently N or O, or C₁₋₆ alkylaryl, wherein the cycloalkyl, the heterocycloalkyl, and the aryl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₄ alkyl, or C₁₋₄ haloalkyl;
R⁴ is hydrogen, C₁₋₄ alkyl, C₂₋₄ alkoxyalkyl or C₁₋₃ haloalkyl;
subscript n is an integer 0, 1, 2, 3, or 4; and
subscript m is an integer 0 or 1.

In some embodiments, provided is a compound of Formula (I):

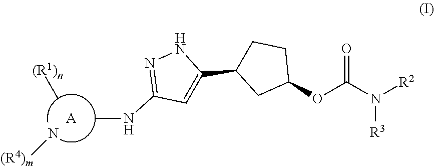

or a pharmaceutically acceptable salt thereof, wherein:
Ring A is a 6-membered heteroaryl having 1 N-ring heteroatom, and optionally having 1 to 2 additional N-ring heteroatoms;
each R¹ is independently C₁₋₆ alkyl, C₁₋₆ alkoxy, C₁₋₆ hydroxyalkyl, C₂₋₆ alkoxyalkyl, halogen, C₁₋₄ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, C₃₋₈ cycloalkyl, a 3, 4, 5, or 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl or a 3 to 6 membered heterocycloalkyl having 1 or 2 heteroatoms each independently N or O, wherein the cycloalkyl and the heterocycloalkyl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, Cia alkyl, C₂₋₄ alkoxyalkyl or C₁₋₃ haloalkyl;
subscript n is an integer 0, 1, 2, 3, or 4; and
subscript m is an integer 0 or 1.

In some embodiments, provided is a compound of Formula (I):

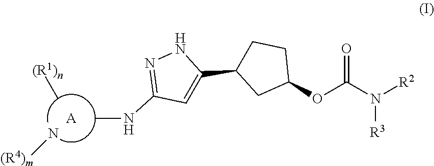

or a pharmaceutically acceptable salt thereof, wherein:
Ring A is a 6-membered heteroaryl having 1 to 2 additional N heteroatoms;
each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, C₃₋₈ cycloalkyl, a 3, 4, 5, or 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl or a 3 to 6 membered heterocycloalkyl having 1 or 2 heteroatoms each independently N or O, wherein the cycloalkyl and the heterocycloalkyl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, Cia alkyl, C₂₋₄ alkoxyalkyl or C₁₋₃ haloalkyl;
subscript n is an integer 0, 1, 2, 3, or 4; and
subscript m is an integer 0 or 1.

As should be understood herein, Ring A is a monocyclic 6-membered heteroaryl ring that has at least one (1) N-ring heteroatom, and optionally 1 to 2 additional N-ring heteroatoms. As used herein “N-ring heteroatom” is understood to be a nitrogen (N) heteroatom which is a member of the 6-membered ring system. In certain embodiments, Ring A is a monocyclic 6-membered heteroaryl ring that has 1 N-ring heteroatom. In certain embodiments, Ring A is a monocyclic 6-membered heteroaryl ring that has 2 N-ring heteroatoms. In certain embodiments, Ring A is a monocyclic 6-membered heteroaryl ring that has 3 N-ring heteroatoms. In certain embodiments, wherein m is 1, and wherein valency permits, the at least 1 N-ring heteroatom, or the optional 1 to 2 additional heteroatoms, is substituted with the group R⁴. In certain embodiments, wherein m is 1, and wherein valency permits, the at least 1 N-ring heteroatom is substituted with the group R⁴. In certain embodiments, wherein m is 1, and wherein valency permits, the at least 1 N-ring heteroatom is substituted with the group R⁴, and Ring A comprises 1 additional N-ring heteroatom.

In certain embodiments of Ring A, the at least one (1) N-ring heteroatom is meta relative to the point of attachment. In certain embodiments, the at least one (1) N-ring heteroatom is para relative to the point of attachment. In certain embodiments, wherein Ring A has 2 N-ring heteroatoms, the N atoms are meta and para relative to the point of attachment. In certain embodiments, Ring A comprises at least one (1) N-ring heteroatom meta relative to the point of attachment and Ring A further comprises an oxo (═O) group para relative to the point of attachment, optionally wherein Ring A further comprises 1 additional N-ring heteroatom.

In some embodiments, Ring A is a 6-membered heteroaryl having 1 N-ring heteroatom, and optionally having 1 to 2 additional N-ring heteroatoms. In some embodiments, Ring A is a 6-membered heteroaryl having at least 1 N-ring heteroatom, and having 0 to 2 additional N-ring heteroatoms. In some embodiments, Ring A is a 6-membered heteroaryl having 1 to 3 N-ring heteroatoms.

In some embodiments, the compound of Formula (I) or a pharmaceutically acceptable salt thereof, is the compound wherein Ring A is

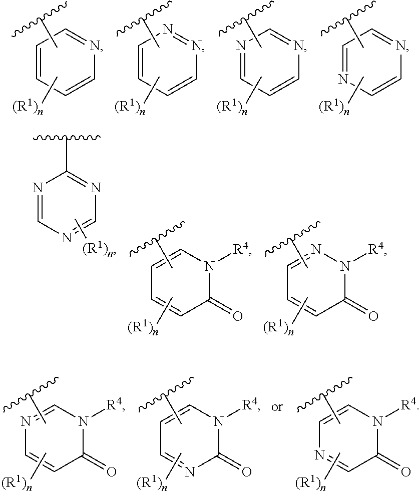

In some embodiments, the compound of Formula (I) or a pharmaceutically acceptable salt thereof, is the compound wherein Ring A is

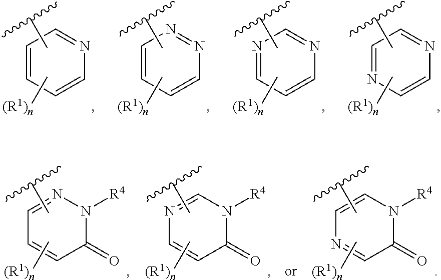

In some embodiments, the compound of Formula (I) or a pharmaceutically acceptable salt thereof, is the compound wherein Ring A is

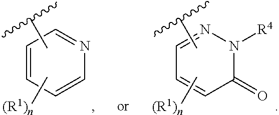

In some embodiments, the compound of Formula (I) or a pharmaceutically acceptable salt thereof, is the compound wherein Ring A is

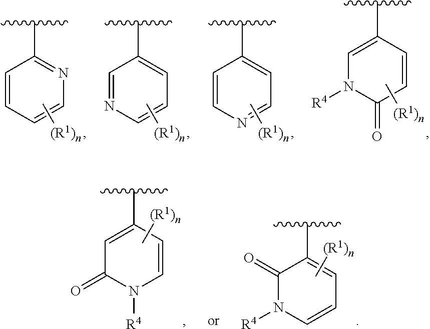

In some embodiments, the compound of Formula (I) or a pharmaceutically acceptable salt thereof, is the compound wherein Ring A is

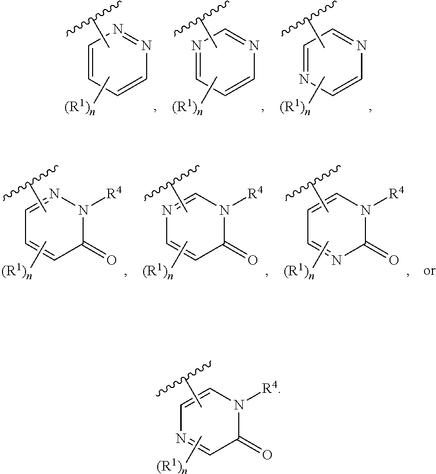

In some embodiments, the compound of Formula (I) or a pharmaceutically acceptable salt thereof, is the compound wherein Ring A is

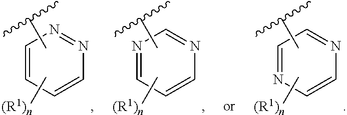

In some embodiments, the compound of Formula (I) or a pharmaceutically acceptable salt thereof, is the compound wherein Ring A is

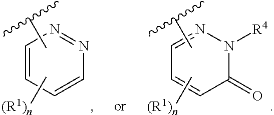

In some embodiments, the compound of Formula (I) or a pharmaceutically acceptable salt thereof, is the compound wherein Ring A is

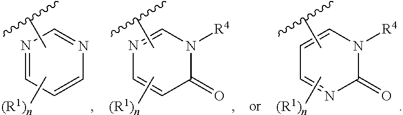

In some embodiments, the compound of Formula (I) or a pharmaceutically acceptable salt thereof, is the compound wherein Ring A is

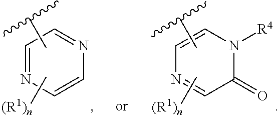

In some embodiments, the compound of Formula (I) or a pharmaceutically acceptable salt thereof, is the compound wherein Ring A is

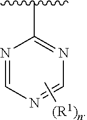

In some embodiments, the compound of Formula (I) or a pharmaceutically acceptable salt thereof, is the compound wherein Ring A has the structure:

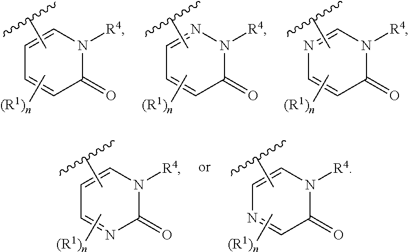

In some embodiments, the compound of Formula (I) or a pharmaceutically acceptable salt thereof, is the compound wherein Ring A has the structure:

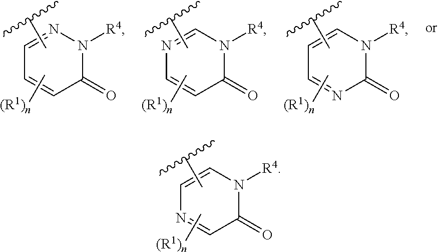

In some embodiments, the compound of Formula (I) or a pharmaceutically acceptable salt thereof, is the compound wherein Ring A has the structure:

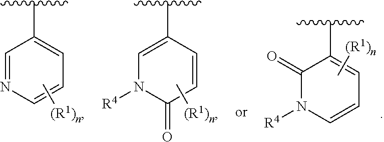

In some embodiments, the compound of Formula (I) or a pharmaceutically acceptable salt thereof, is the compound wherein Ring A has the structure:

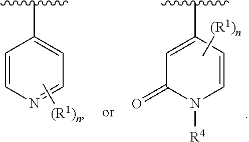

In some embodiments, the compound of Formula (I) or a pharmaceutically acceptable salt thereof, is the compound wherein Ring A has the structure:

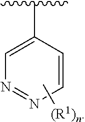

In some embodiments, the compound of Formula (I) or a pharmaceutically acceptable salt thereof, is the compound wherein Ring A has the structure:

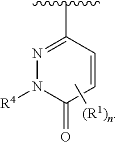

In some embodiments, the compound of Formula (I) or a pharmaceutically acceptable salt thereof, is the compound wherein Ring A is pyridine, pyridazine, pyrimidine, pyrazine, triazine, pyridine-2-one, pyridazine-3-one, pyrimidine-2-one, or pyrazine-2-one.

In some embodiments, provided is a compound of Formula (Ia):

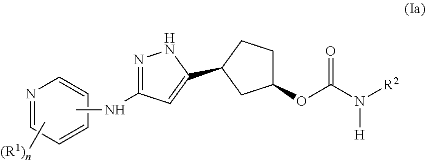

or a pharmaceutically acceptable salt thereof.

In some embodiments, provided is a compound of Formula (Ia-1):

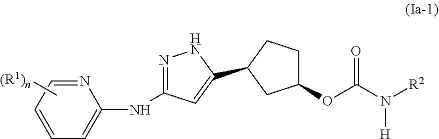

or a pharmaceutically acceptable salt thereof.

In some embodiments, provided is a compound of Formula (Ia-2):

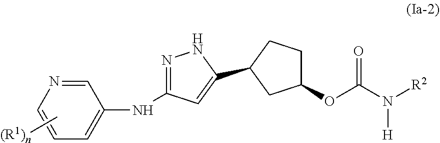

or a pharmaceutically acceptable salt thereof.

In some embodiments, provided is a compound of Formula (Ia-3):

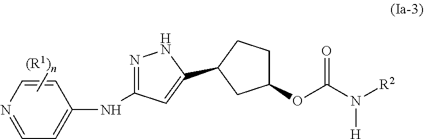

or a pharmaceutically acceptable salt thereof.

In some embodiments, provided is a compound of Formula (Ib):

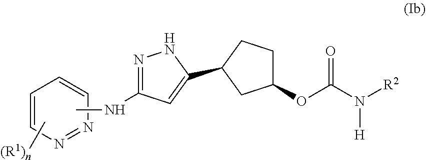

or a pharmaceutically acceptable salt thereof.

In some embodiments, provided is a compound of Formula (Ib-1):

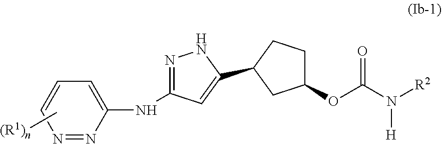

or a pharmaceutically acceptable salt thereof.

In some embodiments, provided is a compound of Formula (Ib-2):

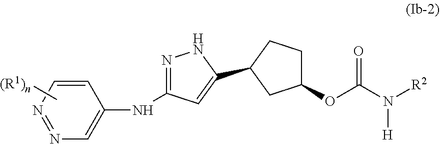

or a pharmaceutically acceptable salt thereof.

In some embodiments, provided is a compound of Formula (Ic):

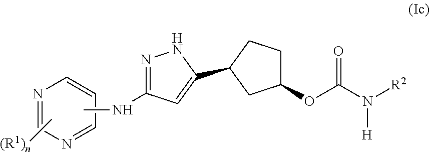

or a pharmaceutically acceptable salt thereof.

In some embodiments, provided is a compound of Formula (Ic-1):

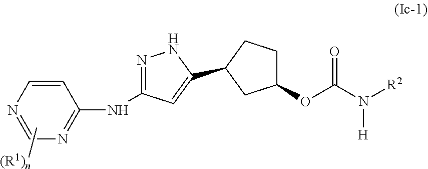

or a pharmaceutically acceptable salt thereof.

In some embodiments, provided is a compound of Formula (Ic-2):

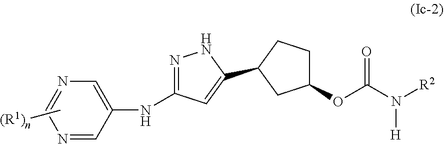

or a pharmaceutically acceptable salt thereof.

In some embodiments, provided is a compound of Formula (Ic-3):

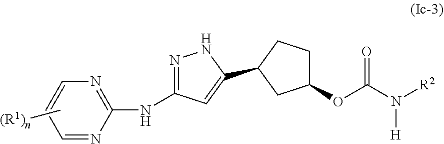

or a pharmaceutically acceptable salt thereof.

In some embodiments, provided is a compound of Formula (Id):

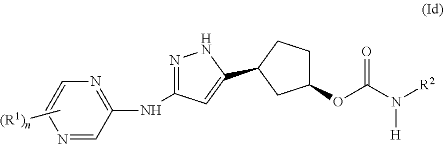

or a pharmaceutically acceptable salt thereof.

In some embodiments, provided is a compound of Formula (Ie):

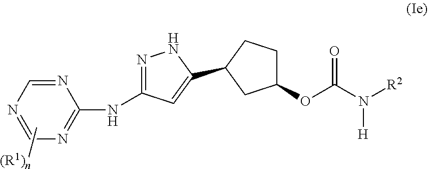

or a pharmaceutically acceptable salt thereof.

In some embodiments, provided is a compound of Formula (If):

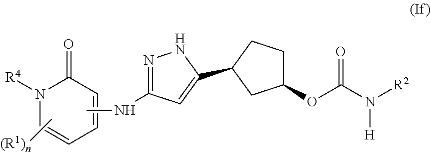

or a pharmaceutically acceptable salt thereof.

In some embodiments, provided is a compound of Formula (If-1):

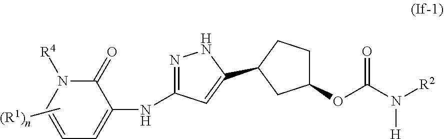

or a pharmaceutically acceptable salt thereof.

In some embodiments, provided is a compound of Formula (If-2):

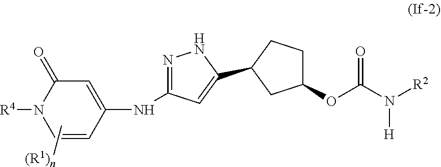

or a pharmaceutically acceptable salt thereof.

In some embodiments, provided is a compound of Formula (If-3):

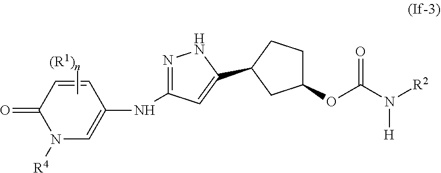

or a pharmaceutically acceptable salt thereof.

In some embodiments, provided is a compound of Formula (If-4):

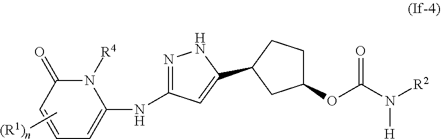

or a pharmaceutically acceptable salt thereof.

In some embodiments, provided is a compound of Formula (Ig):

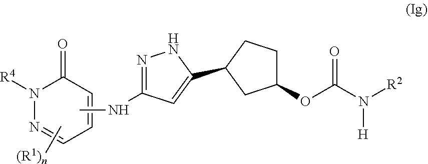

or a pharmaceutically acceptable salt thereof.

In some embodiments, provided is a compound of Formula (Ig-1):

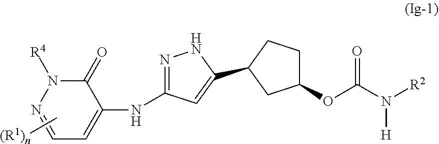

or a pharmaceutically acceptable salt thereof.

In some embodiments, provided is a compound of Formula (Ig-2):

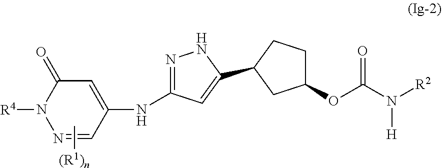

or a pharmaceutically acceptable salt thereof.

In some embodiments, provided is a compound of Formula (Ig-3):

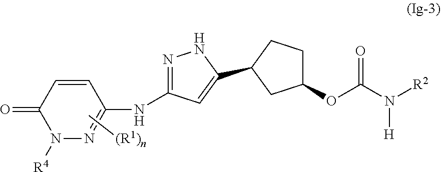

or a pharmaceutically acceptable salt thereof.

In some embodiments, provided is a compound of Formula (Ih):

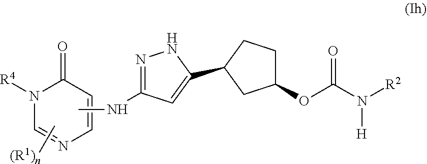

or a pharmaceutically acceptable salt thereof.

In some embodiments, provided is a compound of Formula (Ih-1):

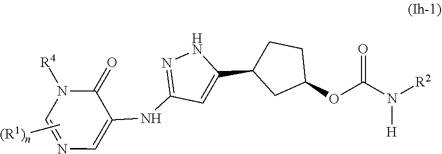

or a pharmaceutically acceptable salt thereof.

In some embodiments, provided is a compound of Formula (Ih-2):

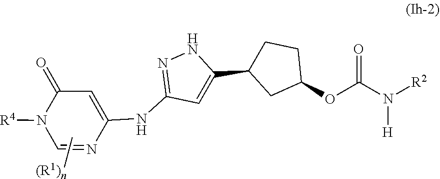

or a pharmaceutically acceptable salt thereof.

In some embodiments, provided is a compound of Formula (Ih-3):

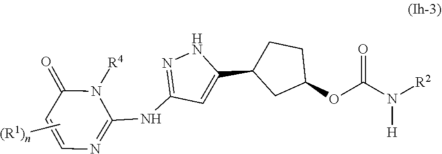

or a pharmaceutically acceptable salt thereof.

In some embodiments, provided is a compound of Formula (Ii):

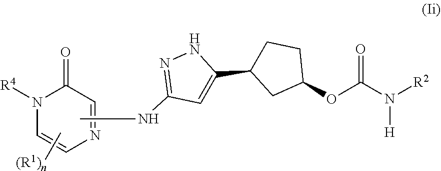

or a pharmaceutically acceptable salt thereof.

In some embodiments, provided is a compound of Formula (Ii-1):

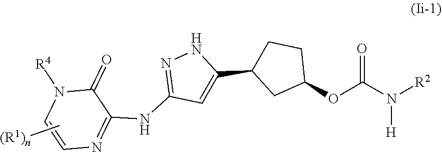

or a pharmaceutically acceptable salt thereof.

In some embodiments, provided is a compound of Formula (Ii-2):

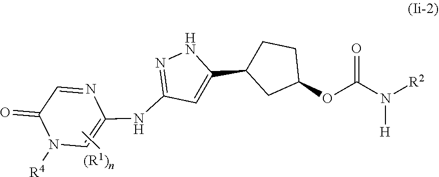

or a pharmaceutically acceptable salt thereof.

In some embodiments, provided is a compound of Formula (Ii-3):

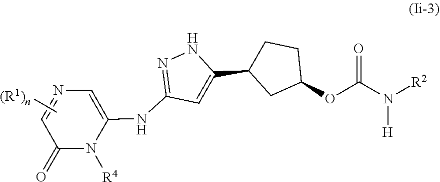

or a pharmaceutically acceptable salt thereof.

In some embodiments, provided is a compound of Formula (Ij):

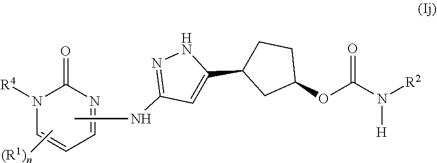

or a pharmaceutically acceptable salt thereof.

In some embodiments, provided is a compound of Formula (Ij-1):

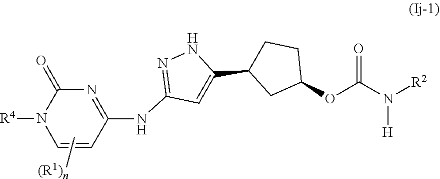

or a pharmaceutically acceptable salt thereof.

In some embodiments, provided is a compound of Formula (Ij-2):

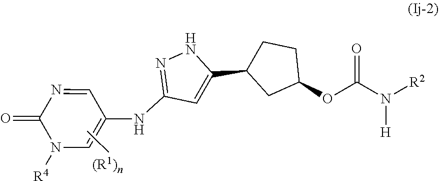

or a pharmaceutically acceptable salt thereof.

In some embodiments, provided is a compound of Formula (Ij-3):

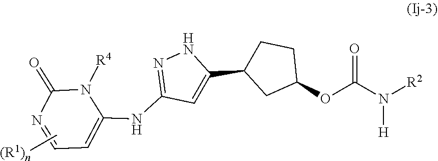

or a pharmaceutically acceptable salt thereof.

In some embodiments, the compound of Formula (I), or a pharmaceutically acceptable salt thereof, is the compound wherein Ring A is

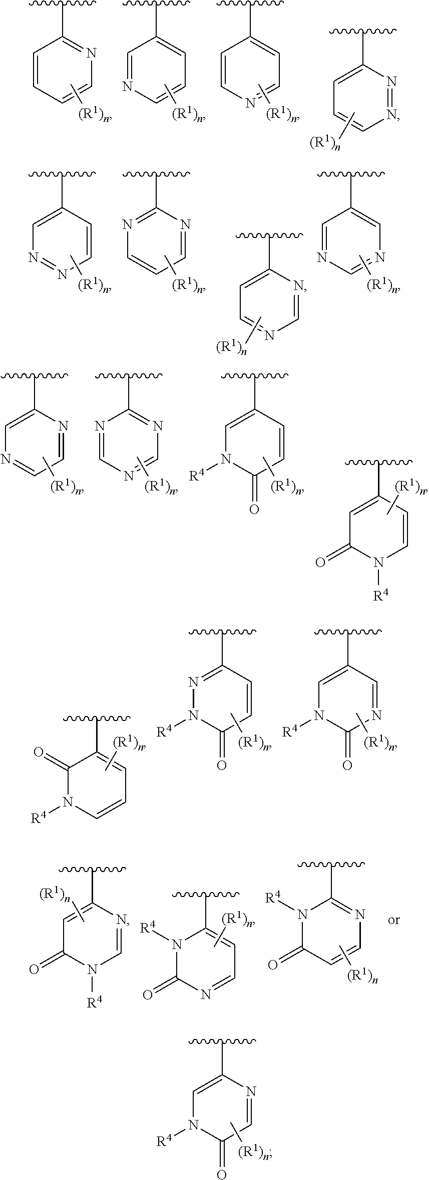

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d},
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and
subscript n is an integer from 0, 1, 2, 3, or 4.

In some embodiments, the compound of Formula (I), or a pharmaceutically acceptable salt thereof, is the compound wherein Ring A is

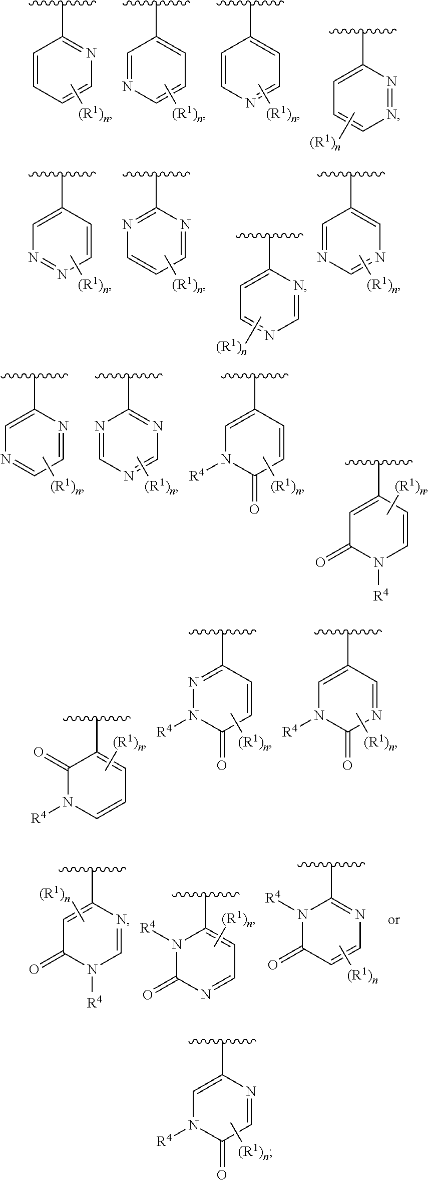

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃alkyl-N(R^{1a})(R^{1b}), —C(O)N(R^{1a})(R^{1b}), —O—R^{1c}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d}.
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and
   subscript n is an integer from 0, 1, 2, 3, or 4.

In some embodiments, the compound of Formula (I), or a pharmaceutically acceptable salt thereof, is the compound wherein Ring A is

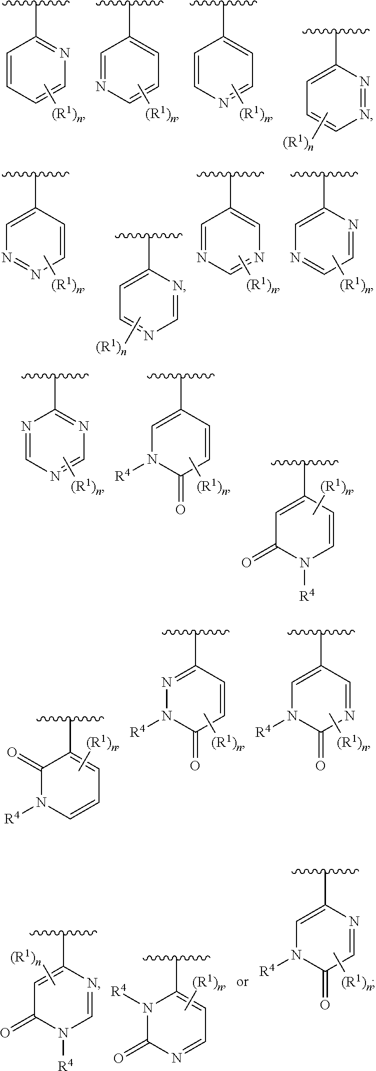

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and
   subscript n is an integer 0, 1, 2, 3, or 4.

In some embodiments, the compound of Formula (I), or a pharmaceutically acceptable salt thereof, is the compound wherein Ring A is

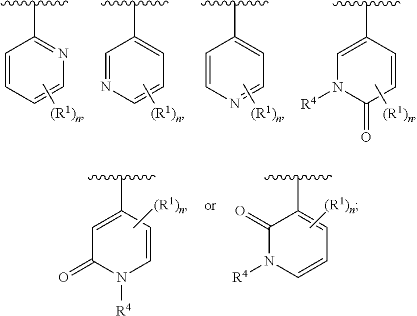

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and
   subscript n is an integer 0, 1, 2, 3, or 4.

In some embodiments, the compound of Formula (I), or a pharmaceutically acceptable salt thereof, is the compound wherein Ring A is

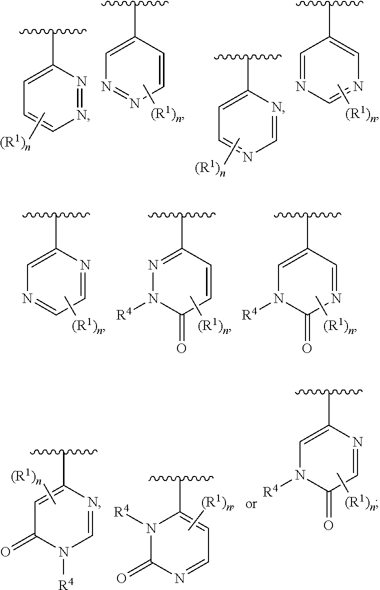

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and subscript n is an integer 0, 1, 2, 3, or 4.

In some embodiments, the compound of Formula (I), or a pharmaceutically acceptable salt thereof, is the compound wherein Ring A is

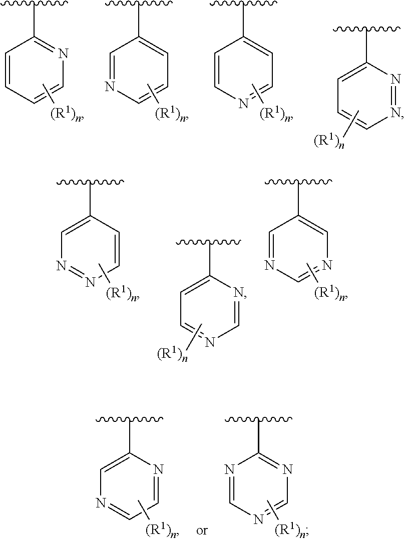

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy; and

subscript n is an integer 0, 1, 2, 3, or 4.

In some embodiments, the compound of Formula (I), or a pharmaceutically acceptable salt thereof, is the compound wherein Ring A is

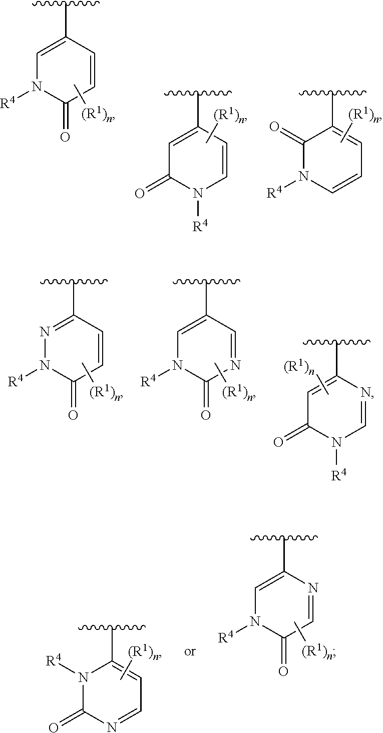

each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₆ alkyl-N(R^{1a})(R^{1b}), —C(O)N(R^{1a})(R^{1b}), —O—R^{1c}, —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d}.
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy;
R⁴ is hydrogen, C₁₋₄ alkyl, C₂₋₄ alkoxyalkyl or C₁₋₃ haloalkyl; and subscript n is an integer 0, 1, 2, 3, or 4.

In some embodiments, the compound of Formula (I), or a pharmaceutically acceptable salt thereof, is the compound wherein Ring A is

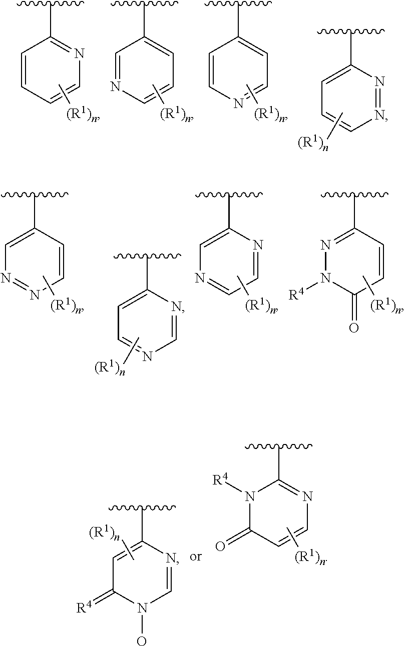

In some embodiments, the compound of Formula (I), or a pharmaceutically acceptable salt thereof, is the compound wherein Ring A is

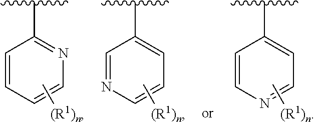

In some embodiments, the compound of Formula (I), or a pharmaceutically acceptable salt thereof, is the compound wherein Ring A is

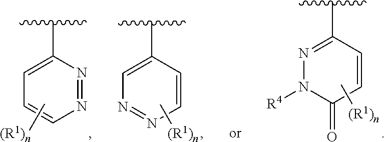

In some embodiments, the compound of Formula (I), or a pharmaceutically acceptable salt thereof, is the compound wherein Ring A is

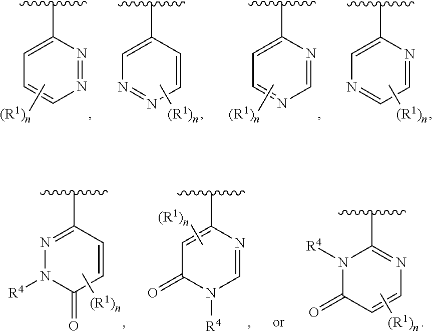

In some embodiments, the compound of Formula (I), or a pharmaceutically acceptable salt thereof, is the compound wherein Ring A is

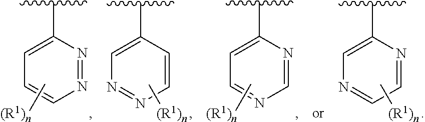

In some embodiments, the compound of Formula (I), or a pharmaceutically acceptable salt thereof, is the compound wherein Ring A is

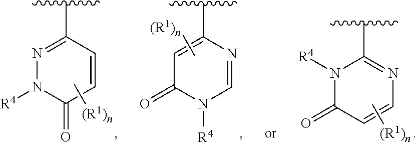

In some embodiments, the compound of Formula (I), or a pharmaceutically acceptable salt thereof, is the compound wherein Ring A is

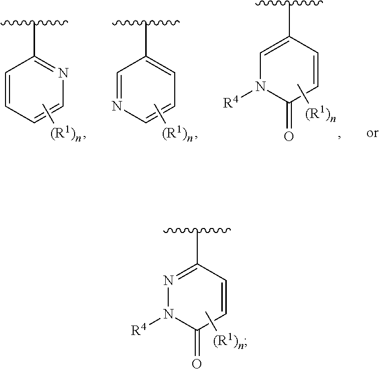

subscript n is an integer from 0, 1, 2, 3, or 4.

In some embodiments, the compound of Formula (I), or a pharmaceutically acceptable salt thereof, is the compound wherein Ring A is

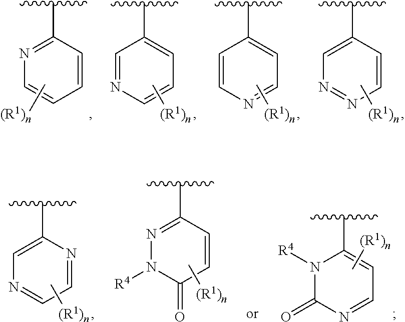

subscript n is an integer from 0, 1, 2, 3, or 4.

In some embodiments, the compound of Formula (I), or a pharmaceutically acceptable salt thereof, is the compound wherein Ring A is

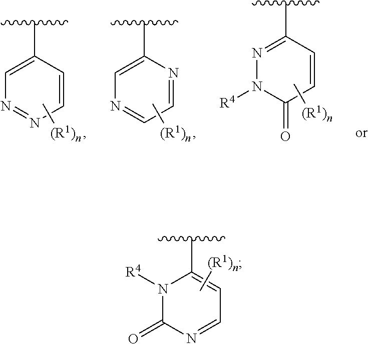

subscript n is an integer from 0, 1, 2, 3, or 4.

In some embodiments, the compound of Formula (I), or a pharmaceutically acceptable salt thereof, is the compound wherein Ring A is

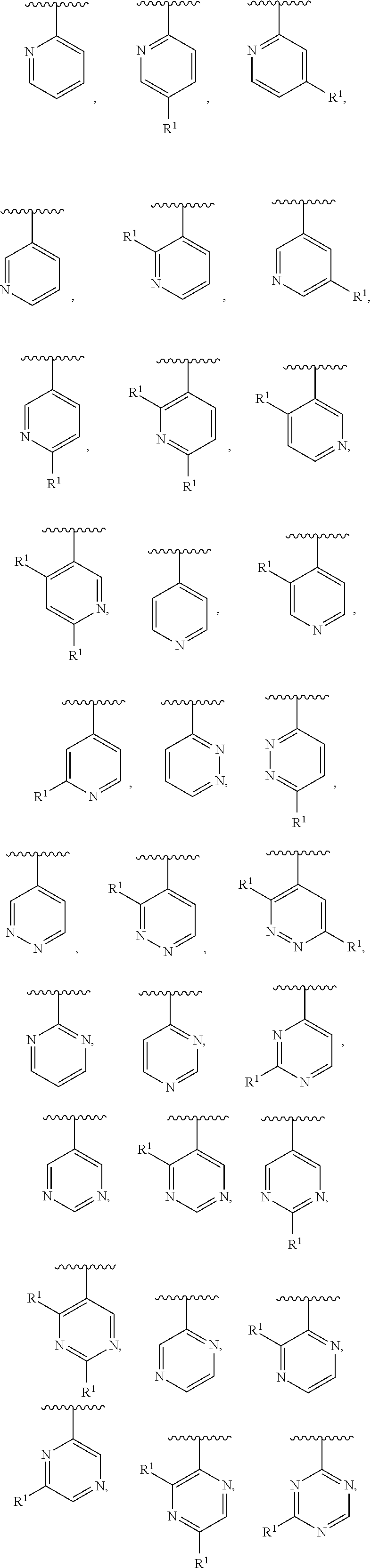

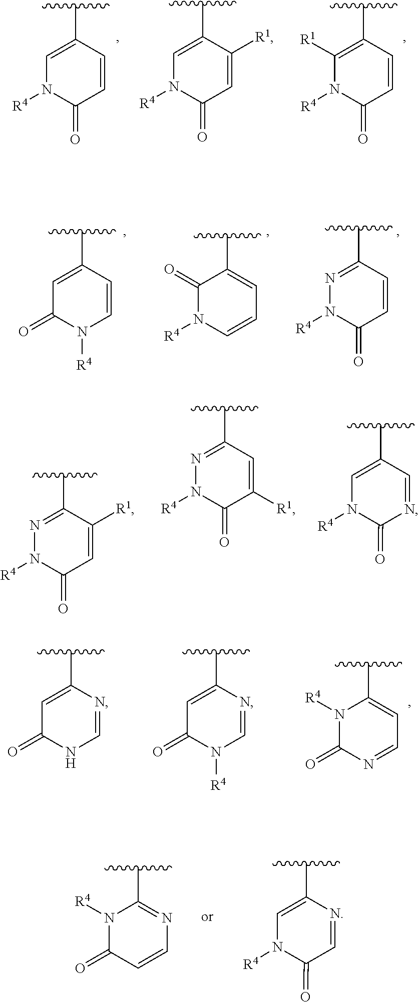

In some embodiments, the compound of Formula (I), or a pharmaceutically acceptable salt thereof, is the compound wherein Ring A is

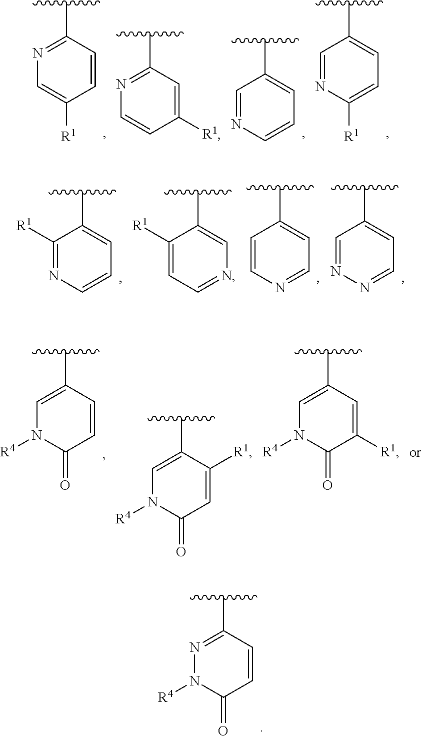

In some embodiments, the compound of Formula (I), or a pharmaceutically acceptable salt thereof, is the compound wherein Ring A is

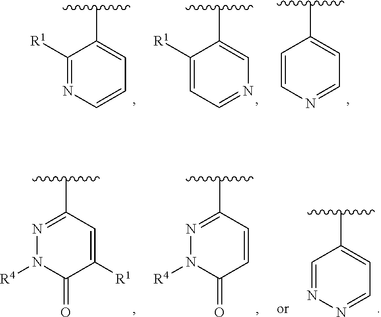

In some embodiments, the compound of Formula (I), or a pharmaceutically acceptable salt thereof, is the compound wherein Ring A is

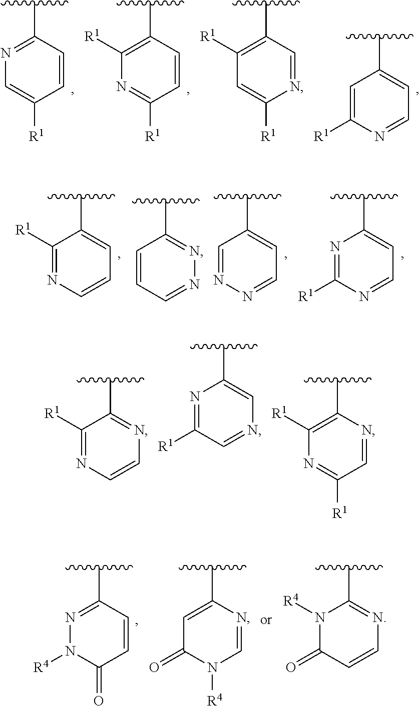

In some embodiments, the compound of Formula (I), or a pharmaceutically acceptable salt thereof, is the compound wherein Ring A is

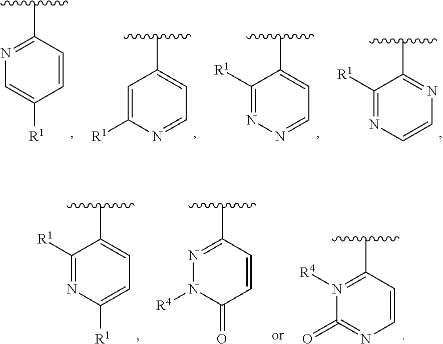

In some embodiments, the compound of Formula (I) or a pharmaceutically acceptable salt thereof, is the compound wherein
each R¹ is independently C₁₋₄ alkyl, C₁₋₃ alkoxy, C₁₋₃ hydroxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl; and
R^{1d} is hydrogen, C₁₋₄ alkyl, halogen, C₁₋₆ haloalkyl or hydroxy.

In some embodiments, the compound of Formula (I) or a pharmaceutically acceptable salt thereof, is the compound wherein
each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O; and
   R^{1d} is hydrogen, C₁₋₄ alkyl, halogen, C₁₋₆ haloalkyl or hydroxy.

In some embodiments, the compound of Formula (I) or a pharmaceutically acceptable salt thereof, is the compound wherein
each R¹ is independently C₁₋₄ alkyl, C₁₋₃ alkoxy, C₁₋₃ hydroxyalkyl, C₁₋₃ haloalkyl, —CN, —N(R^{1a})(R^{1b}), —C₁₋₃ alkyl-N(R^{1a})(R^{1b}), —C(O)N(R^{1a})(R^{1b}), —S(O)₂R^{1a}, cyclopropyl, cyclobutyl, cyclopentyl, piperazine, morpholine, dioxane, pyrazole, or imidazole, wherein the piperazine is substituted with 0 or 1 R^{1d};
each R^{1a} and R^{1b} is independently hydrogen or Me; and
R^{1d} is hydrogen, C₁₋₆ alkyl, halogen, C₁₋₆ haloalkyl or hydroxy.

In some embodiments, the compound of Formula (I) or a pharmaceutically acceptable salt thereof, is the compound wherein
each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, cyclopropyl, cyclobutyl, cyclopentyl, piperazine, morpholine, dioxane, pyrazole, or imidazole, wherein the piperazine is substituted with 0 or 1 R^{1d};
each R^{1a} and R^{1b} is independently hydrogen or Me;
R^{1c} is azetidine or oxetane; and
R^{1d} is hydrogen, C₁₋₆ alkyl, halogen, C₁₋₆ haloalkyl or hydroxyl.

In some embodiments, the compound of Formula (I), or a pharmaceutically acceptable salt thereof, is the compound wherein R¹ is independently Me, —OCH₃, —CH₂OH, —CF₃, —CN, —NH₂, —NHCH₃, —N(CH₃)₂, —CH₂N(CH₃)₂, —C(O)NHCH₃, —C(O)N(CH₃)₂, —S(O)₂CH₃,

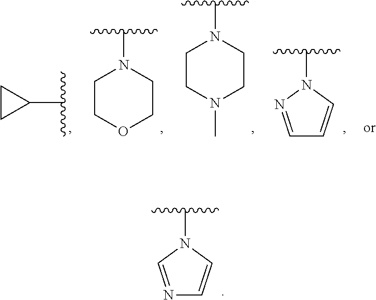

In some embodiments, the compound of Formula (I), or a pharmaceutically acceptable salt thereof, is the compound wherein R¹ is independently Me, Et, iPr, —OCH₃, —CH₂OH, —CH₂CH₂OCH₃, —Cl, —CF₃, —CH₂CHF₂, —CN, —NH₂, —NHCH₃, —N(CH₃)₂, —CH₂N(CH₃)₂, —C(O)NHCH₃, —C(O)N(CH₃)₂,

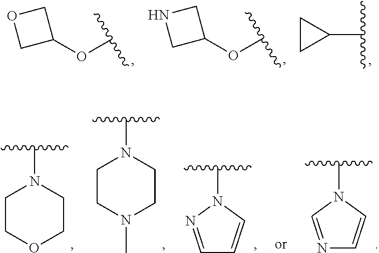

In some embodiments, the compound of Formula (I), or a pharmaceutically acceptable salt thereof, is the compound wherein R⁴ is hydrogenor C₁₋₄ alkyl. In some embodiments, the compound of Formula (I) or a pharmaceutically acceptable salt thereof, is the compound wherein R⁴ hydrogen, Me, Et, or iPr.

In some embodiments, the compound of Formula (I), or a pharmaceutically acceptable salt thereof, is the compound wherein R⁴ is hydrogen, C₁₋₄ alkyl, C₂₋₄ alkoxyalkyl or C₁₋₃ haloalkyl. In some embodiments, the compound of Formula (I) or a pharmaceutically acceptable salt thereof, is the compound wherein R⁴ hydrogen, Me, Et, iPr, —CH₂CH₂OCH₃, or —CH₂CHF₂. In some embodiments, the compound of Formula (I) or a pharmaceutically acceptable salt thereof, is the compound wherein R⁴ is hydrogen or Me.

In some embodiments, the compound of Formula (I), or a pharmaceutically acceptable salt thereof, is the compound wherein
each R¹ is independently C₁₋₄ alkyl, C₁₋₃ alkoxy, C₁₋₃ hydroxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1d} is hydrogen, C₁₋₆ alkyl, halogen, C₁₋₆ haloalkyl or hydroxy; and
R⁴ is hydrogen, or C₁₋₄ alkyl.

In some embodiments, the compound of Formula (I), or a pharmaceutically acceptable salt thereof, is the compound wherein
each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl;
R^{1c} is a 4 membered heterocycloalkyl having 1 heteroatom of N and O;
R^{1d} is hydrogen, C₁₋₆ alkyl, halogen, C₁₋₆ haloalkyl or hydroxy; and
R⁴ is hydrogen, C₁₋₄ alkyl, C₂₋₄ alkoxyalkyl or C₁₋₃ haloalkyl.

In some embodiments, the compound of Formula (I), or a pharmaceutically acceptable salt thereof, is the compound wherein
each R¹ is independently C₁₋₄ alkyl, C₁₋₃ alkoxy, C₁₋₃ hydroxyalkyl, C₁₋₃ haloalkyl, —CN, —N(R^{1a})(R^{1b}), —C₁₋₃ alkyl-N(R^{1a})(R^{1b}), —C(O)N(R^{1a})(R^{1b}), —S(O)₂R^{1a}, cyclopropyl, cyclobutyl, cyclopentyl, piperazine, morpholine, dioxane, pyrazole, or imidazole, wherein the piperazine is substituted with 0 or 1 R^{1d};
each R^{1a} and R^{1b} a is independently hydrogen or Me;
R^{1d} is hydrogen, C₁₋₄ alkyl, halogen, C₁₋₆ haloalkyl or hydroxy; and
R⁴ is hydrogen or C₁₋₄ alkyl.

In some embodiments, the compound of Formula (I), or a pharmaceutically acceptable salt thereof, is the compound wherein
each R¹ is independently C₁₋₄ alkyl, C₁₋₃ alkoxy, C₁₋₃ hydroxyalkyl, C₂₋₄ alkoxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —O—R^{1c}, cyclopropyl, cyclobutyl, cyclopentyl, piperazine, morpholine, dioxane, pyrazole, or imidazole, wherein the piperazine is substituted with 0 or 1 R^{1d};
each R^{1a} and R^{1b} a is independently hydrogen or Me;
R^{1c} is azetidine or oxetane;
R^{1d} is hydrogen, C₁₋₄ alkyl, halogen, C₁₋₆ haloalkyl or hydroxy; and
R⁴ is hydrogen, C₁₋₄ alkyl, C₂₋₄ alkoxyalkyl or C₁₋₃ haloalkyl.

In some embodiments, the compound of Formula (I), or a pharmaceutically acceptable salt thereof, is the compound wherein
R¹ is independently Me, —OCH₃, —CH₂OH, —CF₃, —CN, —NH₂, —NHCH₃, —N(CH₃)₂, —CH₂N(CH₃)₂, —C(O)NHCH₃, —C(O)N(CH₃)₂, —S(O)₂CH₃,

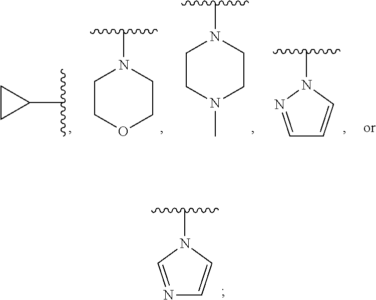

R⁴ is hydrogen or Me.

In some embodiments, the compound of Formula (I), or a pharmaceutically acceptable salt thereof, is the compound wherein
R¹ is independently Me, Et, iPr, —OCH₃, —CH₂OH, —CH₂CH₂OCH₃, —Cl, —CF₃, —CH₂CHF₂, —CN, —NH₂, —NHCH₃, —N(CH₃)₂, —CH₂N(CH₃)₂, —C(O)NHCH₃, —C(O)N(CH₃)₂,

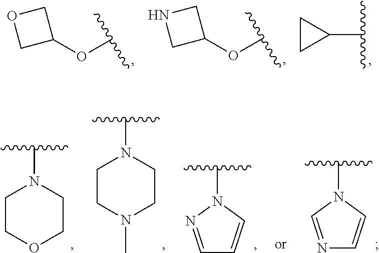

and
R⁴ is hydrogen or Me.

In some embodiments, the compound of Formula (I), or a pharmaceutically acceptable salt thereof, is the compound wherein
Ring A is

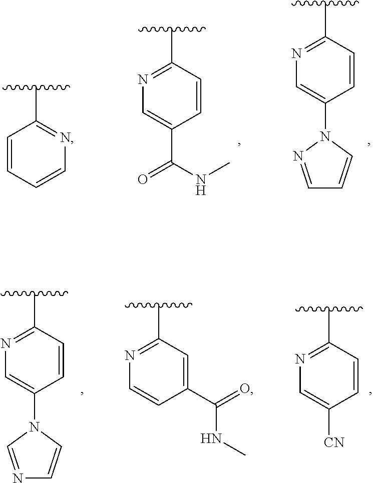 [SMILES: *c1ccccn1 ; *c1ccc(C(=O)NC)cn1 ; *c1ccc(-n2cccn2)cn1 ; *c1ccc(-n2ccnc2)cn1 ; *c1cc(C(=O)NC)ccn1 ; *c1ccc(C#N)cn1]

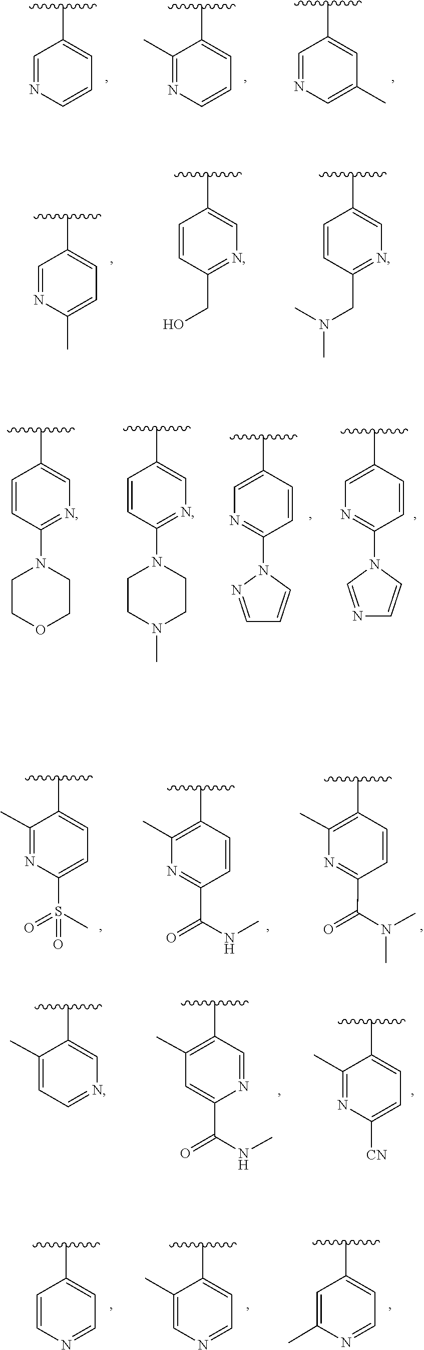

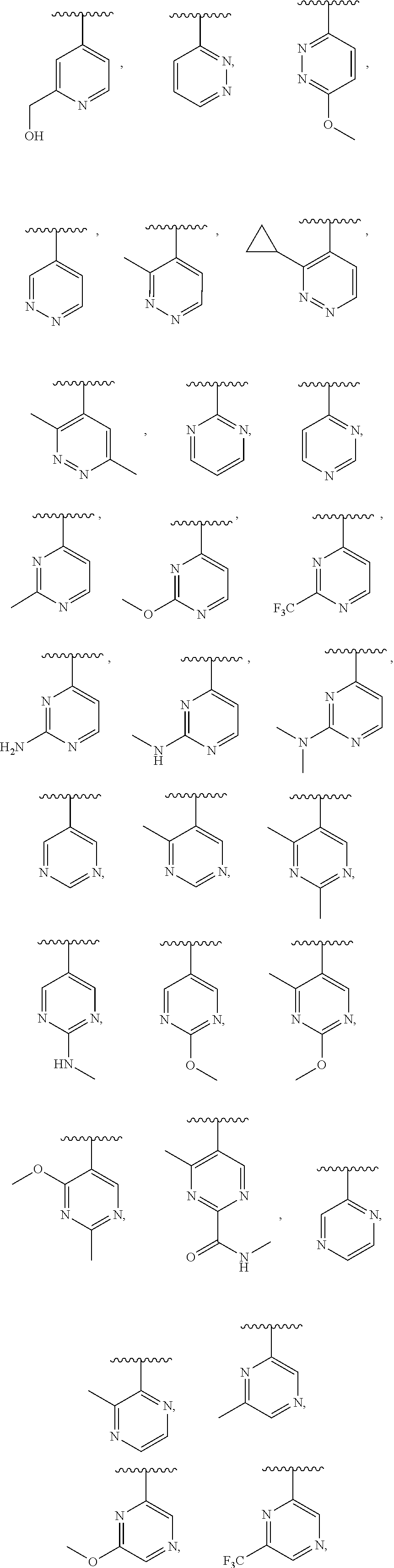

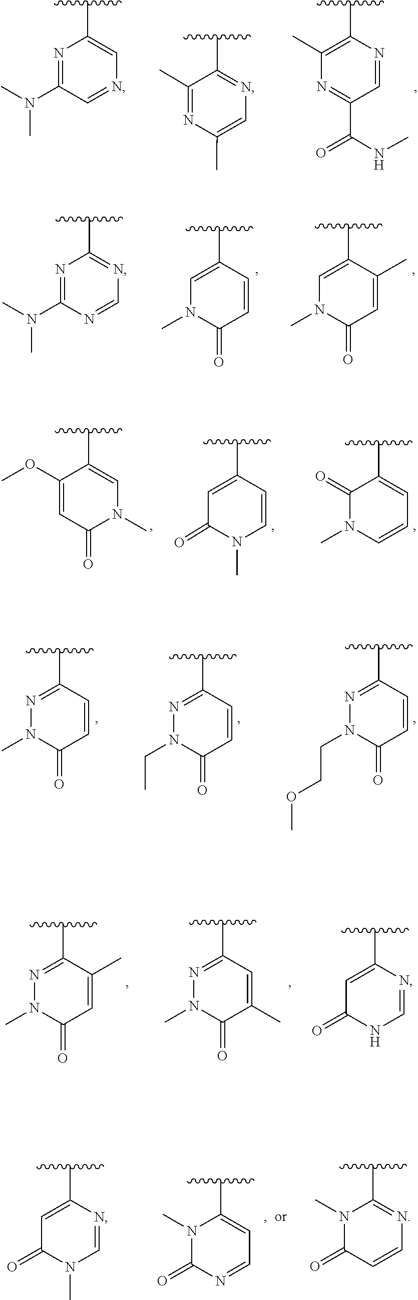

In some embodiments, the compound of Formula (I), or a pharmaceutically acceptable salt thereof, is the compound wherein
Ring A is

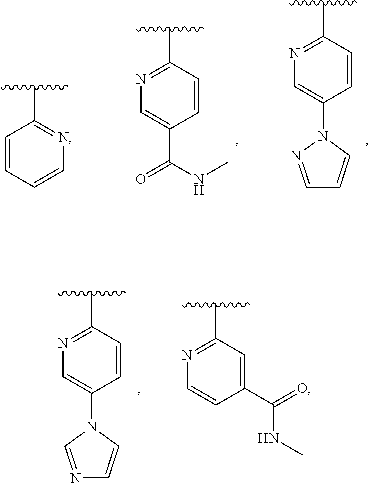 [SMILES: *c1ccccn1 ; *c1ccc(C(=O)NC)cn1 ; *c1ccc(-n2cccn2)cn1 ; *c1ccc(-n2ccnc2)cn1 ; *c1cc(C(=O)NC)ccn1]

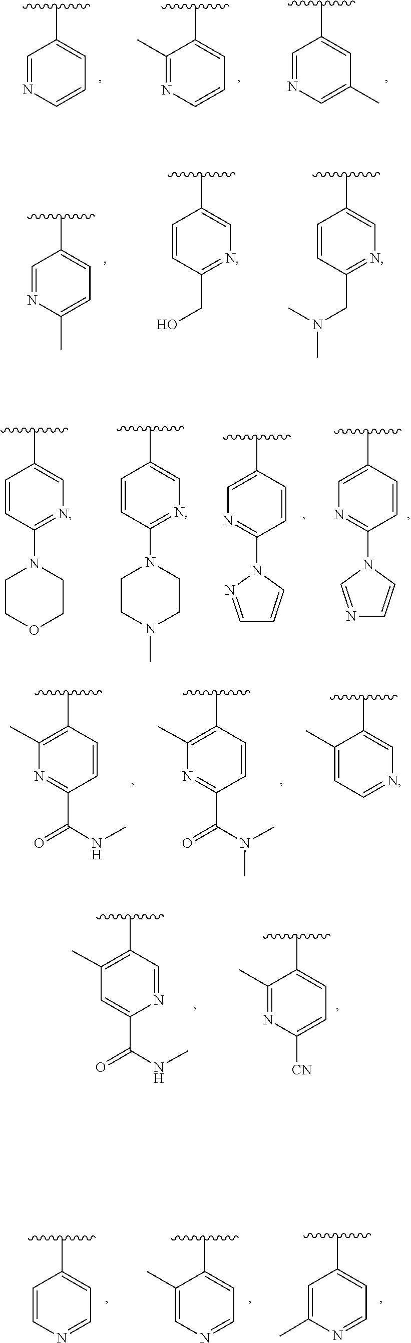

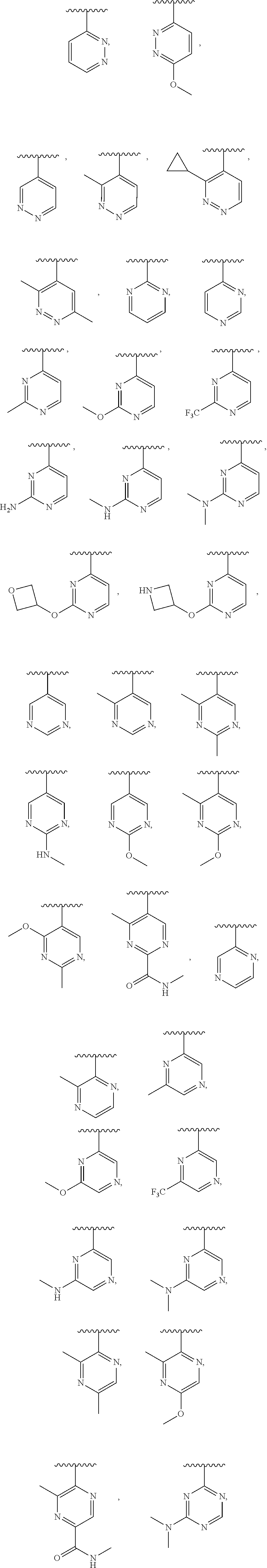

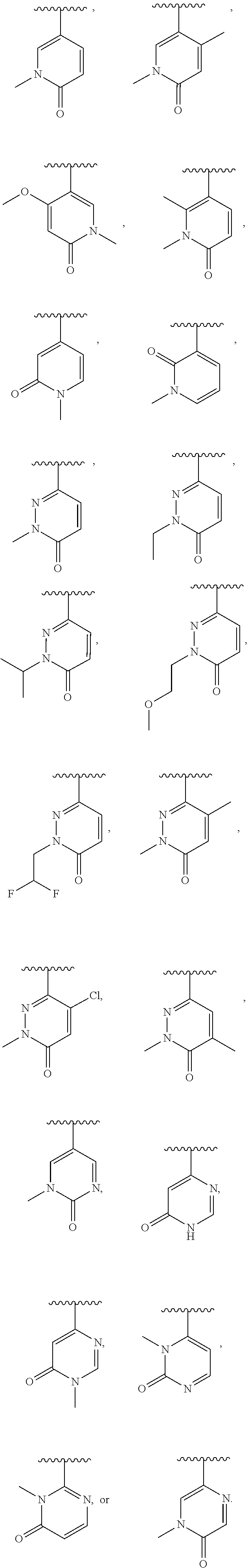

In some embodiments, the compound of Formula (I), or a pharmaceutically acceptable salt thereof, is the compound wherein
R² is independently C₁₋₄ alkyl, C₁₋₄ haloalkyl, C₃₋₆ cycloalkyl, a 4, 5, or 6 membered heterocycloalkyl having 1 heteroatom of N or O, C₁₋₄ alkylaryl, wherein the cycloalkyl, the heterocycloalkyl, and aryl are each independently substituted with 0, 1, 2, or 3 R^{2a} groups; and
each R^{2a} is independently C₁₋₃ alkyl, C₁₋₃ haloalkyl or hydroxyl.

In some embodiments, the compound of Formula (I), or a pharmaceutically acceptable salt thereof, is the compound wherein
R² is independently C₁₋₄ alkyl, C₁₋₄ haloalkyl, C₃₋₆ cycloalkyl or a 4, 5, or 6 membered heterocycloalkyl having 1 heteroatom of N or O, wherein the cycloalkyl and the heterocycloalkyl are each independently substituted with 0, 1, 2, or 3 R^{2a} groups; and
   each R^{2a} is independently C₁₋₃ alkyl, C₁₋₃ haloalkyl or hydroxyl.

In some embodiments, the compound of Formula (I), or a pharmaceutically acceptable salt thereof, is the compound wherein
R² is independently C₁₋₄ alkyl, or C₃₋₆ cycloalkyl, wherein the cycloalkyl is substituted with 0 or 1 R^{2a} group; and
R^{2a} is C₁₋₃ alkyl.

In some embodiments, the compound of Formula (I), or a pharmaceutically acceptable salt thereof, is the compound wherein R² is independently C₁₋₄ alkyl, C₁₋₄ haloalkyl, cyclopropyl, cyclobutyl, bicyclo[1.1.1]pentyl, azetidine, pyrrolidine, piperidine, oxetane, tetrahydrofuran, tetrahydropyran, or phenylethyl, wherein the cyclopropyl and cyclobutyl are each independently substituted with 0 or 1 R^{2a} group, wherein each R^{2a} is Me, CF₃ or —OH, and wherein the azetidine, pyrrolidine, piperidine, oxetane, tetrahydrofuran, or tetrahydropyran are each independently substituted with 0 or 1 Me group.

In some embodiments, the compound of Formula (I), or a pharmaceutically acceptable salt thereof, is the compound wherein R² is independently C₁₋₄ alkyl, C₁₋₄ haloalkyl, cyclopropyl, cyclobutyl, bicyclo[1.1.1]pentyl, azetidine, pyrrolidine, piperidine, oxetane, tetrahydrofuran, or tetrahydropyran, wherein the cyclopropyl and cyclobutyl are each independently substituted with 0 or 1 R^{2a} group, wherein each R^{2a} is Me, CF₃ or —OH, and wherein the azetidine, pyrrolidine, piperidine, oxetane, tetrahydrofuran, or tetrahydropyran are each independently substituted with 0 or 1 Me group.

In some embodiments, the compound of Formula (I) or a pharmaceutically acceptable salt thereof, is the compound wherein R² is independently C₃₋₄ alkyl, cyclopropyl, cyclobutyl, or bicyclo[1.1.1]pentyl, wherein the cyclopropyl and cyclobutyl are each independently substituted with 0 or 1 Me group.

In some embodiments, the compound of Formula (I) or a pharmaceutically acceptable salt thereof, is the compound wherein
R² is independently iPr, s-Bu, t-Bu, —CH(CH₃)CF₃, —CH(CH₃)CH₂CF₃, —C(CH₃)₂CF₂,

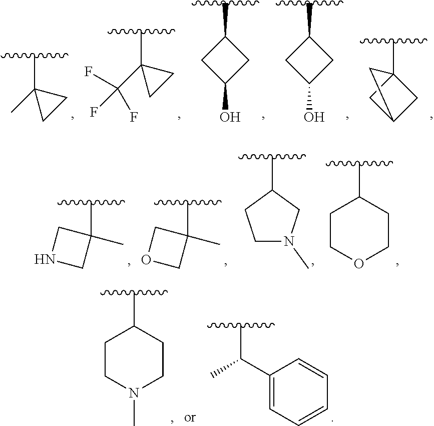

In some embodiments, the compound of Formula (I) or a pharmaceutically acceptable salt thereof, is the compound wherein
R² is independently iPr, s-Bu, t-Bu, —CH(CH₃)CF₃,

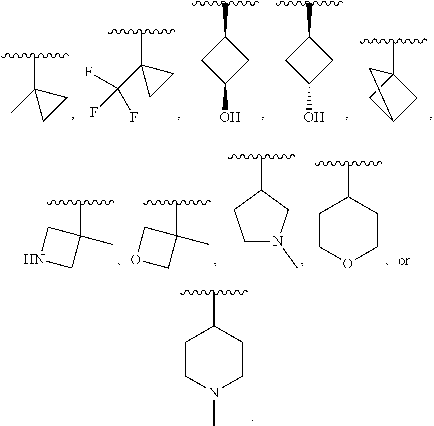

In some embodiments, the compound of Formula (I) or a pharmaceutically acceptable salt thereof, is the compound wherein R² is independently t-Bu,

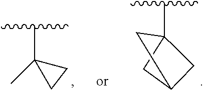

In some embodiments, the compound of Formula (I), or a pharmaceutically acceptable salt thereof, is the compound wherein R³ is hydrogen or C₁₋₃ alkyl. In some embodiments, the compound of Formula (I) or a pharmaceutically acceptable salt thereof, is the compound wherein R³ is hydrogen or Me. In some embodiments, the compound of Formula (I) or a pharmaceutically acceptable salt thereof, is the compound wherein R³ is hydrogen.

In some embodiments, the compound of Formula (I), or a pharmaceutically acceptable salt thereof is the compound wherein Ring A is pyridine, pyridazine, pyrimidine, pyrazine, triazine, pyridine-2-one, pyridazine-3-one, pyrimidine-2-one, or pyrazine-2-one; R² is independently C₁₋₄ alkyl, C₁₋₄ haloalkyl, C₃₋₆ cycloalkyl, a 4, 5, or 6 membered heterocycloalkyl having 1 heteroatom of N or O, or C₁₋₄ alkylaryl, wherein the cycloalkyl, the heterocycloalkyl, and the aryl are each independently substituted with 0, 1, 2, or 3 R^{2a} groups; and each R^{2a} is independently C₁₋₃ alkyl, C₁₋₃ haloalkyl or hydroxyl.

In some embodiments, the compound of Formula (I), or a pharmaceutically acceptable salt thereof is the compound wherein Ring A is pyridine, pyridazine, pyrimidine, pyrazine, triazine, pyridine-2-one, pyridazine-3-one, pyrimidine-2-one, or pyrazine-2-one; R² is independently C₁₋₄ alkyl, C₁₋₄ haloalkyl, C₃₋₆ cycloalkyl or a 4, 5, or 6 membered heterocycloalkyl having 1 heteroatom of N or O, wherein the cycloalkyl and the heterocycloalkyl are each independently substituted with 0, 1, 2, or 3 R^{2a} groups; and each R^{2a} is independently C₁₋₃ alkyl, C₁₋₃ haloalkyl or hydroxyl.

In some embodiments, the compound of Formula (I), or a pharmaceutically acceptable salt thereof is the compound wherein Ring A is pyridine, pyridazine, pyrimidine, pyrazine, triazine, pyridine-2-one, pyridazine-3-one, pyrimidine-2-one, or pyrazine-2-one; R² is independently C₁₋₄ alkyl, or C₃₋₆ cycloalkyl, wherein the cycloalkyl is substituted with 0 or 1 R^{2a} groups; and R^{2a} is C₁₋₃ alkyl.

In some embodiments, the compound of Formula (I), or a pharmaceutically acceptable salt thereof is the compound wherein Ring A is pyridine, pyridazine, pyrimidine, pyrazine, triazine, pyridine-2-one, pyridazine-3-one, pyrimidine-2-one, or pyrazine-2-one and R³ is hydrogen.

In some embodiments, the compound of Formula (I), or a pharmaceutically acceptable salt thereof is the compound wherein Ring A is

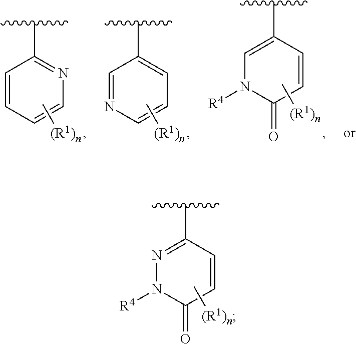

subscript n is an integer from 0, 1, 2, 3, or 4; and R³ is hydrogen.

In some embodiments, the compound of Formula (I), or a pharmaceutically acceptable salt thereof, is the compound wherein Ring A is:

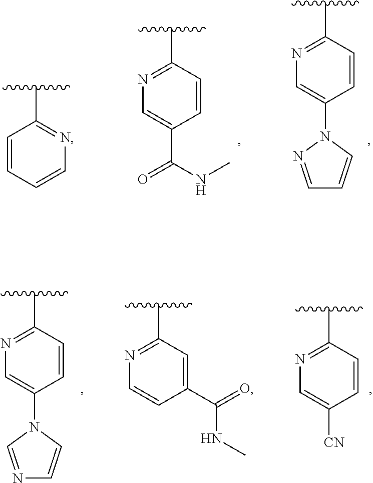 [SMILES: *c1ccccn1 ; *c1ccc(C(=O)NC)cn1 ; *c1ccc(-n2cccn2)cn1 ; *c1ccc(-n2ccnc2)cn1 ; *c1cc(C(=O)NC)ccn1 ; *c1ccc(C#N)cn1]

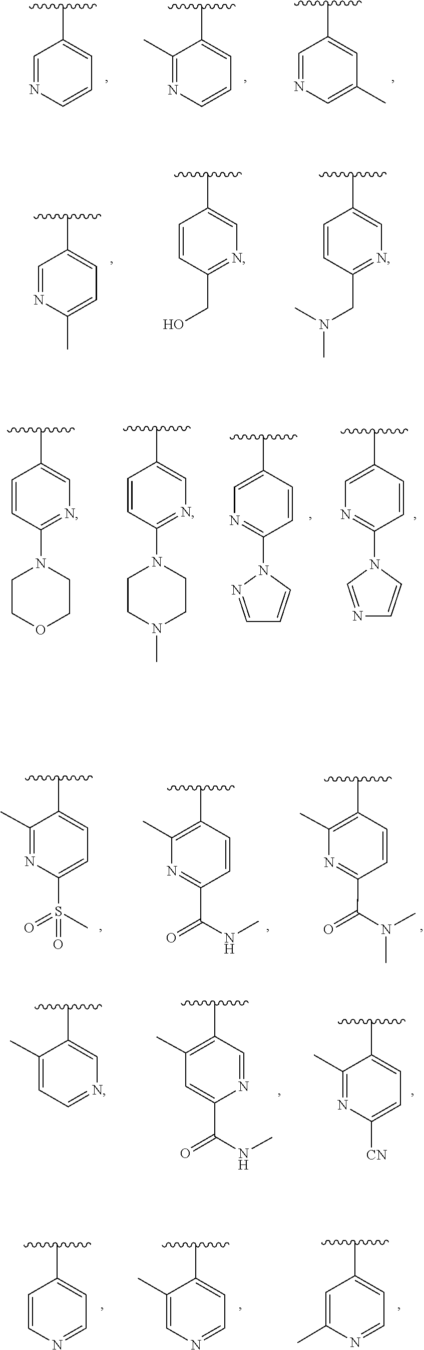

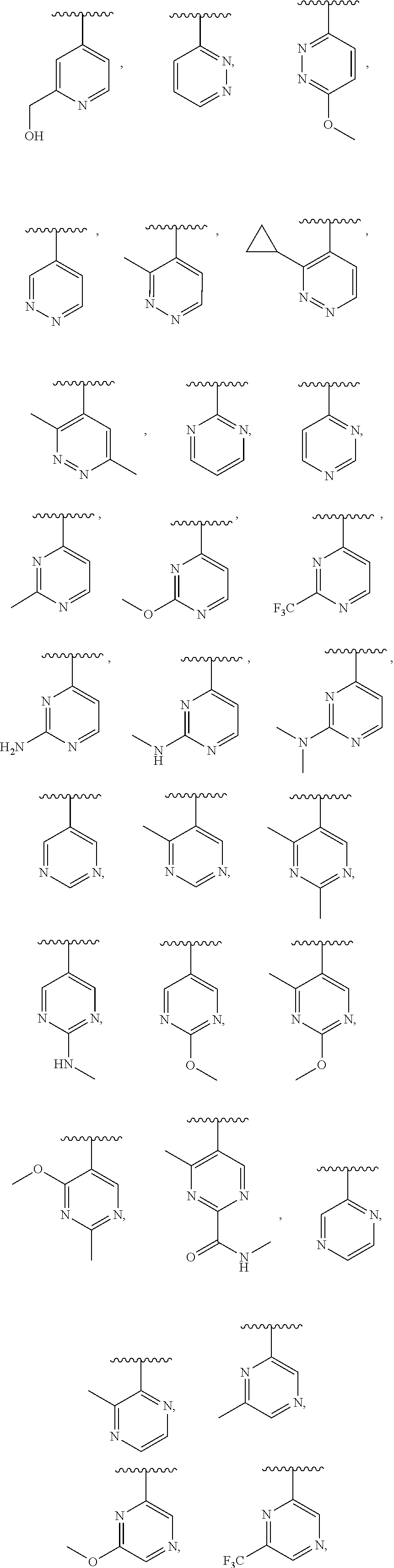

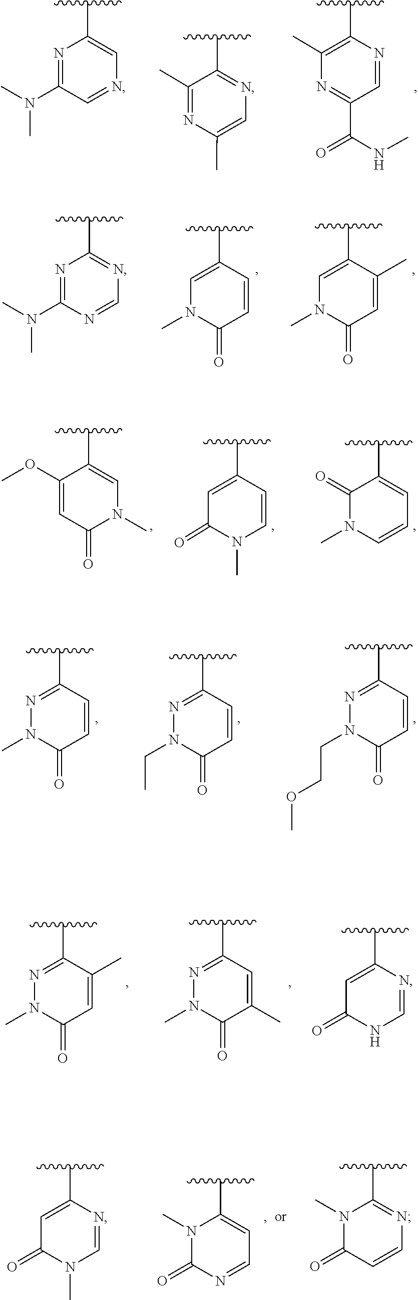

and R³ is hydrogen.

In some embodiments, the compound of Formula (I), or a pharmaceutically acceptable salt thereof, is the compound wherein Ring A is:

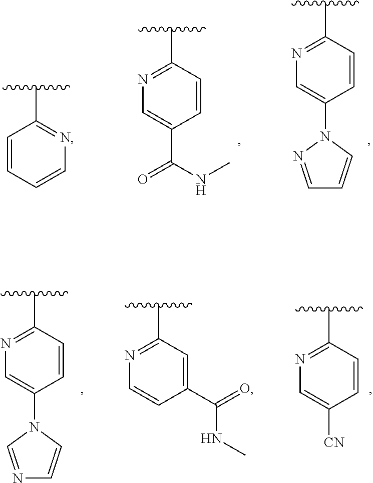 [SMILES: *c1ccccn1 ; *c1ccc(C(=O)NC)cn1 ; *c1ccc(-n2cccn2)cn1 ; *c1ccc(-n2ccnc2)cn1 ; *c1cc(C(=O)NC)ccn1 ; *c1ccc(C#N)cn1]

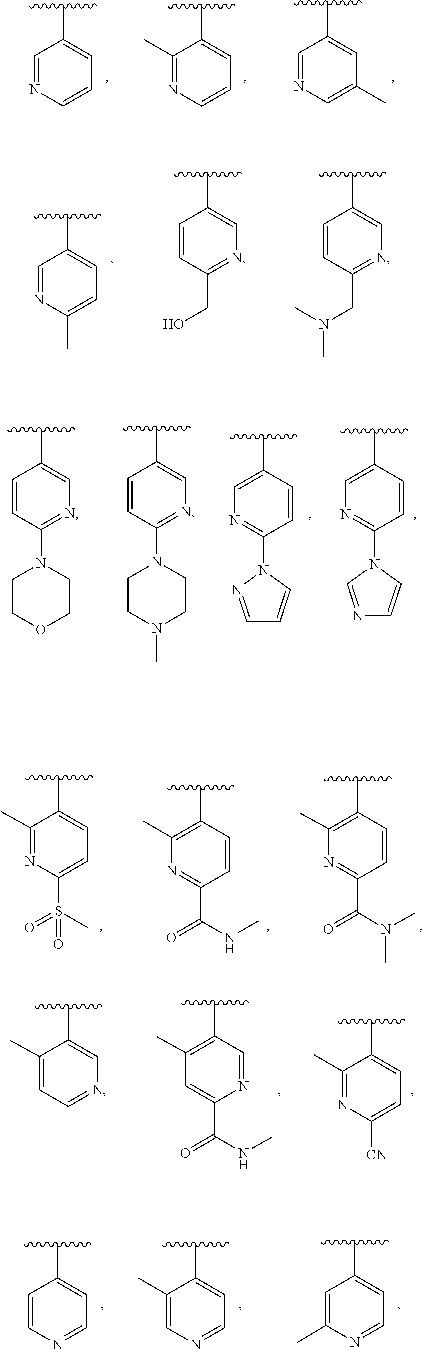

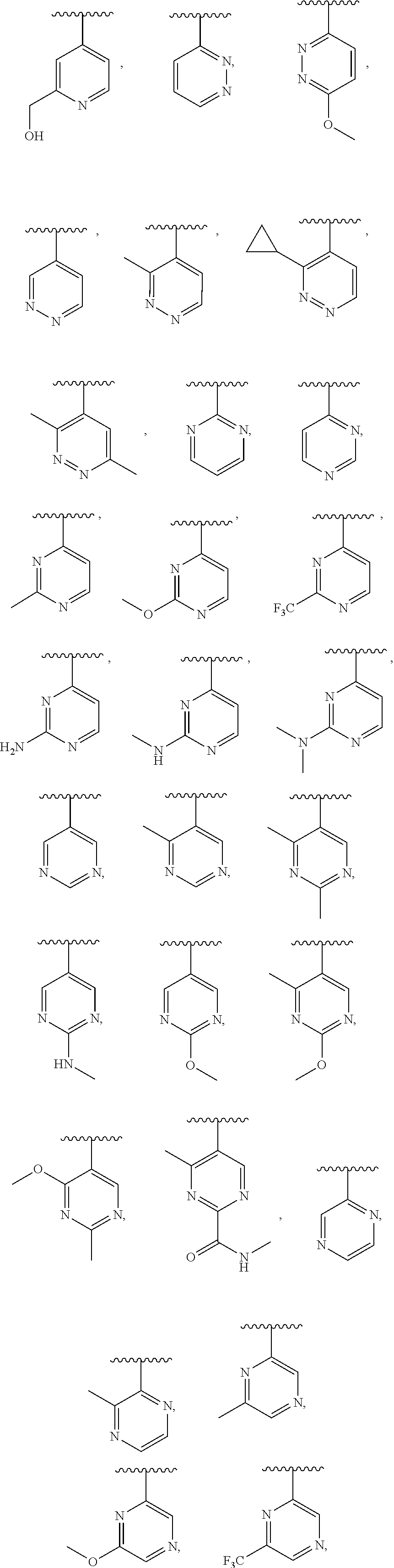

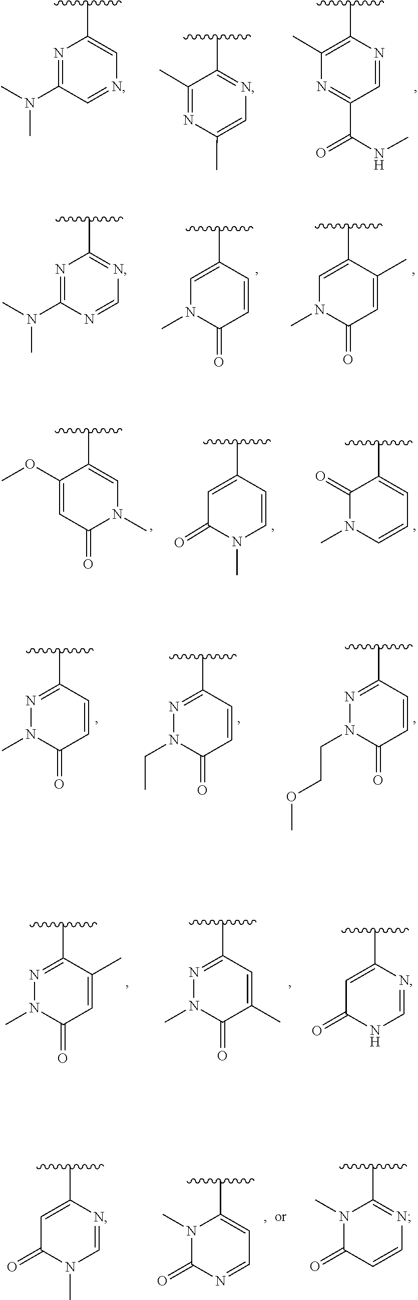

R² is independently iPr, s-Bu, t-Bu, —CH(CH₃)CF₃,

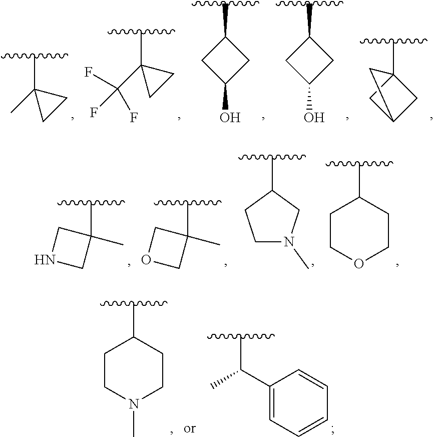

R³ is hydrogen.

In some embodiments, the compound of Formula (I), or a pharmaceutically acceptable salt thereof, is the compound wherein Ring A is:

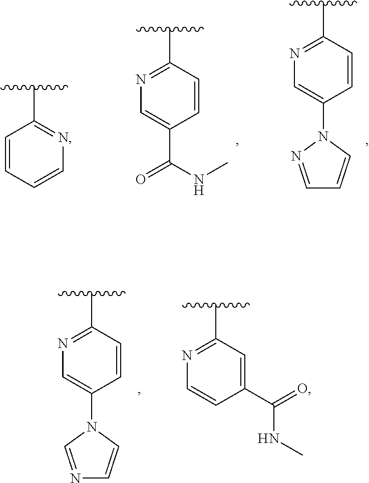 [SMILES: *c1ccccn1 ; *c1ccc(C(=O)NC)cn1 ; *c1ccc(-n2cccn2)cn1 ; *c1ccc(-n2ccnc2)cn1 ; *c1cc(C(=O)NC)ccn1]

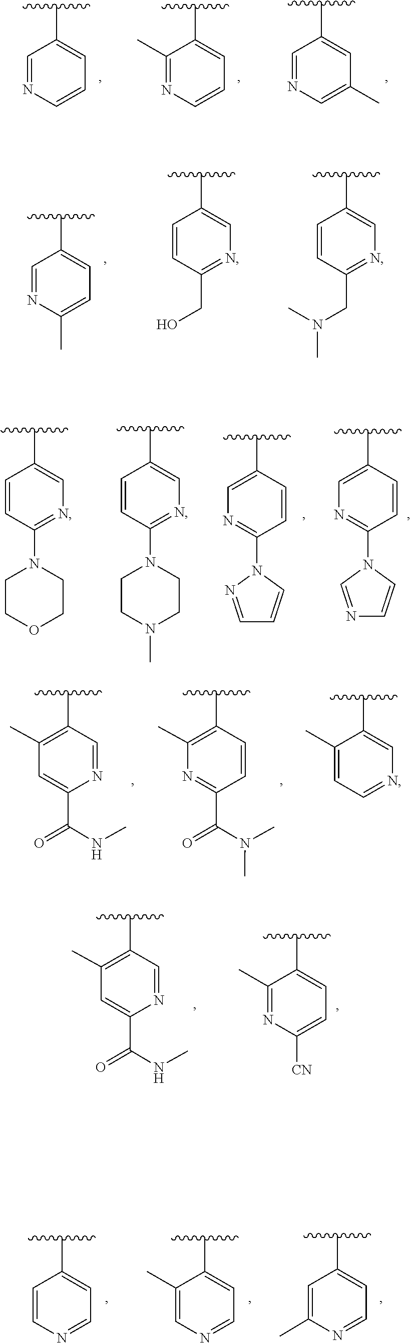

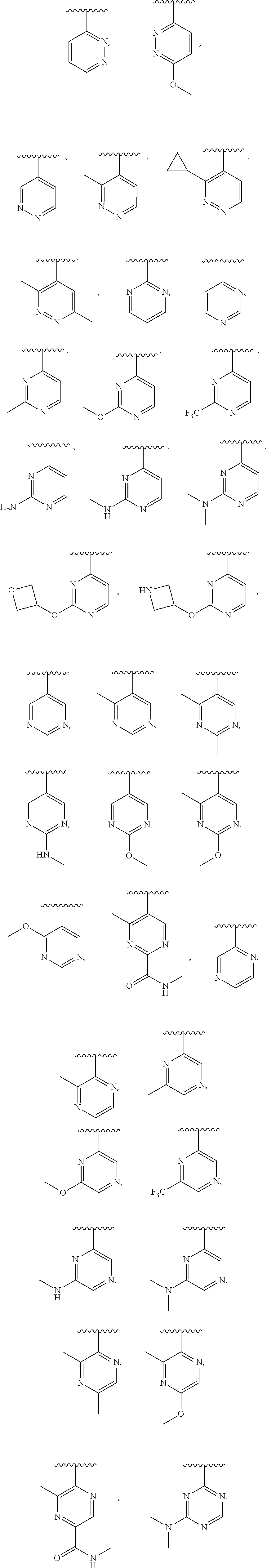

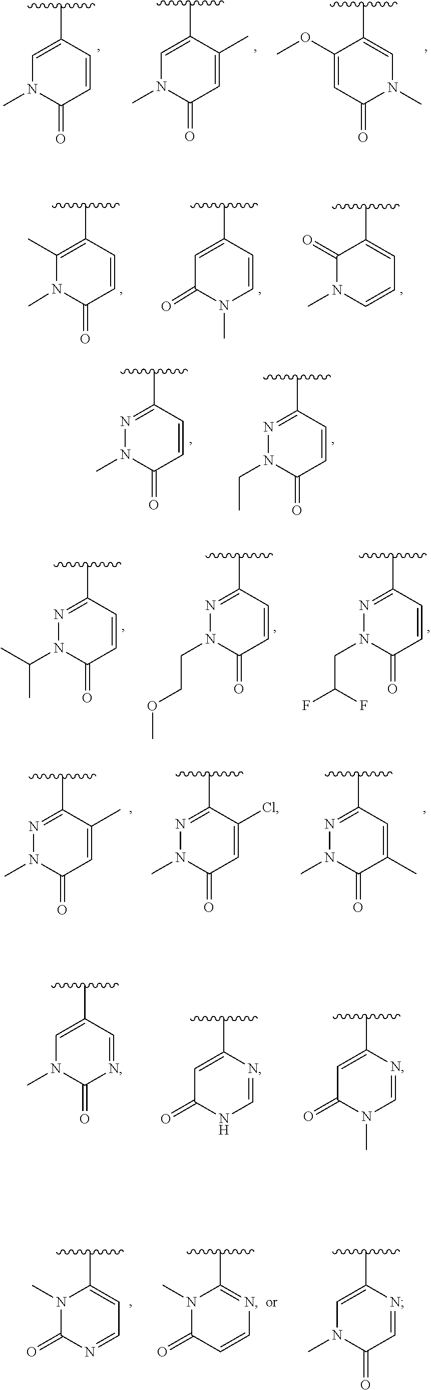

R² is independently iPr, s-Bu, t-Bu, —CH(CH₃)CF₃,

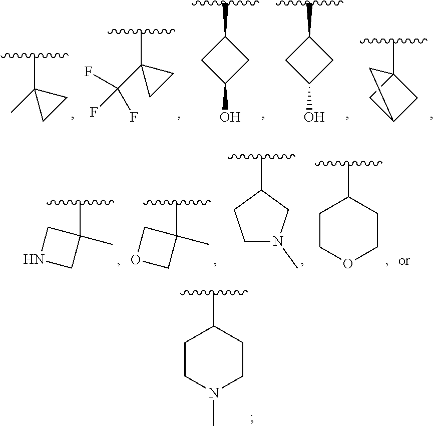

and
   R³ is hydrogen.

In some embodiments, the compound of Formula (I) or a pharmaceutically acceptable salt thereof, is any one compound provided in Table 1 or Table 2:

TABLE 1
Exemplary Compounds of Formula (I)

| | |
|---|---|
| 1 | 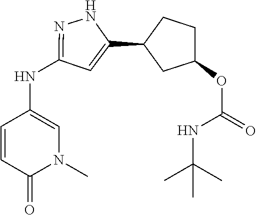 [SMILES: Cn1cc(Nc2cc([C@H]3CC[C@@H](OC(=O)NC(C)(C)C)C3)[nH]n2)ccc1=O] |
| | (1R,3S)-3-(3-((1-methyl-6-oxo-1,6- |
| | dihydropyridin-3-yl)amino)-1H- |
| | pyrazol-5-yl)cyclopentyl tert- |
| | butylcarbamate |
| 2 | 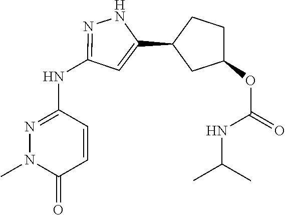 [SMILES: CC(C)NC(=O)O[C@@H]1CC[C@H](c2cc(Nc3ccc(=O)n(C)n3)n[nH]2)C1] |
| | (1R,3S)-3-(3-((1-methyl-6-oxo-1,6- |
| | dihydropyridazin-3-yl)amino)-1H- |
| | pyrazol-5-yl)cyclopentyl |
| | isopropylcarbamate |
| 2A | 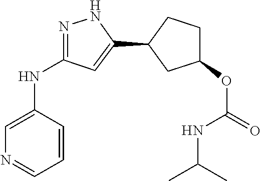 [SMILES: CC(C)NC(=O)O[C@@H]1CC[C@H](c2cc(Nc3cccnc3)n[nH]2)C1] |
| | (1R,3S)-3-(3-(pyridin-3-ylamino)-1H- |
| | pyrazol-5-yl)cyclopentyl |
| | isopropylcarbamate |
| 2B | 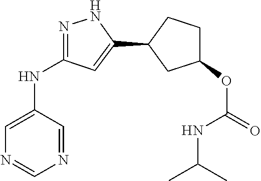 [SMILES: CC(C)NC(=O)O[C@@H]1CC[C@H](c2cc(Nc3cncnc3)n[nH]2)C1] |
| | (1R,3S)-3-(3-(pyrimidin-5-ylamino)- |
| | 1H-pyrazol-5-yl)cyclopentyl |
| | isopropylcarbamate |
| 2C | 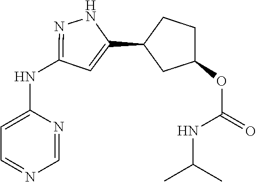 [SMILES: CC(C)NC(=O)O[C@@H]1CC[C@H](c2cc(Nc3ccncn3)n[nH]2)C1] |
| | (1R,3S)-3-(3-(pyrimidin-4-ylamino)- |
| | 1H-pyrazol-5-yl)cyclopentyl |
| | isopropylcarbamate |
| 2D | 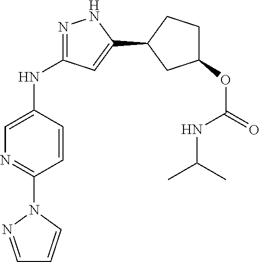 [SMILES: CC(C)NC(=O)O[C@@H]1CC[C@H](c2cc(Nc3ccc(-n4cccn4)nc3)n[nH]2)C1] |
| | (1R,3S)-3-(3-((6-(1H-pyrazol-1- |
| | yl)pyridin-3-yl)amino)-1H-pyrazol-5- |
| | yl)cyclopentyl isopropylcarbamate |
| 2E | 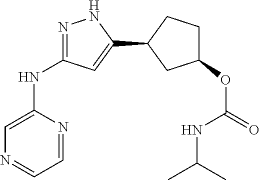 [SMILES: CC(C)NC(=O)O[C@@H]1CC[C@H](c2cc(Nc3cnccn3)n[nH]2)C1] |
| | (1R,3S)-3-(3-(pyrazin-2-ylamino)-1H- |
| | pyrazol-5-yl)cyclopentyl |
| | isopropylcarbamate |
| 2F | 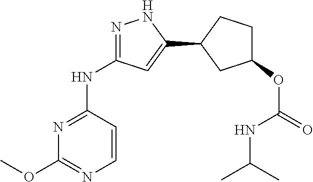 [SMILES: COc1nccc(Nc2cc([C@H]3CC[C@@H](OC(=O)NC(C)C)C3)[nH]n2)n1] |
| | (1R,3S)-3-(3-((2-methoxypyrimidin-4- |
| | yl)amino)-1H-pyrazol-5-yl)cyclopentyl |
| | isopropylcarbamate |
| 2G | 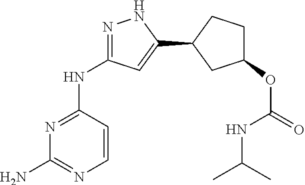 [SMILES: CC(C)NC(=O)O[C@@H]1CC[C@H](c2cc(Nc3ccnc(N)n3)n[nH]2)C1] |
| | (1R,3S)-3-(3-((2-aminopyrimidin-4- |
| | yl)amino)-1H-pyrazol-5-yl)cyclopentyl |
| | isopropylcarbamate |
| 2H | 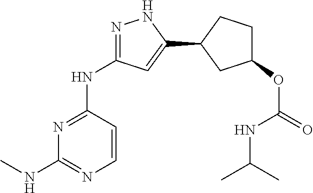 [SMILES: CNc1nccc(Nc2cc([C@H]3CC[C@@H](OC(=O)NC(C)C)C3)[nH]n2)n1] |
| | (1R,3S)-3-(3-((2- |
| | (methylamino)pyrimidin-4-yl)amino)- |
| | 1H-pyrazol-5-yl)cyclopentyl |
| | isopropylcarbamate |
| 2I | 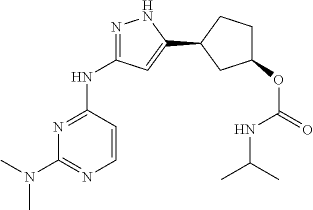 [SMILES: CC(C)NC(=O)O[C@@H]1CC[C@H](c2cc(Nc3ccnc(N(C)C)n3)n[nH]2)C1] |
| | (1R,3S)-3-(3-((2- |
| | (dimethylamino)pyrimidin-4- |
| | yl)amino)-1H-pyrazol-5-yl)cyclopentyl |
| | isopropylcarbamate |
| 2J | 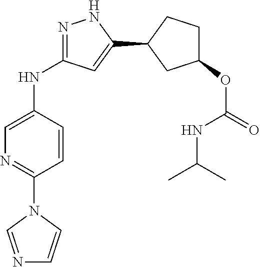 [SMILES: CC(C)NC(=O)O[C@@H]1CC[C@H](c2cc(Nc3ccc(-n4ccnc4)nc3)n[nH]2)C1] |
| | (1R,3S)-3-(3-((6-(1H-imidazol-1- |
| | yl)pyridin-3-yl)amino)-1H-pyrazol-5- |
| | yl)cyclopentyl isopropylcarbamate |
| 2K | 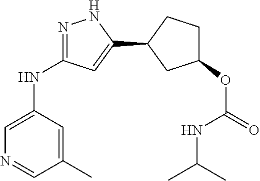 [SMILES: Cc1cncc(Nc2cc([C@H]3CC[C@@H](OC(=O)NC(C)C)C3)[nH]n2)c1] |
| | (1R,3S)-3-(3-((5-methylpyridin-3- |
| | yl)amino)-1H-pyrazol-5-yl)cyclopentyl |
| | isopropylcarbamate |
| 2L | 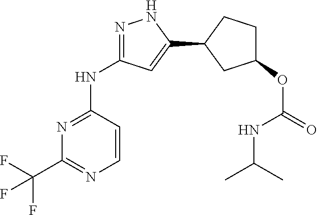 [SMILES: CC(C)NC(=O)O[C@@H]1CC[C@H](c2cc(Nc3ccnc(C(F)(F)F)n3)n[nH]2)C1] |
| | (1R,3S)-3-(3-((2- |
| | (trifluoromethyl)pyrimidin-4- |
| | yl)amino)-1H-pyrazol-5-yl)cyclopentyl |
| | isopropylcarbamate |
| 2M | 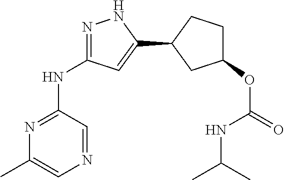 [SMILES: Cc1cncc(Nc2cc([C@H]3CC[C@@H](OC(=O)NC(C)C)C3)[nH]n2)n1] |
| | (1R,3S)-3-(3-((6-methylpyrazin-2- |
| | yl)amino)-1H-pyrazol-5-yl)cyclopentyl |
| | isopropylcarbamate |
| 2N | 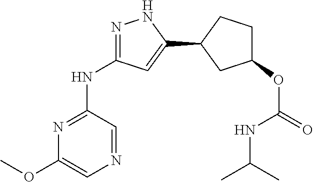 [SMILES: COc1cncc(Nc2cc([C@H]3CC[C@@H](OC(=O)NC(C)C)C3)[nH]n2)n1] |
| | (1R,3S)-3-(3-((6-methoxypyrazin-2- |
| | yl)amino)-1H-pyrazol-5-yl)cyclopentyl |
| | isopropylcarbamate |
| 2O | 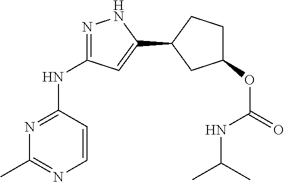 [SMILES: Cc1nccc(Nc2cc([C@H]3CC[C@@H](OC(=O)NC(C)C)C3)[nH]n2)n1] |
| | (1R,3S)-3-(3-((2-methylpyrimidin-4- |
| | yl)amino)-1H-pyrazol-5-yl)cyclopentyl |
| | isopropylcarbamate |
| 2P | 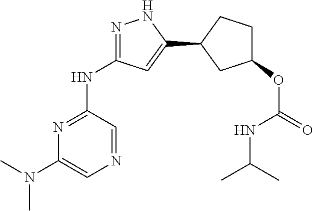 [SMILES: CC(C)NC(=O)O[C@@H]1CC[C@H](c2cc(Nc3cncc(N(C)C)n3)n[nH]2)C1] |
| | (1R,3S)-3-(3-((6- |
| | (dimethylamino)pyrazin-2-yl)amino)- |
| | 1H-pyrazol-5-yl)cyclopentyl |
| | isopropylcarbamate |
| 2Q | 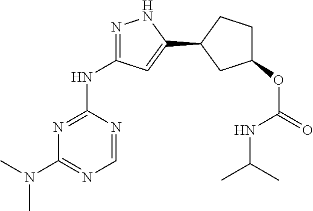 [SMILES: CC(C)NC(=O)O[C@@H]1CC[C@H](c2cc(Nc3ncnc(N(C)C)n3)n[nH]2)C1] |
| | (1R,3S)-3-(3-((4-(dimethylamino)- |
| | 1,3,5-triazin-2-yl)amino)-1H-pyrazol- |
| | 5-yl)cyclopentyl isopropylcarbamate |
| 2R | 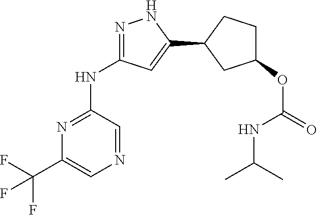 [SMILES: CC(C)NC(=O)O[C@@H]1CC[C@H](c2cc(Nc3cncc(C(F)(F)F)n3)n[nH]2)C1] |
| | (1R,3S)-3-(3-((6- |
| | (trifluoromethyl)pyrazin-2-yl)amino)- |
| | 1H-pyrazol-5-yl)cyclopentyl |
| | isopropylcarbamate |
| 2S | 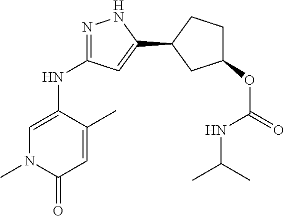 [SMILES: Cc1cc(=O)n(C)cc1Nc1cc([C@H]2CC[C@@H](OC(=O)NC(C)C)C2)[nH]n1] |
| | (1R,3S)-3-(3-((1,4-dimethyl-6-oxo-1,6- |
| | dihydropyridin-3-yl)amino)-1H- |
| | pyrazol-5-yl)cyclopentyl |
| | isopropylcarbamate |
| 2T | 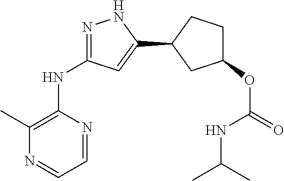 [SMILES: Cc1nccnc1Nc1cc([C@H]2CC[C@@H](OC(=O)NC(C)C)C2)[nH]n1] |
| | (1R,3S)-3-(3-((3-methylpyrazin-2- |
| | yl)amino)-1H-pyrazol-5-yl)cyclopentyl |
| | isopropylcarbamate |
| 2U | 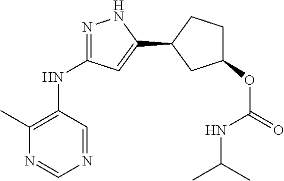 [SMILES: Cc1ncncc1Nc1cc([C@H]2CC[C@@H](OC(=O)NC(C)C)C2)[nH]n1] |
| | (1R,3S)-3-(3-((4-methylpyrimidin-5- |
| | yl)amino)-1H-pyrazol-5-yl)cyclopentyl |
| | isopropylcarbamate |
| 2V | 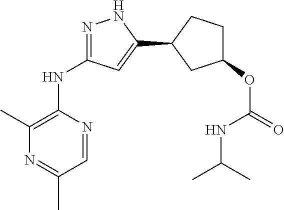 [SMILES: Cc1cnc(Nc2cc([C@H]3CC[C@@H](OC(=O)NC(C)C)C3)[nH]n2)c(C)n1] |
| | (1R,3S)-3-(3-((3,5-dimethylpyrazin-2- |
| | yl)amino)-1H-pyrazol-5-yl)cyclopentyl |
| | isopropylcarbamate |
| 2W | 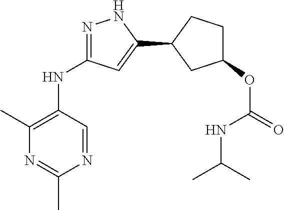 [SMILES: Cc1ncc(Nc2cc([C@H]3CC[C@@H](OC(=O)NC(C)C)C3)[nH]n2)c(C)n1] |
| | (1R,3S)-3-(3-((2,4-dimethylpyrimidin- |
| | 5-yl)amino)-1H-pyrazol-5- |
| | yl)cyclopentyl isopropylcarbamate |
| 2X | 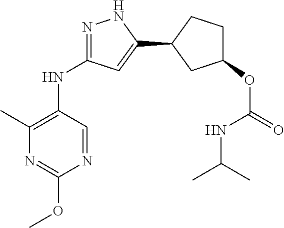 [SMILES: COc1ncc(Nc2cc([C@H]3CC[C@@H](OC(=O)NC(C)C)C3)[nH]n2)c(C)n1] |
| | (1R,3S)-3-(3-((2-methoxy-4- |
| | methylpyrimidin-5-yl)amino)-1H- |
| | pyrazol-5-yl)cyclopentyl |
| | isopropylcarbamate |
| 2Y | 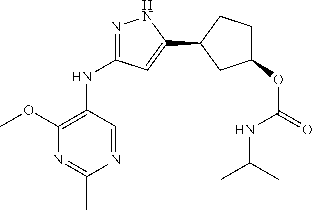 [SMILES: COc1nc(C)ncc1Nc1cc([C@H]2CC[C@@H](OC(=O)NC(C)C)C2)[nH]n1] |
| | (1R,3S)-3-(3-((4-methoxy-2- |
| | methylpyrimidin-5-yl)amino)-1H- |
| | pyrazol-5-yl)cyclopentyl |
| | isopropylcarbamate |
| 2Z | 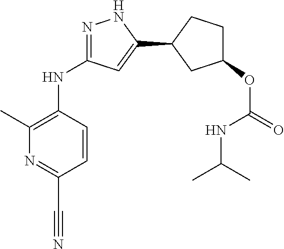 [SMILES: Cc1nc(C#N)ccc1Nc1cc([C@H]2CC[C@@H](OC(=O)NC(C)C)C2)[nH]n1] |
| | (1R,3S)-3-(3-((6-cyano-2- |
| | methylpyridin-3-yl)amino)-1H- |
| | pyrazol-5-yl)cyclopentyl |
| | isopropylcarbamate |
| 2AA | 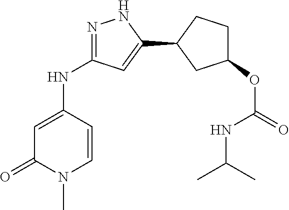 [SMILES: CC(C)NC(=O)O[C@@H]1CC[C@H](c2cc(Nc3ccn(C)c(=O)c3)n[nH]2)C1] |
| | (1R,3S)-3-(3-((1-methyl-2-oxo-1,2- |
| | dihydropyridin-4-yl)amino)-1H- |
| | pyrazol-5-yl)cyclopentyl |
| | isopropylcarbamate |
| 2BB | 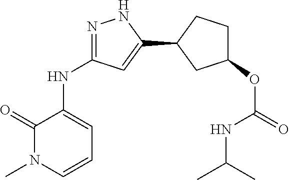 [SMILES: CC(C)NC(=O)O[C@@H]1CC[C@H](c2cc(Nc3cccn(C)c3=O)n[nH]2)C1] |
| | (1R,3S)-3-(3-((1-methyl-2-oxo-1,2- |
| | dihydropyridin-3-yl)amino)-1H- |
| | pyrazol-5-yl)cyclopentyl |
| | isopropylcarbamate |
| 2CC | 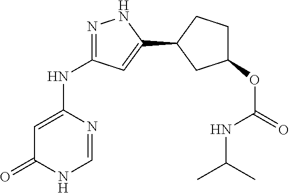 [SMILES: CC(C)NC(=O)O[C@@H]1CC[C@H](c2cc(Nc3cc(=O)[nH]cn3)n[nH]2)C1] |
| | (1R,3S)-3-(3-((6-oxo-1,6- |
| | dihydropyrimidin-4-yl)amino)-1H- |
| | pyrazol-5-yl)cyclopentyl |
| | isopropylcarbamate |
| 2DD | 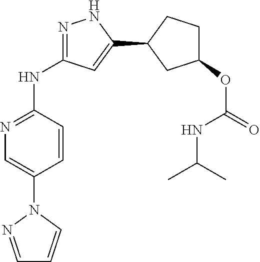 [SMILES: CC(C)NC(=O)O[C@@H]1CC[C@H](c2cc(Nc3ccc(-n4cccn4)cn3)n[nH]2)C1] |
| | (1R,3S)-3-(3-((5-(1H-pyrazol-1- |
| | yl)pyridin-2-yl)amino)-1H-pyrazol-5- |
| | yl)cyclopentyl isopropylcarbamate |
| 2EE | 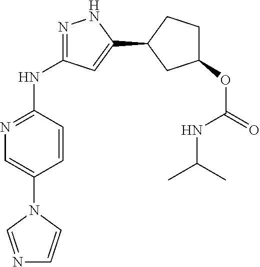 [SMILES: CC(C)NC(=O)O[C@@H]1CC[C@H](c2cc(Nc3ccc(-n4ccnc4)cn3)n[nH]2)C1] |
| | (1R,3S)-3-(3-((5-(1H-imidazol-1- |
| | yl)pyridin-2-yl)amino)-1H-pyrazol-5- |
| | yl)cyclopentyl isopropylcarbamate |
| 2FF | 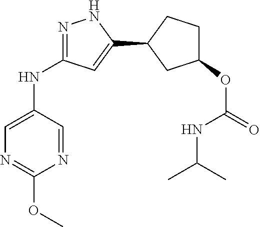 [SMILES: COc1ncc(Nc2cc([C@H]3CC[C@@H](OC(=O)NC(C)C)C3)[nH]n2)cn1] |
| | (1R,3S)-3-(3-((2-methoxypyrimidin-5- |
| | yl)amino)-1H-pyrazol-5-yl)cyclopentyl |
| | isopropylcarbamate |
| 2GG | 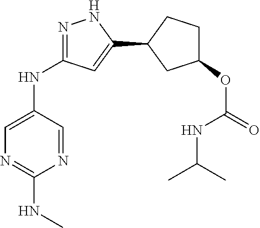 [SMILES: CNc1ncc(Nc2cc([C@H]3CC[C@@H](OC(=O)NC(C)C)C3)[nH]n2)cn1] |
| | (1R,3S)-3-(3-((2- |
| | (methylamino)pyrimidin-5-yl)amino)- |
| | 1H-pyrazol-5-yl)cyclopentyl |
| | isopropylcarbamate |
| 2HH | 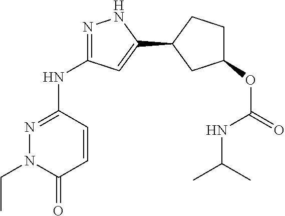 [SMILES: CCn1nc(Nc2cc([C@H]3CC[C@@H](OC(=O)NC(C)C)C3)[nH]n2)ccc1=O] |
| | (1R,3S)-3-(3-((1-ethyl-6-oxo-1,6- |
| | dihydropyridazin-3-yl)amino)-1H- |
| | pyrazol-5-yl)cyclopentyl |
| | isopropylcarbamate |
| 2II | 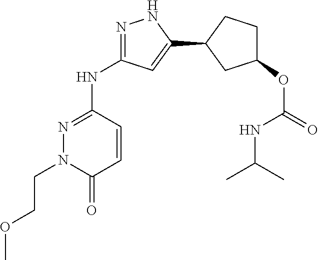 [SMILES: COCCn1nc(Nc2cc([C@H]3CC[C@@H](OC(=O)NC(C)C)C3)[nH]n2)ccc1=O] |
| | (1R,3S)-3-(3-((1-(2-methoxyethyl)-6- |
| | oxo-1,6-dihydropyridazin-3-yl)amino)- |
| | 1H-pyrazol-5-yl)cyclopentyl |
| | isopropylcarbamate |
| 2JJ | 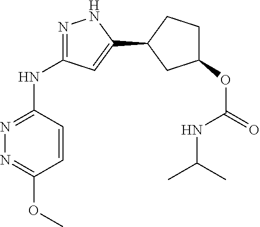 [SMILES: COc1ccc(Nc2cc([C@H]3CC[C@@H](OC(=O)NC(C)C)C3)[nH]n2)nn1] |
| | (1R,3S)-3-(3-((6-methoxypyridazin-3- |
| | yl)amino)-1H-pyrazol-5-yl)cyclopentyl |
| | isopropylcarbamate |
| 3 | 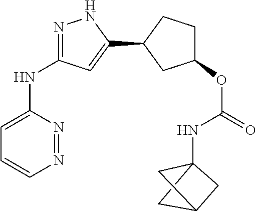 [SMILES: O=C(NC12CC(C1)C2)O[C@@H]1CC[C@H](c2cc(Nc3cccnn3)n[nH]2)C1] |
| | (1R,3S)-3- |
| | (3-(pyridazin-3-ylamino)-1H-pyrazol- |
| | 5-yl)cyclopentyl bicyclo[1.1.1]pentan- |
| | 1-ylcarbamate |
| 3A | 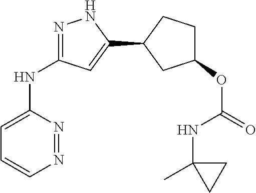 [SMILES: CC1(NC(=O)O[C@@H]2CC[C@H](c3cc(Nc4cccnn4)n[nH]3)C2)CC1] |
| | (1R,3S)-3-(3-(pyridazin-3-ylamino)- |
| | 1H-pyrazol-5-yl)cyclopentyl (1- |
| | methylcyclopropyl)carbamate |
| 3B* | 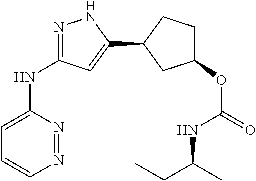 [SMILES: CC[C@H](C)NC(=O)O[C@@H]1CC[C@H](c2cc(Nc3cccnn3)n[nH]2)C1] |
| | (1R,3S)-3-(3-(pyridazin-3-ylamino)- |
| | 1H-pyrazol-5-yl)cyclopentyl ((S)-sec- |
| | butyl)carbamate |
| 3C* | 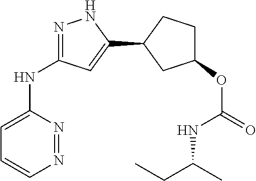 [SMILES: CC[C@@H](C)NC(=O)O[C@@H]1CC[C@H](c2cc(Nc3cccnn3)n[nH]2)C1] |
| | (1R,3S)-3-(3-(pyridazin-3-ylamino)- |
| | 1H-pyrazol-5-yl)cyclopentyl ((R)-sec- |
| | butyl)carbamate |
| 3D | 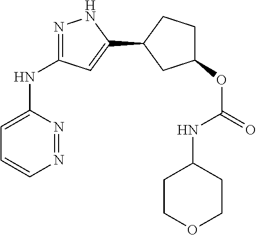 [SMILES: O=C(NC1CCOCC1)O[C@@H]1CC[C@H](c2cc(Nc3cccnn3)n[nH]2)C1] |
| | (1R,3S)-3-(3-(pyridazin-3-ylamino)- |
| | 1H-pyrazol-5-yl)cyclopentyl |
| | (tetrahydro-2H-pyran-4-yl)carbamate |
| 3F | 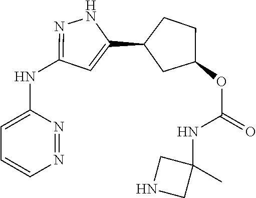 [SMILES: CC1(NC(=O)O[C@@H]2CC[C@H](c3cc(Nc4cccnn4)n[nH]3)C2)CNC1] |
| | (1R,3S)-3-(3-(pyridazin-3-ylamino)-1H- |
| | pyrazol-5-yl)cyclopentyl (3- |
| | methylazetidin-3-yl)carbamate |
| 3G | 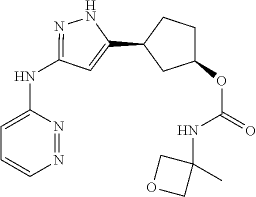 [SMILES: CC1(NC(=O)O[C@@H]2CC[C@H](c3cc(Nc4cccnn4)n[nH]3)C2)COC1] |
| | (1R,3S)-3-(3-(pyridazin-3-ylamino)-1H- |
| | pyrazol-5-yl)cyclopentyl (3-methyloxetan- |
| | 3-yl)carbamate |
| 4 | 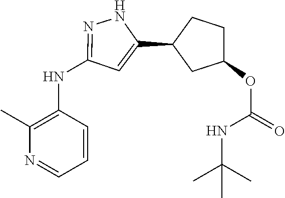 [SMILES: Cc1ncccc1Nc1cc([C@H]2CC[C@@H](OC(=O)NC(C)(C)C)C2)[nH]n1] |
| | (1R,3S)-3-(3-((4-methylpyridin-3- |
| | yl)amino)-1H-pyrazol-5-yl)cyclopentyl |
| | tert-butylcarbamate |
| 4A | 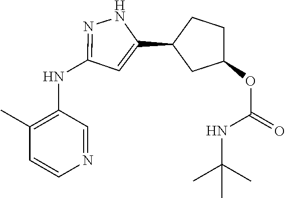 [SMILES: Cc1ccncc1Nc1cc([C@H]2CC[C@@H](OC(=O)NC(C)(C)C)C2)[nH]n1] |
| | (1R,3S)-3-(3-((4-methylpyridin-3- |
| | yl)amino)-1H-pyrazol-5-yl)cyclopentyl |
| | tert-butylcarbamate |
| 4B | 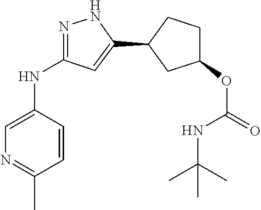 [SMILES: Cc1ccc(Nc2cc([C@H]3CC[C@@H](OC(=O)NC(C)(C)C)C3)[nH]n2)cn1] |
| | (1R,3S)-3-(3-((6-methylpyridin-3- |
| | yl)amino)-1H-pyrazol-5-yl)cyclopentyl |
| | tert-butylcarbamate |
| 4C | 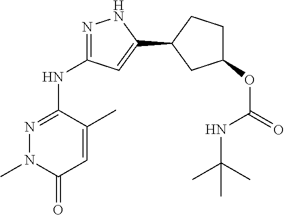 [SMILES: Cc1cc(=O)n(C)nc1Nc1cc([C@H]2CC[C@@H](OC(=O)NC(C)(C)C)C2)[nH]n1] |
| | (1R,3S)-3-(3-((1,4-dimethyl-6-oxo-1,6- |
| | dihydropyridazin-3-yl)amino)-1H-pyrazol- |
| | 5-yl)cyclopentyl tert-butylcarbamate |
| 4D | 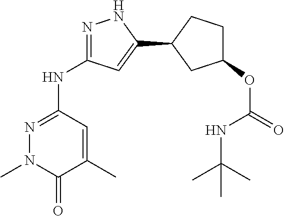 [SMILES: Cc1cc(Nc2cc([C@H]3CC[C@@H](OC(=O)NC(C)(C)C)C3)[nH]n2)nn(C)c1=O] |
| | (1R,3S)-3-(3-((1,5-dimethyl-6-oxo-1,6- |
| | dihydropyridazin-3-yl)amino)-1H-pyrazol- |
| | 5-yl)cyclopentyl tert-butylcarbamate |
| 4E | 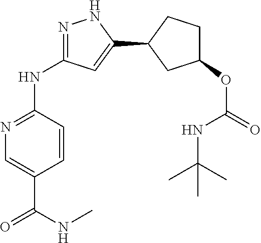 [SMILES: CNC(=O)c1ccc(Nc2cc([C@H]3CC[C@@H](OC(=O)NC(C)(C)C)C3)[nH]n2)nc1] |
| | (1R,3S)-3-(3-((5- |
| | (methylcarbamoyl)pyridin-2-yl)amino)- |
| | 1H-pyrazol-5-yl)cyclopentyl tert- |
| | butylcarbamate |
| 4F | 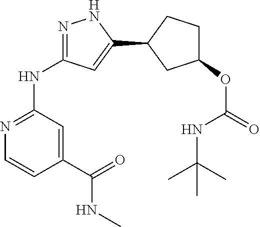 [SMILES: CNC(=O)c1ccnc(Nc2cc([C@H]3CC[C@@H](OC(=O)NC(C)(C)C)C3)[nH]n2)c1] |
| | (1R,3S)-3-(3-((4- |
| | (methylcarbamoyl)pyridin-2-yl)amino)- |
| | 1H-pyrazol-5-yl)cyclopentyl tert- |
| | butylcarbamate |
| 4G | 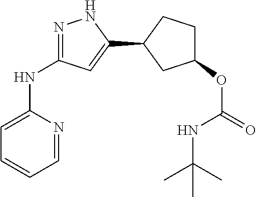 [SMILES: CC(C)(C)NC(=O)O[C@@H]1CC[C@H](c2cc(Nc3ccccn3)n[nH]2)C1] |
| | (1R,3S)-3-(3-(pyridin-2-ylamino)-1H- |
| | pyrazol-5-yl)cyclopentyl tert- |
| | butylcarbamate |
| 4H | 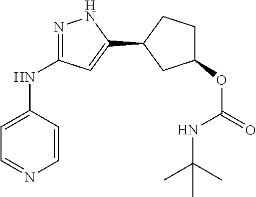 [SMILES: CC(C)(C)NC(=O)O[C@@H]1CC[C@H](c2cc(Nc3ccncc3)n[nH]2)C1] |
| | (1R,3S)-3-(3-(pyridin-4-ylamino)-1H- |
| | pyrazol-5-yl)cyclopentyl tert- |
| | butylcarbamate |
| 4I | 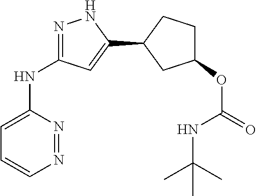 [SMILES: CC(C)(C)NC(=O)O[C@@H]1CC[C@H](c2cc(Nc3cccnn3)n[nH]2)C1] |
| | (1R,3S)-3-(3-(pyridazin-3-ylamino)-1H- |
| | pyrazol-5-yl)cyclopentyl tert- |
| | butylcarbamate |
| 4J | 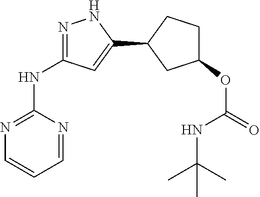 [SMILES: CC(C)(C)NC(=O)O[C@@H]1CC[C@H](c2cc(Nc3ncccn3)n[nH]2)C1] |
| | (1R,3S)-3-(3-(pyrimidin-2-ylamino)-1H- |
| | pyrazol-5-yl)cyclopentyl tert- |
| | butylcarbamate |
| 4K | 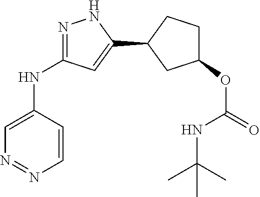 [SMILES: CC(C)(C)NC(=O)O[C@@H]1CC[C@H](c2cc(Nc3ccnnc3)n[nH]2)C1] |
| | (1R,3S)-3-(3-(pyridazin-4-ylamino)-1H- |
| | pyrazol-5-yl)cyclopentyl tert- |
| | butylcarbamate |
| 5 | 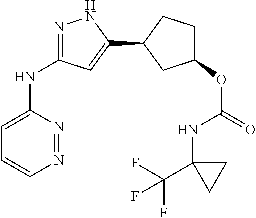 [SMILES: O=C(NC1(C(F)(F)F)CC1)O[C@@H]1CC[C@H](c2cc(Nc3cccnn3)n[nH]2)C1] |
| | (1R,3S)-3-(3-(pyridazin-3-ylamino)-1H- |
| | pyrazol-5-yl)cyclopentyl (1- |
| | (trifluoromethyl)cyclopropyl)carbamate |
| 5A* | 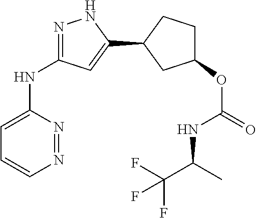 [SMILES: C[C@H](NC(=O)O[C@@H]1CC[C@H](c2cc(Nc3cccnn3)n[nH]2)C1)C(F)(F)F] |
| | (1R,3S)-3-(3-(pyridazin-3-ylamino)-1H- |
| | pyrazol-5-yl)cyclopentyl ((S)-1,1,1- |
| | trifluoropropan-2-yl)carbamate |
| 5B* | 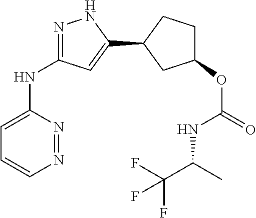 [SMILES: C[C@@H](NC(=O)O[C@@H]1CC[C@H](c2cc(Nc3cccnn3)n[nH]2)C1)C(F)(F)F] |
| | (1R,3S)-3-(3-(pyridazin-3-ylamino)-1H- |
| | pyrazol-5-yl)cyclopentyl ((R)-1,1,1- |
| | trifluoropropan-2-yl)carbamate |
| 6 | 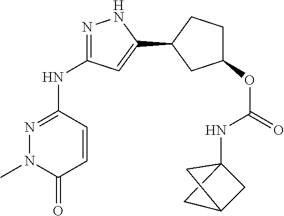 [SMILES: Cn1nc(Nc2cc([C@H]3CC[C@@H](OC(=O)NC45CC(C4)C5)C3)[nH]n2)ccc1=O] |
| | (1R,3S)-3-(5-((1-methyl-6-oxo-1,6- |
| | dihydropyridazin-3-yl)amino)-1H-pyrazol- |
| | 3-yl)cyclopentyl bicyclo[1.1.1]pentan-1- |
| | ylcarbamate |
| 6A | 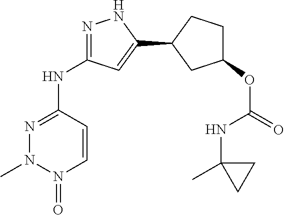 |
| | (1R,3S)-3-(3-((1-methyl-6-oxo-1,6- |
| | dihydropyridazin-3-yl)amino)-1H-pyrazol- |
| | 5-yl)cyclopentyl (1- |
| | methylcyclopropyl)carbamate |
| 6B | 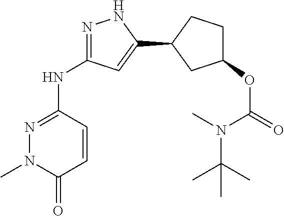 [SMILES: CN(C(=O)O[C@@H]1CC[C@H](c2cc(Nc3ccc(=O)n(C)n3)n[nH]2)C1)C(C)(C)C] |
| | (1R,3S)-3-(3-((1-methyl-6-oxo-1,6- |
| | dihydropyridazin-3-yl)amino)-1H-pyrazol- |
| | 5-yl)cyclopentyl tert- |
| | butyl(methyl)carbamate |
| 6C | 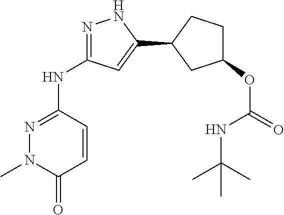 [SMILES: Cn1nc(Nc2cc([C@H]3CC[C@@H](OC(=O)NC(C)(C)C)C3)[nH]n2)ccc1=O] |
| | (1R,3S)-3-(3-((1-methyl-6-oxo-1,6- |
| | dihydropyridazin-3-yl)amino)-1H-pyrazol- |
| | 5-yl)cyclopentyl tert-butylcarbamate |
| 7 | 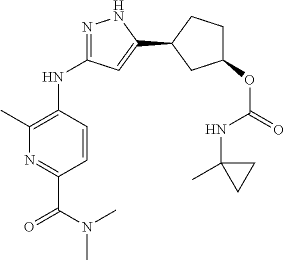 [SMILES: Cc1nc(C(=O)N(C)C)ccc1Nc1cc([C@H]2CC[C@@H](OC(=O)NC3(C)CC3)C2)[nH]n1] |
| | (1R,3S)-3-(3-((6-(dimethylcarbamoyl)-2- |
| | methylpyridin-3-yl)amino)-1H-pyrazol-5- |
| | yl)cyclopentyl (1- |
| | methylcyclopropyl)carbamate |
| 7A | 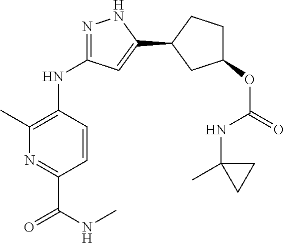 [SMILES: CNC(=O)c1ccc(Nc2cc([C@H]3CC[C@@H](OC(=O)NC4(C)CC4)C3)[nH]n2)c(C)n1] |
| | (1R,3S)-3-(3-((2-methyl-6- |
| | (methylcarbamoyl)pyridin-3-yl)amino)- |
| | 1H-pyrazol-5-yl)cyclopentyl (1- |
| | methylcyclopropyl)carbamate |
| 7B | 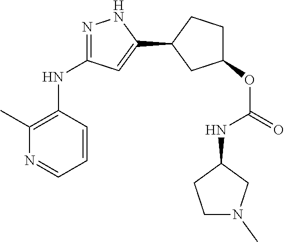 [SMILES: Cc1ncccc1Nc1cc([C@H]2CC[C@@H](OC(=O)N[C@@H]3CCN(C)C3)C2)[nH]n1] |
| | (1R,3S)-3-(3-((2-methylpyridin-3- |
| | yl)amino)-1H-pyrazol-5-yl)cyclopentyl |
| | ((R)-1-methylpyrrolidin-3-yl)carbamate |
| 7C | 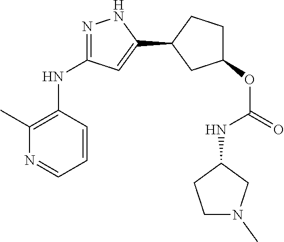 [SMILES: Cc1ncccc1Nc1cc([C@H]2CC[C@@H](OC(=O)N[C@H]3CCN(C)C3)C2)[nH]n1] |
| | (1R,3S)-3-(3-((2-methylpyridin-3- |
| | yl)amino)-1H-pyrazol-5-yl)cyclopentyl |
| | ((S)-1-methylpyrrolidin-3-yl)carbamate |
| 7D | 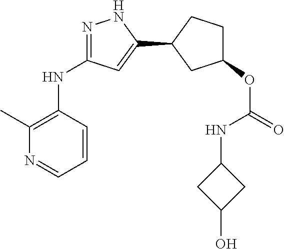 [SMILES: Cc1ncccc1Nc1cc([C@H]2CC[C@@H](OC(=O)NC3CC(O)C3)C2)[nH]n1] |
| | (1R,3S)-3-(3-((2-methylpyridin-3- |
| | yl)amino)-1H-pyrazol-5-yl)cyclopentyl |
| | ((1s,3s)-3-hydroxycyclobutyl)carbamate |
| 8 | 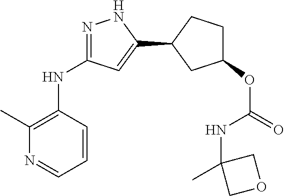 [SMILES: Cc1ncccc1Nc1cc([C@H]2CC[C@@H](OC(=O)NC3(C)COC3)C2)[nH]n1] |
| | (1R,3S)-3-(3-((6-(dimethylcarbamoyl)-2- |
| | methylpyridin-3-yl)amino)-1H-pyrazol-5- |
| | yl)cyclopentyl (1- |
| | methylcyclopropyl)carbamate |
| 9 | 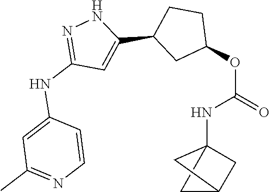 [SMILES: Cc1cc(Nc2cc([C@H]3CC[C@@H](OC(=O)NC45CC(C4)C5)C3)[nH]n2)ccn1] |
| | (1R,3S)-3-(3-((2-methylpyridin-4- |
| | yl)amino)-1H-pyrazol-5-yl)cyclopentyl |
| | bicyclo[1.1.1]pentan-1-ylcarbamate |
| 9A | 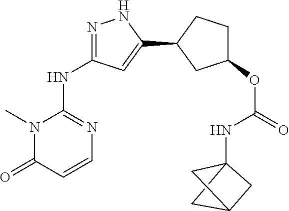 [SMILES: Cn1c(Nc2cc([C@H]3CC[C@@H](OC(=O)NC45CC(C4)C5)C3)[nH]n2)nccc1=O] |
| | (1R,3S)-3-(3-((1-methyl-6-oxo-1,6- |
| | dihydropyrimidin-2-yl)amino)-1H-pyrazol- |
| | 5-yl)cyclopentyl bicyclo[1.1.1]pentan-1- |
| | ylcarbamate |
| 9B | 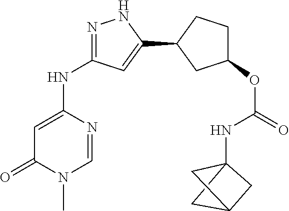 [SMILES: Cn1cnc(Nc2cc([C@H]3CC[C@@H](OC(=O)NC45CC(C4)C5)C3)[nH]n2)cc1=O] |
| | (1R,3S)-3-(3-((1-methyl-6-oxo-1,6- |
| | dihydropyrimidin-4-yl)amino)-1H-pyrazol- |
| | 5-yl)cyclopentyl bicyclo[1.1.1]pentan-1- |
| | ylcarbamate |
| 9C | 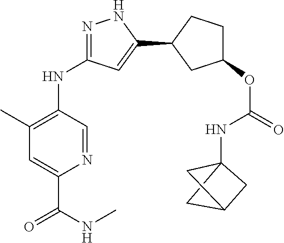 [SMILES: CNC(=O)c1cc(C)c(Nc2cc([C@H]3CC[C@@H](OC(=O)NC45CC(C4)C5)C3)[nH]n2)cn1] |
| | (1R,3S)-3-(3-((4-methyl-6- |
| | (methylcarbamoyl)pyridin-3-yl)amino)- |
| | 1H-pyrazol-5-yl)cyclopentyl |
| | bicyclo[1.1.1]pentan-1-ylcarbamate |
| 9D | 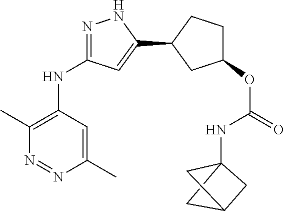 [SMILES: Cc1cc(Nc2cc([C@H]3CC[C@@H](OC(=O)NC45CC(C4)C5)C3)[nH]n2)c(C)nn1] |
| | (1R,3S)-3-(3-((3,6-dimethylpyridazin-4- |
| | yl)amino)-1H-pyrazol-5-yl)cyclopentyl |
| | bicyclo[1.1.1]pentan-1-ylcarbamate |
| 9E | 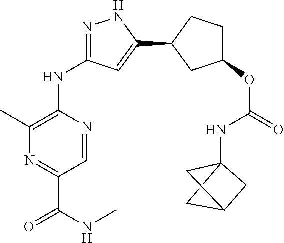 [SMILES: CNC(=O)c1cnc(Nc2cc([C@H]3CC[C@@H](OC(=O)NC45CC(C4)C5)C3)[nH]n2)c(C)n1] |
| | (1R,3S)-3-(3-((3-methyl-5- |
| | (methylcarbamoyl)pyrazin-2-yl)amino)- |
| | 1H-pyrazol-5-yl)cyclopentyl |
| | bicyclo[1.1.1]pentan-1-ylcarbamate |
| 9F | 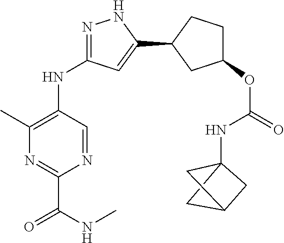 [SMILES: CNC(=O)c1ncc(Nc2cc([C@H]3CC[C@@H](OC(=O)NC45CC(C4)C5)C3)[nH]n2)c(C)n1] |
| | (1R,3S)-3-(3-((4-methyl-2- |
| | (methylcarbamoyl)pyrimidin-5-yl)amino)- |
| | 1H-pyrazol-5-yl)cyclopentyl |
| | bicyclo[1.1.1]pentan-1-ylcarbamate |
| 9G | 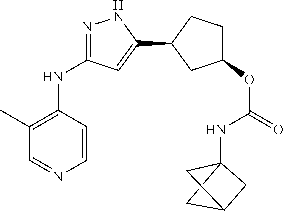 [SMILES: Cc1cnccc1Nc1cc([C@H]2CC[C@@H](OC(=O)NC34CC(C3)C4)C2)[nH]n1] |
| | (1R,3S)-3-(3-((3-methylpyridin-4- |
| | yl)amino)-1H-pyrazol-5-yl)cyclopentyl |
| | bicyclo[1.1.1]pentan-1-ylcarbamate |
| 9H | 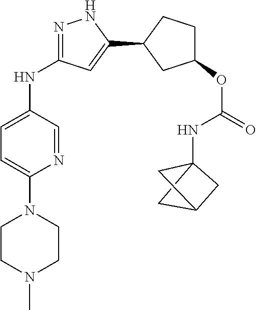 [SMILES: CN1CCN(c2ccc(Nc3cc([C@H]4CC[C@@H](OC(=O)NC56CC(C5)C6)C4)[nH]n3)cn2)CC1] |
| | (1R,3S)-3-(3-((6-(4-methylpiperazin-1- |
| | yl)pyridin-3-yl)amino)-1H-pyrazol-5- |
| | yl)cyclopentyl bicyclo[1.1.1]pentan-1- |
| | ylcarbamate |
| 9I | 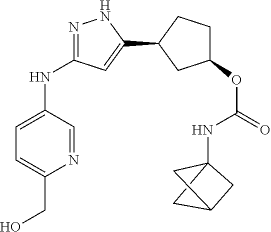 [SMILES: O=C(NC12CC(C1)C2)O[C@@H]1CC[C@H](c2cc(Nc3ccc(CO)nc3)n[nH]2)C1] |
| | (1R,3S)-3-(3-((6-(hydroxymethyl)pyridin- |
| | 3-yl)amino)-1H-pyrazol-5-yl)cyclopentyl |
| | bicyclo[1.1.1]pentan-1-ylcarbamate |
| 9J | 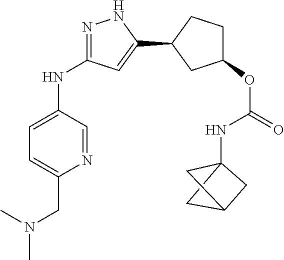 [SMILES: CN(C)Cc1ccc(Nc2cc([C@H]3CC[C@@H](OC(=O)NC45CC(C4)C5)C3)[nH]n2)cn1] |
| | (1R,3S)-3-(3-((6- |
| | ((dimethylamino)methyl)pyridin-3- |
| | yl)amino)-1H-pyrazol-5-yl)cyclopentyl |
| | bicyclo[1.1.1]pentan-1-ylcarbamate |
| 9K | 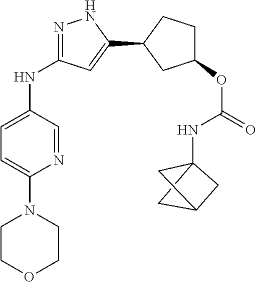 [SMILES: O=C(NC12CC(C1)C2)O[C@@H]1CC[C@H](c2cc(Nc3ccc(N4CCOCC4)nc3)n[nH]2)C1] |
| | (1R,3S)-3-(3-((6-morpholinopyridin-3- |
| | yl)amino)-1H-pyrazol-5-yl)cyclopentyl |
| | bicyclo[1.1.1]pentan-1-ylcarbamate |
| 10 | 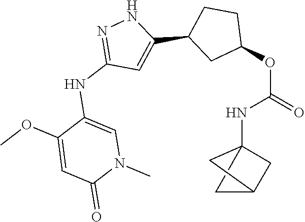 [SMILES: COc1cc(=O)n(C)cc1Nc1cc([C@H]2CC[C@@H](OC(=O)NC34CC(C3)C4)C2)[nH]n1] |
| | (1R,3S)-3-(3-((4-methoxy-1-methyl-6-oxo- |
| | 1,6-dihydropyridin-3-yl)amino)-1H- |
| | pyrazol-5-yl)cyclopentyl |
| | bicyclo[1.1.1]pentan-1-ylcarbamate |
| 11 | 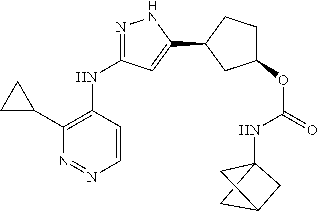 [SMILES: O=C(NC12CC(C1)C2)O[C@@H]1CC[C@H](c2cc(Nc3ccnnc3C3CC3)n[nH]2)C1] |
| | (1R,3S)-3-(3-((3-cyclopropylpyridazin-4- |
| | yl)amino)-1H-pyrazol-5-yl)cyclopentyl |
| | bicyclo[1.1.1]pentan-1-ylcarbamate |
| 3E | 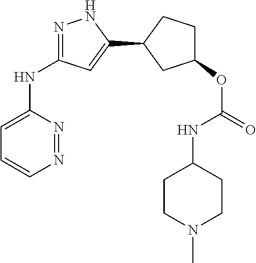 [SMILES: CN1CCC(NC(=O)O[C@@H]2CC[C@H](c3cc(Nc4cccnn4)n[nH]3)C2)CC1] |
| | (1R,3S)-3-(3-(pyridazin-3-ylamino)-1H- |
| | pyrazol-5-yl)cyclopentyl (1- |
| | methylpiperidin-4-yl)carbamate |

or a pharmaceutically acceptable salt thereof.

TABLE 2
Exemplary Compounds of Formula (I)

| | | | |
|---|---|---|---|
| 4L | 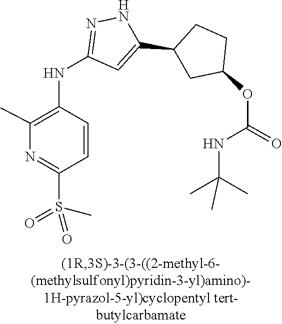 [SMILES: Cc1nc(S(C)(=O)=O)ccc1Nc1cc([C@H]2CC[C@@H](OC(=O)NC(C)(C)C)C2)[nH]n1] | 8B | 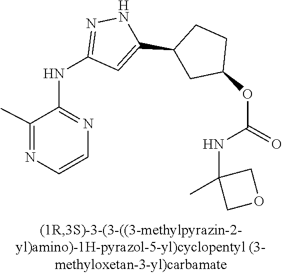 [SMILES: Cc1nccnc1Nc1cc([C@H]2CC[C@@H](OC(=O)NC3(C)COC3)C2)[nH]n1] |
| 5C | 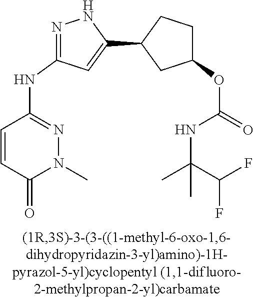 [SMILES: Cn1nc(Nc2cc([C@H]3CC[C@@H](OC(=O)NC(C)(C)C(F)F)C3)[nH]n2)ccc1=O] | 10C | 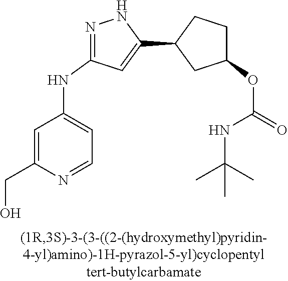 [SMILES: CC(C)(C)NC(=O)O[C@@H]1CC[C@H](c2cc(Nc3ccnc(CO)c3)n[nH]2)C1] |
| 6D | 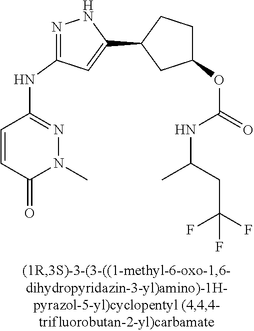 [SMILES: CC(CC(F)(F)F)NC(=O)O[C@@H]1CC[C@H](c2cc(Nc3ccc(=O)n(C)n3)n[nH]2)C1] | 10D | 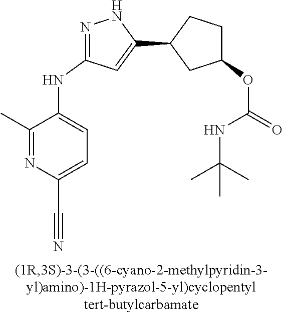 [SMILES: Cc1nc(C#N)ccc1Nc1cc([C@H]2CC[C@@H](OC(=O)NC(C)(C)C)C2)[nH]n1] |
| 6E | 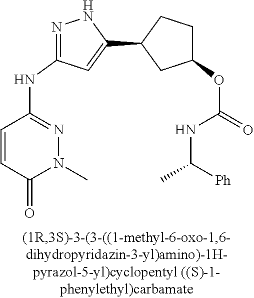 [SMILES: C[C@H](NC(=O)O[C@@H]1CC[C@H](c2cc(Nc3ccc(=O)n(C)n3)n[nH]2)C1)c1ccccc1] | 10E | 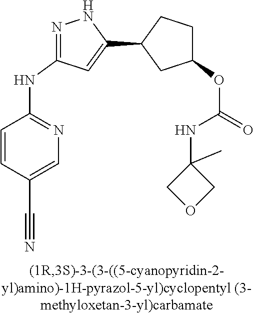 [SMILES: CC1(NC(=O)O[C@@H]2CC[C@H](c3cc(Nc4ccc(C#N)cn4)n[nH]3)C2)COC1] |
| 8A | 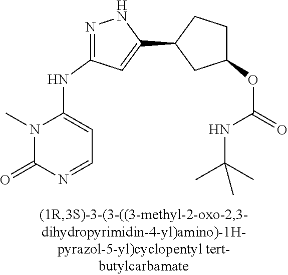 [SMILES: Cn1c(Nc2cc([C@H]3CC[C@@H](OC(=O)NC(C)(C)C)C3)[nH]n2)ccnc1=O] | 11A | 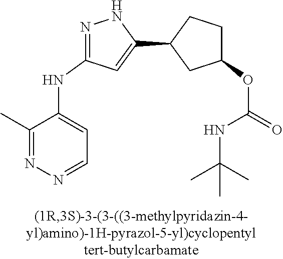 [SMILES: Cc1nnccc1Nc1cc([C@H]2CC[C@@H](OC(=O)NC(C)(C)C)C2)[nH]n1] |

or a pharmaceutically acceptable salt thereof.

In some embodiments, the compound of Formula (I) or a pharmaceutically acceptable salt thereof, is a compound having the structure:

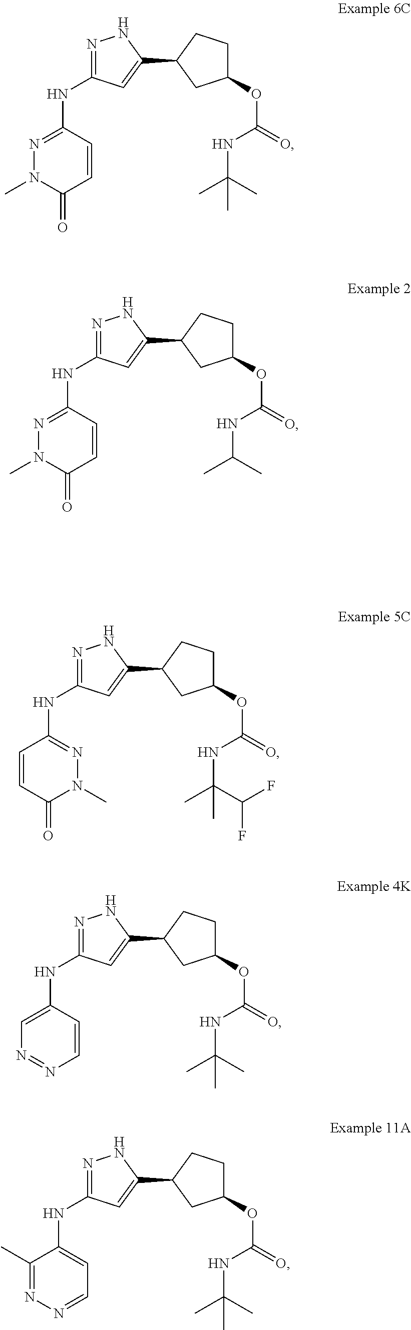 [SMILES: Cn1nc(Nc2cc([C@H]3CC[C@@H](OC(=O)NC(C)(C)C)C3)[nH]n2)ccc1=O ; CC(C)NC(=O)O[C@@H]1CC[C@H](c2cc(Nc3ccc(=O)n(C)n3)n[nH]2)C1 ; Cn1nc(Nc2cc([C@H]3CC[C@@H](OC(=O)NC(C)(C)C(F)F)C3)[nH]n2)ccc1=O ; CC(C)(C)NC(=O)O[C@@H]1CC[C@H](c2cc(Nc3ccnnc3)n[nH]2)C1 ; Cc1nnccc1Nc1cc([C@H]2CC[C@@H](OC(=O)NC(C)(C)C)C2)[nH]n1]

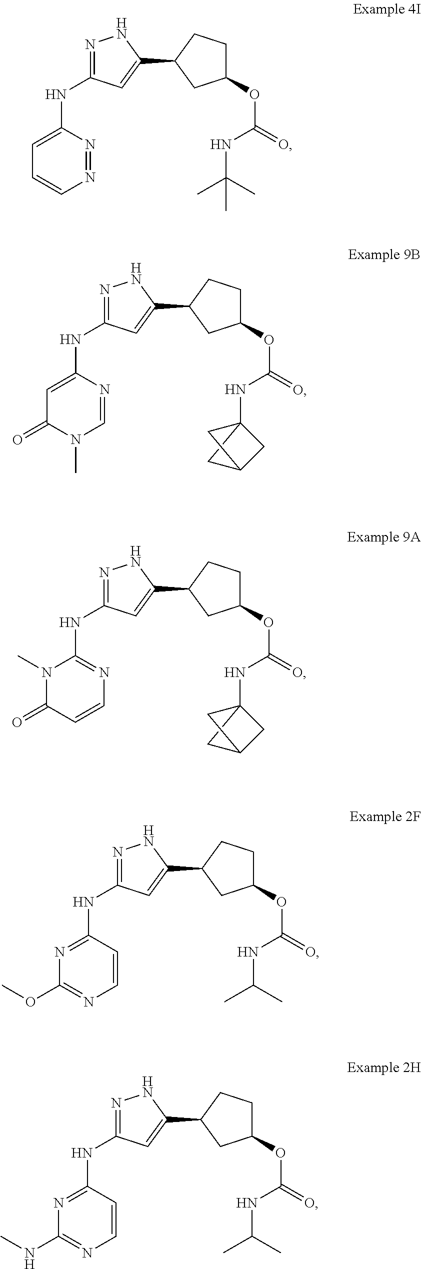 [SMILES: CC(C)(C)NC(=O)O[C@@H]1CC[C@H](c2cc(Nc3cccnn3)n[nH]2)C1 ; Cn1cnc(Nc2cc([C@H]3CC[C@@H](OC(=O)NC45CC(C4)C5)C3)[nH]n2)cc1=O ; Cn1c(Nc2cc([C@H]3CC[C@@H](OC(=O)NC45CC(C4)C5)C3)[nH]n2)nccc1=O ; COc1nccc(Nc2cc([C@H]3CC[C@@H](OC(=O)NC(C)C)C3)[nH]n2)n1 ; CNc1nccc(Nc2cc([C@H]3CC[C@@H](OC(=O)NC(C)C)C3)[nH]n2)n1]

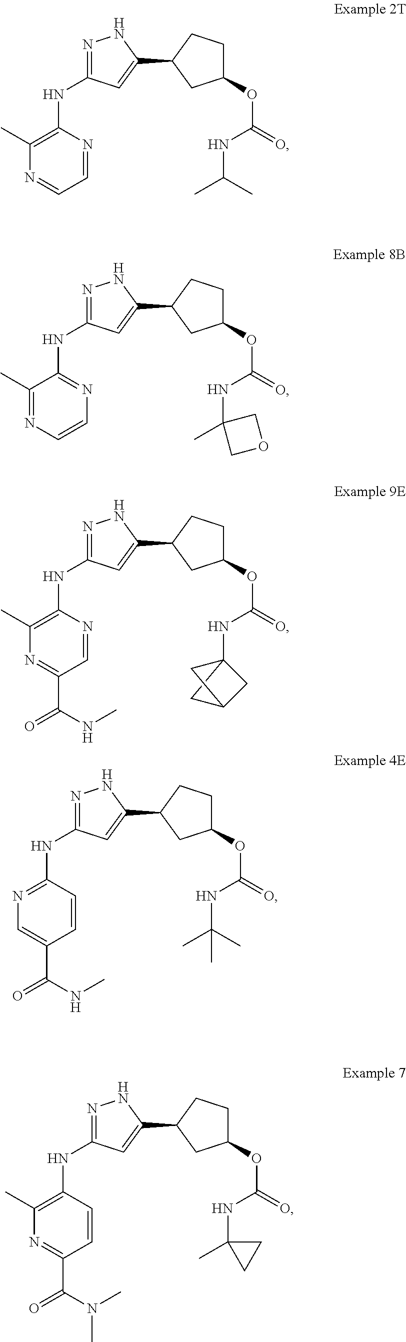 [SMILES: Cc1nccnc1Nc1cc([C@H]2CC[C@@H](OC(=O)NC(C)C)C2)[nH]n1 ; Cc1nccnc1Nc1cc([C@H]2CC[C@@H](OC(=O)NC3(C)COC3)C2)[nH]n1 ; CNC(=O)c1cnc(Nc2cc([C@H]3CC[C@@H](OC(=O)NC45CC(C4)C5)C3)[nH]n2)c(C)n1 ; CNC(=O)c1ccc(Nc2cc([C@H]3CC[C@@H](OC(=O)NC(C)(C)C)C3)[nH]n2)nc1 ; Cc1nc(C(=O)N(C)C)ccc1Nc1cc([C@H]2CC[C@@H](OC(=O)NC3(C)CC3)C2)[nH]n1]

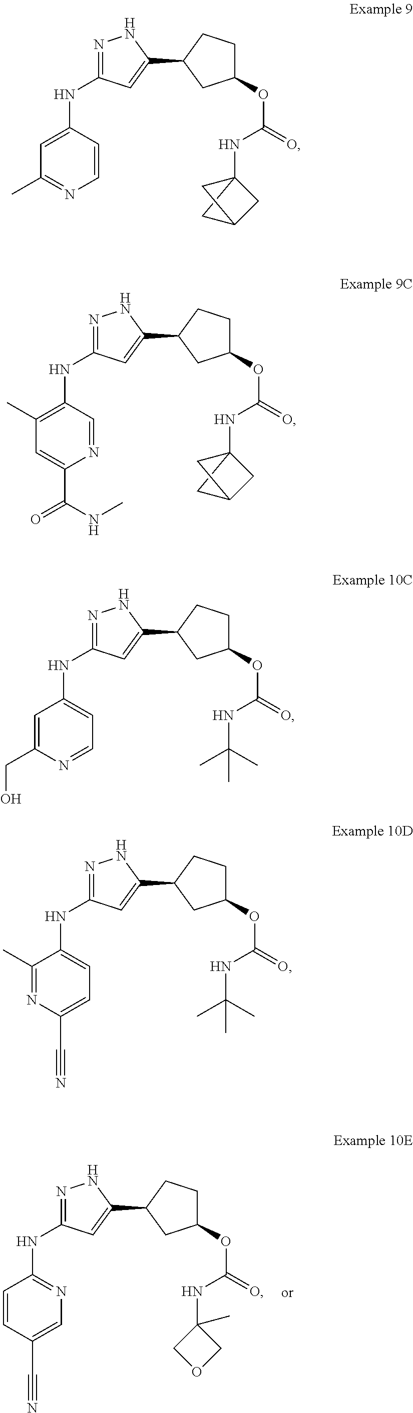 [SMILES: Cc1cc(Nc2cc([C@H]3CC[C@@H](OC(=O)NC45CC(C4)C5)C3)[nH]n2)ccn1 ; CNC(=O)c1cc(C)c(Nc2cc([C@H]3CC[C@@H](OC(=O)NC45CC(C4)C5)C3)[nH]n2)cn1 ; CC(C)(C)NC(=O)O[C@@H]1CC[C@H](c2cc(Nc3ccnc(CO)c3)n[nH]2)C1 ; Cc1nc(C#N)ccc1Nc1cc([C@H]2CC[C@@H](OC(=O)NC(C)(C)C)C2)[nH]n1 ; CC1(NC(=O)O[C@@H]2CC[C@H](c3cc(Nc4ccc(C#N)cn4)n[nH]3)C2)COC1]

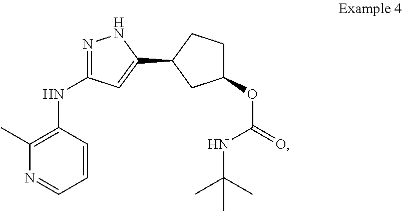 [SMILES: Cc1ncccc1Nc1cc([C@H]2CC[C@@H](OC(=O)NC(C)(C)C)C2)[nH]n1]

or a pharmaceutically acceptable salt thereof.

In some embodiments, the compound has the formula:

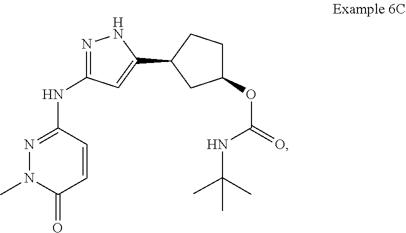 [SMILES: Cn1nc(Nc2cc([C@H]3CC[C@@H](OC(=O)NC(C)(C)C)C3)[nH]n2)ccc1=O]

or a pharmaceutically acceptable salt thereof.

In some embodiments, the compound has the formula:

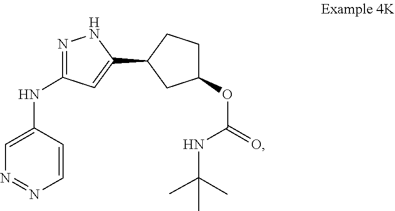 [SMILES: CC(C)(C)NC(=O)O[C@@H]1CC[C@H](c2cc(Nc3ccnnc3)n[nH]2)C1]

or a pharmaceutically acceptable salt thereof.

In some embodiments, the compound has the formula:

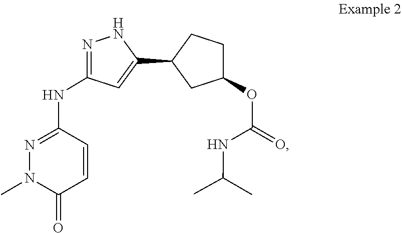 [SMILES: CC(C)NC(=O)O[C@@H]1CC[C@H](c2cc(Nc3ccc(=O)n(C)n3)n[nH]2)C1]

or a pharmaceutically acceptable salt thereof.

In some embodiments, the compound has the formula:

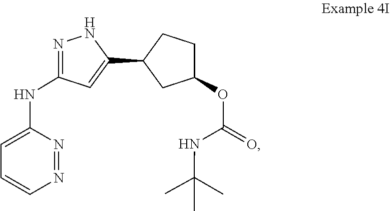 [SMILES: CC(C)(C)NC(=O)O[C@@H]1CC[C@H](c2cc(Nc3cccnn3)n[nH]2)C1]

or a pharmaceutically acceptable salt thereof.

In some embodiments, the compound has the formula:

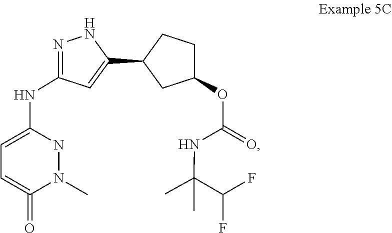

or a pharmaceutically acceptable salt thereof.

In some embodiments, the compound has the formula:

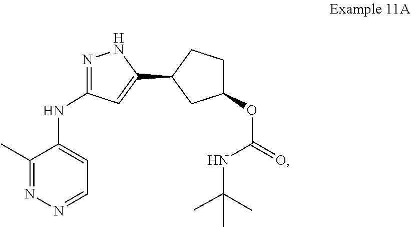 [SMILES: Cc1nnccc1Nc1cc([C@H]2CC[C@@H](OC(=O)NC(C)(C)C)C2)[nH]n1]

or a pharmaceutically acceptable salt thereof.

In some embodiments, the compound has the formula:

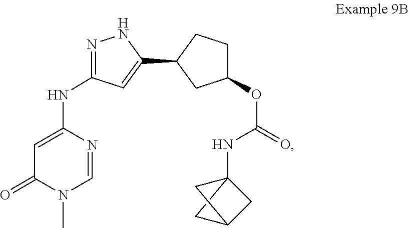 [SMILES: Cn1cnc(Nc2cc([C@H]3CC[C@@H](OC(=O)NC45CC(C4)C5)C3)[nH]n2)cc1=O]

or a pharmaceutically acceptable salt thereof.

In some embodiments, the compound has the formula:

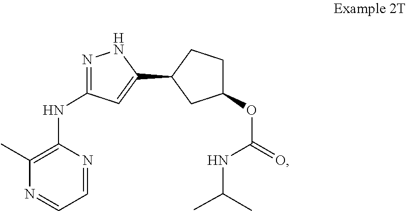 [SMILES: Cc1nccnc1Nc1cc([C@H]2CC[C@@H](OC(=O)NC(C)C)C2)[nH]n1]

or a pharmaceutically acceptable salt thereof.

In some embodiments, the compound has the formula:

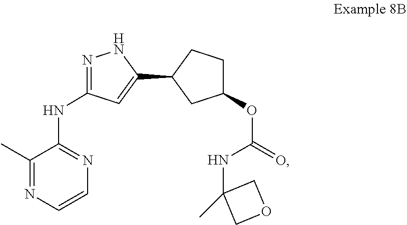 [SMILES: Cc1nccnc1Nc1cc([C@H]2CC[C@@H](OC(=O)NC3(C)COC3)C2)[nH]n1]

or a pharmaceutically acceptable salt thereof.

In some embodiments, the compound has the formula:

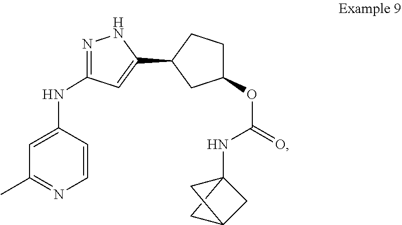 [SMILES: Cc1cc(Nc2cc([C@H]3CC[C@@H](OC(=O)NC45CC(C4)C5)C3)[nH]n2)ccn1]

or a pharmaceutically acceptable salt thereof.

In some embodiments, the compound has the formula:

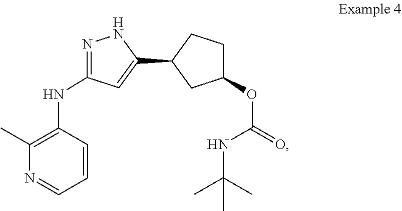 [SMILES: Cc1ncccc1Nc1cc([C@H]2CC[C@@H](OC(=O)NC(C)(C)C)C2)[nH]n1]

or a pharmaceutically acceptable salt thereof.

In some embodiments, the compound of Formula (I) or a pharmaceutically acceptable salt thereof, is the compound wherein Ring A is a monocyclic 6-membered heteroaryl ring consisting of 1 N-ring heteroatom.

In some embodiments, the compound of Formula (I) or a pharmaceutically acceptable salt thereof, is the compound wherein Ring A is a monocyclic 6-membered heteroaryl ring consisting of 2 N-ring heteroatoms.

In some embodiments, the compound of Formula (I) or a pharmaceutically acceptable salt thereof, is the compound wherein Ring A is a monocyclic 6-membered heteroaryl ring consisting of 3 N-ring heteroatoms.

In some embodiments, the compound of Formula (I) or a pharmaceutically acceptable salt thereof, is the compound wherein m is 1.

In some embodiments, the compound of Formula (I) or a pharmaceutically acceptable salt thereof, is the compound m is 1, the at least 1 N-ring heteroatom is substituted with the group R⁴, and Ring A comprises 1 additional N-ring heteroatom.

In some embodiments, the compound of Formula (I) or a pharmaceutically acceptable salt thereof, is the compound wherein at least 1 N-ring heteroatom is meta relative to the point of attachment.

In some embodiments, the compound of Formula (I) or a pharmaceutically acceptable salt thereof, is the compound wherein at least 1 N-ring heteroatom is para relative to the point of attachment.

In some embodiments, the compound of Formula (I) or a pharmaceutically acceptable salt thereof, is the compound wherein Ring A has 2 N-ring heteroatoms, and the 2 N atoms are meta and para relative to the point of attachment.

In some embodiments, the compound of Formula (I) or a pharmaceutically acceptable salt thereof, is the compound wherein at least 1 N-ring heteroatom is meta relative to the point of attachment, and the Ring A further comprises an oxo (═O) group para relative to the point of attachment.

In some embodiments, the compound of Formula (I) or a pharmaceutically acceptable salt thereof, is the compound wherein Ring A comprises 1 additional N-ring heteroatom.

In some embodiments, the compound of Formula (I) is a compound of Example 1, Example 2, Example 3, Example 4, Example 5, Example 6, Example 7, Example 8, Example 9, Example 10, or Example 11, or a pharmaceutically acceptable salt thereof. In some embodiments, the compound of Formula (I) is a compound of Table G, Table H, Table I, Table J, Table K, Table L1, Table L2, Table M, or Table N (as provided in the Examples), or a pharmaceutically acceptable salt thereof.

The compounds as described herein may exist as salts. The present disclosure includes such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds as described herein contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds as described herein contain both basic and acidic functionalities that allow the compounds to be converted into either base or acid addition salts.

Other salts include acid or base salts of the compounds used in the methods as described herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts includes salts of the active compounds which are prepared with relatively nontoxic acids or bases, depending on the particular substituents found on the compounds described herein. When compounds as described herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds as described herein contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes as described herein.

Certain compounds as described herein possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope as described herein. The compounds as described herein do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, the compounds as described herein may also contain unnatural proportions of atomic isotopes at one or more of the atoms that constitute such compounds. For example, the compounds as described herein may be labeled with radioactive or stable isotopes, such as for example deuterium (²H), tritium (3H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds as described herein, whether radioactive or not, are encompassed within the scope as described herein.

### IV. Pharmaceutical Formulations

In some embodiments, provided is a pharmaceutical composition comprising a compound of any one of the compounds as described herein and a pharmaceutically acceptable excipient.

In some embodiments, provided is a pharmaceutical composition comprising a compound of Formula (I) or a pharmaceutically acceptable salt thereof, and a pharmaceutically acceptable excipient.

The compounds as described herein can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds as described herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds as described herein can be administered transdermally. The compounds as described herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). The present disclosure also provides pharmaceutical compositions including one or more pharmaceutically acceptable carriers and/or excipients and either a compound of the present disclosure, or a pharmaceutically acceptable salt of a compound of the present disclosure.

For preparing pharmaceutical compositions from the compounds as described herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical compositions as described herein can also be used orally using, for example, push-fit capsules made of gelatin, as well as soft, sealed capsules made of gelatin and a coating such as glycerol or sorbitol. Push-fit capsules can contain the compounds of the present disclosure mixed with a filler or binders such as lactose or starches, lubricants such as talc or magnesium stearate, and, optionally, stabilizers. In soft capsules, the compounds of the present disclosure may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present disclosure in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations as described herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical compositions can be delivered by transdermally, by a topical route, formulated as applicator sticks, solutions, suspensions, emulsions, gels, creams, ointments, pastes, jellies, paints, powders, and aerosols.

The pharmaceutical compositions can also be delivered as microspheres for slow release in the body. For example, microspheres can be administered via intradermal injection of drug—containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The compounds of the present disclosure can be provided in a pharmaceutical composition as a salt formed with an acid, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The compounds of the present disclosure of the invention can be provided in a pharmaceutical composition as a salt formed with a base, including but not limited to cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

In some embodiments, the pharmaceutical composition can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical compositions is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR and/or MR modulator and disease or condition treated.

Single or multiple administrations of the pharmaceutical composition can be administered depending on the dosage and frequency as required and tolerated by the patient. The composition should provide a sufficient quantity of active agent to effectively treat the disease state. Thus, in some embodiments, the pharmaceutical composition for oral administration of the compound of the present disclosure is administered between about 0.5 to about 30 mg per kilogram of body weight per day. In some embodiments, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of the present disclosure for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents, or with adjunctive agents that may not be effective alone but may contribute to the efficacy of the active agent, in a single pharmaceutical composition (by co-formulation) or separate pharmaceutical compositions.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a pharmaceutical composition has been formulated in one or more acceptable carriers, it can be placed in an appropriate container and labeled for treatment of an indicated condition. For administration of the compounds of the present disclosure, such labeling would include, e.g., instructions concerning the amount, frequency and method of administration.

In some embodiments, the compositions of the present disclosure are useful for parenteral administration, such as intravenous (IV) administration or administration into a body cavity or lumen of an organ. The pharmaceutical compositions for administration will commonly comprise a solution of the compositions of the present disclosure dissolved in one or more pharmaceutically acceptable carriers. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the pharmaceutical composition can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### V. Methods

In some embodiments, provided is a method of treating a disorder or condition in a human in need thereof, the method comprising administering to the human a therapeutically effective amount of a compound as described herein, or a pharmaceutically acceptable salt thereof, or a pharmaceutical composition as described herein.

In some embodiments, provided is a method of treating a CDK2-mediated disorder in a human in need thereof, the method comprising administering to the human a therapeutically effective amount of a compound of Formula (I) or a pharmaceutically acceptable salt thereof, or a pharmaceutical composition as described herein.

A method for manufacturing a medicament for treating a CDK2-mediated disorder in a human in need thereof, characterized in that the compound of Formula (I), or a pharmaceutically acceptable salt thereof, or a pharmaceutical composition of any one as described herein, is used.

Use of a compound of Formula (I) or a pharmaceutically acceptable salt thereof, or a pharmaceutical composition as described herein, for the manufacture of a medicament for the treatment in a human of a CDK2-mediated disorder.

The compound of Formula (I) or a pharmaceutically acceptable salt thereof, or a pharmaceutical composition as described herein, for use in the treatment of a CDK2-mediated disorder in a human in need thereof.

In some embodiments, the disorder is cancer. Exemplary cancers include, but are not limited to, leukemia (e.g., acute myelocytic leukemia), bladder cancer, brain cancer, breast cancer (e.g., hormone receptor positive breast cancer, triple negative breast cancer, HER2+ breast cancer), cervical cancer, colorectal cancer (e.g., including colon cancer and/or rectal cancer), endometrial cancer, esophageal cancer, gastric cancer (e.g. stomach adenocarcinoma), kidney cancer (e.g., renal cell carcinoma), liver cancer (e.g., hepatocellular cancer), lung cancer (e.g., non-small cell lung cancer, small cell lung cancer), neuroblastoma, ovarian cancer (e.g., serous ovarian cancer), prostate cancer, skin cancer (e.g., melanoma), thyroid cancer, and uterine cancer (e.g., uterine carcinosarcoma).

In certain embodiments, the cancer is ovarian cancer, gastric cancer, uterine cancer, esophageal cancer, lung cancer, or breast cancer. In certain embodiments, the cancer is ovarian cancer. In certain embodiments, the cancer is gastric cancer. In certain embodiments, the cancer is uterine cancer. In certain embodiments, the cancer is esophageal cancer. In certain embodiments, the cancer is lung cancer. In certain embodiments, the cancer is breast cancer.

G1/s-specific cyclin-E1 is encoded by the CCNE1 gene, and since CDK2 drives the G1/S transition, CDK2 inhibition would be useful in the treatment of cancers having CCNE1 amplification or overexpression. Exemplary cancers with CCNE1 amplification or overexpression include, but are not limited to, ovarian cancer, gastric cancer, uterine cancer, esophageal cancer, and breast cancer. In certain embodiments, the cancer is breast cancer having CCNE1 amplification or overexpression. In certain embodiments, the breast cancer having CCNE1 amplification or overexpression is hormone receptor positive (HR+).

In certain embodiments, the cancer is breast cancer. In certain embodiments, the breast cancer is HR+, and the subject in need thereof has progressed on CDK4/6 inhibitors. In certain embodiments, the breast cancer is HER2+ and the subject has progressed on trastuzumab.

In certain embodiments, the cancer is lung cancer. In certain embodiments, the cancer is non-small cell lung cancer (NSCLC), and the subject has progressed on an epidermal growth factor receptor (EGFR) inhibitor.

In certain embodiments, treatment may be administered after one or more symptoms have developed. Treatment may also be continued after symptoms have resolved, for example to prevent or delay their recurrence.

The compounds as described herein (e.g., of any one of Formula (I) and (Ia)-(Ij)), or a pharmaceutically acceptable salt thereof), are inhibitors of cyclin-dependent kinase 2 (CDK2). For example, the inhibition constant (Ki) of the compounds as described herein can be less than about 50 μM, or less than about 40, 30, 20, 10, 9, 8, 7, 6, 5, 4, 3, 2, or less than about 1 μM. The inhibition constant (Ki) of the compounds as described herein can be less than about 1,000 nM, or less than about 900, 800, 700, 600, 500, 400, 300, 200, 100, 90, 80, 70, 60, 50, 40, 30, 20, 10, 9, 8, 7, 6, 5, 4, 3, 2, or less than about 1 nM. The inhibition constant (Ki) of the compounds as described herein can be less than about 1 nM, or less than about 0.9, 0.8, 0.7, 0.6, 0.5, 0.4, 0.3, 0.2, or less than about 0.1 nM. as described herein.

The compounds as described herein (e.g., of any one of Formula (I), and (Ia)-(Ij)), or a pharmaceutically acceptable salt thereof), can be selective inhibitors of cyclin-dependent kinase 2 (CDK2). For example, CDK2 inhibition constant (Ki) of the compounds as described herein can be at least 2-fold less than the inhibition constant of one or more of CDK1, CDK4 and CDK6, or at least 3, 4, 5, 6, 7, 8, 9, 10, 20, 30, 40, 50, 60, 70, 80, 90 or 100-fold less. The CDK2 inhibition constant (Ki) of the compounds as described herein can also be at least 100-fold less than the inhibition constant of one or more of CDK1, CDK4 and CDK6, or at least 200, 300, 400, 500, 600, 700, 800, 900, 1000, or 10,000-fold less.

### a) Cancer Combination Therapies

The compounds as described herein or pharmaceutically acceptable salts thereof may be employed alone or in combination with other agents for treatment. For example, the second agent of the pharmaceutical combination formulation or dosing regimen may have complementary activities to the compound as described herein such that they do not adversely affect each other. The compounds may be administered together in a unitary pharmaceutical composition or separately. In one embodiment a compound or a pharmaceutically acceptable salt can be co-administered with a chemotherapeutic agent to treat proliferative diseases and cancer.

The term “co-administering” refers to either simultaneous administration, or any manner of separate sequential administration, of a compound as described herein or a salt thereof, and a further active pharmaceutical ingredient or ingredients, including a chemotherapeutic agent. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound as described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound as described herein may be administered with another therapeutic agent simultaneously or sequentially in separate unit dosage forms or together in a single unit dosage form. Accordingly, provided is a single unit dosage form comprising a compound of Formula I, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In some embodiments, compositions as described herein are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In some embodiments, the treatment method includes the co-administration of a compound as described herein or a pharmaceutically acceptable salt thereof and at least one chemotherapeutic agent.

The term “chemotherapeutic agent” is an agent useful in the treatment of cancer, and includes, but is not limited to, cytotoxic agents such as radioactive isotopes (e.g., At²¹¹, I¹¹³, I¹²⁵, Y⁹⁰, Re¹⁸⁶, Re¹⁸⁸, Sm¹⁵³, Bi²²¹, P³², Pb²¹² and radioactive isotopes of Lu); growth inhibitory agents; anti-microtubule agents; platinum analogs; topoisomerase II inhibitors; anti-metabolites; topoisomerase I inhibitors; hormones and hormonal analogues; signal transduction pathway inhibitors; non-receptor tyrosine kinase angiogenesis inhibitors; immunotherapeutic agents; proapoptotic agents; cell cycle signalling inhibitors; nitrogen mustards; alkylating agents; toxoids or taxanes; aromatase inhibitors; chromoprotein enediyne antibiotic chromophores; mitomycins; anti-hormonal agents; anti-androgens; protein kinase inhibitors; lipid kinase inhibitors; antisense oligonucleotides; tyrosine kinase inhibitors; Raf-1 inhibitors; EGFR inhibitors; camptothecins; anthracycline antibiotics; nucleoside metabolic inhibitors, and other CDK inhibitors.

Exemplary chemotherapeutic agents include, but are not limited to, EGFR inhibitors such as lapatinib (TYKERB®, GSK572016, Glaxo Smith Kline) or gefitinib (IRESSA®, AstraZeneca); alkylating agents such as thiotepa, CYTOXAN® cyclosphosphamide, or chloranmbucil; camptothecins such as topotecan and irinotecan; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, or uracil mustard; neocarzinostatin chromophore and related chromoprotein enediyne antibiotic chromophores; anthracycline antibiotics such as ADRIAMYCIN® (doxorubicin); mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, or zorubicin; anti-metabolites such as methotrexate, 5-fluorouracil (5-FU), or hydroxyurea; toxoids or taxanes such as TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); nucleoside metabolic inhibitor such as GEMZAR® (gemcitabine); platinum analogs such as cisplatin, carboplatin, oxaliplatin (ELOXATIN®, Sanofi); etoposide (VP-16); capecitabine (XELODA*); retinoids such as retinoic acid; anti-hormonal agents that act to regulate or inhibit hormone action on tumors such as anti-estrogens and selective estrogen receptor modulators/degraders (SERDs), such as tamoxifen (including NOLVADEX®; tamoxifen citrate), raloxifene, droloxifene, iodoxyfene, 4-hydroxytamoxifen, trioxifene, keoxifene, LY117018, onapristone, FARESTON® (toremifine citrate), fulvestrant, brilanestrant, elacestrant or giredestrant (GDC-9545); aromatase inhibitors that inhibit the enzyme aromatase, which regulates estrogen production in the adrenal glands, such as letrozole, exemestane, anastozole, aminoglutethimide, MEGASE® (megestrol acetate), formestanie, fadrozole, or RIVISOR® (vorozole); anti-androgens such as flutamide, nilutamide, bicalutamide, leuprolide goserelin, buserelin, tripterelin, medroxyprogesterone acetate, diethylstilbestrol, premarin, fluoxymesterone, all-trans retionic acid, or fenretinide; antisense oligonucleotides which inhibit expression of genes in signaling pathways implicated in aberrant cell proliferation, such as PKC-alpha, Ralf and H-Ras; tyrosine kinase inhibitors such as erlotinib (TARCEVA®, Genentech/OSI Pharm.); CDK4/6 inhibitors such as palbociclib, ribociclib or abemaciclib; and antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia) gemtuzumab ozogamicin (MYLOTARG®, Wyeth), and antibody conjugates thereof.

Additionally, chemotherapeutic agents include pharmaceutically acceptable salts, acids or derivatives of any of chemotherapeutic agents, described herein, as well as combinations of two or more of them.

In some embodiments, the compounds as described herein can be administered in combination with CDK4/6 inhibitors such as palbociclib, ribociclib or abemaciclib, e.g., for the treatment of breast cancer.

In other embodiments, the compounds as described herein can be administered with aromatase inhibitors such as letrozole, exemestane or anastozole, e.g., for the treatment of breast cancer.

In still other embodiments, the compounds as described herein can be administered in combination with selective estrogen receptor degraders (SERD) such as fulvestrant, brilanestrant, elacestrant or giredestrant (GDC-9545), e.g., for the treatment of breast cancer.

In some embodiments, the compounds as described herein can be administered in combination with a CDK4/6 inhibitor and a selective estrogen receptor degrader (SERD), e.g., for the treatment of breast cancer (e.g., HR2+ breast cancer).

In yet other embodiments, the compounds as described herein can be administered in combination with an antibody such as trastuzumab (HERCEPTIN®, Genentech), e.g., for the treatment of breast cancer (e.g., HER+ breast cancer).

In yet other embodiments, the compounds as described herein can be administered in combination with toxoids or taxanes (such as paclitaxel, albumin-engineered nanoparticle formulations of paclitaxel, and docetaxel/doxetaxel) and/or platinum analogs (such as cisplatin, carboplatin, and oxaliplatin), e.g., for the treatment of ovarian cancer.

### VI. Examples

### General Experimental Details

All solvents and commercial reagents were used as received unless otherwise stated. Where products were purified by chromatography on silica gel this was carried out using either a glass column manually packed with silica gel (Kieselgel 60, 220-440 mesh, 35-75 μm) or an Isolute SPE Si II cartridge. ‘Isolute SPE Si cartridge’ refers to a pre-packed polypropylene column containing unbonded activated silica with irregular particles with average size of 50 μm and nominal 60 Å (angstrom) porosity. Where an Isolute® SCX2 cartridge was used, ‘Isolute® SCX-2 cartridge’ refers to a pre-packed polypropylene column containing a non-end-capped propylsulphonic acid functionalized silica strong cation exchange sorbent.

### Nuclear Magnetic Resonance Spectroscopy (NMR) Analytical Methods

¹H NMR spectra were recorded at ambient temperature, unless otherwise specified, using one of the following: Bruker AVIII 400 MHz with 2 RF channels, 5 mm Prodigy BBI (Broadband Inverse Ag-P detectable) probe; Bruker Avance 400 MHz with 2 RF channels, 5 mm Prodigy BBFO (Broadband Inverse Ag-P detectable) probe; Bruker Nanobay 400 MHz, 5 mm Prodigy BBFO probe. br=broad; s=singlet; d=doublet; t=triplet; q=quartet; m=multiplet.

### High Pressure Liquid Chromatography/Liquid Chromatography-Mass Spectrometry (HPLC/LC-MS) Analytical Methods

HPLC/LC-MS chromatograms were recorded using one of the following instruments and conditions.

### LC-MS Method 1

Shimadzu LCMS-2020, Waters Acquity BEH C18-reverse-phase column (50 mm×2.1 mm×1.7 μm), elution with A: water+0.1% formic acid; B: acetonitrile; Detection: MS, ELS, UV (100 μL split to MS with in-line UV detector); MS ionization method: Electrospray (positive and negative ion). ES-API=electrospray-atmospheric pressure ionization.

TABLE A
Gradient

| Time | Flow (mL/min) | % A | % B |
|---|---|---|---|
| 0 | 0.8 | 95 | 5 |
| 1.6 | 0.8 | 0 | 100 |
| 1.8 | 0.8 | 0 | 100 |
| 2.0 | 0.8 | 95 | 5 |

### LC-MS Method 2

Shimadzu LCMS-2020, Waters Acquity BEH C18-reverse-phase column (50 mm×2.1 mm×1.7 μm), elution with A: water+0.1% trifluoroacetic acid; B: acetonitrile; Detection: MS, ELS, UV (100 μL split to MS with in-line UV detector); MS ionization method: Electrospray (positive and negative ion). ES-API=electrospray-atmospheric pressure ionization.

TABLE B
Gradient

| Time | Flow (mL/min) | % A | % B |
|---|---|---|---|
| 0 | 0.8 | 95 | 5 |
| 1.6 | 0.8 | 0 | 100 |
| 1.8 | 0.8 | 0 | 100 |
| 2.0 | 0.8 | 95 | 5 |

### LC-MS Method 3

Shimadzu LCMS-2020, Waters Acquity BEH C18-reverse-phase column (50 mm×2.1 mm×1.7 μm), elution with A: water+0.1% ammonium hydroxide; B: acetonitrile; Detection: MS, ELS, UV (100 μL split to MS with in-line UV detector); MS ionization method: Electrospray (positive and negative ion). ES-API=electrospray-atmospheric pressure ionization.

TABLE C
Gradient

| Time | Flow (mL/min) | % A | % B |
|---|---|---|---|
| 0 | 0.8 | 95 | 5 |
| 1.6 | 0.8 | 0 | 100 |
| 1.8 | 0.8 | 0 | 100 |
| 2.0 | 0.8 | 95 | 5 |

### LC-MS Method 4

Agilent 1290 UHPLC; Phenomenex XB C18 column (50 mm×2.1 mm×1.7 μm), elution with A: water+0.1% formic acid; B: acetonitrile+0.1% formic acid; Detection: MS, ELS, UV (100 μL split to MS with in-line UV detector) at 220 nm and 254 nm; MS ionization method: Electrospray (positive and negative ion). ES-API=electrospray-atmospheric pressure ionization.

TABLE D
Gradient

| Time | Flow (mL/min) | % A | % B |
|---|---|---|---|
| 0 | 0.4 | 98 | 2 |
| 7.0 | 0.4 | 2 | 98 |
| 8.5 | 0.4 | 2 | 98 |
| 10.0 | 0.4 | 98 | 2 |

### LC-MS Method 5

Agilent 1290 UHPLC; Agilent Zorbax Eclipse XDB C18 column (100 mm×3.0 mm×3.5 μm), elution with A: water+0.1% formic acid; B: acetonitrile+0.1% formic acid; Detection: MS, ELS, UV (100 μL split to MS with in-line UV detector) at 220 nm and 254 nm; MS ionization method: Electrospray (positive and negative ion). ES-API=electrospray-atmospheric pressure ionization.

TABLE E
Gradient

| Time | Flow (mL/min) | % A | % B |
|---|---|---|---|
| 0 | 0.7 | 98 | 2 |
| 25.5 | 0.7 | 2 | 98 |
| 28.0 | 0.7 | 2 | 98 |
| 29.5 | 0.7 | 98 | 2 |

### HPLC Purification Procedures

TABLE F
HPLC Methods

| # | Method |
|---|---|
| A | Instrument = Interchim BH; |
| | Column = Boston Prime C18; |
| | Column dimensions = 80 mm × 40 mm × 3 μm; |
| | Detection wavelength = 250 nM; |
| | Flow rate = 30 mL/min; |
| | Run time = 8 min; |
| | Column temperature = 25° C. |
| B | Instrument = Interchim DH; |
| | Column = Boston Prime C18; |
| | Column dimensions = 75 mm × 30 mm × 3 μm; |
| | Detection wavelength = 250 nM; |
| | Flow rate = 25 mL/min; |
| | Run time = 7 min; |
| | Column temperature = 25° C. |
| C | Instrument = Interchim HPLC; |
| | Column = Phenomenex C18; |
| | Column dimensions = 75 × 30 mm × 3 um; |
| | Detection wavelength = 220 nm; |
| | Flow rate = 25 mL/min; |
| | Run time = 7 min; |
| | Column temperature = 25° C. |
| D | Instrument = DA; |
| | Column = Boston Green ODS; |
| | Column dimensions = 150 * 30 mm * 5 μm; |
| | Detection wavelength = 220 nm; |
| | Flow rate = 35 mL/min; |
| | Run time = 6 min; |
| | Column temperature = 25° C. |
| E | Instrument = AA; |
| | Column = Boston Green ODS; |
| | Welch Xtimate C18 = 150 * 25 mm * 5 μm; |
| | Detection wavelength = 220 nm; |
| | Flow rate = 25 mL/min; |
| | Run time = 6 min; |
| | Column temperature = 25° C. |
| F | Column = Daicel CHIRALPAK IG; |
| | Column dimensions = 250 mm × 30 mm × 10 μm; |
| | Detection wavelength = 250 nM; |
| | Flow rate = 70 mL/min; |
| | Run time = 7 min; |
| | Column temperature = 25° C. |
| G | Instrument = BH; |
| | Column = Phenomenex C18; |
| | Column dimensions = 80 mm × 40 mm × 3 μm; |
| | Detection wavelength = 250 nM; |
| | Flow rate = 30 mL/min; |
| | Run time = 8 min; |
| | Column temperature = 25° C. |
| H | Instrument = Interchim HPLC; |
| | Column = XSelect CSH Prep C18; |
| | Column dimensions = 50 × 30 mm, 5 μm; |
| | Detection wavelength = 260 nm; |
| | Flow rate = 60 mL/min; |
| | Run time = 10 min; |
| | Column temperature = 25° C. |
| I | Instrument = Interchim HPLC; |
| | Column = Gemini-NX C18; |
| | Column dimensions = 50 × 30 mm, 5 μm; |
| | Detection wavelength = 280 nm; |
| | Flow rate = 60 mL/min; |
| | Run time = 10 min; |
| | Column temperature = 25° C. |
| J | Instrument = Interchim BH; |
| | Column = Welch Xtimate C18; |
| | Column dimensions = 150 mm × 30 mm × 5 μm; |
| | Detection wavelength = 250 nM; |
| | Flow rate = 30 mL/min; |
| | Run time = 8 min; |
| | Column temperature = 25° C. |
| K | Instrument = Interchim BH; |
| | Column = Boston Green ODS; |
| | Column dimensions = 150 mm × 30 mm × 5 μm; |
| | Detection wavelength = 250 nM; |
| | Flow rate = 35 mL/min; |
| | Run time = 8 min; |
| | Column temperature = 25° C. |
| L | Instrument = Interchim DH; |
| | Column = Phenomenex Gemini-NX C18; |
| | Column dimensions = 75 mm × 30 mm × 3 μm; |
| | Detection wavelength = 250 nM; |
| | Flow rate = 25 mL/min; |
| | Run time = 6 min; |
| | Column temperature = 25° C. |
| M | Instrument = SFC 17; |
| | Column = Phenomenex-Cellulose-2 |
| | dimensions = 250 mm × 30 mm × 5 μm; |
| | Flow rate = 70 mL/min; |
| | Column temperature = 25° C. |
| N | Instrument = Interchim HPLC; |
| | Column = Phenomenex Gemini-NX C18; |
| | Column dimensions = 75 mm × 30 mm × 3 μm; |
| | Detection wavelength = 250 nM; |
| | Flow rate = 25 mL/min; |
| | Run time = 6 min; |
| | Column temperature = 25° C. |
| O | Instrument = Interchim HPLC; |
| | Column = Boston Prime C18; |
| | Column dimensions = 150 mm × 30 mm × 5 μm; |
| | Detection wavelength = 250 nM; |
| | Flow rate = 60 mL/min; |
| | Run time = 8 min; |
| | Column temperature = 25° C. |
| P | Column = DAICEL CHIRALPAK AD; |
| | Column dimensions = 250 mm × 30 mm × 10 μm; |
| | Detection wavelength = 250 nM; |
| | Flow rate = 80 mL/min; |
| | Run time = 8 min; |
| | Column temperature = 25° C. |
| Q | Instrument = Interchim HPLC; |
| | Column = Phenomenex C18; |
| | Column dimensions = 75 × 30 mm × 3 um; |
| | Detection wavelength = 220 nm; |
| | Flow rate = 25 mL/min; |
| | Run time = 7 min; |
| | Column temperature = 25° C. |
| R | Instrument = Interchim HPLC; |
| | Column = Phenomenex C18; |
| | Column dimensions = 75 × 30 mm × 3 um; |
| | Detection wavelength = 220 nm; |
| | Flow rate = 25 mL/min; |
| | Run time = 7 min; |
| | Column temperature = 25° C. |
| S | Instrument = Interchim HPLC; |
| | Column = Boston Prime C18; |
| | Column dimensions = 150 mm × 30 mm × 5 μm; |
| | Detection wavelength = 250 nM; |
| | Flow rate = 25 mL/min; |
| | Run time = 7 min; |
| | Column temperature = 25° C. |
| T | Column = DAICEL CHIRALAPK AD; |
| | Column dimensions = 250 mm × 30 mm × 10 μm; |
| | Flow rate = 70 mL/min; |
| | Column temperature = 35° C. |
| U | Instrument = Interchim HPLC; |
| | Column = Boston Prime C18; |
| | Column dimensions = 250 mm × 50 mm × 10 μm; |
| | Detection wavelength = 250 nM; |
| | Flow rate = 60 mL/min; |
| | Run time = 8 min; |
| | Column temperature = 25° C. |

### PREPARATION OF INTERMEDIATES

### Intermediate A. 3-bromo-5-(3-((tert-butyldimethylsilyl)oxy)cyclopentyl)-1-((2-(trimethylsilyl)ethoxy)methyl)-1H-pyrazole

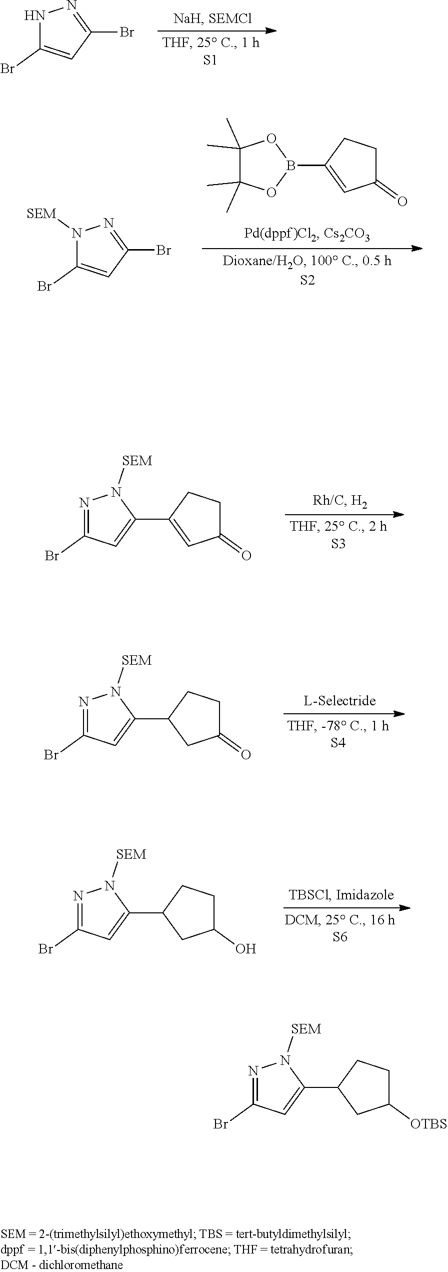

Step 1: 3,5-dibromo-1-((2-(trimethylsilyl)ethoxy)methyl)-1H-pyrazole. To a solution of 3,5-dibromo-1H-pyrazole (10.0 g, 44 mmol) in tetrahydrofuran (100 mL) was added sodium hydride (60%, 3.5 g, 89 mmol) slowly. The mixture was stirred at 20° C. for 30 min, and then 2-(trimethylsilyl)ethoxymethyl chloride (9.4 mL, 53 mmol) was added. The resulting mixture was stirred at 25° C. for 1 h and quenched by addition of water (200 mL). The mixture was extracted with ethyl acetate (3×100 mL). The combined organic layers were washed with brine (200 mL), dried over anhydrous sodium sulfate and concentrated under reduced pressure. The residue was purified by column chromatography (silica gel, 100-200 mesh, 0-5% ethyl acetate: petroleum ether) to afford 3,5-dibromo-1-((2-(trimethylsilyl)ethoxy)methyl)-1H-pyrazole (11.6 g, 73%). ¹H NMR (400 MHz, CDCl₃) δ 6.38-6.33 (s, 1H), 5.45 (s, 2H), 3.66-3.62 (m, 2H), 0.94-0.90 (m, 2H), 0.00 (s, 9H).

Step 2: 3-(3-bromo-1-((2-(trimethylsilyl)ethoxy)methyl)-1H-pyrazol-5-yl)cyclopent-2-enone. A mixture of 3,5-dibromo-1-((2-(trimethylsilyl)ethoxy)methyl)-1H-pyrazole (7.5 g, 21 mmol), 3-(4,4,5,5-tetramethyl-1,3,2-dioxaborolan-2-yl)cyclopent-2-enone (4.4 g, 21 mmol), cesium carbonate (20.9 g, 63 mmol) and 1,1′-bis(diphenylphosphino)ferrocene palladium dichloride (1.5 g, 2.2 mmol) in 1,4-dioxane (150 mL) and water (30 mL) was stirred at 100° C. under nitrogen atmosphere for 30 min. The mixture was cooled to ambient temperature and diluted with ethyl acetate (400 mL), washed with brine (400 mL), dried over anhydrous sodium sulfate and concentrated under reduced pressure. The residue was purified by column chromatography (silica gel, 100-200 mesh, 20% ethyl acetate: petroleum ether) to afford 3-(3-bromo-1-((2-(trimethylsilyl)ethoxy)methyl)-1H-pyrazol-5-yl)cyclopent-2-enone (3.6 g, 48%). LCMS (ES-API, m/z): 357.1 [M+H]+; ¹H NMR (400 MHz, CDCl₃) δ 6.73 (s, 1H), 6.66 (s, 1H), 5.50 (s, 2H), 3.68-3.64 (m, 2H), 2.99-2.97 (m, 2H), 2.57-2.55 (m, 2H), 0.92-0.88 (m, 2H), 0.00 (s, 9H).

Step 3: 3-(3-bromo-1-((2-(trimethylsilyl)ethoxy)methyl)-1H-pyrazol-5-yl)cyclopentanone. A mixture of 3-(3-bromo-1-((2-(trimethylsilyl)ethoxy)methyl)-1H-pyrazol-5-yl)cyclopent-2-enone (3.0 g, 8.4 mmol) and rhodium (10% on carbon, 3.4 g, 0.84 mmol) in tetrahydrofuran (30 mL) was stirred under hydrogen atmosphere (15 psi) at ambient temperature for 2 h and then filtered. The filtrate was concentrated under reduced pressure. The residue was purified by column chromatography (silica gel, 100-200 mesh, 0-20% ethyl acetate: petroleum ether) to afford 3-(3-bromo-1-((2-(trimethylsilyl)ethoxy)methyl)-1H-pyrazol-5-yl)cyclopentanone (2.3 g, 76%). ¹H NMR (400 MHz, CDCl₃): δ 6.17 (s, 1H), 5.43 (d, J=2.4 Hz, 2H), 3.60-3.56 (m, 3H), 2.74-2.69 (m, 1H), 2.49-2.46 (m, 2H), 2.34-2.28 (m, 2H), 2.04-2.02 (m, 1H), 0.91-0.87 (m, 2H), 0.00 (s, 9H).

Step 4: 3-(3-bromo-1-((2-(trimethylsilyl)ethoxy)methyl)-1H-pyrazol-5-yl)cyclopentanol. To a solution of 3-(3-bromo-1-((2-(trimethylsilyl)ethoxy)methyl)-1H-pyrazol-5-yl)cyclopentanone (2.3 g, 6.4 mmol) in tetrahydrofuran (40 mL) was added lithium tri-sec-butylborohydride (1.0 M in tetrahydrofuran, 7.7 mL, 7.7 mmol) at −78° C. under nitrogen atmosphere. The mixture was stirred at −78° C. for 2 h and subsequently poured into water (50 mL). The mixture was extracted with ethyl acetate (2×50 mL). The combined organic layers were dried over anhydrous sodium sulfate and concentrated under reduced pressure. The crude was purified by column chromatography (silica gel, 100-200 mesh, 0-30% ethyl acetate: petroleum ether) to afford 3-(3-bromo-1-((2-(trimethylsilyl)ethoxy)methyl)-1H-pyrazol-5-yl)cyclopentanol (2.1 g, 91%. LCMS (ES-API, m/z): 363.1 [M+H]⁺.

Step 5: 3-bromo-5-(3-((tert-butyldimethylsilyl)oxy)cyclopentyl)-1-((2-(trimethylsilyl)ethoxy)methyl)-1H-pyrazole. To solution of 3-(3-bromo-1-((2-(trimethylsilyl)ethoxy)methyl)-1H-pyrazol-5-yl)cyclopentanol (2.1 g, 5.8 mmol) and imidazole (1.6 g, 23 mmol) in dichloromethane (30 mL) was added tert-butyldimethylchlorosilane (1.3 g, 8.7 mmol). The mixture was stirred at 25° C. for 16 h and concentrated under reduced pressure. The residue was purified by column chromatography (silica gel, 100-200 mesh, 0-5% ethyl acetate:petroleum ether) to afford 3-bromo-5-(3-((tert-butyldimethylsilyl)oxy)cyclopentyl)-1-((2-(trimethylsilyl)ethoxy)methyl)-1H-pyrazole (2.3 g, 83%). LCMS (ES-API, m/z): 475.2 [M+H]⁺.

### Intermediate B. (1R,3S)-3-(5-amino-1-(tert-butyl)-1H-pyrazol-3-yl)cyclopentyl tert-butylcarbamate

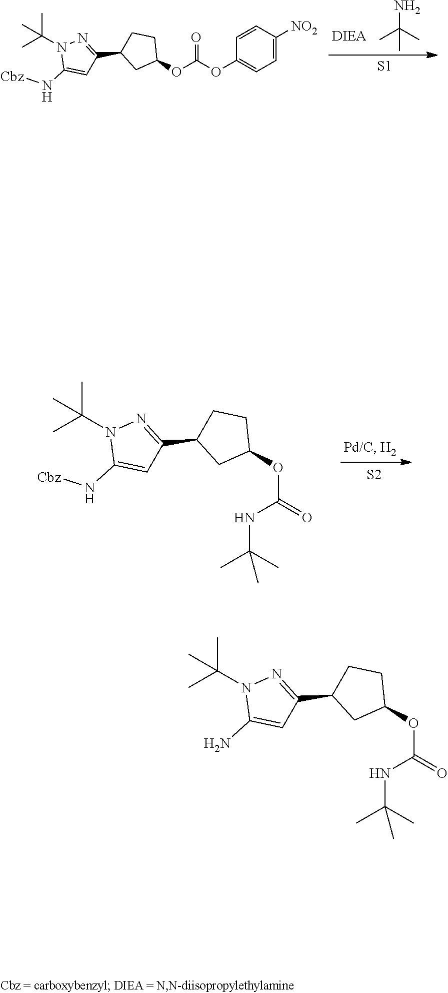

Step 1: benzyl (1-(tert-butyl)-3-((1S,3R)-3-((tert-butylcarbamoyl)oxy)-cyclopentyl)-1H-pyrazol-5-yl)carbamate. The starting material, (4-nitrophenyl) [(1R,3S)-3-[5-(benzyloxycarbonylamino)-1-tert-butyl-pyrazol-3-yl]cyclopentyl] carbonate, may be prepared following the procedure provided in Example 1 of PCT Publication WO 202157652. To a solution of (4-nitrophenyl) [(1R,3S)-3-[5-(benzyloxycarbonylamino)-1-tert-butyl-pyrazol-3-yl]cyclopentyl]carbonate (60 g, 114 mmol) in tetrahydrofuran (420 mL) was added N,N-diisopropylethylamine (74.2 g, 574 mmol) and 2-methylpropan-2-amine (10.9 g, 149 mmol). The mixture was stirred at 25° C. for 16 hours and concentrated under reduced pressure. The residue was purified by column chromatography (silica gel, 100-200 mesh, 30% ethyl acetate in petroleum ether) to give benzyl (1-(tert-butyl)-3-((1S,3R)-3-((tert-butylcarbamoyl)oxy)cyclopentyl)-1H-pyrazol-5-yl)carbamate (90 g, 84.9%).

Step 2: (1R,3S)-3-(5-amino-1-(tert-butyl)-1H-pyrazol-3-yl)cyclopentyl tert-butylcarbamate. To a solution of benzyl benzyl (1-(tert-butyl)-3-((1S,3R)-3-((tert-butylcarbamoyl)oxy)cyclopentyl)-1H-pyrazol-5-yl)carbamate (30 g, 65.7 mmol) in tetrahydrofuran (90 mL) and ethyl acetate (90 mL) was added Palladium (10% on carbon, 5.5 g). The mixture was stirred at 25° C. for 16 hours under hydrogen atmosphere (15 psi) and filtered. The filtrate was concentrated to dryness under reduced pressure to give (1R,3S)-3-(5-amino-1-(tert-butyl)-1H-pyrazol-3-yl)cyclopentyl tert-butylcarbamate (16 g, 74.1%). ¹H NMR (400 MHz, methanol-d4) δ 5.46 (s, 1H), 5.04 (br s, 1H), 3.05-2.92 (m, 1H), 2.50-2.37 (m, 1H), 2.04-1.65 (m, 5H), 1.61 (s, 9H), 1.36-1.25 (m, 9H).

### Intermediate C. (1R,3S)-3-(5-amino-1-(tert-butyl)-1H-pyrazol-3-yl)cyclopentanol

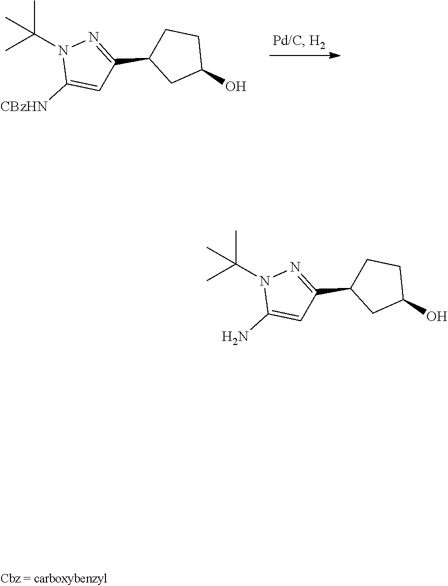

To a solution of benzyl N-[2-tert-butyl-5-[(1S,3R)-3-hydroxycyclopentyl]pyrazol-3-yl]carbamate (5.0 g, 13.99 mmol) in tetrahydrofuran (30 mL) and ethyl acetate (30 mL) was added Palladium (10% on carbon, 3.0 g). The mixture was stirred at 25° C. for 16 hours under hydrogen atmosphere (15 psi) and filtered. The filtrate was concentrated to dryness under reduced pressure to give (1R,3S)-3-(5-amino-1-(tert-butyl)-1H-pyrazol-3-yl)cyclopentanol (3.0 g, 96%). LCMS (ES-API, M/Z): 224.2 [M+H]⁺.

### GENERAL PROCEDURES AND EXAMPLES

The stereochemical configuration of the cyclopentanyl ring of Examples 2, 2L, 3E, 4, 4A, 4K, 4E was determined by X-ray crystallographic analysis. The configuration of all other examples was assigned by analogy.

### General Procedure 1

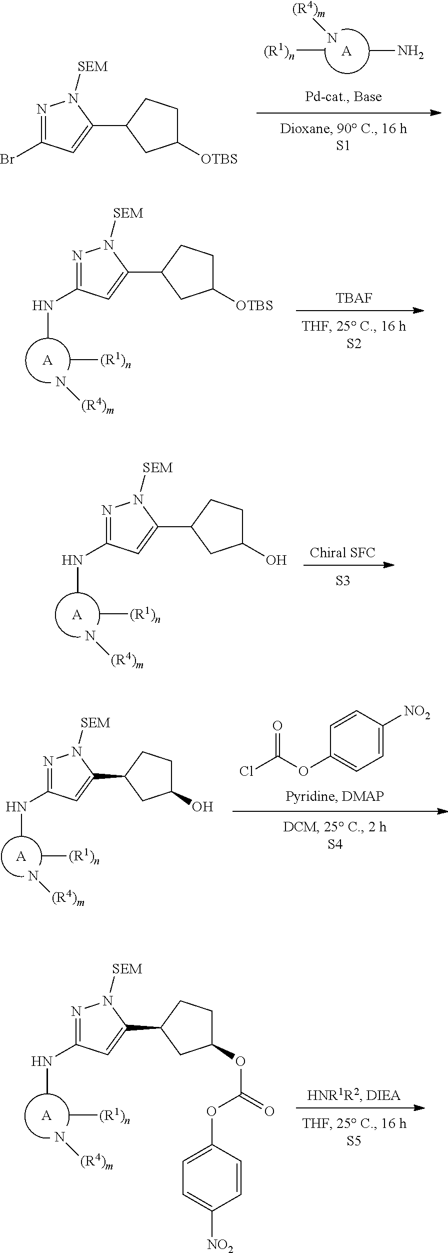

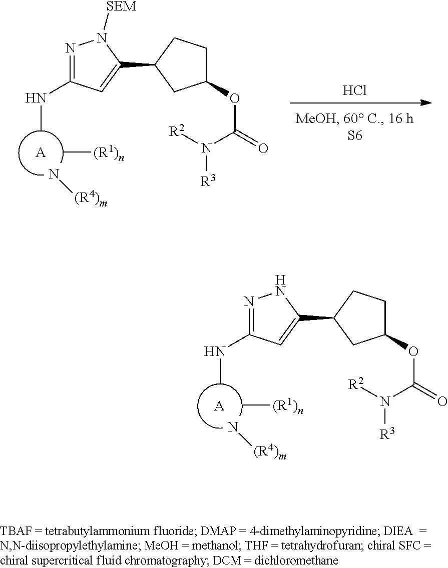

### Example 1. (1R,3S)-3-(3-((1-methyl-6-oxo-1,6-dihydropyridin-3-yl)amino)-1H-pyrazol-5-yl)cyclopentyl tert-butylcarbamate

The title compound was prepared according to General Procedure 1 as described below.

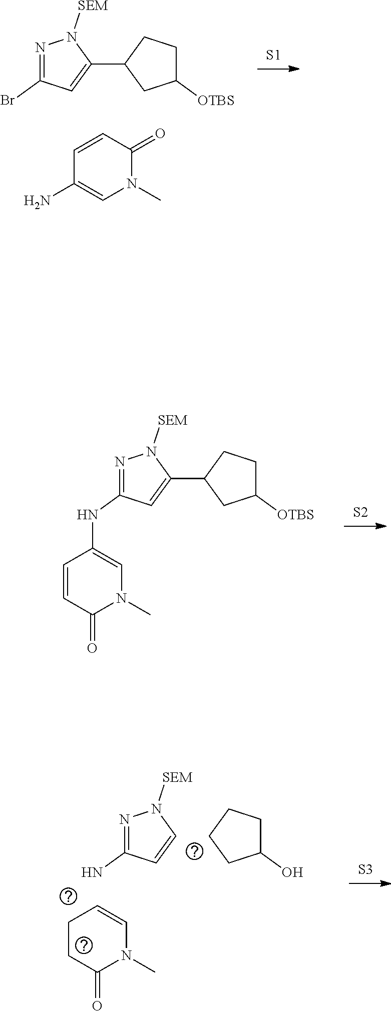

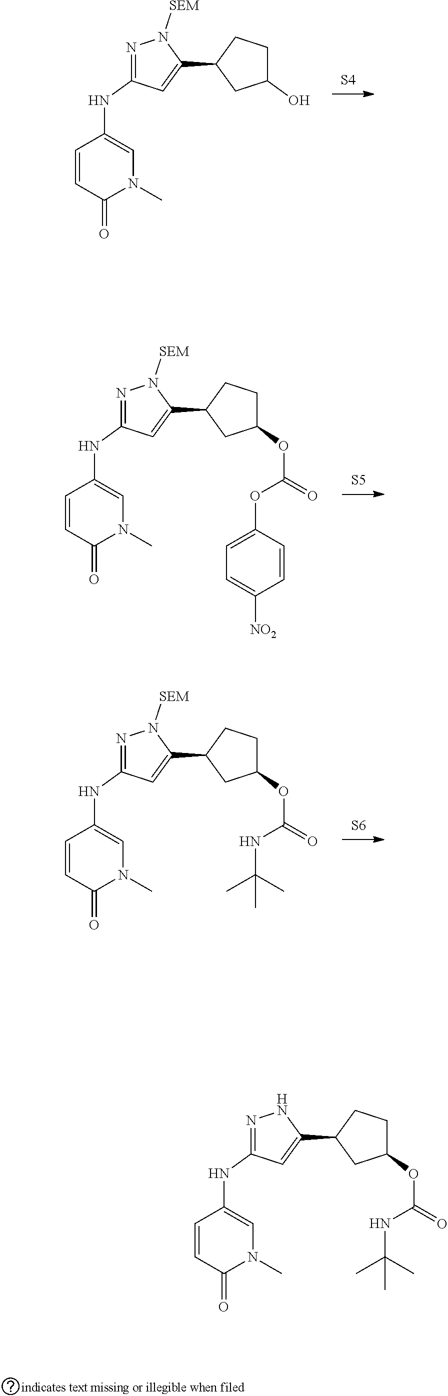

Step 1: 5-((5-(3-((tert-butyldimethylsilyl)oxy)cyclopentyl)-1-((2-(trimethylsilyl)ethoxy)methyl)-1H-pyrazol-3-yl)amino)-1-methylpyridin-2(1H)-one. A mixture of [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (180 mg, 0.21 mmol), 5-amino-1-methylpyridin-2(1H)-one (522 mg, 4.2 mmol), 3-bromo-5-(3-((tert-butyldimethylsilyl)oxy)cyclopentyl)-1-((2-(trimethylsilyl)ethoxy)methyl)-1H-pyrazole (Intermediate A) (1.00 g, 2.1 mmol) and cesium carbonate (2100 mg, 6.3 mmol) in 1,4-dioxane (15 mL) was stirred at 90° C. for 16 h under nitrogen atmosphere and then concentrated under reduced pressure. The residue was purified by column chromatography (silica gel, 100-200 mesh, 0-2% methanol:ethyl acetate) to obtain 5-((5-(3-((tert-butyldimethylsilyl)oxy)cyclopentyl)-1-((2-(trimethylsilyl)ethoxy) methyl)-1H-pyrazol-3-yl)amino)-1-methylpyridin-2(1H)-one (1.00 g, 92%). LCMS (ES-API, m/z): 519.3 [M+H]⁺.

Step 2: 5-((5-(3-hydroxycyclopentyl)-1-((2-(trimethylsilyl)ethoxy)methyl)-1H-pyrazol-3-yl)amino)-1-methylpyridin-2(1H)-one. To a solution of 5-((5-(3-((tert-butyldimethylsilyl)oxy)cyclopentyl)-1-((2-(trimethylsilyl)ethoxy)methyl)-1H-pyrazol-3-yl)amino)-1-methylpyridin-2(1H)-one (1.0 g, 1.9 mmol) in tetrahydrofuran (15 mL) was added tetrabutylammoniumfluoride (1.0 M in tetrahydrofuran, 2.9 mL, 2.9 mmol). The reaction was stirred at 25° C. for 16 h and concentrated under reduced pressure. The residue was purified by column chromatography (silica gel, 100-200 mesh, 0-5% methanol:dichloromethane) to obtain 5-((5-(3-hydroxycyclopentyl)-1-((2-(trimethylsilyl)ethoxy)methyl)-1H-pyrazol-3-yl)amino)-1-methylpyridin-2(1H)-one (750 mg, 96%). LCMS (ES-API, m/z): 405.3 [M+H]⁺.

Step 3: 5-((5-((1S,3R)-3-hydroxycyclopentyl)-1-((2-(trimethylsilyl)ethoxy)-methyl)-1H-pyrazol-3-yl)amino)-1-methylpyridin-2(1H)-one. A racemic mixture containing the title compound and its enantiomer (750 mg, 1.8 mmol) were purified by Purification Procedure F (chiral supercritical fluid chromatography (SFC)) with 0.1% ammonium hydroxide-30% ethanol-carbon dioxide to obtain 5-((5-((1S,3R)-3-hydroxycyclopentyl)-1-((2-(trimethylsilyl)ethoxy)methyl)-1H-pyrazol-3-yl)amino)-1-methylpyridin-2(1H)-one (Peak 1, retention time=4.912 min) (220 mg, 29%). LCMS (ES-API, M/Z): 405.3 [M+H]⁺.

Step 4: (1R,3S)-3-(3-((1-methyl-6-oxo-1,6-dihydropyridin-3-yl)amino)-1-((2-(trimethylsilyl)ethoxy)methyl)-1H-pyrazol-5-yl)cyclopentyl (4-nitrophenyl) carbonate. To a solution of 5-((5-((1S,3R)-3-hydroxycyclopentyl)-1-((2-(trimethylsilyl)ethoxy)methyl)-1H-pyrazol-3-yl)amino)-1-methylpyridin-2(1H)-one (160 mg, 0.40 mmol) in dichloromethane (4 mL) was added 4-nitrophenylchloroformate (199 mg, 0.99 mmol), pyridine (0.16 mL, 2.0 mmol) and 4-dimethylaminopyridine (4.8 mg, 0.04 mmol). The mixture was stirred at 25° C. for 2 h and concentrated under reduced pressure. The residue was purified by column chromatography (silica gel, 100-200 mesh, 0-5% methanol:ethyl acetate) to afford (1R,3S)-3-(3-((1-methyl-6-oxo-1,6-dihydropyridin-3-yl)amino)-1-((2-(trimethylsilyl) ethoxy)methyl)-1H-pyrazol-5-yl)cyclopentyl (4-nitrophenyl) carbonate (90 mg, 40%). LCMS (ES-API, m/z): 570.2 [M+H]⁺.

Step 5: (1R,3S)-3-(3-((1-methyl-6-oxo-1,6-dihydropyridin-3-yl)amino)-1-((2-(trimethylsilyl)ethoxy)methyl)-1H-pyrazol-5-yl)cyclopentyl tert-butylcarbamate. To a solution of (1R,3S)-3-(3-((1-methyl-6-oxo-1,6-dihydropyridin-3-yl)amino)-1-((2-(trimethylsilyl)ethoxy)methyl)-1H-pyrazol-5-yl)cyclopentyl (4-nitrophenyl) carbonate (90 mg, 0.16 mmol) in tetrahydrofuran (4 mL) were added N,N-diisopropylethylamine (0.08 mL, 0.5 mmol) and tert-butylamine (0.02 mL, 0.2 mmol). The reaction was stirred at 25° C. for 16 h and concentrated under reduced pressure. The residue was purified by preparative thin layer chromatography (TLC) (10% methanol:ethyl acetate) to obtain (1R,3S)-3-(3-((1-methyl-6-oxo-1,6-dihydropyridin-3-yl)amino)-1-((2-(trimethylsilyl)ethoxy)methyl)-1H-pyrazol-5-yl)cyclopentyl tert-butylcarbamate (60 mg, 75%). LCMS (ES-API, m/z): 504.3 [M+H]⁺.

Step 6: (1R,3S)-3-(3-((1-methyl-6-oxo-1,6-dihydropyridin-3-yl)amino)-1H-pyrazol-5-yl)cyclopentyl tert-butylcarbamate. To a solution of (1R,3S)-3-(3-((1-methyl-6-oxo-1,6-dihydropyridin-3-yl)amino)-1-((2-(trimethylsilyl)ethoxy)methyl)-1H-pyrazol-5-yl)cyclopentyl tert-butylcarbamate (40 mg, 0.08 mmol) in methanol (5 mL) was added hydrochloric acid (1.0 M in methanol, 2.0 mL, 2.0 mmol). The solution was stirred at 60° C. for 16 h and adjusted to pH=8 with aqueous ammonium hydroxide solution. The mixture was concentrated under reduced pressure. The residue was purified by Purification Procedure A with 0.05% ammonium hydroxide in water-acetonitrile (28-58%) to afford (1R,3S)-3-(3-((1-methyl-6-oxo-1,6-dihydropyridin-3-yl)amino)-1H-pyrazol-5-yl)cyclopentyl tert-butylcarbamate (5.9 mg, 19%). LCMS (ES-API, M/Z): 374.1 [M+H]⁺; ¹H NMR (400 MHz, DMSO-d₆): δ 11.59 (s, 1H), 7.94 (s, 1H), 7.86 (s, 1H), 7.33-7.30 (m, 1H), 6.77 (s, 1H), 6.34 (d, J=9.6 Hz, 1H), 5.47 (s, 1H), 4.95 (s, 1H), 3.38 (s, 3H), 3.02-2.98 (m, 1H), 2.41-2.38 (m, 1H), 1.99-1.87 (m, 2H), 1.71-1.55 (m, 3H), 1.20 (s, 9H).

### General Procedure 2

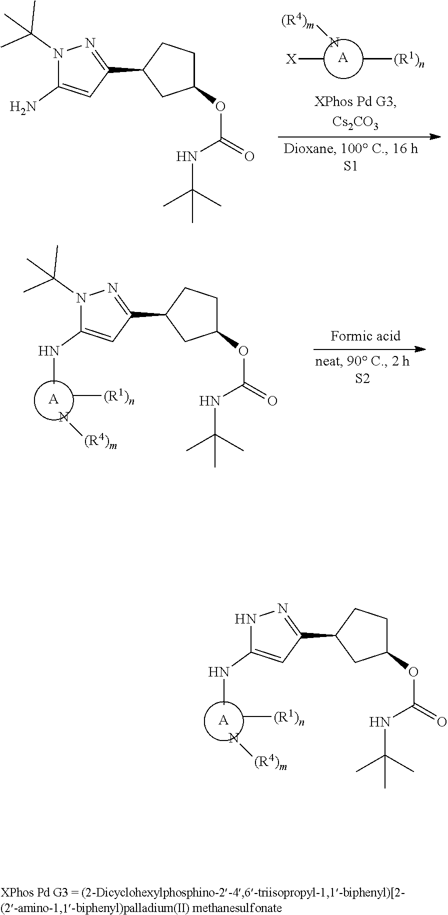

### Example 2. (1R,3S)-3-(3-((1-methyl-6-oxo-1,6-dihydropyridazin-3-yl)amino)-1H-pyrazol-5-yl)cyclopentyl isopropylcarbamate

The title compound was prepared according to General Procedure 2 as described below.

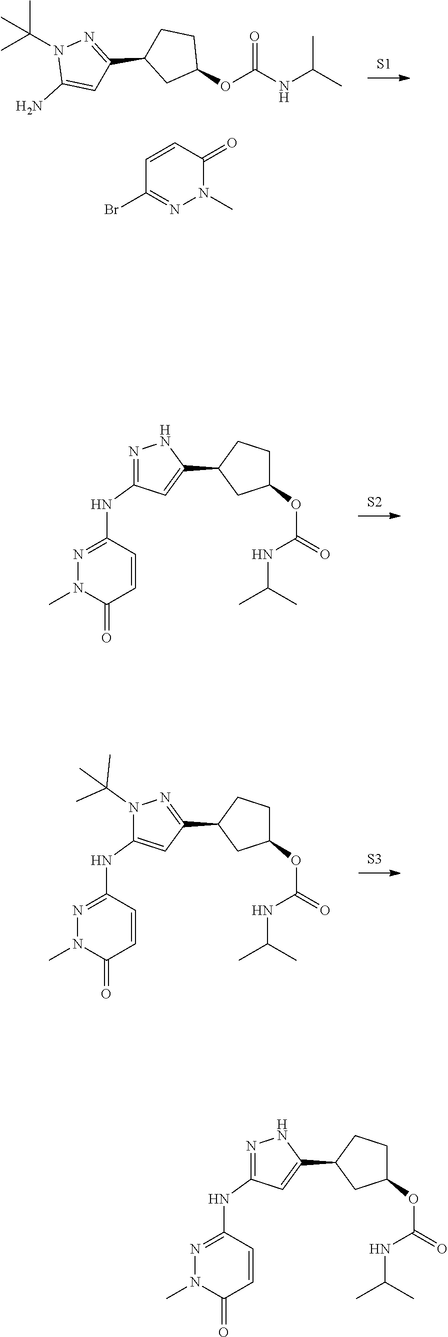

Step 1: (1R,3S)-3-(1-(tert-butyl)-5-((1-methyl-6-oxo-1,6-dihydropyridazin-3-yl)amino)-1H-pyrazol-3-yl)cyclopentyl isopropylcarbamate. 6-bromo-2-methyl-3(2H)-pyridazinone may be prepared following the procedure provided in Example 2 of PCT Publication WO 202157652.

A mixture of cesium carbonate (158 mg, 0.49 mmol), [(1R,3S)-3-(5-amino-1-tert-butyl-pyrazol-3-yl)cyclopentyl] N-isopropylcarbamate (50 mg, 0.16 mmol), methanesulfonato(2-dicyclohexylphosphino-2′,4′,6′-tri-isopropyl-1,1′-biphenyl)(2′-amino-1,1′-biphenyl-2-yl)palladium(II) (14 mg, 0.02 mmol) and 6-bromo-2-methyl-3(2H)-pyridazinone (61 mg, 0.32 mmol) in 1,4-dioxane (3 mL) was stirred at 100° C. for 16 h under nitrogen atmosphere and concentrated under reduced pressure. The residue was purified by preparative thin layer chromatography (TLC) (ethyl acetate) to afford (1R,3S)-3-(1-(tert-butyl)-5-((1-methyl-6-oxo-1,6-dihydropyridazin-3-yl)amino)-1H-pyrazol-3-yl)cyclopentyl isopropylcarbamate (53 mg, 80%). LCMS (ES-API, m/z): 417.3 [M+H]⁺.

Step 2: (1R,3S)-3-(3-((1-methyl-6-oxo-1,6-dihydropyridazin-3-yl)amino)-1H-pyrazol-5-yl)cyclopentyl isopropylcarbamate. A solution of (1R,3S)-3-(1-(tert-butyl)-5-((1-methyl-6-oxo-1,6-dihydropyridazin-3-yl)amino)-1H-pyrazol-3-yl)cyclopentyl isopropylcarbamate (80 mg, 0.19 mmol) in formic acid (3.7 mL) was stirred at 90° C. for 2 h and concentrated under reduced pressure. The residue was purified by Purification Procedure B with 0.05% ammonium hydroxide-10 mM ammonium bicarbonate in water-acetonitrile (30-60%) to afford (1R,3S)-3-(3-((1-methyl-6-oxo-1,6-dihydropyridazin-3-yl)amino)-1H-pyrazol-5-yl)cyclopentyl isopropylcarbamate (40 mg, 57%). LCMS (ES-API, m/z): 361.2 [M+H]⁺; ¹H NMR (400 MHz, DMSO-d₆) δ 11.91 (s, 1H), 9.18 (s, 1H), 7.30-7.28 (m, 1H), 6.96-6.94 (m, 1H), 6.82 (d, J=9.6 Hz, 1H), 6.23 (s, 1H), 4.99-4.98 (m, 1H), 3.60-3.55 (m, 1H), 3.52 (s, 3H), 3.34-3.04 (m, 1H), 2.47-2.44 (m, 1H), 2.01-1.90 (m, 2H), 1.72-1.60 (m, 3H), 1.02 (d, J=6.4 Hz, 6H).

### Example 2T: (1R,3S)-3-(3-((3-methylpyrazin-2-yl)amino)-1H-pyrazol-5-yl)cyclopentyl isopropylcarbamate

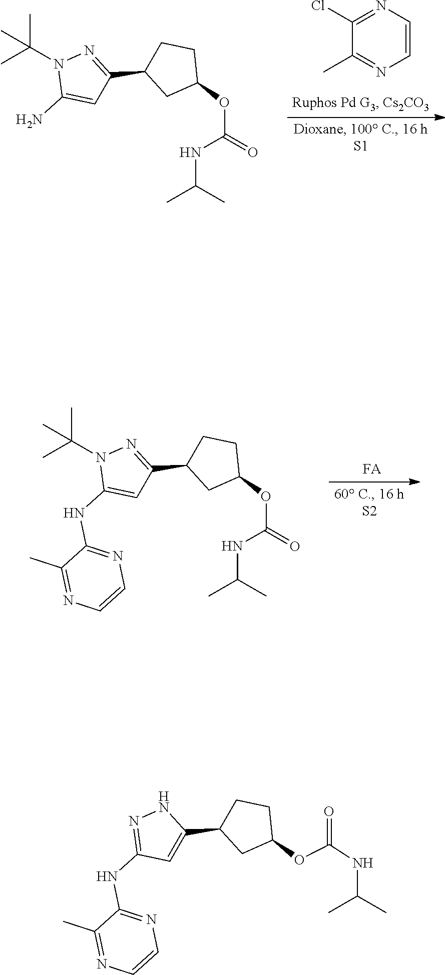

Step 1: (1R,3S)-3-(1-(tert-butyl)-5-((3-methylpyrazin-2-yl)amino)-1H-pyrazol-3-yl)cyclopentyl isopropylcarbamate. A mixture of [(1R,3S)-3-(5-amino-1-tert-butyl-pyrazol-3-yl)cyclopentyl] N-isopropylcarbamate (60 mg, 0.19 mmol), 2-chloro-3-methylpyrazine (30 mg, 0.23 mmol), cesium carbonate (190 mg, 0.58 mmol) and Methanesulfonato (2-dicyclohexylphosphino-2′,6′-di-i-propoxy-1,1′-biphenyl) (2′-aMino-1,1′-biphenyl-2-yl) palladium (II) (16 mg, 0.02 mmol) in 1,4-dioxane (3 mL) was stirred at 100° C. for 16 hours under nitrogen atmosphere. The mixture was added water (10 mL), then extracted with ethyl acetate (3×10 mL) and water (20 mL). The combined organic layers were washed with brine (15 mL), dried over anhydrous sodium sulfate, filtered and concentracted under reduced pressure. The residue was purified by preparative TLC (100% ethyl acetate in petroleum ether) to give (1R,3S)-3-(1-(tert-butyl)-5-((3-methylpyrazin-2-yl)amino)-1H-pyrazol-3-yl)cyclopentyl isopropylcarbamate (88 mg, 86%). LCMS (ES-API, m/z): 401.3 [M+H]⁺.

Step 2: (1R,3S)-3-(3-((3-methylpyrazin-2-yl)amino)-1H-pyrazol-5-yl)cyclopentyl isopropylcarbamate. A solution of (1R,3S)-3-(1-(tert-butyl)-5-((3-methylpyrazin-2-yl)amino)-1H-pyrazol-3-yl)cyclopentyl isopropylcarbamate (88 mg, 0.22 mmol) in formic acid (3 mL) was stirred at 90° C. for 16 hours. The mixture was concentrated under reduced pressure and purified by preparative TLC (100% ethyl acetate in petroleum ether) to give the crude (30 mg). The crude was purified by Purification Procedure S with 0.05% ammonium hydroxide and 10 mM ammonium bicarbonate in water-acetonitrile (30-60%).to afford (1R,3S)-3-(3-((3-methylpyrazin-2-yl)amino)-1H-pyrazol-5-yl)cyclopentyl isopropylcarbamate (6.9 mg, 9%). LCMS (ES-API, M/Z): 345.1 [M+H]⁺˜; ¹H NMR (400 MHz, DMSO-d6) δ 12.02 (br s, 1H), 8.54 (br s, 1H), 7.94 (s, 1H), 7.77 (s, 1H), 6.95-6.94 (m, 1H), 6.38 (br s, 1H), 5.00 (br s, 1H), 3.58-3.55 (m, 1H), 3.05 (br s, 1H), 2.50-2.46 (in, 1H), 2.45 (s, 3H), 2.01-1.86 (m, 2H), 1.74-1.63 (m, 3H), 1.02 (br d, J=5.6 Hz, 6H).

Other compounds were synthesized according to General Procedure and Example 2, as set forth in the below Table G.

TABLE G
General Procedure 2 and Example 2 Compounds

| | | Starting | | |
|---|---|---|---|---|
| Ex | Structure/Name | Material | ¹H NMR | MS (m/z) |
| 2A | 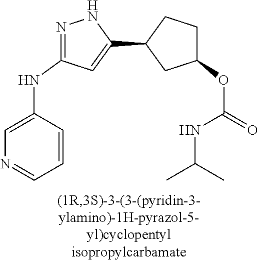 [SMILES: CC(C)NC(=O)O[C@@H]1CC[C@H](c2cc(Nc3cccnc3)n[nH]2)C1] | 3- bromopyridine | ¹H NMR (400 MHz, methanol- d₄) δ 8.41 (s, 1H), 7.91-7.90 (m, 1H), 7.74-7.72 (m, 1H), 7.25-7.22 (m, 1H), 5.78 (s, 1H), 5.09 (s, 1H), 3.69 (s, 1H), 3.17 - 3.15 (m, 1H), 2.57-2.50 (m, 1H), 2.13-2.11 (m, 1H), 1.95-1.78 (m, 4H), 1.11-1.10 (m, 6H). | 330.2 [M + H]⁺ |
| 2B | 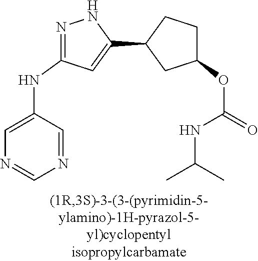 [SMILES: CC(C)NC(=O)O[C@@H]1CC[C@H](c2cc(Nc3cncnc3)n[nH]2)C1] | 5-bromo- pyrimidine | ¹H NMR (400 MHz, DMSO-d₆) δ 11.92 (s, 1H), 8.84 (s, 2H), 8.81 (s, 1H), 8.53 (s, 1H), 6.94 (d, J = 7.6 Hz, 1H), 5.66 (d, J = 2.0 Hz, 1H), 4.99 (s, 1H), 3.60 - 3.55 (m, 1H), 3.07-3.03 (m, 1H), 2.46-2.44 (m, 1H), 2.03- 2.01 (m, 1H), 1.92-1.90 (m, 1H), 1.75-1.71 (m, 2H), 1.65- 1.56 (m, 1H), 1.03 (d, J = 6.8 Hz, 6H). | 331.2 [M + H]⁺ |
| 2C | 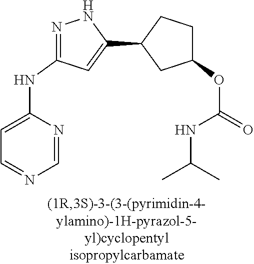 [SMILES: CC(C)NC(=O)O[C@@H]1CC[C@H](c2cc(Nc3ccncn3)n[nH]2)C1] | 4-chloro- pyrimidine | ¹H NMR (400 MHz, DMSO-d₆) δ 12.08 (br s, 1H), 9.92-9.59 (m, 1H), 8.64-8.45 (m, 1H), 8.24 (d, J = 6.0 Hz, 1H), 7.17 (br s, 1H), 6.95 (d, J = 7.0 Hz, 1H), 6.32-5.91 (m, 1H), 5.12-4.81 (m, 1H), 3.71-3.45 (m, 1H), 3.33 (br s, 2H), 3.15-2.90 (m, 1H), 2.44 (br s, 1H), 2.16-1.51 (m, 5H), 1.03 (d, J = 6.0 Hz, 6H). | 331.1 [M + H]⁺ |
| 2D | 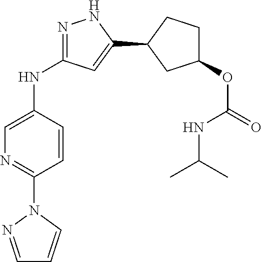 [SMILES: CC(C)NC(=O)O[C@@H]1CC[C@H](c2cc(Nc3ccc(-n4cccn4)nc3)n[nH]2)C1] | 5-bromo-2- (1H-pyrazol-1- yl)-pyridine | ¹H NMR (400 MHz, DMSO-d₆) δ 11.83 (br s, 1H), 8.72 (s, 1H), 8.45 (d, J = 2.4 Hz, 2H), 8.00 (dd, J = 8.8, 2.4 Hz, 1H), 7.76- 7.71 (m, 2H), 6.95 (d, J = 7.2 Hz, 1H), 6.56-6.44 (m, 1H), 5.65 (s, 1H), 5.00 (br s, 1H), 3.59-3.55 (m, 1H), 3.07-3.03 (m, 1H), 2.46-2.42 (m, 1H), 2.03-1.90 (m, 2H), 1.74-1.61 (m, 3H), 1.03 (d, J = 6.4 Hz, 6H). | 396.1 [M + H]⁺ |
| | (1R,3S)-3-(3-((6-(1H-pyrazol-1- | | | |
| | yl)pyridin-3-yl)amino)-1H- | | | |
| | pyrazol-5-yl)cyclopentyl | | | |
| | isopropylcarbamate | | | |
| 2E | 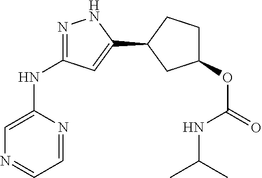 [SMILES: CC(C)NC(=O)O[C@@H]1CC[C@H](c2cc(Nc3cnccn3)n[nH]2)C1] | 2-iodopyrazine | ¹H NMR (400 MHz, methanol- d₄) δ 8.38 (s, 1H), 8.12 (s, 1H), 7.86 (s, 1H), 6.28 (s, 1H), 5.09 (s, 1H), 3.73-3.68 (m, 1H), 3.20-3.13 (m, 1H), 2.55-2.50 (m, 1H), 2.12-1.77 (m, 5H), 1.12 (d, J = 6.0 Hz, 6H). | 331.2 [M + H]⁺ |
| | (1R,3S)-3-(3-(pyrazin-2- | | | |
| | ylamino)-1H-pyrazol-5- | | | |
| | yl)cyclopentyl | | | |
| | isopropylcarbamate | | | |
| 2F | 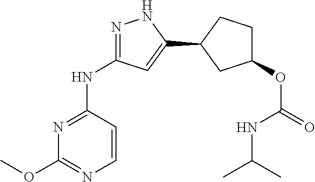 [SMILES: COc1nccc(Nc2cc([C@H]3CC[C@@H](OC(=O)NC(C)C)C3)[nH]n2)n1] | 4-chloro-2- methoxy- pyrimidine | ¹H NMR (400 MHz, DMSO-d₆) δ 12.05 (br s, 1H), 9.83 (s, 1H), 8.03 (d, J = 5.2 Hz, 1H), 6.94 (d, J = 7.6 Hz, 1H), 6.69 (s, 1H), 6.29 (s, 1H), 5.00 (s, 1H), 3.83 (s, 3H), 3.60-3.55 (m, 1H), 3.08-3.04 (m, 1H), 2.48-2.43 (m, 1H), 2.03-2.01 (m, 1H), 1.92-1.89 (m, 1H), 1.88-1.59 (m, 3H), 1.02 (d, J = 6.4 Hz, 6H). | 361.2 [M + H]⁺ |
| | (1R,3S)-3-(3-((2- | | | |
| | methoxypyrimidin-4-yl)amino)- | | | |
| | 1H-pyrazol-5-yl)cyclopenty1 | | | |
| | isopropylcarbamate | | | |
| 2G | 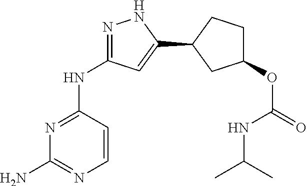 [SMILES: CC(C)NC(=O)O[C@@H]1CC[C@H](c2cc(Nc3ccnc(N)n3)n[nH]2)C1] | 4-chloro- pyrimidin-2- amine | ¹H NMR (400 MHz, methanol- d₄) δ 11.93 (br s, 1H), 9.32 (s, 1H), 7.78 (d, J = 5.6 Hz, 1H), 6.93 (d, J = 6.8 Hz, 1H), 6.15 (s, 3H), 4.99 (s, 1H), 3.57 (dd, J = 13.2, 6.4 Hz, 1H), 2.90-3.12 (m, 1H), 2.39-2.48 (m, 1H), 1.54-2.07 (m, 4H), 1.54-2.07 (m, 1H), 1.02 (d, J = 6.4 Hz, 6H). | 346.2 [M + H]⁺ |
| | (1R,3S)-3-(3-((2- | | | |
| | aminopyrimidin-4-yl)amino)-1H- | | | |
| | pyrazol-5-yl)cyclopentyl | | | |
| | isopropylcarbamate | | | |
| 2H | 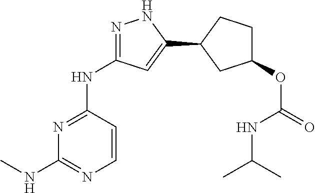 [SMILES: CNc1nccc(Nc2cc([C@H]3CC[C@@H](OC(=O)NC(C)C)C3)[nH]n2)n1] | 4-chloro-N- methyl- pyrimidin-2- amine | ¹H NMR (400 MHz, methanol- d₄) δ 7.81 (br s, 1H), 6.00-6.59 (m, 1H), 5.09 (br s, 1H), 3.55- 3.80 (m, 1H), 3.06-3.24 (m, 1H), 2.92 (s, 3H), 2.45-2.59 (m, 1H), 2.10 (br s, 1H), 1.74- 2.01 (m, 4H), 1.11 (d, J = 6.0 Hz, 6H). | 360.3 [M + H]⁺ |
| | (1R,3S)-3-(3-((2- | | | |
| | (methylamino)pyrimidin-4- | | | |
| | yl)amino)-1H-pyrazol-5- | | | |
| | yl)cyclopentyl | | | |
| | isopropylcarbamate | | | |
| 2I | 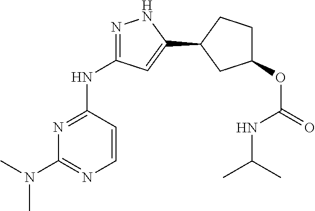 [SMILES: CC(C)NC(=O)O[C@@H]1CC[C@H](c2cc(Nc3ccnc(N(C)C)n3)n[nH]2)C1] | 2,4-dichloro- pyrimidine | ¹H NMR (400 MHz, methanol- d₄) δ 7.85 (s, 1H), 6.44 (s, 1H), 6.15 (s, 1H), 5.09 (s, 1H), 3.72- 3.64 (m, 1H), 3.15 (s, 6H), 2.56- 2.51 (m, 1H), 2.11-1.79 (m, 5H), 1.16 -1.03 (d, J = 6.4 Hz, 6H). | 374.3 [M + H]⁺ |
| | (1R,3S)-3-(3-((2- | | | |
| | (dimethylamino)pyrimidin-4- | | | |
| | yl)amino)-1H-pyrazol-5- | | | |
| | yl)cyclopentyl | | | |
| | isopropylcarbamate | | | |
| 2J | 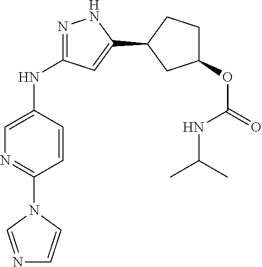 [SMILES: CC(C)NC(=O)O[C@@H]1CC[C@H](c2cc(Nc3ccc(-n4ccnc4)nc3)n[nH]2)C1] | 5-bromo-2- (1H-imidazol- 1-yl)pyridine | ¹H NMR (400 MHz, DMSO-d₆) δ 11.85 (br s, 1H), 8.77 (s, 1H), 8.47 (s, 1H), 8.34 (s, 1H), 8.00 (dd, J = 8.8, 2.4 Hz, 1H), 7.80 (s, 1H), 7.61 (d, J = 8.8 Hz, 1H), 7.07 (s, 1H), 6.96 (d, J = 7.6 Hz, 1H), 5.64 (d, J = 2.0 Hz, 1H), 4.99 (br s, 1H), 3.60-3.56 (m, 1H), 3.07-3.03 (m, 1H), 2.48- 2.42 (m, 1H), 2.03-1.88 (m, 2H), 1.73-1.60 (m, 3H), 1.03 (d, J = 6.4 Hz, 6H). | 396.1 [M + H]⁺ |
| | (1R,3S)-3-(3-((6-(1H-imidazol-1- | | | |
| | yl)pyridin-3-yl)amino)-1H- | | | |
| | pyrazol-5-yl)cyclopentyl | | | |
| | isopropylcarbamate | | | |
| 2K | 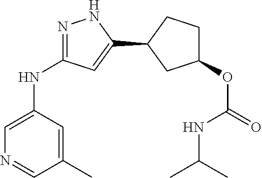 [SMILES: Cc1cncc(Nc2cc([C@H]3CC[C@@H](OC(=O)NC(C)C)C3)[nH]n2)c1] | 3-bromo-5- methylpyridine | ¹H NMR (400 MHz, DMSO-d₆) δ 8.46 (s, 1H), 8.31 (d, J = 2.4 Hz, 1H), 8.14 (s, 1H), 7.74 (s, 1H), 7.65 (s, 1H), 6.95-6.93 (m, 1H), 5.62 (s, 1H), 4.99 (s, 1H), 3.05-3.01 (m, 1H), 2.45- 2.43 (m, 1H), 2.21 (s, 3H), 2.02- 1.87 (m, 2H), 1.72-1.59 (m, 3H), 1.03 (d, J =6.4 Hz, 6H). | 344.1 [M + H]⁺ |
| | (1R,3S)-3-(3-((5-methylpyridin- | | | |
| | 3-yl)amino)-1H-pyrazol-5- | | | |
| | yl)cyclopentyl | | | |
| | isopropylcarbamate | | | |
| 2L | 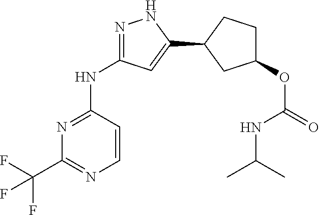 [SMILES: CC(C)NC(=O)O[C@@H]1CC[C@H](c2cc(Nc3ccnc(C(F)(F)F)n3)n[nH]2)C1] | 4-chloro-2- (trifluoromethyl) pyrimidine | ¹H NMR (400 MHz, methanol- d4) § 8.36 (d, J = 6.0 Hz, 1H), 7.24 (br s, 1H), 6.34 (br s, 1H), 5.10 (br s, 1H), 3.79-3.60 (m, 1H), 3.27-3.14 (m, 1H), 2.62- 2.50 (m, 1H), 2.21-2.07 (m, 1H), 2.04-1.73 (m, 4H), 1.17- 1.00 (m, 6H). | 399.1 [M + H]⁺ |
| | (1R,3S)-3-(3-((2- | | | |
| | (trifluoromethyl)pyrimidin-4- | | | |
| | yl)amino)-1H-pyrazol-5- | | | |
| | yl)cyclopentyl | | | |
| | isopropylcarbamate | | | |
| 2M | 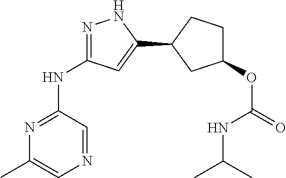 [SMILES: Cc1cncc(Nc2cc([C@H]3CC[C@@H](OC(=O)NC(C)C)C3)[nH]n2)n1] | 2-chloro-6- methylpyrazine | ¹H NMR (400 MHz, CDCl₃) δ 9.78-9.48 (m, 1H), 8.43-7.91 (m, 3H), 6.31 (br s, 1H), 5.89 (br s, 1H), 5.26 (br s, 1H), 3.83 (d, J = 1.2 Hz, 1H), 3.35 (d, J = 2.4 Hz, 1H), 2.59 (br s, 3H), 2.45 (br s, 1H), 2.22 (br s, 1H), 2.07 (d, J = 5.2 Hz, 1H), 1.98-1.85 (m, 3H), 1.17 (br s, 6H). | 345.1 [M + H]⁺ |
| | (1R,3S)-3-(3-((6-methylpyrazin- | | | |
| | 2-y1)amino)-1H-pyrazol-5- | | | |
| | yl)cyclopentyl | | | |
| | isopropylcarbamate | | | |
| 2N | 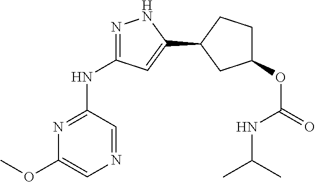 [SMILES: COc1cncc(Nc2cc([C@H]3CC[C@@H](OC(=O)NC(C)C)C3)[nH]n2)n1] | 2-chloro-6- methoxy- pyrazine | ¹H NMR (400 MHz, methanol- d₄) δ 7.94 (s, 1H), 7.61 (s, 1H), 6.35 (s, 1H), 5.12 (s, 1H), 4.01 (s, 3H), 3.72-3.69 (m, 1H), 3.24-3.20 (m, 1H), 2.60-2.56 (m, 1H), 2.18-1.81 (m, 5H), 1.14-1.11 (m, 6H). | 361.1 [M + H]⁺ |
| | (1R,3S)-3-(3-((6- | | | |
| | methoxypyrazin-2-yl)amino)- | | | |
| | 1H-pyrazol-5-yl)cyclopenty1 | | | |
| | isopropylcarbamate | | | |
| 2O | 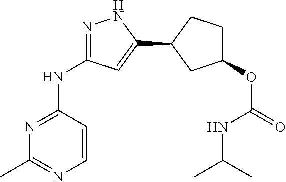 [SMILES: Cc1nccc(Nc2cc([C@H]3CC[C@@H](OC(=O)NC(C)C)C3)[nH]n2)n1] | 4-chloro-2- methyl- pyrimidine | ¹H NMR (400 MHz, DMSO-d₆) δ 8.27 (d, J = 7.20 Hz, 1H), 7.26 (s, 1H), 6.53 (s, 1H), 6.25 (s, 1H), 5.14-4.99 (m, 1H), 3.63- 3.54 (m, 1H), 3.18-3.09 (m, 1H), 2.57 (s, 3H), 2.07-1.64 (m, 6H), 1.06 (d, J = 6.80 Hz, 6H). | 345.1 [M + H]⁺ |
| | (1R,3S)-3-(3-((2- | | | |
| | methylpyrimidin-4-yl)amino)- | | | |
| | 1H-pyrazol-5-yl)cyclopenty1 | | | |
| | isopropylcarbamate | | | |
| 2P | 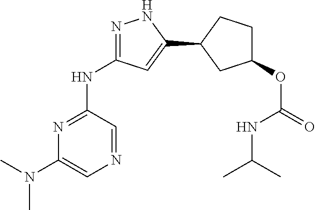 [SMILES: CC(C)NC(=O)O[C@@H]1CC[C@H](c2cc(Nc3cncc(N(C)C)n3)n[nH]2)C1] | 6-chloro-N,N- dimethyl- pyrazin-2- amine | ¹H NMR (400 MHz, CDCl₃) δ 7.89 (br s, 1H), 7.32 (br s, 1H), 6.45 (br s, 1H), 5.37-5.22 (m, 2H), 3.90-3.74 (m, 1H), 3.41- 3.25 (m, 1H), 3.19 (s, 6H), 2.59- 2.43 (m, 1H), 2.29-2.17 (m, 1H), 2.06-1.81 (m, 4H), 1.14 (d, J = 6.4 Hz, 6H). | 374.1 [M + H]⁺ |
| | (1R,3S)-3-(3-((6- | | | |
| | (dimethylamino)pyrazin-2- | | | |
| | yl)amino)-1H-pyrazol-5- | | | |
| | yl)cyclopentyl | | | |
| | isopropylcarbamate | | | |
| 2Q | 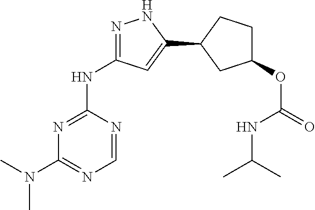 [SMILES: CC(C)NC(=O)O[C@@H]1CC[C@H](c2cc(Nc3ncnc(N(C)C)n3)n[nH]2)C1] | 4-chloro-N,N- dimethyl- 1,3,5-triazin-2- amine | ¹H NMR (400 MHz, methanol- d₄) δ 8.29 (s, 1H), 6.37 (s, 1H), 5.33-5.26 (m, 2H), 3.82 (br s, 1H), 3.40-3.13 (m, 7H), 2.46 (br s, 1H), 2.21-2.16 (m, 1H), 2.02-1.86 (m, 4H), 1.16-1.14 (m, 6H). | 375.1 [M + H]⁺ |
| | (1R,3S)-3-(3-((4- | | | |
| | (dimethylamino)-1,3,5-triazin-2- | | | |
| | yl)amino)-1H-pyrazol-5- | | | |
| | yl)cyclopentyl | | | |
| | isopropylcarbamate | | | |
| 2R | 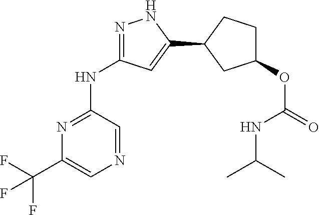 [SMILES: CC(C)NC(=O)O[C@@H]1CC[C@H](c2cc(Nc3cncc(C(F)(F)F)n3)n[nH]2)C1] | 2-chloro-6- (trifluoromethyl) pyrazine | ¹H NMR (400 MHz, methanol- d₄) δ 8.59 (s, 1H), 8.30 (s, 1H), 6.42 (s, 1H), 3.69 (br s, 1H), 3.32-3.20 (m, 1H), 2.58-2.55 (m, 1H), 2.14-1.98 (m, 1H), 1.96-1.88 (m, 4H), 1.11-1.09 (m, 6H). | 399.1 [M + H]⁺ |
| | (1R,3S)-3-(3-((6- | | | |
| | (trifluoromethyl)pyrazin-2- | | | |
| | yl)amino)-1H-pyrazol-5- | | | |
| | yl)cyclopentyl | | | |
| | isopropylcarbamate | | | |
| 2S | 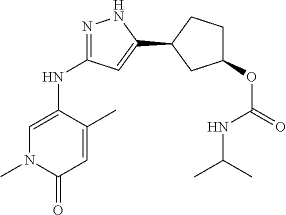 [SMILES: Cc1cc(=O)n(C)cc1Nc1cc([C@H]2CC[C@@H](OC(=O)NC(C)C)C2)[nH]n1] | 5-bromo-1,4- dimethylpyridin- 2(1H)-one | ¹H NMR (400 MHz, methanol- d₄) δ 7.71 (s, 1H), 6.45 (s, 1H), 5.57 (s, 1H), 5.07 (s, 1H), 3.71- 3.69 (m, 1H), 3.54 (s, 3H), 3.15- 3.09 (m, 1H), 2.53-2.49 (m, 1H), 2.19 (s, 3H), 2.07-1.92 (m, 2H), 1.90-1.73 (m, 3H), 1.11 (d, J = 6.4 Hz, 6H). | 374.1 [M + H]⁺ |
| | (1R,3S)-3-(3-((1,4-dimethyl-6- | | | |
| | oxo-1,6-dihydropyridin-3- | | | |
| | yl)amino)-1H-pyrazol-5- | | | |
| | yl)cyclopentyl | | | |
| | isopropylcarbamate | | | |
| 2T | 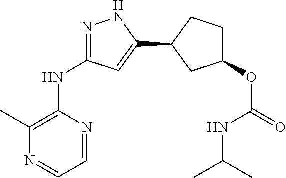 [SMILES: Cc1nccnc1Nc1cc([C@H]2CC[C@@H](OC(=O)NC(C)C)C2)[nH]n1] | 2-chloro-3- methylpyrazine | ¹H NMR (400 MHz, DMSO-d₆) δ 12.02 (s, 1H), 8.54 (s, 1H), 7.94 (s, 1H), 7.77 (s, 1H), 6.95- 6.94 (m, 1H), 6.38 (s, 1H), 5.00 (s, 1H), 3.58-3.55 (m, 1H), 3.10-3.02 (m, 1H), 2.50-2.45 (m, 4H), 2.01-1.86 (m, 2H), 1.74-1.63 (m, 3H), 1.02 (d, J = 5.6 Hz, 6H). | 345.1 [M + H]⁺ |
| | (1R,3S)-3-(3-((3-methylpyrazin- | | | |
| | 2-yl)amino)-1H-pyrazol-5- | | | |
| | yl)cyclopentyl | | | |
| | isopropylcarbamate | | | |
| 2U | 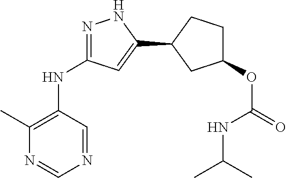 [SMILES: Cc1ncncc1Nc1cc([C@H]2CC[C@@H](OC(=O)NC(C)C)C2)[nH]n1] | 5-bromo-4- methyl- pyrimidine | ¹H NMR (400 MHz, DMSO-d₆) δ 11.91 (s, 1H), 9.19 (s, 1H), 8.43 (s, 1H), 7.78 (s, 1H), 6.94 (d, J = 8.0 Hz, 1H), 5.79 (s, 1H), 4.99 (br s, 1H), 3.60-3.55 (m, 1H), 3.07-3.03 (m, 1H), 2.48- 2.45 (m, 1H), 2.44 (s, 3H), 2.03- 2.01 (m, 1H), 1.92-1.90 (m, 1H), 1.73-1.59 (m, 3H), 1.02 (d, J = 6.4 Hz, 6H). | 345.1 [M + H]⁺ |
| | (1R,3S)-3-(3-((4- | | | |
| | methylpyrimidin-5-yl)amino)- | | | |
| | 1H-pyrazol-5-yl)cyclopentyl | | | |
| | isopropylcarbamate | | | |
| 2V | 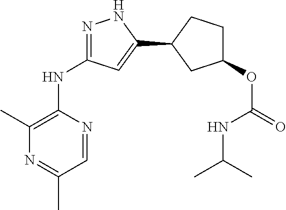 [SMILES: Cc1cnc(Nc2cc([C@H]3CC[C@@H](OC(=O)NC(C)C)C3)[nH]n2)c(C)n1] | 2-chloro-3,5- dimethyl- pyrazine | ¹H NMR (400 MHz, DMSO-d₆) δ 11.87 (s, 1H), 8.44 (s, 1H), 7.82 (s, 1H), 6.94 (d, J = 7.2 Hz, 1H), 6.26 (s, 1H), 4.99 (s, 1H), 3.60-3.54 (m, 1H), 3.11-3.04 (m, 1H), 2.50-2.44 (m, 1H), 2.42 (s, 3H), 2.29 (s, 3H), 2.01- 1.89 (m, 2H), 1.73-1.59 (m, 3H), 1.02 (d, J = 3.2 Hz, 6H). | 359.1 [M + H]⁺ |
| | (1R,3S)-3-(3-((3,5- | | | |
| | dimethylpyrazin-2-yl)amino)- | | | |
| | 1H-pyrazol-5-y1)cyclopentyl | | | |
| | isopropylcarbamate | | | |
| 2W | 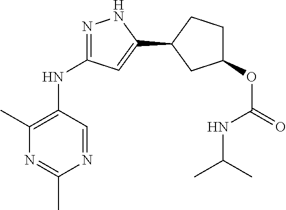 [SMILES: Cc1ncc(Nc2cc([C@H]3CC[C@@H](OC(=O)NC(C)C)C3)[nH]n2)c(C)n1] | 5-bromo-2,4- dimethyl- pyrimidine | ¹H NMR (400 MHz, DMSO-d₆) δ 11.82 (s, 1H), 9.07 (s, 1H), 7.62 (s, 1H), 6.95-6.93 (m, 1H), 5.73 (s, 1H), 4.99 (s, 1H), 3.58-3.55 (m, 1H), 3.05-3.01 (m, 1H), 2.44 (s, 3H), 2.39 (s, 3H), 2.02-1.88 (m, 2H), 1.72- 1.58 (m, 3H), 1.02 (d, J = 5.6 Hz, 6H). | 359.1 [M + H]⁺ |
| | (1R,3S)-3-(3-((2,4- | | | |
| | dimethylpyrimidin-5-yl)amino)- | | | |
| | 1H-pyrazol-5-y1)cyclopentyl | | | |
| | isopropylcarbamate | | | |
| 2X | 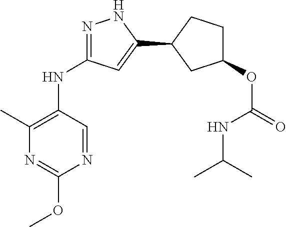 [SMILES: COc1ncc(Nc2cc([C@H]3CC[C@@H](OC(=O)NC(C)C)C3)[nH]n2)c(C)n1] | 5-bromo-2- methoxy-4- methyl- pyrimidine | ¹H NMR (500 MHz, DMSO-d₆) δ 11.69 (s, 1H), 8.84 (s, 1H), 7.43 (s, 1H), 6.93-6.91 (m, 1H), 5.62 (s, 1H), 4.98 (s, 1H), 3.81 (s, 3H), 3.59-3.56 (m, 1H), 3.06-2.96 (m, 1H), 2.45- 2.42 (m, 1H), 2.37 (s, 3H), 2.00- 1.88 (m, 2H), 1.72-1.60 (m, 3H), 1.03-1.01 (m, 6H). | 375.1 [M + H]⁺ |
| | (1R,3S)-3-(3-((2-methoxy-4- | | | |
| | methylpyrimidin-5-yl)amino)- | | | |
| | 1H-pyrazol-5-yl)cyclopenty1 | | | |
| | isopropylcarbamate | | | |
| 2Y | 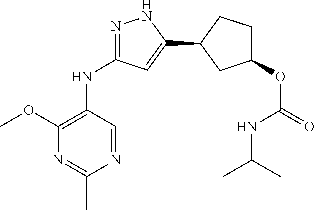 [SMILES: COc1nc(C)ncc1Nc1cc([C@H]2CC[C@@H](OC(=O)NC(C)C)C2)[nH]n1] | 5-bromo-4- methoxy-2- methyl- pyrimidine | ¹H NMR (400 MHz, DMSO-d₆) 11.80 (s, 1H), 9.07 (s, 1H), 7.80 (s, 1H), 6.95-6.94 (m, 1H), 5.77 (s, 1H), 4.99 (s, 1H), 3.97 (s, 3H), 3.60-3.55 (m, 1H), 3.36-3.01 (m, 1H), 2.46- 2.44 (m, 1H), 2.42 (s, 3H), 2.01- 1.88 (m, 2H), 1.72-1.67 (m, 3H), 1.04-1.02 (m, 1H). | 375.1 [M + H]⁺ |
| | (1R,3S)-3-(3-((4-methoxy-2- | | | |
| | methylpyrimidin-5-yl)amino)- | | | |
| | 1H-pyrazol-5-yl)cyclopentyl | | | |
| | isopropylcarbamate | | | |
| 2Z | 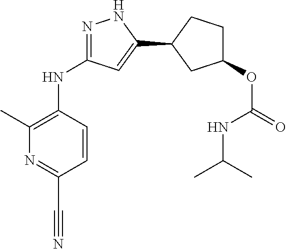 [SMILES: Cc1nc(C#N)ccc1Nc1cc([C@H]2CC[C@@H](OC(=O)NC(C)C)C2)[nH]n1] | 5-bromo-6- methyl- picolinonitrile | ¹H NMR (400 MHz, DMSO-d₆) δ 12.11 (s, 1H), 8.25 (d, J = 6.4 Hz, 2H), 7.66 (d, J = 8.4 Hz, 1H), 6.94 (d, J = 7.6 Hz, 1H), 5.91 (s, 1H), 4.99 (br s, 1H), 3.60-3.55 (m, 1H), 3.09-3.04 (m, 1H), 2.48 (s, 3H), 2.47- 2.42 (m, 1H), 2.04-2.02 (m, 1H), 1.92-1.90 (m, 1H), 1.76- 1.60 (m, 3H), 1.02 (d, J = 6.4 Hz, 6H). | 369.1 [M + H]⁺ |
| | (1R,3S)-3-(3-((6-cyano-2- | | | |
| | methylpyridin-3-yl)amino)-1H- | | | |
| | pyrazol-5-yl)cyclopentyl | | | |
| | isopropylcarbamate | | | |
| 2AA | 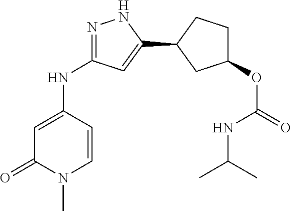 [SMILES: CC(C)NC(=O)O[C@@H]1CC[C@H](c2cc(Nc3ccn(C)c(=O)c3)n[nH]2)C1] | 4-bromo-1- methylpyridin- 2(1H)-one | ¹H NMR (400 MHz, DMSO-d₆) δ (ppm) 11.99 (br s, 1H), 8.77 (s, 1H), 7.36 (d, J = 7.2 Hz, 1H), 6.95 (d, J = 7.2 Hz, 1H), 6.32 (br s, 1H), 6.10 (d, J = 7.2 Hz, 1H), 5.68 (s, 1H), 4.99 (br s, 1H), 3.58-3.56 (m, 1H), 3.26 (s, 3H), 3.06-3.02 (m, 1H), 2.02- 2.00 (m, 1H), 1.91-1.87 (m, 1H), 1.72-1.58 (m, 4H), 1.02 (d, J = 7.2 Hz, 6H). | 360.1 [M + H]⁺ |
| | (1R,3S)-3-(3-((1-methyl-2-oxo- | | | |
| | 1,2-dihydropyridin-4-yl)amino)- | | | |
| | 1H-pyrazol-5-y1)cyclopenty1 | | | |
| | isopropylcarbamate | | | |
| 2BB | 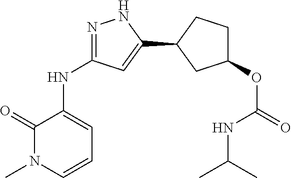 [SMILES: CC(C)NC(=O)O[C@@H]1CC[C@H](c2cc(Nc3cccn(C)c3=O)n[nH]2)C1] | 3-bromo-1- methylpyridin- 2(1H)-one | ¹H NMR (400 MHz, DMSO-d₆) δ 11.76 (s, 1H), 7.83 (br s, 2H), 7.04 (d, J = 5.6 Hz, 1H), 6.96 (d, J = 7.6 Hz, 1H), 6.18 (t, J = 7.2 Hz, 1H), 5.86 (s, 1H), 4.99 (br s, 1H), 3.60-3.56 (m, 1H), 3.50 (s, 3H), 3.04-3.00 (m, 1H), 2.45-2.41 (m, 1H), 2.01-1.98 (m, 1H), 1.89-1.86 (m, 1H), 1.72-1.58 (m, 3H), 1.03 (d, J = 6.4 Hz, 6H). | 360.1 [M + H]⁺ |
| | (1R,3S)-3-(3-((1-methyl-2-oxo- | | | |
| | 1,2-dihydropyridin-3-yl)amino)- | | | |
| | 1H-pyrazol-5-y1)cyclopenty1 | | | |
| | isopropylcarbamate | | | |
| 2CC | 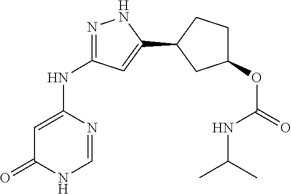 [SMILES: CC(C)NC(=O)O[C@@H]1CC[C@H](c2cc(Nc3cc(=O)[nH]cn3)n[nH]2)C1] | 2-chloro-5- (1H-pyrazol-1- yl)pyridine | ¹H NMR (400 MHz, DMSO-d₆) δ 9.26 (s, 1H), 7.91 (s, 1H), 6.93 (d, 1H), 6.23 (s, 1H), 5.81 (s, 1H), 4.99 (s, 1H), 3.05-3.01 (m, 1H), 1.99-1.86 (m, 2H), 1.72-1.57 (m, 3H), 1.02 (d, J = 6.4 Hz, 6H). | 347.1 [M + H]⁺ |
| | (1R,3S)-3-(3-((6-oxo-1,6- | | | |
| | dihydropyrimidin-4-yl)amino)- | | | |
| | 1H-pyrazol-5-yl)cyclopentyl | | | |
| | isopropylcarbamate | | | |
| 2DD | 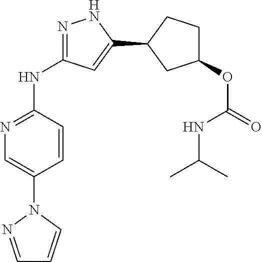 [SMILES: CC(C)NC(=O)O[C@@H]1CC[C@H](c2cc(Nc3ccc(-n4cccn4)cn3)n[nH]2)C1] | S1 and 6-chloro- pyrimidin- 4(3H)-one | ¹H NMR (400 MHz, DMSO-d₆) δ 11.86 (br s, 1H), 9.31 (s, 1H), 8.55 (d, J = 2.4 Hz, 1H), 8.34 (d, J = 2.0 Hz, 1H), 7.96 (dd, J = 8.8, 2.4 Hz, 1H), 7.70 (s, 1H), 7.41 (d, J = 9.2 Hz, 1H), 6.96 (d, J = 7.2 Hz, 1H), 6.51 (s, 1H), 6.13 (s, 1H), 5.00 (br s, 1H), 3.59-3.55 (m, 1H), 3.07-3.03 (m, 1H), 2.48-2.44 (m, 1H), 2.03-1.88 (m, 2H), 1.80-1.60 (m, 3H), 1.03 (d, J = 6.4 Hz, 6H) | 396.1 [M + H]⁺ |
| | (1R,3S)-3-(3-((5-(1H-pyrazol-1- | | | |
| | yl)pyridin-2-yl)amino)-1H- | | | |
| | pyrazol-5-yl)cyclopentyl | | | |
| | isopropylcarbamate | | | |
| 2EE | 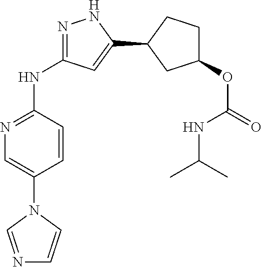 [SMILES: CC(C)NC(=O)O[C@@H]1CC[C@H](c2cc(Nc3ccc(-n4ccnc4)cn3)n[nH]2)C1] | 2-chloro-5- (1H-imidazol- 1-yl)pyridine | ¹H NMR (400 MHz, DMSO-d₆) δ 11.87 (br s, 1H), 9.36 (s, 1H), 8.38 (d, J = 2.4 Hz, 1H), 8.09 (s, 1H), 7.81 (dd, J = 8.8, 2.4 Hz, 1H), 7.61 (s, 1H), 7.39 (d, J = 8.8 Hz, 1H), 7.09 (s, 1H), 6.95 (d, J = 7.2 Hz, 1H), 6.15 (s, 1H), 5.00 (br s, 1H), 3.60-3.55 (m, 1H), 3.05-3.02 (m, 1H), 2.46- 2.42 (m, 1H), 2.03-1.88 (m, 2H), 1.74-1.60 (m, 3H), 1.03 (d, J = 6.4 Hz, 6H). | 396.1 |
| | (1R,3S)-3-(3-((5-(1H-imidazol-1- | | | |
| | yl)pyridin-2-yl)amino)-1H- | | | |
| | pyrazol-5-yl)cyclopentyl | | | |
| | isopropylcarbamate | | | |
| 2FF | 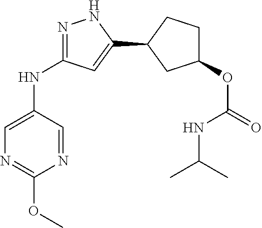 [SMILES: COc1ncc(Nc2cc([C@H]3CC[C@@H](OC(=O)NC(C)C)C3)[nH]n2)cn1] | 5-bromo-2- methoxy- pyrimidine | ¹H NMR (400 MHz, Methanol- d₄) δ 8.57 (s, 2H), 5.09-5.08 (m, 1H), 3.98 (s, 3H), 3.70- 3.67 (m, 1H), 3.16-3.15 (m, 1H), 2.57-2.52 (m, 1H), 2.15- 1.66 (m, 6H), 1.12-1.09 (m, 6H). | 361.1 [M + H]⁺ |
| | (1R,3S)-3-(3-((2- | | | |
| | methoxypyrimidin-5-yl)amino)- | | | |
| | 1H-pyrazol-5-yl)cyclopentyl | | | |
| | isopropylcarbamate | | | |
| 2GG | 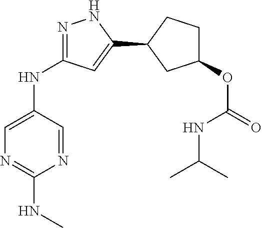 [SMILES: CNc1ncc(Nc2cc([C@H]3CC[C@@H](OC(=O)NC(C)C)C3)[nH]n2)cn1] | 5-bromo-N- methyl- pyrimidin-2- amine | ¹H NMR (400 MHz, Methanol- d₄) δ 8.61-8.60 (m, 2H), 5.72 5.70 (m, 1H), 4.56-4.51 (m, 1H), 3.69 (s, 1H), 3.15-3.01 (m, 1H), 3.00-2.99 (m, 3H), 2.56-2.53 (m, 1H), 2.13-2.10 (m, 1H), 1.95-1.93 (m, 2H), 1.68-1.64 (m, 3H), 1.30-1.29 (m, 1H), 1.12 (s, 6H). | 360.2 [M + H]⁺ |
| | (1R,3S)-3-(3-((2- | | | |
| | (methylamino)pyrimidin-5- | | | |
| | yl)amino)-1H-pyrazol-5- | | | |
| | yl)cyclopentyl | | | |
| | isopropylcarbamate | | | |
| 2HH | 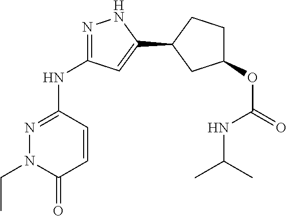 [SMILES: CCn1nc(Nc2cc([C@H]3CC[C@@H](OC(=O)NC(C)C)C3)[nH]n2)ccc1=O] | 6-bromo-2- ethylpyridazin- 3(2H)-one | ¹H NMR (400 MHz, CDCl₃) δ 9.03 (br s, 1H), 7.09 (br d, J = 9.6 Hz, 1H), 6.97 (d, J = 9.8 Hz, 1H), 6.32 (br s, 1H), 5.67 (br s, 1H), 5.27 (br s, 1H), 4.25 (br d, J = 6.8 Hz, 2H), 3.81 (br s, 1H), 3.35 (br s, 1H), 2.20-2.54 (m, 2H), 1.86-2.14 (m, 4H), 1.40 (t, J = 7.2 Hz, 3H), 1.15 (br d, J = 6.4 Hz, 6H). | 375.1 [M + H]⁺ |
| | (1R,3S)-3-(3-((1-ethyl-6-oxo-1,6- | | | |
| | dihydropyridazin-3-y1)amino)- | | | |
| | 1H-pyrazol-5-y1)cyclopenty1 | | | |
| | isopropylcarbamate | | | |
| 2II | 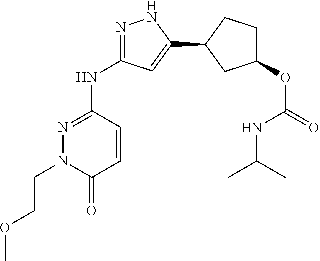 [SMILES: COCCn1nc(Nc2cc([C@H]3CC[C@@H](OC(=O)NC(C)C)C3)[nH]n2)ccc1=O] | 6-bromo-2-(2- methoxyethyl)- pyridazin- 3(2H)-one | ¹H NMR (400 MHz, CDCl₃) δ 10.40 (br s, 1H), 7.18 (d, J = 9.6 Hz, 1H), 6.95 (d, J = 9.8 Hz, 1H), 6.17 (s, 1H), 5.90 (br d, J = 5.5 Hz, 1H), 5.22 (br s, 1H), 4.48 (br s, 2H), 3.73-3.94 (m, 3H), 3.38 (s, 4H), 2.13-2.56 (m, 1H), 2.13-2.56 (m, 1H), 2.60 (s, 1H), 1.77-2.13 (m, 4H), 1.05-1.21 (m, 6H). | 405.1 [M + H]⁺ |
| | (1R,3S)-3-(3-((1-(2- | | | |
| | methoxyethyl)-6-oxo-1,6- | | | |
| | dihydropyridazin-3-y1)amino)- | | | |
| | 1H-pyrazol-5-y1)cyclopentyl | | | |
| | isopropylcarbamate | | | |
| 2JJ | 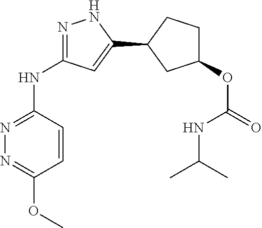 [SMILES: COc1ccc(Nc2cc([C@H]3CC[C@@H](OC(=O)NC(C)C)C3)[nH]n2)nn1] | 3-bromo-6- methoxy- pyridazine | ¹H NMR (400 MHz, Methanol- d₄) δ 7.65-7.57 (d, J = 32.0 Hz, 1H), 6.02 (s, 1H), 5.11-5.10 (m, 1H), 4.04 (s, 3H), 3.71- 3.68 (m, 1H), 3.23-3.21 (m, 1H), 2.63-2.58 (m, 1H), 2.17- 1.77 (m, 5H), 1.12-1.10 (m, 6H). | 361.1 [M + H]⁺ |
| | (1R,3S)-3-(3-((6- | | | |
| | methoxypyridazin-3-yl)amino)- | | | |
| | 1H-pyrazol-5-yl)cyclopentyl | | | |
| | isopropylcarbamate | | | |

### General Procedure 3

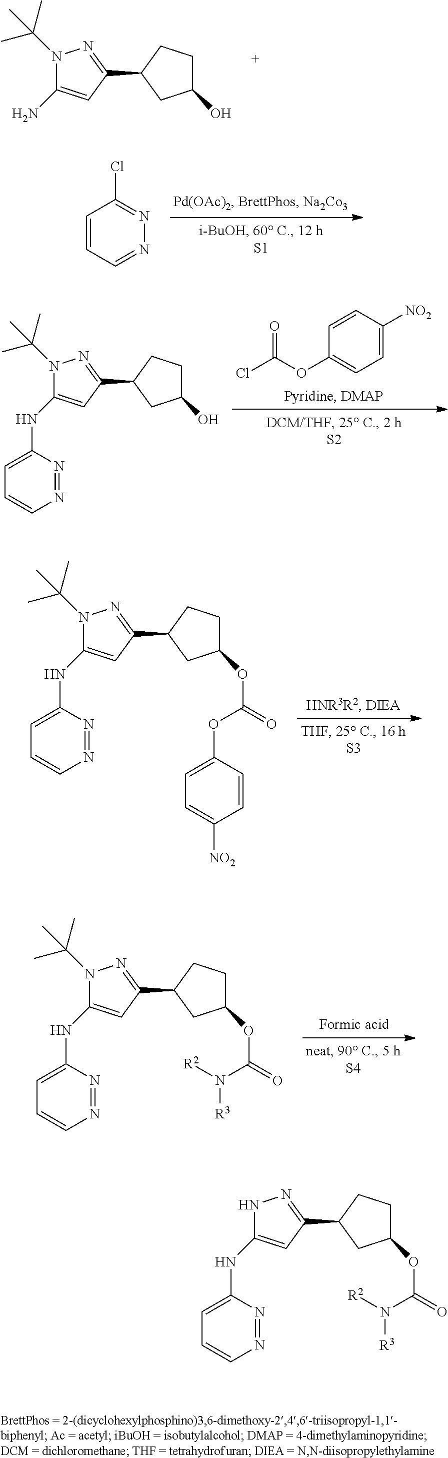

### Example 3: (1R,3S)-3-(3-(pyridazin-3-ylamino)-1H-pyrazol-5-yl)cyclopentyl bicyclo[1.1.1]pentan-1-ylcarbamate

The title compound was prepared according to General Procedure 3 as described below.

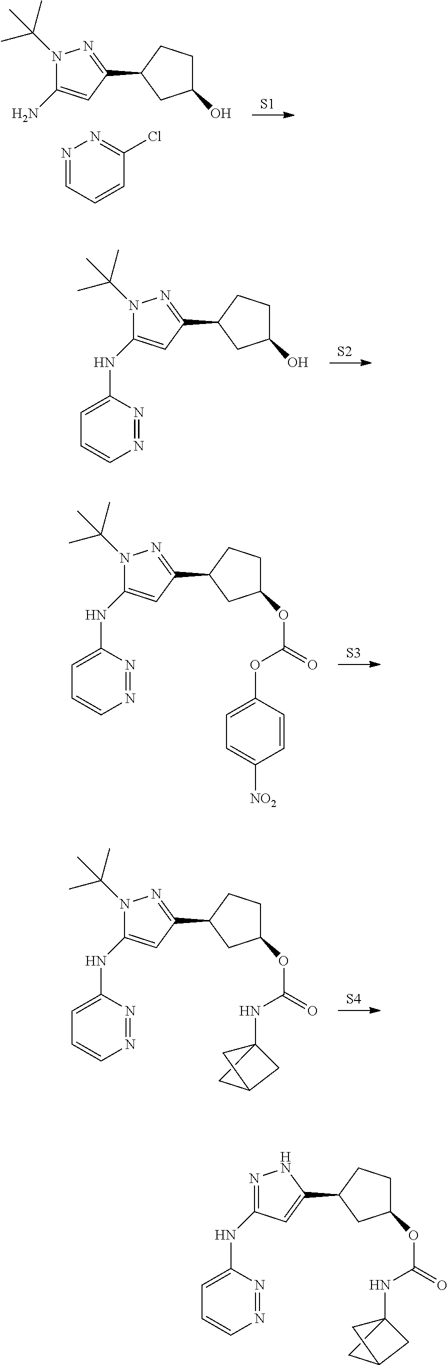

Step 1: (1R,3S)-3-(1-(tert-butyl)-5-(pyridazin-3-ylamino)-1H-pyrazol-3-yl)cyclopentan-1-ol. A mixture of (1R,3S)-3-(5-amino-1-(tert-butyl)-1H-pyrazol-3-yl)cyclopentanol (Intermediate C) (28.4 g, 127 mmol), 3-chloropyridazine hydrochloride (16.0 g, 106 mmol), 2-(dicyclohexylphosphino)3,6-dimethoxy-2′,4′,6′-tri-isopropyl-1,1′biphenyl (11.4 g, 21.2 mmol), palladium(II) acetate (2.38 g, 10.6 mmol) and sodium carbonate (33.7 g, 318 mmol) in isobutyl alcohol was stirred at 60° C. for 12 h under a nitrogen atmosphere. The reaction mixture was cooled to room temperature and filtered. The filtrate was concentrated under reduced pressure. The residue was purified by column chromatography (silica gel, 100-200 mesh, 1-2% methanol:dichloromethane) to afford (1R,3S)-3-(1-(tert-butyl)-5-(pyridazin-3-ylamino)-1H-pyrazol-3-yl)cyclopentan-1-ol (25.0 g, 65%). ¹H NMR (400 MHz, DMSO-d₆) δ 8.66 (s, 1H), 8.60 (br s, 1H), 7.40-7.36 (m, 1H), 6.75-6.73 (br s, 1H), 5.98 (s, 1H), 4.58-4.57 (br s, 1H), 4.15-4.14 (m, 1H), 2.95-2.90 (m, 1H), 2.50-2.49 (m, 1H), 1.57-1.51 (m, 5H), 1.50 (s, 9H).

Step 2: (1R,3S)-3-(1-(tert-butyl)-5-(pyridazin-3-ylamino)-1H-pyrazol-3-yl)cyclopentyl (4-nitrophenyl) carbonate. To a solution of (1R,3S)-3-[1-tert-butyl-5-(pyridazin-3-ylamino)pyrazol-3-yl]cyclopentanol (2.0 g, 6.6 mmol) in dichloromethane (15 mL) and tetrahydrofuran (15 mL) was added 4-nitrophenylchloroformate (2.7 g, 13 mmol), pyridine (1.6 mL, 20 mmol) and 4-dimethylaminopyridine (81 mg, 0.66 mmol). The mixture was stirred at 25° C. for 16 h and concentrated under reduced pressure. The residue was purified by column chromatography (silica gel, 100-200 mesh, 0-60% ethyl acetate:petroleum ether) to afford (1R,3S)-3-(1-(tert-butyl)-5-(pyridazin-3-ylamino)-1H-pyrazol-3-yl)cyclopentyl (4-nitrophenyl) carbonate (3.0 g, 97%). LCMS (ES-API, m/z): 467.2 [M+H]⁺.

Step 3: (1R,3S)-3-(1-(tert-butyl)-5-(pyridazin-3-ylamino)-1H-pyrazol-3-yl)cyclopentyl bicyclo[1.1.1]pentan-1-ylcarbamate. To a solution of (1R,3S)-3-(1-(tert-butyl)-5-(pyridazin-3-ylamino)-1H-pyrazol-3-yl)cyclopentyl (4-nitrophenyl) carbonate (400 mg, 0.86 mmol) in tetrahydrofuran (10 mL) was added N,N-diisopropylethylamine (0.90 mL, 5.1 mmol) and bicyclo[1.1.1]pentan-1-amine hydrochloride (154 mg, 1.3 mmol). The mixture was stirred at 25° C. for 16 h and concentrated under reduced pressure. The residue was purified by preparative thin layer chromatography (TLC) (ethyl acetate, Rf=0.5) to afford (1R,3S)-3-[1-tert-butyl-5-(pyridazin-3-ylamino)pyrazol-3-yl]cyclopentyl] N-(1-bicyclo[1.1.1]pentanyl)carbamate (110 mg, 31%). LCMS (ES-API, m/z): 411.2 [M+H]⁺.

Step 4: (1R,3S)-3-(3-(pyridazin-3-ylamino)-1H-pyrazol-5-yl)cyclopentyl bicyclo[1.1.1]pentan-1-ylcarbamate. A solution of (1R,3S)-3-[1-tert-butyl-5-(pyridazin-3-ylamino)pyrazol-3-yl]cyclopentyl] N-(1-bicyclo[1.1.1]pentanyl)carbamate (110 mg, 0.27 mmol) in formic acid (10 mL) was stirred at 90° C. for 5 h and concentrated under reduced pressure. The residue was purified by Purification Procedure C with 0.05% ammonium hydroxide and 10 mM ammonium hydrogen carbonate in water-acetonitrile (20-50%) to afford (1R,3S)-3-(3-(pyridazin-3-ylamino)-1H-pyrazol-5-yl)cyclopentyl bicyclo[1.1.1]pentan-1-ylcarbamate (40 mg, 42%). LCMS (ES-API, m/z): 355.1 [M+H]; ¹H NMR (400 MHz, DMSO-d₆) δ 11.95 (s, 1H), 9.52 (s, 1H), 8.59 (s, 1H), 7.79 (s, 1H), 7.55-7.52 (m, 1H), 7.41-7.37 (m, 1H), 6.20 (s, 1H), 5.00 (s, 1H), 3.08-3.04 (m, 1H), 2.47-2.34 (m, 2H), 2.07-1.99 (m, 2H), 1.89 (s, 6H), 1.84-1.60 (m, 3H).

### Example 3A: (1R,3S)-3-(3-(pyridazin-3-ylamino)-1H-pyrazol-5-yl)cyclopentyl (1-methylcyclopropyl)carbamate

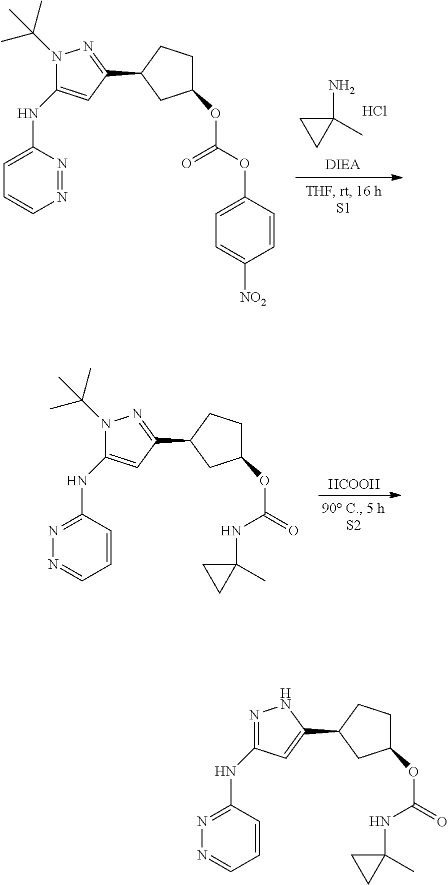

Step 1: [(1R,3S)-3-[1-tert-butyl-5-(pyridazin-3-ylamino)pyrazol-3-yl]cyclopentyl] N-(1-methylcyclopropyl)carbamate. A solution of (4-nitrophenyl) [(1R,3S)-3-[1-tert-butyl-5-(pyridazin-3-ylamino)pyrazol-3-yl]cyclopentyl] carbonate (280 mg, 0.60 mmol), N,N-diisopropylethylamine (0.63 mL, 3.6 mmol) and 1-methylcyclopropanamine hydrochloride (77 mg, 0.72 mmol) in tetrahydrofuran (10 mL) was stirred at 25° C. for 16 hours. The mixture was concentrated to dryness and diluted with ethyl acetate (30 mL), washed with 1 M sodium hydroxide solution (30 mL), brine (50 mL) and the organic extracts were dried over anhydrous sodium sulfate, filtered and concentrated under reduced pressure. The residue was purified by preparative TLC (100% ethyl acetate in petroleum ether, Rf=0.4) to afford [(1R,3S)-3-[1-tert-butyl-5-(pyridazin-3-ylamino)pyrazol-3-yl]cyclopentyl] N-(1-methylcyclopropyl)carbamate (30 mg, 13%). LCMS (ES-API, M/Z): 399.2 [M+H]⁺.

Step 2: (1R,3S)-3-(3-(pyridazin-3-ylamino)-1H-pyrazol-5-yl)cyclopentyl (1-methylcyclopropyl)carbamate. A solution of [(1R,3S)-3-[1-tert-butyl-5-(pyridazin-3-ylamino)pyrazol-3-yl]cyclopentyl] N-(1-methylcyclopropyl)carbamate (30 mg, 0.08 mmol) in formic acid (5 mL) was stirred at 90° C. for 5 hours. The mixture was concentrated to remove formic acid and adjusted to pH=8 by the addition of ammonium hydroxide. The crude was purified by Purification Procedure Q with 0.05% ammonium hydroxide and 10 mM ammonium hydrogen carbonate in water-acetonitrile (18-48%) to afford (1R,3 S)-3-(3-(pyridazin-3-ylamino)-1H-pyrazol-5-yl)cyclopentyl (1-methylcyclopropyl)carbamate (9.8 mg, 37%). LCMS (ES-API, m/z): 343.0 [M+H]⁺; ¹H NMR (400 MHz, DMSO-d₆) δ 11.93 (s, 1H), 9.51 (s, 1H), 8.58 (d, J=3.6 Hz, 1H), 7.55-7.40 (i, 1H), 7.39-7.36 (m, 2H), 6.20 (s, 1H), 5.00 (br s, 1H), 3.05-3.03 (m, 1H), 2.47-2.45 (m, 1H), 2.07-1.84 (m, 2H), 1.72-1.59 (m, 3H), 1.23 (s, 3H), 0.59 (br s, 2H), 0.47 (br s, 2H).

Other compounds were synthesized according to General Procedure and Example 3, as set forth in the below Table H.

TABLE H
General Procedure 3 and Example 3 Compounds

| | | | | MS |
|---|---|---|---|---|
| Ex | Structure/Name | Starting Material | ¹H NMR | (m/z) |
| 3A | 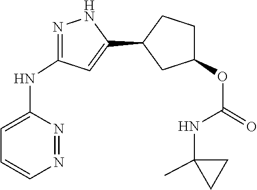 [SMILES: CC1(NC(=O)O[C@@H]2CC[C@H](c3cc(Nc4cccnn4)n[nH]3)C2)CC1] | 1-methyl- cyclopropanamine hydrochloride | ¹H NMR (400 MHz, DMSO-d₆) δ 11.93 (br s, 1H), 9.51 (s, 1H), 8.58 (d, J = 3.6 Hz, 1H), 7.55-7.52 (m, 1H), 7.40-7.36 (m, 2H), 6.20 (s, 1H), 5.00 (br s, 1H), 3.05-3.02 (m, 1H), 2.48-2.44 (m, 1H), 2.07- 2.00 (m, 1H), 1.91-1.84 (m, 1H), 1.80-1.58 (m, 3H), 1.23 (s, 3H), 0.59 (br s, 2H), 0.47 (br s, 2H). | 343.0 [M + H]⁺ |
| | (1R,3S)-3-(3-(pyridazin-3- | | | |
| | ylamino)-1H-pyrazol-5- | | | |
| | yl)cyclopentyl (1- | | | |
| | methylcyclopropyl)carbamate | | | |
| 3B* | 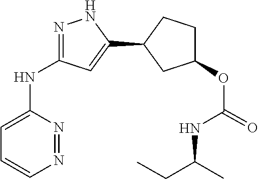 [SMILES: CC[C@H](C)NC(=O)O[C@@H]1CC[C@H](c2cc(Nc3cccnn3)n[nH]2)C1] | butan-2-amine hydrochloride | ¹H NMR (400 MHz, DMSO-d₆) δ 11.95 (br s, 1H), 9.53 (s, 1H), 8.58 (d, J = 3.6 Hz, 1H), 7.51 (s, 1H), 7.41-7.37 (m, 1H), 6.91 (d, J = 8.4 Hz, 1H), 6.21 (s, 1H), 5.01 (br s, 1H), 3.39-3.37 (m, 1H), 3.07- 3.05 (m, 1H), 2.45-2.41 (m, 1H), 2.03-1.98 (m, 1H), 1.90-1.84 (m, 1H), 1.76-1.56 (m, 3H), 1.37- 1.33 (m, 2H), 1.00 (d, J = 6.8 Hz, 3H), 0.82-0.79 (m, 3H). | 345.1 [M + H]⁺ |
| | (1R,3S)-3-(3-(pyridazin-3- | | | |
| | ylamino)-1H-pyrazol-5- | | | |
| | yl)cyclopentyl ((S)-sec- | | | |
| | butyl)carbamate | | | |
| 3C* | 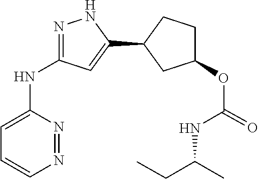 [SMILES: CC[C@@H](C)NC(=O)O[C@@H]1CC[C@H](c2cc(Nc3cccnn3)n[nH]2)C1] | butan-2-amine hydrochloride | ¹H NMR (400 MHz, methanol-d₄) δ 8.57 (br s, 1H), 7.45 (s, 2H), 6.29 (s, 1H), 5.10 (br s, 1H), 3.51-3.47 (m, 1H), 3.20-3.17 (m, 1H), 2.57- 2.52 (m, 1H), 2.14-2.08 (m, 1H), 1.97-1.81 (m, 4H), 1.43- 1.39 (m, 2H), 1.09 (d, J = 6.8 Hz, 3H), 0.86 (t, J = 7.2 Hz, 3H). | 345.1 [M + H]⁺ |
| | (1R,3S)-3-(3-(pyridazin-3- | | | |
| | ylamino)-1H-pyrazol-5- | | | |
| | yl)cyclopentyl ((R)-sec- | | | |
| | butyl)carbamate | | | |
| 3D | 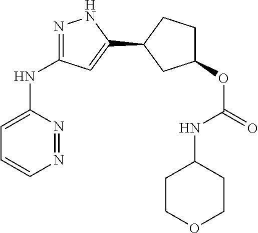 [SMILES: O=C(NC1CCOCC1)O[C@@H]1CC[C@H](c2cc(Nc3cccnn3)n[nH]2)C1] | tetrahydro-2H- pyran-4-amine | ¹H NMR (400 MHz, DMSO-d₆) δ 11.95 (s, 1H), 9.53 (s, 1H), 8.58 (d, J = 4.4 Hz, 1H), 7.51 (s, 1H), 7.41- 7.37 (m, 1H), 7.14 (d, J = 7.8 Hz, 1H), 6.21 (s, 1H), 5.02 (s, 1H), 3.80- 3.78 (m, 2H), 3.47-3.42 (m, 2H), 3.39-3.27 (m, 2H), 3.08- 3.06 (m, 1H), 2.04-1.90 (m, 2H), 1.75-1.66 (m, 5H), 1.38-1.36 (m, 2H). | 373.2 [M + H]⁺ |
| | (1R,3S)-3-(3-(pyridazin-3- | | | |
| | ylamino)-1H-pyrazol-5- | | | |
| | yl)cyclopentyl (tetrahydro-2H- | | | |
| | pyran-4-yl)carbamate | | | |
| 3E | 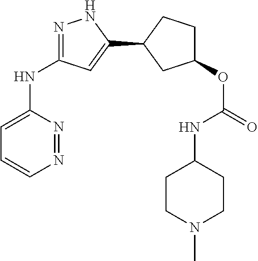 [SMILES: CN1CCC(NC(=O)O[C@@H]2CC[C@H](c3cc(Nc4cccnn4)n[nH]3)C2)CC1] | 1-methyl- piperidin-4-amine | ¹H NMR (400 MHz, DMSO-d₆) δ 11.94 (br s, 1H), 9.53 (s, 1H), 8.58 (d, J = 3.6 Hz, 1H), 7.52 (d, J = 8.0 Hz, 1H), 7.41-7.37 (m, 1H), 7.04 (d, J = 7.6 Hz, 1H), 6.20 (s, 1H), 5.01 (br s, 1H), 3.20 (br s, 1H), 3.12-2.97 (m, 1H), 2.66-2.61 (m, 2H), 2.45-2.41 (m, 1H), 2.11 (s, 3H), 2.04-1.98 (m, 1H), 1.96- 1.83 (m, 3H), 1.74-1.65 (m, 5H), 1.39-1.36 (m, 2H). | 386.1 [M + H]⁺ |
| | (1R,3S)-3-(3-(pyridazin-3- | | | |
| | ylamino)-1H-pyrazol-5- | | | |
| | yl)cyclopentyl (1- | | | |
| | methylpiperidin-4- | | | |
| | yl)carbamate | | | |
| 3F | 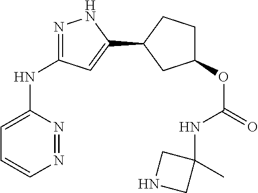 [SMILES: CC1(NC(=O)O[C@@H]2CC[C@H](c3cc(Nc4cccnn4)n[nH]3)C2)CNC1] | tert-butyl 3- amino-3- methylazetidine-1- carboxylate | ¹H NMR (400 MHz, methanol-d₄) δ 8.58-8.57 (m, 1H), 7.47-7.42 (m, 2H), 6.29 (br s, 1H), 5.15 (br s, 1H), 4.17-4.07 (m, 2H), 3.65- 3.63 (m, 2H), 3.22-3.20 (m, 1H), 2.57-2.53 (m, 1H), 2.15-2.01 (m, 1H), 1.97-1.87 (m, 4H), 1.53 (s, 3H). | 357.8 [M + H]⁺ |
| | (1R,3S)-3-(3-(pyridazin-3- | | | |
| | ylamino)-1H-pyrazol-5- | | | |
| | yl)cyclopentyl (3- | | | |
| | methylazetidin-3-yl)carbamate | | | |
| 3G | 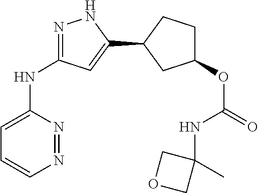 [SMILES: CC1(NC(=O)O[C@@H]2CC[C@H](c3cc(Nc4cccnn4)n[nH]3)C2)COC1] | 3-methyloxetan-3- amine | ¹H NMR (400 MHz, DMSO-d₆) δ 11.95 (s, 1H), 9.54 (s, 1H), 8.59- 8.58 (m, 1H), 7.65-7.50 (m, 2H), 7.61-7.38 (m, 1H), 7.44-7.35 (m, 1H), 6.20 (s, 1H), 5.03-5.01 (m, 1H), 4.56 (s, 1H), 4.24-4.23 (m, 1H), 3.11-3.02 (m, 1H), 2.47- 2.41 (m, 1H), 2.04-2.02 (m, 2H), 1.76-1.64 (m, 3H), 1.47 (s, 3H). | 359.0 [M + H]⁺ |
| | (1R,3S)-3-(3-(pyridazin-3- | | | |
| | ylamino)-1H-pyrazol-5- | | | |
| | yl)cyclopentyl (3- | | | |
| | methyloxetan-3-yl)carbamate | | | |

*Examples 3B, 3C: stereochemical configuration of the carbamate N-substituent was assigned arbitrarily

### General Procedure 4

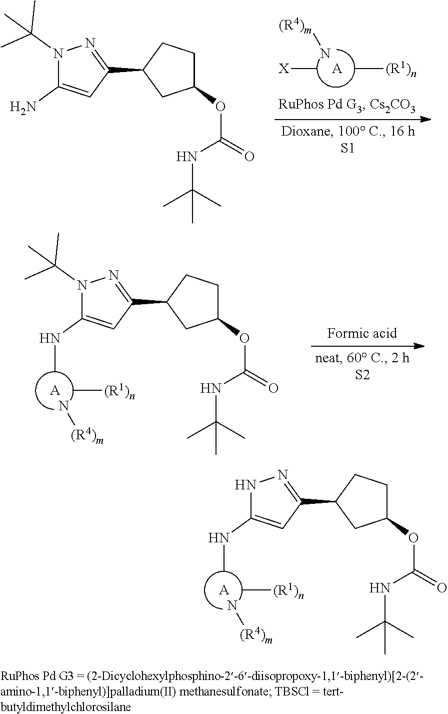

### Example 4: (1R,3S)-3-(3-((2-methylpyridin-3-yl)amino)-1H-pyrazol-5-yl)cyclopentyl tert-butylcarbamate

The title compound was prepared according to General Procedure 4 as described below.

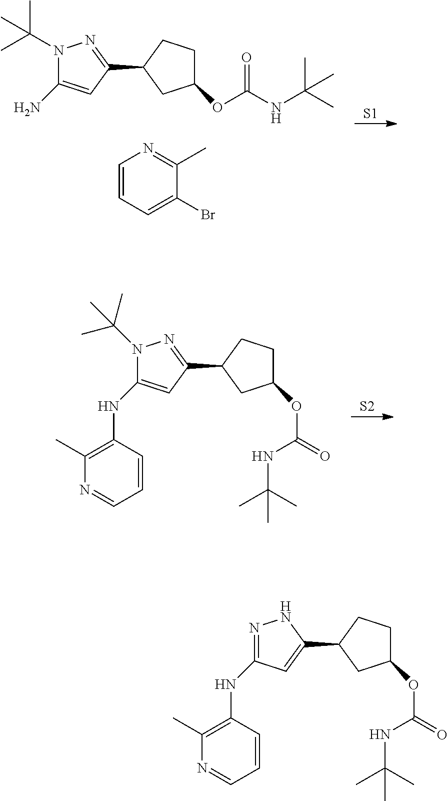

Step 1: (1R,3S)-3-(1-(tert-butyl)-5-((2-methylpyridin-3-yl)amino)-1H-pyrazol-3-yl)cyclopentyl tert-butylcarbamate. A mixture of (1R,3S)-3-(5-amino-1-(tert-butyl)-1H-pyrazol-3-yl)cyclopentyl tert-butylcarbamate (Intermediate B) (200 mg, 0.62 mmol), 3-bromo-2-methylpyridine (0.14 mL, 1.2 mmol), cesium carbonate (606 mg, 1.9 mmol) and (2-dicyclohexylphosphino-2′,6′-diisopropoxy-1,1′-biphenyl)[2-(2′-amino-1,1′-biphenyl)]palladium(II) methanesulfonate (52 mg, 0.06 mmol) in 1,4-dioxane (10 mL) was stirred at 100° C. for 16 h under nitrogen atmosphere and concentrated under reduced pressure. The residue was purified by column chromatography (silica gel, 100-200 mesh, 0-60% ethyl acetate:petroleum ether) to afford (1R,3S)-3-[1-tert-butyl-5-[(2-methyl-3-pyridyl)amino]pyrazol-3-yl]cyclopentyl-N-tert-butylcarbamate (200 mg, 78%). LCMS (ES-API, m/z): 414.2 [M+H]⁺.

Step 2: (1R,3S)-3-(3-((2-methylpyridin-3-yl)amino)-1H-pyrazol-5-yl)cyclopentyl tert-butylcarbamate. A solution of (1R,3S)-3-[1-tert-butyl-5-[(2-methyl-3-pyridyl)amino]pyrazol-3-yl]cyclopentyl-N-tert-butylcarbamate (200 mg, 0.48 mmol) in formic acid (5 mL) was stirred at 60° C. for 16 h and concentrated under reduced pressure. The residue was purified by Purification Procedure D with 0.225% formic acid in water-acetonitrile (15-45%) to afford (1R,3S)-3-(3-((4-methylpyridin-3-yl)amino)-1H-pyrazol-5-yl)cyclopentyl tert-butylcarbamate (45 mg, 26%). LCMS (ES-API, m/z): 358.1 [M+H]⁺; ¹H NMR (400 MHz, DMSO-d₆) δ 12.11 (s, 1H), 8.14 (s, 1H), 8.05 (d, J=8.4 Hz, 1H), 7.83-7.82 (m, 1H), 7.45 (s, 1H), 7.06-7.03 (m, 1H), 6.76 (s, 1H), 5.77 (s, 1H), 4.97 (s, 1H), 3.08-3.01 (m, 1H), 2.46-2.44 (m, 1H), 2.43 (s, 3H), 2.02-1.89 (m, 2H), 1.75-1.58 (m, 3H), 1.20 (s, 9H).

### Example 4K: (1R,3S)-3-(3-(pyridazin-4-ylamino)-1H-pyrazol-5-yl)cyclopentyl tert-butylcarbamate

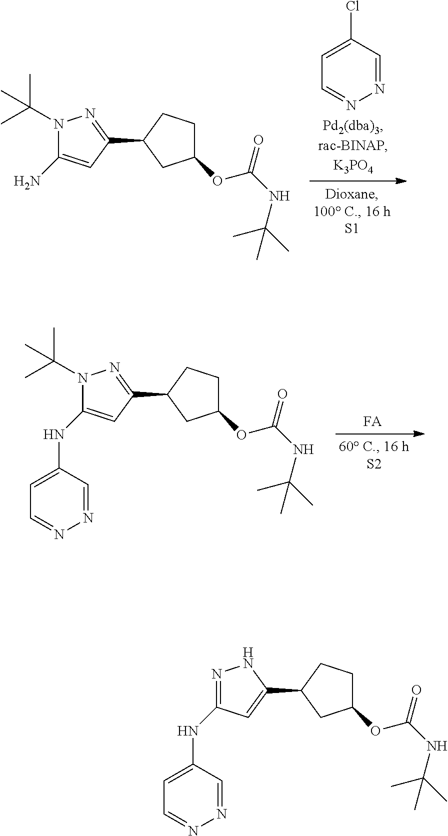

Step 1: (1R,3S)-3-(1-(tert-butyl)-5-(pyridazin-4-ylamino)-1H-pyrazol-3-yl)cyclopentyl tert-butylcarbamate. A mixture of (1R,3S)-3-(5-amino-1-(tert-butyl)-1H-pyrazol-3-yl)cyclopentyl tert-butylcarbamate (200 mg, 0.62 mmol), 4-chloropyridazine (85 mg, 0.74 mmol), potassium phosphate (395 mg, 1.86 mmol), 2,2′-bis(diphenylphosphino)-1,1′-binaphthyl (39 mg, 0.06 mmol) and tris (dibenzylideneacetone) dipalladium (0) (34 mg, 0.04 mmol) in 1,4-dioxane (10 mL) was stirred at 100° C. for 16 hours under nitrogen atmosphere. To the reaction was added methanol (10 mL). The mixture was filtered and the filtrate was concentrated under reduced pressure. The residue was purified by column chromatography (silica gel, 100-200 mesh, 0 to 10% methanol in dichloromethane) to obtain (1R,3S)-3-(1-(tert-butyl)-5-(pyridazin-4-ylamino)-1H-pyrazol-3-yl)cyclopentyl tert-butylcarbamate (75 mg, 30%). LCMS (ES-API, M/Z): 401.3 [M+H]⁺.

Step 2: (1R,3S)-3-(3-(pyridazin-4-ylamino)-1H-pyrazol-5-yl)cyclopentyl tert-butylcarbamate. A solution of (1R,3S)-3-(1-(tert-butyl)-5-(pyridazin-4-ylamino)-1H-pyrazol-3-yl)cyclopentyl tert-butylcarbamate (75 mg, 0.19 mmol) in formic acid (3 mL) was stirred at 60° C. for 16 hours. The mixture was concentrated to remove formic acid and the crude was purified by Purification Procedure O with 0.225% formic acid in water-acetonitrile (11-41%) to afford (1R,3S)-3-(3-(pyridazin-4-ylamino)-1H-pyrazol-5-yl)cyclopentyl tert-butylcarbamate (13.1 mg, 19.5%). LCMS (ES-API, M/Z): 345.0 [M+H]⁺; ¹H NMR (400 MHz, DMSO-d6) δ 12.10 (s, 1H), 9.38 (s, 1H), 8.97 (s, 1H), 8.68 (d, J=6.0 Hz, 1H), 8.14 (s, 1H), 7.58-7.56 (m, 1H), 6.76 (br s, 1H), 5.76 (s, 1H), 4.97 (s, 1H), 3.11-3.04 (m, 1H), 2.47-2.43 (m, 1H), 2.03-2.01 (m, 1H), 1.91-1.89 (m, 1H), 1.73-1.59 (m, 3H), 1.20 (s, 9H).

Other compounds were synthesized according to General Procedure and Example 4, as set forth in the below in Table I.

TABLE I
General Procedure 4 and Example 4 Compounds

| | | Starting | | |
|---|---|---|---|---|
| Ex | Structure/Name | Material | ¹H NMR | MS (m/z) |
| 4A | 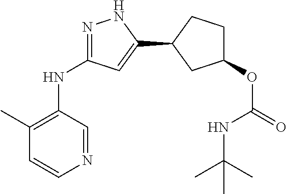 [SMILES: Cc1ccncc1Nc1cc([C@H]2CC[C@@H](OC(=O)NC(C)(C)C)C2)[nH]n1] | 3-bromo-4- methyl- pyridine | ¹H NMR (400 MHz, DMSO-d₆) δ 11.86 (s, 1H), 8.89 (s, 1H), 7.87- 7.86 (m, 1H), 7.40 (s, 1H), 7.06- 7.05 (m, 1H), 6.76 (s, 1H), 5.74 (s, 1H), 4.97 (s, 1H), 3.05-3.01 (m, 1H), 2.46-2.45 (m, 1H), 2.22 (s, 3H), 2.02-1.87 (m, 2H), 1.72-1.59 (m, 3H), 1.20 (s, 9H). | 358.1 [M + H]⁺ |
| | (1R,3S)-3-(3-((4-methylpyridin-3- | | | |
| | yl)amino)-1H-pyrazol-5- | | | |
| | yl)cyclopentyl tert-butylcarbamate | | | |
| 4B | 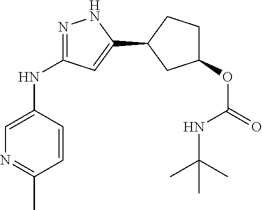 [SMILES: Cc1ccc(Nc2cc([C@H]3CC[C@@H](OC(=O)NC(C)(C)C)C3)[nH]n2)cn1] | 5-bromo-2- methyl- pyridine | ¹H NMR (400 MHz, DMSO-d₆) δ 11.73 (br s, 1H), 8.41 (d, J = 2.4 Hz, 1H), 8.35 (s, 1H), 8.15 (s, 1H), 7.69 (dd, J = 8.8, 2.8 Hz, 1H), 7.02 (d, J = 8.4 Hz, 1H), 6.76 (s, 1H), 5.59 (s, 1H), 4.97 (s, 1H), 3.04-2.99 (m, 1H), 2.50- 2.43 (m, 1H), 2.33 (s, 3H), 2.01- 1.98 (m, 1H), 1.89-1.86 (m, 1H), 1.71-1.69 (m, 2H), 1.63- 1.55 (m, 1H), 1.20 (s, 9H). | 358.1 [M + H]⁺ |
| | (1R,3S)-3-(3-((6-methylpyridin-3- | | | |
| | yl)amino)-1H-pyrazol-5- | | | |
| | yl)cyclopentyl tert-butylcarbamate | | | |
| 4C | 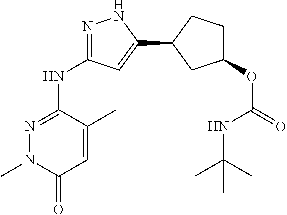 [SMILES: Cc1cc(=O)n(C)nc1Nc1cc([C@H]2CC[C@@H](OC(=O)NC(C)(C)C)C2)[nH]n1] | 6-chloro-2,5- dimethyl- pyridazin-3- one | ¹H NMR (400 MHz, DMSO-d₆) δ 11.89 (s,1H) 8.13 (s, 1H), 6.73 (s, 1H), 6.71-6.66 (m, 1H), 6.21 (s, 1H), 5.01-4.93 (m, 1H), 3.51 (s, 3H), 3.08-2.98 (m, 1H), 2.45 (m, 1H), 2.18 (d, J = 1.3 Hz, 3H), 2.06-1.95 (m, 1H) 1.94-1.83 (m, 1H), 1.78-1.66 (m, 2H), 1.66-1.56 (m, 1H), 1.20 (s, 9H). | 389.2 [M + H]⁺ |
| | (1R,3S)-3-(3-((1,4-dimethyl-6- | | | |
| | oxo-1,6-dihydropyridazin-3- | | | |
| | yl)amino)-1H-pyrazol-5- | | | |
| | yl)cyclopentyl tert-butylcarbamate | | | |
| 4D | 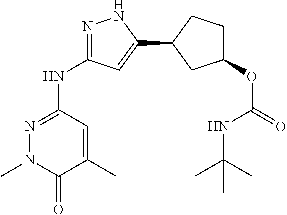 [SMILES: Cc1cc(Nc2cc([C@H]3CC[C@@H](OC(=O)NC(C)(C)C)C3)[nH]n2)nn(C)c1=O] | 6-chloro-2,4- dimethyl- pyridazin-3- one | ¹H NMR (400 MHz, DMSO-d₆) δ 11.85 (s, 1H), 9.02 (s, 1H), 7.15 (s, 1H), 6.74 (s, 1H), 6.19 (s, 1H), 4.97 (s, 1H), 3.53 (s, 3H), 3.09- 2.97 (m, 1H), 2.50-2.39 (m, 1H), 2.04 (d, J = 1.3 Hz, 3H), 2.02-1.94 (m, 1H), 1.94-1.82 (m, 1H), 1.78-1.65 (m, 2H), 1.63 (m, 1H), 1.20 (s, 9H). | 389.2 [M + H]⁺ |
| | (1R,3S)-3-(3-((1,5-dimethyl-6- | | | |
| | oxo-1,6-dihydropyridazin-3- | | | |
| | yl)amino)-1H-pyrazol-5- | | | |
| | yl)cyclopentyl tert-butylcarbamate | | | |
| 4E | 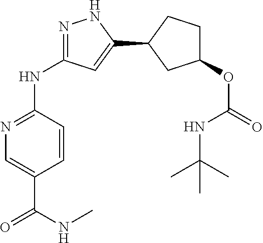 [SMILES: CNC(=O)c1ccc(Nc2cc([C@H]3CC[C@@H](OC(=O)NC(C)(C)C)C3)[nH]n2)nc1] | 6-chloro-N- methyl- nicotinamide | ¹H NMR (400 MHz, DMSO-d₆) δ 11.95 (s, 1H), 9.53 (s, 1H), 8.59 (s, 1H), 8.22-8.21 (m, 1H), 7.95- 7.93 (m, 1H), 7.25-7.23 (m, 1H), 6.76 (s, 1H), 6.11 (s, 1H), 4.98 (s, 1H), 3.07-3.02 (m, 1H), 2.76 (s, 3H), 2.50-2.46 (m, 1H), 2.00-1.84 (m, 2H), 1.73-1.59 (m, 3H), 1.21 (s, 9H). | 401.1 [M + H]⁺ |
| | (1R,3S)-3-(3-((5- | | | |
| | (methylcarbamoyl)pyridin-2- | | | |
| | yl)amino)-1H-pyrazol-5- | | | |
| | yl)cyclopentyl tert-butylcarbamate | | | |
| 4F | 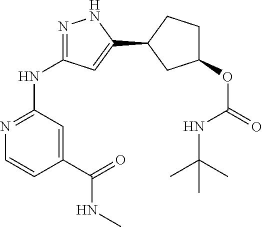 [SMILES: CNC(=O)c1ccnc(Nc2cc([C@H]3CC[C@@H](OC(=O)NC(C)(C)C)C3)[nH]n2)c1] | 2-chloro-N- methyliso- nicotinamide | ¹H NMR (400 MHz, DMSO-d₆) δ 11.86 (br s, 1H), 9.26 (s, 1H), 8.49 (s, 1H), 8.17 (d, J = 5.2 Hz, 1H), 7.62 (s, 1H), 6.96 (d, J = 4.8 Hz, 1H), 6.77 (br s, 1H), 6.11 (br s, 1H), 4.98 (br s, 1H), 3.06-3.01 (m, 1H), 2.76 (d, J = 4.4 Hz, 3H), 2.45-2.41 (m, 1H), 2.00-1.90 (m, 1H), 1.89-1.85 (m, 1H), 1.73- 1.59 (m, 3H), 1.21 (s, 9H). | 401.1 [M + H]⁺ |
| | (1R,3S)-3-(3-((4- | | | |
| | (methylcarbamoyl)pyridin-2- | | | |
| | yl)amino)-1H-pyrazol-5- | | | |
| | yl)cyclopentyl tert-butylcarbamate | | | |
| 4G | 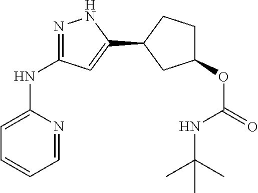 [SMILES: CC(C)(C)NC(=O)O[C@@H]1CC[C@H](c2cc(Nc3ccccn3)n[nH]2)C1] | 2- chloropyridine | ¹H NMR (400 MHz, DMSO-d₆) δ 9.09 (s, 1H), 8.17 (s, 1H), 8.09- 8.08 (m, 1H), 7.53-7.49 (m, 1H), 7.21-7.19 (m, 1H), 6.78 (s, 1H), 6.68-6.65 (m, 1H), 6.07 (s, 1H), 4.97 (s, 1H), 3.04-2.98 (m, 1H), 2.46-2.42 (m, 1H), 2.06- 1.81 (m, 2H), 1.78 (m, 1H), 1.78- 1.55 (m, 1H), 1.55-1.53 (m, 1H), 1.20 (s, 9H). | 344.1 [M + H]⁺ |
| | (1R,3S)-3-(3-(pyridin-2-ylamino)- | | | |
| | 1H-pyrazol-5-yl)cyclopentyl tert- | | | |
| | butylcarbamate | | | |
| 4H | 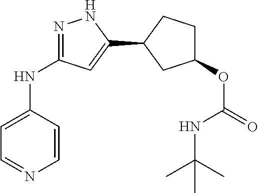 [SMILES: CC(C)(C)NC(=O)O[C@@H]1CC[C@H](c2cc(Nc3ccncc3)n[nH]2)C1] | 4-bromo- pyridine | ¹H NMR (400 MHz, DMSO-d₆) δ 11.96 (s, 1H), 8.95 (s, 1H), 8.15 (d, J = 6.4 Hz, 2H), 7.19 (d, J = 4.8 Hz, 2H), 6.80 (s, 1H), 5.71 (s, 1H), 4.97 (s, 1H), 3.09-3.00 (m, 1H), 2.46-2.44 (m, 1H), 1.99- 1.87 (m, 2H), 1.72-1.59 (m, 3H), 1.20 (s, 9H). | 344.1 [M + H]⁺ |
| | (1R,3S)-3-(3-(pyridin-4-ylamino)- | | | |
| | 1H-pyrazol-5-yl)cyclopentyl tert- | | | |
| | butylcarbamate | | | |
| 4I | 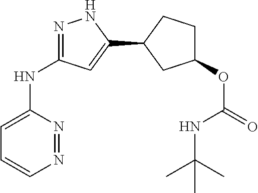 [SMILES: CC(C)(C)NC(=O)O[C@@H]1CC[C@H](c2cc(Nc3cccnn3)n[nH]2)C1] | 3-chloro- pyridazine | ¹H NMR (400 MHz, DMSO-d₆) δ 9.53 (s, 1H), 8.58-8.57 (m, 1H), 8.25 (s, 1H), 7.51-7.49 (m, 1H), 7.40-7.37 (m, 1H), 6.78 (s, 1H), 6.19 (s, 1H), 4.97 (br s, 1H), 3.09- 3.02 (m, 1H), 2.00-1.85 (m, 3H), 1.73-1.59 (m, 3H), 1.20 (s, 9H). | 345.1 [M + H]⁺ |
| | (1R,3S)-3-(3-(pyridazin-3- | | | |
| | ylamino)-1H-pyrazol-5- | | | |
| | yl)cyclopentyl tert-butylcarbamate | | | |
| 4J | 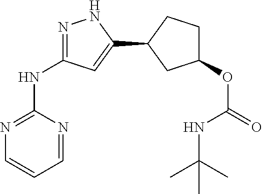 [SMILES: CC(C)(C)NC(=O)O[C@@H]1CC[C@H](c2cc(Nc3ncccn3)n[nH]2)C1] | 2-chloro- pyrimidine | ¹H NMR (400 MHz, DMSO-d₆) δ 9.59 (s, 1H), 8.41-8.36 (m, 2H), 6.77-6.75 (m, 2H), 6.30 (s, 1H), 4.97 (s, 1H), 3.03-2.99 (m, 1H), 2.46-2.43 (m, 1H), 2.01-1.88 (m, 2H), 1.73-1.61 (m, 3H), 1.20 (s, 9H). | 345.1 [M + H]⁺ |
| | (1R,3S)-3-(3-(pyrimidin-2- | | | |
| | ylamino)-1H-pyrazol-5- | | | |
| | yl)cyclopentyl tert-butylcarbamate | | | |
| 4K | 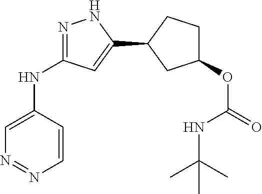 [SMILES: CC(C)(C)NC(=O)O[C@@H]1CC[C@H](c2cc(Nc3ccnnc3)n[nH]2)C1] | 4-chloro- pyridazine | ¹H NMR (400 MHz, DMSO-d₆) δ 12.10 (br s, 1H), 9.38 (s, 1H), 8.97 (s, 1H), 8.68 (d, J = 6.0 Hz, 1H), 8.14 (s, 1H), 7.57 (dd, J = 6.0, 2.8 Hz, 1H), 6.76 (br s, 1H), 5.76 (s, 1H), 4.97 (br s, 1H), 3.11- 3.04 (m, 1H), 2.47-2.43 (m, 1H), 2.04-2.01 (m, 1H), 1.91- 1.86 (m, 1H), 1.73-1.69 (m, 2H), 1.65-1.59 (m, 1H), 1.20 (s, 9H). | 345.0 [M + H]⁺ |
| | (1R,3S)-3-(3-(pyridazin-4- | | | |
| | ylamino)-1H-pyrazol-5- | | | |
| | yl)cyclopentyl tert-butylcarbamate | | | |
| 4L | 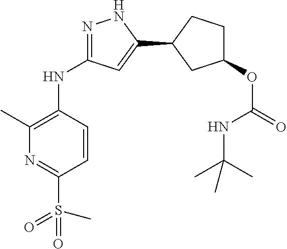 [SMILES: Cc1nc(S(C)(=O)=O)ccc1Nc1cc([C@H]2CC[C@@H](OC(=O)NC(C)(C)C)C2)[nH]n1] | Prepared by General Procedure 4 from 3- bromo-2- methyl-6- (methyl- sulfonyl) pyridine | ¹H NMR (400 MHz, DMSO-d₆) δ 12.07 (br s, 1H), 8.33 (d, J = 8.8 Hz, 1H), 8.19 (s, 1H), 7.74 (d, J = 8.8 Hz, 1H), 6.77 (s, 1H), 5.90 (s, 1H), 4.98 (s, 1H), 3.13 (s, 3H), 3.09-3.05 (m, 1H), 2.53-2.50 (m, 3H), 2.04-2.02 (m, 1H), 1.92-1.89 (m, 1H), 1.73-1.71 (m, 2H), 1.70-1.59 (m, 1H), 1.20 (s, 9H). | 436.1 [M + H]⁺ |
| | (1R,3S)-3-(3-((2-methyl-6- | | | |
| | (methylsulfonyl)pyridin-3- | | | |
| | yl)amino)-1H-pyrazol-5- | | | |
| | yl)cyclopentyl tert-butylcarbamate | | | |

### General Procedure 5

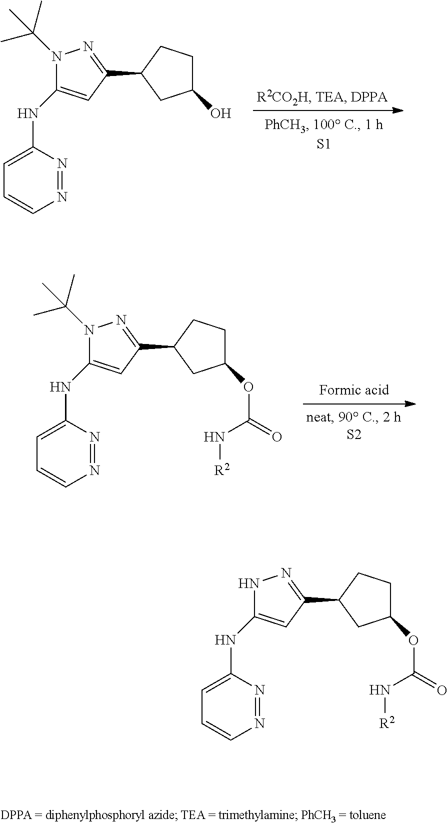

### Example 5: (1R,3S)-3-(3-(pyridazin-3-ylamino)-1H-pyrazol-5-yl)cyclopentyl (1-(trifluoromethyl)cyclopropyl)carbamate

The title compound was prepared according to General Procedure 5 as described below.

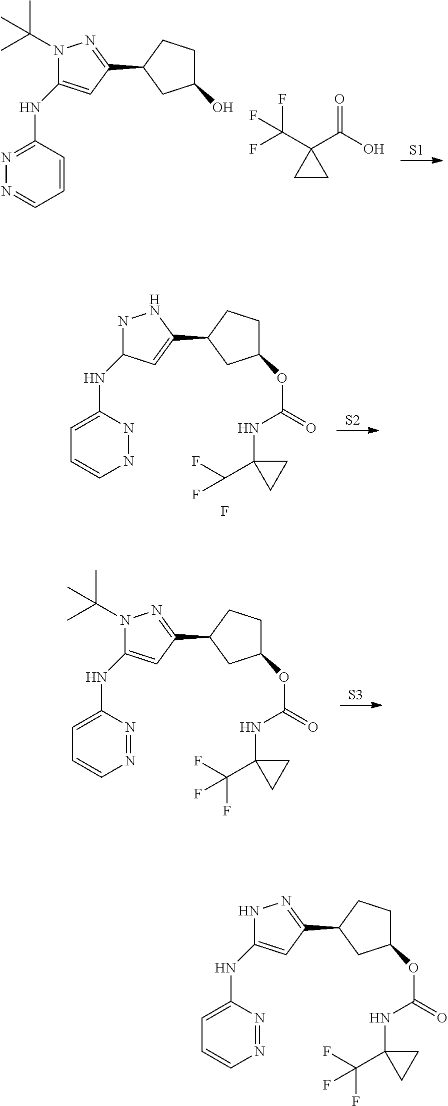

Step 1: (1R,3S)-3-(1-(tert-butyl)-5-(pyridazin-3-ylamino)-1H-pyrazol-3-yl)cyclopentyl (1-(trifluoromethyl)cyclopropyl)carbamate. A mixture of 1-(trifluoromethyl)cyclopropanecarboxylic acid (100 mg, 0.65 mmol), triethylamine (0.11 mL, 0.78 mmol) and diphenylphosphoryl azide (0.15 mL, 0.71 mmol) in toluene (5 mL) was heated at 100° C. for 1 h, followed by addition of (1R,3S)-3-[1-tert-butyl-5-(pyridazin-3-ylamino)pyrazol-3-yl]cyclopentanol (Prepared by the method of Example 2, Step 3) (391 mg, 1.30 mmol). The resulting mixture was stirred at 100° C. for an additional 16 h and concentracted under reduced pressure. The residue was purified by column chromatography (silica gel, 100-200 mesh, 0-60% ethyl acetate:petroleum ether) to afford (1R,3S)-3-(1-(tert-butyl)-5-(pyridazin-3-ylamino)-1H-pyrazol-3-yl)cyclopentyl (1-(trifluoromethyl)cyclopropyl)carbamate (120 mg, 41%). LCMS (ES-API, m/z): 453.1 [M+H]⁺.

Step 2: (1R,3S)-3-(3-(pyridazin-3-ylamino)-1H-pyrazol-5-yl)cyclopentyl (1-(trifluoromethyl)cyclopropyl)carbamate. A solution of (1R,3S)-3-(1-(tert-butyl)-5-(pyridazin-3-ylamino)-1H-pyrazol-3-yl)cyclopentyl (1-(trifluoromethyl)cyclopropyl)carbamate (100 mg, 0.22 mmol) in formic acid (2 mL) was stirred at 90° C. for 2 h and concentrated under reduced pressure. The residue was purified by Purification Procedure D with 0.225% formic acid in water-acetonitrile (20-45%) to afford (1R,3S)-3-(3-(pyridazin-3-ylamino)-1H-pyrazol-5-yl)cyclopentyl (1-(trifluoromethyl)cyclopropyl)carbamate (30 mg, 34%). LCMS (ES-API, m/z): 397.0 [M+H]⁺; H NMR (400 MHz, DMSO-d₆) δ 12.00 (s, 1H), 9.53 (s, 1H), 8.59-8.58 (m, 1H), 8.16-8.11 (m, 1H), 7.52-7.50 (m, 1H), 7.41-7.37 (m, 1H), 6.19 (s, 1H), 5.03 (s, 1H), 3.08-3.04 (m, 1H), 2.04-1.90 (m, 2H), 1.75-1.61 (m, 3H), 1.20-1.18 (m, 2H), 1.17-1.03 (m, 2H).

### Example 5A: (1R,3S)-3-(3-(pyridazin-3-ylamino)-1H-pyrazol-5-yl)cyclopentyl ((S)-1,1,1-trifluoropropan-2-yl)carbamate

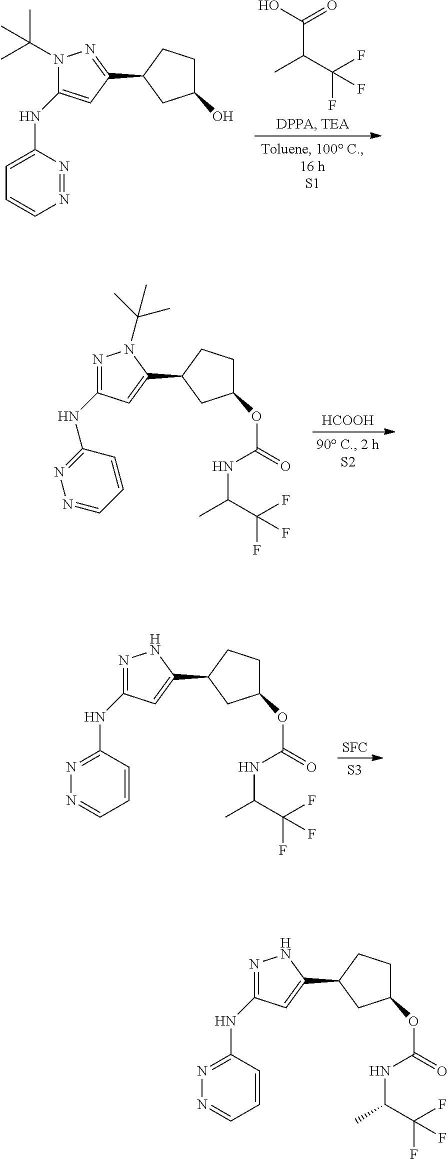

Step 1: (1R,3S)-3-(1-(tert-butyl)-3-(pyridazin-3-ylamino)-1H-pyrazol-5-yl)cyclopentyl (1,1,1-trifluoropropan-2-yl)carbamate. A mixture of 3,3,3-trifluoro-2-methyl-propanoic acid (150 mg, 1.06 mmol), triethylamine (0.18 mL, 1.27 mmol) and diphenylphoshorylazide (0.25 mL, 1.16 mmol) in toluene (5 mL) was heated at 100° C. for 1 hour, followed by addition of (1R,3S)-3-[1-tert-butyl-5-(pyridazin-3-ylamino)pyrazol-3-yl]cyclopentanol (318 mg, 1.06 mmol). The resulting mixture was stirred at 100° C. for an additional 16 h and concentracted under reduced pressure. The residue was purified by column chromatography (silica gel, 100-200 mesh, 0 to 10% ethyl acetate in petroleum ether) to afford (1R,3S)-3-(1-(tert-butyl)-3-(pyridazin-3-ylamino)-1H-pyrazol-5-yl)cyclopentyl (1,1,1-trifluoropropan-2-yl)carbamate (130 mg, 28%). LCMS (ES-API, M/Z): 441.3 [M+H]⁺.

Step 2: (1R,3S)-3-(3-(pyridazin-3-ylamino)-1H-pyrazol-5-yl)cyclopentyl (1,1,1-trifluoropropan-2-yl)carbamate. A solution of (1R,3S)-3-(1-(tert-butyl)-3-(pyridazin-3-ylamino)-1H-pyrazol-5-yl)cyclopentyl (1,1,1-trifluoropropan-2-yl)carbamate (130 mg, 0.3000 mmol) in formic acid (2.67 mL) was stirred at 90° C. for 2 hours. After concentrated, the residue was adjusted to pH=8 by the addition of ammonium hydroxide. The crude product was purified by column chromatography (silica gel, 100-200 mesh, 0 to 6% methanol in dichloromethane) to obtain (1R,3S)-3-(3-(pyridazin-3-ylamino)-1H-pyrazol-5-yl)cyclopentyl (1,1,1-trifluoropropan-2-yl)carbamate (80 mg, 71%) LCMS (ES-API, M/Z): 385.1 [M+H]⁺.

Step 3: (1R,3S)-3-(3-(pyridazin-3-ylamino)-1H-pyrazol-5-yl)cyclopentyl ((S)-1,1,1-trifluoropropan-2-yl)carbamate. The title compound (1R,3S)-3-(3-(pyridazin-3-ylamino)-1H-pyrazol-5-yl)cyclopentyl (1,1,1-trifluoropropan-2-yl)carbamate (80 mg, 0.21 mmol) was purified by Purification Procedure T (SFC separation) with 0.1% ammonium hydroxide-40% ethanol-carbon dioxide to afford (1R,3S)-3-(3-(pyridazin-3-ylamino)-1H-pyrazol-5-yl)cyclopentyl ((S)-1,1,1-trifluoropropan-2-yl)carbamate (25.5 mg, 31%) (peak 1, retention time=4.454 min). LCMS (ES-API, M/Z): 385.0 [M+H]⁺; ¹H NMR (400 MHz, DMSO-d6) δ 11.97 (br s, 1H), 9.53 (s, 1H), 8.59-8.58 (m, 1H), 7.85 (d, J=8.8 Hz, 1H), 7.51 (d, J=7.6 Hz, 1H), 7.41-7.38 (m, 1H), 6.21 (s, 1H), 5.07 (br s, 1H), 4.29-4.24 (m, 1H), 3.09-3.05 (m, 1H), 2.55-2.50 (m, 1H), 2.05-1.91 (m, 2H), 1.77-1.62 (m, 3H). 1.22 (br d, J=6.8 Hz, 3H).

Other compounds were synthesized according to General Procedure and Example 5, as set forth in the below Table J.

TABLE J
General Procedure 5 and Example 5 Compounds

| | | | | MS |
|---|---|---|---|---|
| Ex | Structure/Name | Starting Material | ¹H NMR | (m/z) |
| 5A* | 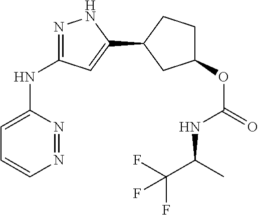 [SMILES: C[C@H](NC(=O)O[C@@H]1CC[C@H](c2cc(Nc3cccnn3)n[nH]2)C1)C(F)(F)F] | 3,3,3-trifluoro-2- methyl-propanoic acid | ¹H NMR (400 MHz, DMSO-d₆) δ 11.97 (s, 1H), 9.53 (s, 1H), 8.59- 8.58 (m, 1H), 7.86-7.84 (m, 1H), 7.52-7.50 (m, 1H), 7.41-7.38 (m, 1H), 6.21 (s, 1H), 5.07-5.04 (m, 1H), 4.29-4.24 (m, 1H), 3.09-3.05 (m, 1H), 2.57-2.50 (m, 1H), 2.05- 1.91 (m, 2H), 1.77-1.62 (m, 3H), 1.22 (d, J = 6.8 Hz, 3H). | 385.0 [M + H]⁺ |
| | (1R,3S)-3-(3-(pyridazin-3- | | | |
| | ylamino)-1H-pyrazol-5- | | | |
| | yl)cyclopentyl ((S)-1,1,1- | | | |
| | trifluoropropan-2- | | | |
| | yl)carbamate | | | |
| 5B* | 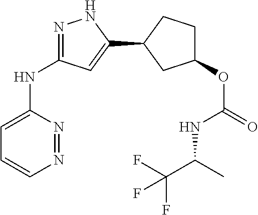 [SMILES: C[C@@H](NC(=O)O[C@@H]1CC[C@H](c2cc(Nc3cccnn3)n[nH]2)C1)C(F)(F)F] | 3,3,3-trifluoro-2- methyl-propanoic acid | ¹H NMR (400 MHz, DMSO-d₆) δ 11.96 (s, 1H), 9.53 (s, 1H), 8.59- 8.58 (m, 1H), 7.86-7.83 (m, 1H), 7.50-7.41 (m, 1H), 7.40-7.37 (m, 1H), 6.21 (s, 1H), 5.07-5.04 (m, 1H), 4.29-4.24 (m, 1H), 3.12-3.07 (m, 1H), 2.50-2.05 (m, 1H), 2.03- 1.92 (m, 2H), 1.78-1.65 (m, 3H), 1.21 (d, J = 6.8 Hz, 3H). | 385.1 [M + H]⁺ |
| | (1R,3S)-3-(3-(pyridazin-3- | | | |
| | ylamino)-1H-pyrazol-5- | | | |
| | yl)cyclopentyl ((R)-1,1,1- | | | |
| | trifluoropropan-2- | | | |
| | yl)carbamate | | | |
| 5C | 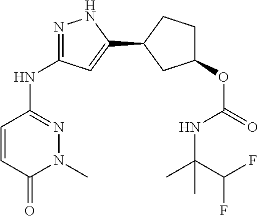 [SMILES: Cn1nc(Nc2cc([C@H]3CC[C@@H](OC(=O)NC(C)(C)C(F)F)C3)[nH]n2)ccc1=O] | 3,3-difluoro-2,2- dimethylpropanoic acid | ¹H NMR (400 MHz, DMSO-d6) δ 11.93 (br s, 1H), 9.21 (br s, 1H), 7.32- 7.27 (m, 2H), 6.84-6.82 (m, 1H), 6.34-6.05 (m, 2H), 5.0 (s, 1H), 3.52 (s, 3H), 3.07-3.0 (m, 1H), 2.46- 2.44 (m, 1H), 2.0-1.91 (m, 2H), 1.74-1.62 (m, 3H), 1.23 (s, 6H). | 411.0 [M + H]⁺ |
| | (1R,3S)-3-(3-((1-methyl-6- | | | |
| | oxo-1,6-dihydropyridazin-3- | | | |
| | yl)amino)-1H-pyrazol-5- | | | |
| | yl)cyclopentyl (1,1-difluoro- | | | |
| | 2-methylpropan-2- | | | |
| | yl)carbamate | | | |

*Examples 5A, 5B: stereochemical configuration of the carbamate N-substituent assigned arbitrarily

### General Procedure 6

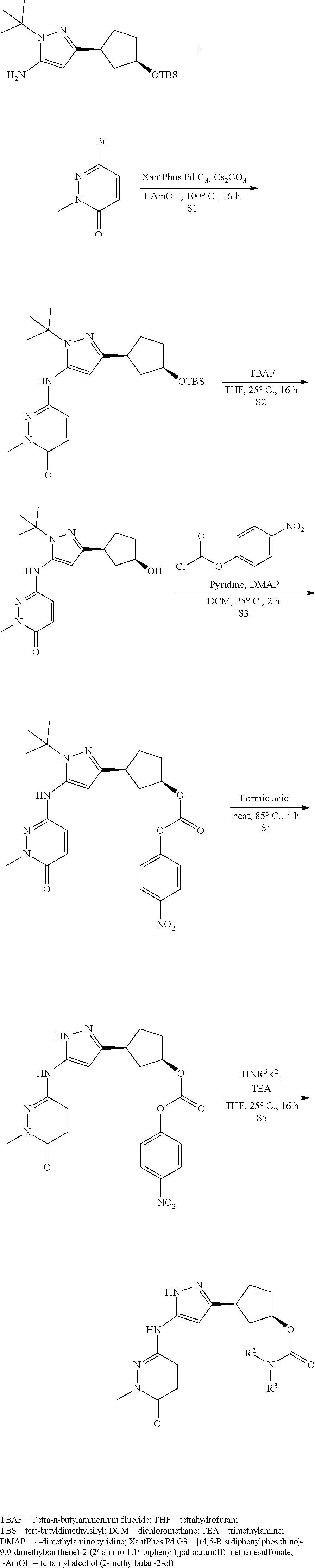

### Example 6: (1R,3S)-3-(5-((1-methyl-6-oxo-1,6-dihydropyridazin-3-yl)amino)-1H-pyrazol-3-yl)cyclopentyl bicyclo[1.1.1]pentan-1-ylcarbamate

The title compound was prepared according to General Procedure 6 as described below.

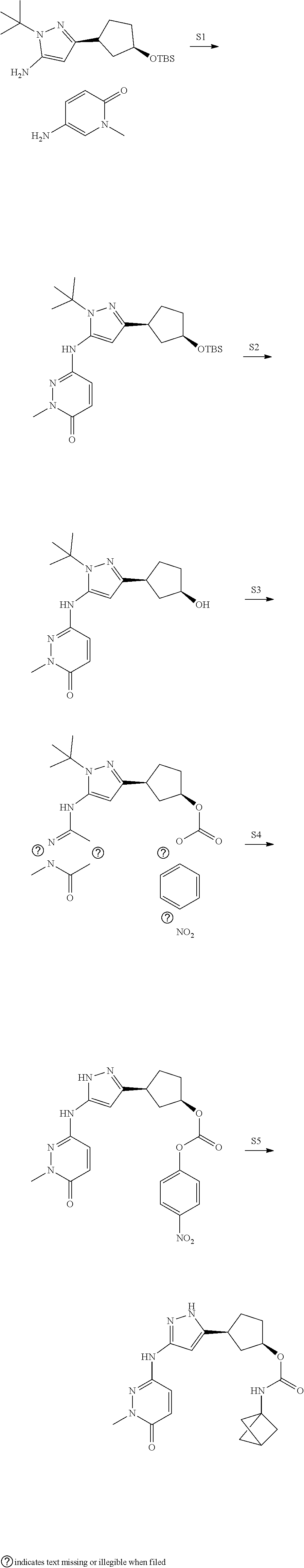

Step 1: 6-((1-(tert-butyl)-3-((1S,3R)-3-((tert-butyldimethylsilyl)oxy)cyclopentyl)-1H-pyrazol-5-yl)amino)-2-methylpyridazin-3(2H)-one. 6-bromo-2-methyl-3(2H)-pyridazinone may be prepared following the procedure provided in Example 11 of PCT Publication WO 202157652.

A mixture of 1-(tert-butyl)-3-((1S,3R)-3-((tert-butyldimethylsilyl)oxy)cyclopentyl)-1H-pyrazol-5-amine (1.38 g, 4.1 mmol), 6-bromo-2-methyl-3(2H)-pyridazinone (975 mg, 4.9 mmol), cesium carbonate (3.99 g, 12 mmol), and [(4,5-bis(diphenylphosphino)-9,9-dimethylxanthene)-2-(2′-amino-1,1′-biphenyl)]palladium(II) methanesulfonate (815 mg, 0.82 mmol) in 2-methyl-2-butanol (20 mL) was stirred at 100° C. for 16 h under nitrogen atmosphere. The reaction mixture was cooled to ambient temperature and concentrated under reduced pressure. The residue was purified by column chromatography (silica gel, 100-200 mesh, 0-50% (25% methanol: 75% isopropylacetate):heptane) to afford 6-((1-(tert-butyl)-3-((1S,3R)-3-((tert-butyldimethylsilyl)oxy)cyclopentyl)-1H-pyrazol-5-yl)amino)-2-methylpyridazin-3(2H)-one (854 mg, 47%). LCMS (ES-API, m/z): 446.2 [M+H]⁺.

Step 2: 6-((1-(tert-butyl)-3-((1S,3R)-3-hydroxycyclopentyl)-1H-pyrazol-5-yl)amino)-2-methylpyridazin-3(2H)-one. To a solution of 6-((1-(tert-butyl)-3-((1S,3R)-3-((tert-butyldimethylsilyl)oxy)cyclopentyl)-1H-pyrazol-5-yl)amino)-2-methylpyridazin-3(2H)-one (854 mg, 1.9 mmol) in tetrahydrofuran (5 mL) was added tetrabutylammoniumfluoride (1.0 M in tetrahydrofuran, 2.3 mL, 2.3 mmol). The reaction was stirred at 25° C. for 16 h and concentrated under reduced pressure. The residue was purified by column chromatography (silica gel, 100-200 mesh, 0-70% (25% methanol: 75% isopropylacetate):heptane) to afford 6-((1-(tert-butyl)-3-((1S,3R)-3-hydroxycyclopentyl)-1H-pyrazol-5-yl)amino)-2-methylpyridazin-3(2H)-one (595 mg, 92%). LCMS (ES-API, m/z): 332.1 [M+H]⁺.

Step 3: (1R,3S)-3-(1-(tert-butyl)-5-((1-methyl-6-oxo-1,6-dihydropyridazin-3-yl)amino)-1H-pyrazol-3-yl)cyclopentyl (4-nitrophenyl) carbonate. To a solution of 6-((1-(tert-butyl)-3-((1S,3R)-3-hydroxycyclopentyl)-1H-pyrazol-5-yl)amino)-2-methylpyridazin-3(2H)-one (595 mg, 1.8 mmol) in dichloromethane (18 mL) was added 4-nitrophenylchloroformate (470 mg, 2.3 mmol), pyridine (380 μL, 4.7 mmol) and 4-dimethylaminopyridine (22 mg, 0.18 mmol). The mixture was stirred at 25° C. for 16 h and then concentrated under reduced pressure. The residue was purified by column chromatography (silica gel, 100-200 mesh, 0-50% (25% methanol: 75% isopropylacetate) heptane) to afford (1R,3S)-3-(1-(tert-butyl)-5-((1-methyl-6-oxo-1,6-dihydropyridazin-3-yl)amino)-1H-pyrazol-3-yl)cyclopentyl (4-nitrophenyl) carbonate (714 mg, 80%). LCMS (ES-API, m/z): 497.2 [M+H]⁺.

Step 4: (1R,3S)-3-(5-((1-methyl-6-oxo-1,6-dihydropyridazin-3-yl)amino)-1H-pyrazol-3-yl)cyclopentyl (4-nitrophenyl) carbonate. A solution of (1R,3S)-3-(1-(tert-butyl)-5-((1-methyl-6-oxo-1,6-dihydropyridazin-3-yl)amino)-1H-pyrazol-3-yl)cyclopentyl (4-nitrophenyl) carbonate (66.1 mg, 0.13 mmol) in formic acid (1 mL) was stirred at 90° C. for 2 h. The reaction mixture was cooled to ambient temperature and concentrated under reduced pressure. The crude residue containing (1R,3S)-3-(5-((1-methyl-6-oxo-1,6-dihydropyridazin-3-yl)amino)-1H-pyrazol-3-yl)cyclopentyl (4-nitrophenyl) carbonate was used directly in the following step without purification.

Step 5: (1R,3S)-3-(5-((1-methyl-6-oxo-1,6-dihydropyridazin-3-yl)amino)-1H-pyrazol-3-yl)cyclopentyl bicyclo[1.1.1]pentan-1-ylcarbamate. To a solution of (1R,3S)-3-(5-((1-methyl-6-oxo-1,6-dihydropyridazin-3-yl)amino)-1H-pyrazol-3-yl)cyclopentyl (4-nitrophenyl) carbonate (57 mg, 0.13 mmol) in tetrahydrofuran (1.3 mL) was added triethylamine (70 μL, 0.52 mmol) and bicyclo[1.1.1]pentan-1-amine hydrochloride (65 mg, 0.52 mmol). The mixture was stirred at 25° C. for 16 h and concentrated under reduced pressure. The residue was purified by Purification Procedure H with 0.1% formic acid in water-acetonitrile to afford (1R,3S)-3-(5-((1-methyl-6-oxo-1,6-dihydropyridazin-3-yl)amino)-1H-pyrazol-3-yl)cyclopentyl bicyclo[1.1.1]pentan-1-ylcarbamate (12.3 mg, 25%). LCMS (ES-API, m/z): 385.1 [M+H]; ¹H NMR (400 MHz, DMSO-d₆) δ 11.90 (br, s, 1H), 9.16 (s, 1H), 7.74 (s, 1H), 7.28 (d, J=9.8 Hz, 1H), 6.82 (d, J=9.8 Hz, 1H), 6.23 (s, 1H), 4.99 (m, 1H), 3.52 (s, 3H), 3.05 (m, 1H), 2.44 (m, 1H), 2.34 (m, 1H), 2.02 (m, 1H), 1.89 (s, 6H), 1.87 (m, 1H), 1.72 (m, 2H), 1.61 (m, 1H).

### Example 6C: (1R,3S)-3-(3-((1-methyl-6-oxo-1,6-dihydropyridazin-3-yl)amino)-1H-pyrazol-5-yl)cyclopentyl tert-butylcarbamate

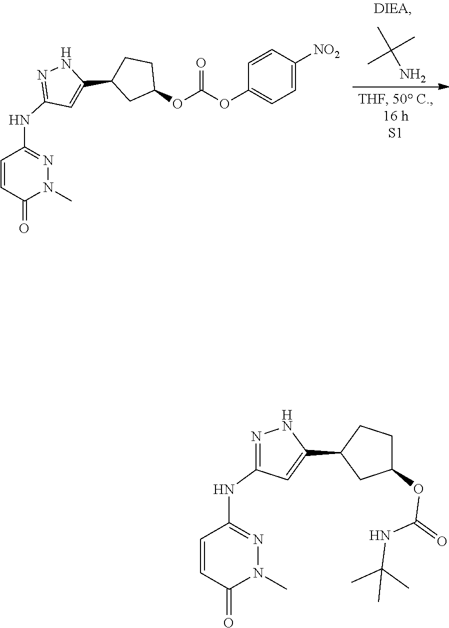

To a solution of (1R,3S)-3-(3-((1-methyl-6-oxo-1,6-dihydropyridazin-3-yl)amino)-1H-pyrazol-5-yl)cyclopentyl (4-nitrophenyl) carbonate (950 mg, 2.16 mmol) in tetrahydrofuran (15 mL) was added N,N-diisopropylethylamine (1.4 g, 10.79 mmol) and tert-butylamine (315.5 mg, 4.31 mmol). The reaction was stirred at 50° C. for 16 hours. The mixture was concentrated under reduced pressure and purified by column chromatography (silica gel, 100-200 mesh, 0 to 5% methyl alcohol in dichloromethane) to give the crude (200 mg). The crude product was purified by Purification Procedure U with 0.05% ammonium hydroxide and 10 mM ammonium bicarbonate in water-acetonitrile (30-60%) to give (1R,3S)-3-(3-((1-methyl-6-oxo-1,6-dihydropyridazin-3-yl)amino)-1H-pyrazol-5-yl)cyclopentyl tert-butylcarbamate (105 mg, 18%). LCMS (ES-API, m/z): 375.3 [M+H]⁺; ¹H NMR (400 MHz, DMSO-d6) δ 11.91 (br s, 1H), 9.19 (s, 1H), 7.30 (br d, J=9.8 Hz, 1H), 6.85-6.79 (m, 2H), 6.24 (s, 1H), 4.98 (br s, 1H), 3.52 (s, 3H), 3.06-3.02 (m, 1H), 2.45-2.43 (m, 1H), 1.92-1.89 (m, 2H), 1.72-1.60 (m, 3H), 1.21 (s, 9H).

Other compounds were synthesized according to General Procedure and Example 6, as set forth in the below Table K.

TABLE K
General Procedure 6 and Example 6 Compounds

| | | Starting | | |
|---|---|---|---|---|
| Ex | Structure/Name | Material | ¹H NMR | MS (m/z) |
| 6A | 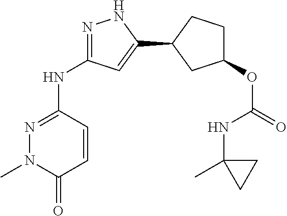 | N-methyl tert- butylamine | ¹H NMR (400 MHz, DMSO- d₆) δ 11.93 (br, s, 1H), 9.17 (s, 1H), 7.28 (d, J = 9.8 Hz, 1H), 6.82 (d, J = 9.8 Hz, 1H), 6.22 (s, 1H), 5.01 (m, 1H), 3.51 (s, 3H), 3.07 (m, 1H), 2.80 (s, 3H), 2.45 (m, 1H), 2.02 (m, 1H), 1.88 (m, 1H), 1.77 (m, 2H), 1.68 (m, 1H), 1.78-1.62 (m, 1H), 1.31 (s, 9H). | 389.2 [M + H]⁺ |
| | (1R,3S)-3-(3-((1-methyl-6-oxo-1,6- | | | |
| | dihydropyridazin-3-yl)amino)-1H- | | | |
| | pyrazol-5-yl)cyclopentyl (1- | | | |
| | methylcyclopropyl)carbamate | | | |
| 6B | 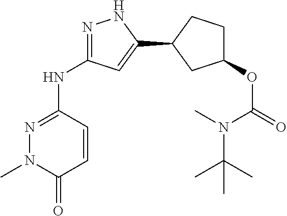 [SMILES: CN(C(=O)O[C@@H]1CC[C@H](c2cc(Nc3ccc(=O)n(C)n3)n[nH]2)C1)C(C)(C)C] | N-methyl tert- butylamine | ¹H NMR (400 MHz, DMSO- d₆) δ 11.93 (br, s, 1H), 9.17 (s, 1H), 7.28 (d, J = 9.8 Hz, 1H), 6.82 (d, J = 9.8 Hz, 1H), 6.22 (s, 1H), 5.01 (m, 1H), 3.51 (s, 3H), 3.07 (m, 1H), 2.80 (s, 3H), 2.45 (m, 1H), 2.02 (m, 1H), 1.88 (m, 1H), 1.77 (m, 2H), 1.68 (m, 1H), 1.78-1.62 (m, 1H), 1.31 (s, 9H). | 389.2 [M + H]⁺ |
| | (1R,3S)-3-(3-((1-methyl-6-oxo-1,6- | | | |
| | dihydropyridazin-3-yl)amino)-1H- | | | |
| | pyrazol-5-yl)cyclopentyl tert- | | | |
| | butyl(methyl)carbamate | | | |
| 6C | 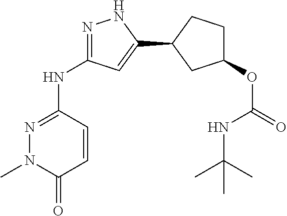 [SMILES: Cn1nc(Nc2cc([C@H]3CC[C@@H](OC(=O)NC(C)(C)C)C3)[nH]n2)ccc1=O] | N-methyl tert- butylamine | ¹H NMR (400 MHz, DMSO- d₆) δ 11.90 (br, s, 1H), 9.18 (s, 1H), 7.29 (d, J = 9.8 Hz, 1H), 6.82 (d, J = 9.8 Hz, 1H), 6.74 (s, 1H), 6.20 (s, 1H), 4.97 (m, 1H), 3.52 (s, 3H), 3.04 (p, J = 9.0 Hz, 1H), 2.45 (m, 1H), 2.00 (q, J = 8.9 Hz, 1H), 1.98-1.81 (m, 1H), 1.80-1.65 (m, 2H), 1.60 (m, 1H), 1.20 (s, 9H). | 375.2 [M + H]⁺ |
| | (1R,3S)-3-(3-((1-methyl-6-oxo-1,6- | | | |
| | dihydropyridazin-3-yl)amino)-1H- | | | |
| | pyrazol-5-yl)cyclopentyl tert- | | | |
| | butylcarbamate | | | |
| 6D | 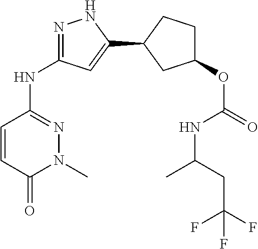 [SMILES: CC(CC(F)(F)F)NC(=O)O[C@@H]1CC[C@H](c2cc(Nc3ccc(=O)n(C)n3)n[nH]2)C1] | 4,4,4- trifluorobutan- 2-amine hydrochloride | ¹H NMR (400 MHz, DMSO- d6) δ 11.94 (br s, 1H), 9.21 (br s, 1H), 7.28 (d, J = 9.6 Hz, 1H), 7.21-7.19 (m, 1H), 6.83 (d, J = 9.6 Hz, 1H), 6.20 (s, 1H), 5.01-5.0 (m, 1H), 3.86- 3.82 (m, 1H), 3.52 (s, 3H), 3.06- 3.02 (m, 1H), 2.47-2.33 (m, 3H), 2.0-1.80 (m, 2H), 1.74- 1.70 (m, 3H), 1.14-1.11 (m, 3H). | 429.1 [M + H]⁺ |
| | (1R,3S)-3-(3-((1-methyl-6-oxo-1,6- | | | |
| | dihydropyridazin-3-yl)amino)-1H- | | | |
| | pyrazol-5-yl)cyclopentyl (4,4,4- | | | |
| | trifluorobutan-2-yl)carbamate | | | |
| 6E | 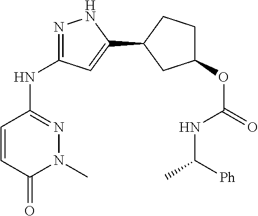 [SMILES: C[C@H](NC(=O)O[C@@H]1CC[C@H](c2cc(Nc3ccc(=O)n(C)n3)n[nH]2)C1)c1ccccc1] | Prepared by General Procedure 6 from (S)-1- phenylethan- 1-amine | ¹H NMR (400 MHz, DMSO- d₆) δ 11.91 (br, s, 1H), 9.20 (s, 1H), 7.64 (d, J = 8.4 Hz, 1H), 7.28-7.27 (m, 5H), 7.21- 7.17 (m, 1H), 6.83 (d, J = 10.0 Hz, 1H), 6.21 (s, 1H), 4.97 (m, 1H), 4.68-4.60 (m, 1H), 3.50 (s, 3H), 3.00 (m, 1H), 2.45 (m, 1H), 2.00 (m, 1H), 1.91 (m, 1H), 1.76-1.70 (m, 2H), 1.60 (m, 1H), 1.31 (d, J = 6.8 Hz, 3H). | 423.2 [M + H]⁺ |
| | (1R,3S)-3-(3-((1-methyl-6-oxo-1,6- | | | |
| | dihydropyridazin-3-yl)amino)-1H- | | | |
| | pyrazol-5-yl)cyclopentyl ((S)-1- | | | |
| | phenylethyl)carbamate | | | |

### General Procedure 7

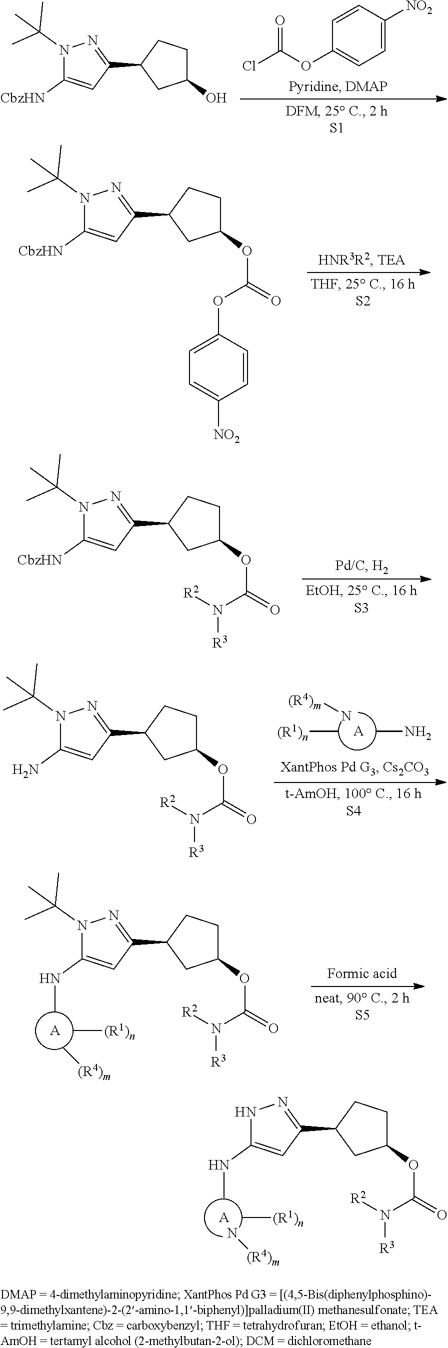

### Example 7: (1R,3S)-3-(3-((6-(dimethylcarbamoyl)-2-methylpyridin-3-yl)amino)-1H-pyrazol-5-yl)cyclopentyl (1-methylcyclopropyl)carbamate

The title compound was prepared according to General Procedure 7 as described below.

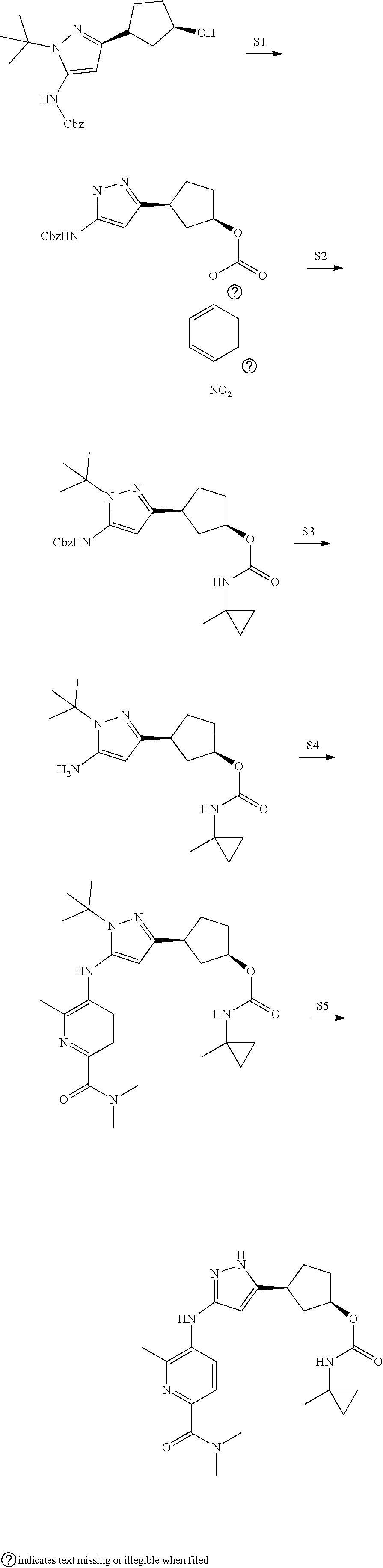

Step 1: benzyl (1-(tert-butyl)-3-((1S,3R)-3-(((4-nitrophenoxy)carbonyl)oxy)cyclopentyl)-1H-pyrazol-5-yl)carbamate. Benzyl (1-(tert-butyl)-3-((1S,3R)-3-hydroxycyclopentyl)-1H-pyrazol-5-yl)carbamate may be prepared following the procedure provided in the Preparation of Synthetic Intermediates of PCT Publication WO 202157652.

To a solution of benzyl (1-(tert-butyl)-3-((1S,3R)-3-hydroxycyclopentyl)-1H-pyrazol-5-yl)carbamate (500 mg, 1.4 mmol) in dichloromethane (14 mL) was added 4-nitrophenylchloroformate (367 mg, 1.8 mmol), pyridine (290 μL, 3.6 mmol) and 4-dimethylaminopyridine (17 mg, 0.14 mmol). The mixture was stirred at 25° C. for 16 h and concentrated under reduced pressure. The residue was purified by column chromatography (silica gel, 100-200 mesh, 0-30% (25% methanol: 75% isopropylacetate):heptane) to afford benzyl (1-(tert-butyl)-3-((1S,3R)-3-(((4-nitrophenoxy)carbonyl)oxy)cyclopentyl)-1H-pyrazol-5-yl)carbamate (730 mg, 100%). LCMS (ES-API, m/z): 523.2 [M+H]⁺.

Step 2: benzyl (1-(tert-butyl)-3-((1S,3R)-3-(((1-methylcyclopropyl)carbamoyl)oxy)cyclopentyl)-1H-pyrazol-5-yl)carbamate. To a solution of benzyl (1-(tert-butyl)-3-((1S,3R)-3-(((4-nitrophenoxy)carbonyl)oxy)cyclopentyl)-1H-pyrazol-5-yl)carbamate (730 mg, 1.4 mmol) in tetrahydrofuran (16 mL) was added triethylamine (890 μL, 6.4 mmol) and 1-methylcyclopropanamine hydrochloride (706 mg, 6.4 mmol). The mixture was stirred at 25° C. for 16 h and concentrated under reduced pressure. The residue was purified by column chromatography (silica gel, 100-200 mesh, 0-80% isopropylacetate:heptane) to afford benzyl (1-(tert-butyl)-3-((1S,3R)-3-(((1-methylcyclopropyl)carbamoyl)oxy)cyclopentyl)-1H-pyrazol-5-yl)carbamate (635 mg, 99%). LCMS (ES-API, m/z): 455.2 [M+H]⁺.

Step 3: (1R,3S)-3-(5-amino-1-(tert-butyl)-1H-pyrazol-3-yl)cyclopentyl (1-methylcyclopropyl)carbamate. To a solution of benzyl (1-(tert-butyl)-3-((1S,3R)-3-(((1-methylcyclopropyl)carbamoyl)oxy)cyclopentyl)-1H-pyrazol-5-yl)carbamate (656 mg, 1.4 mmol) and palladium on carbon (5 wt %, 614 mg, 0.29 mmol) in ethanol (10 mL) was stirred under hydrogen atmosphere (15 psi) at 25° C. for 16 h. The reaction mixture was then filtered and the filtrate was concentrated under reduced pressure to afford (1R,3S)-3-(5-amino-1-(tert-butyl)-1H-pyrazol-3-yl)cyclopentyl (1-methylcyclopropyl)carbamate (471 mg, 100%). LCMS (ES-API, m/z): 321.1 [M+H]⁺.

Step 4: (1R,3S)-3-(1-(tert-butyl)-5-((6-(dimethylcarbamoyl)-2-methylpyridin-3-yl)amino)-1H-pyrazol-3-yl)cyclopentyl (1-methylcyclopropyl)carbamate. A mixture of (1R,3S)-3-(5-amino-1-(tert-butyl)-1H-pyrazol-3-yl)cyclopentyl (1-methylcyclopropyl)carbamate (52 mg, 0.16 mmol), 5-bromo-N,N,6-trimethylpicolinamide (43 mg, 0.18 mmol), cesium carbonate (157 mg, 0.48 mmol), and [(4,5-bis(diphenylphosphino)-9,9-dimethylxanthene)-2-(2′-amino-1,1′-biphenyl)]palladium(II) methanesulfonate (32 mg, 0.032 mmol) in 2-methyl-2-butanol (800 μL) was stirred at 100° C. for 16 h under nitrogen atmosphere and concentrated under reduced pressure. The residue was purified by column chromatography (silica gel, 100-200 mesh, 0-90% (25% methanol: 75% isopropylacetate):heptane) to afford (1R,3S)-3-(1-(tert-butyl)-5-((6-(dimethylcarbamoyl)-2-methylpyridin-3-yl)amino)-1H-pyrazol-3-yl)cyclopentyl (1-methylcyclopropyl)carbamate (40.4 mg, 52%). LCMS (ES-API, m/z): 483.2 [M+H]⁺.

Step 5: (1R,3S)-3-(3-((6-(dimethylcarbamoyl)-2-methylpyridin-3-yl)amino)-1H-pyrazol-5-yl)cyclopentyl (1-methylcyclopropyl)carbamate. A solution of afford (1R,3S)-3-(1-(tert-butyl)-5-((6-(dimethylcarbamoyl)-2-methylpyridin-3-yl)amino)-1H-pyrazol-3-yl)cyclopentyl (1-methylcyclopropyl)carbamate (40 mg, 0.084 mmol) in formic acid (1 mL) was stirred at 90° C. for 2 h and concentrated under reduced pressure. The residue was purified by Purification Procedure I with 0.1% formic acid in water-acetonitrile to afford (1R,3S)-3-(3-((6-(dimethylcarbamoyl)-2-methylpyridin-3-yl)amino)-1H-pyrazol-5-yl)cyclopentyl (1-methylcyclopropyl)carbamate (17 mg, 46%). LCMS (ES-API, m/z): 427.2 [M+H]⁺; ¹H NMR (400 MHz, DMSO-d₆) δ 11.90 (br, s, 1H), 8.15 (br, s, 1H), 7.74 (s, 1H), 7.33 (d, J=8.5 Hz, 2H), 5.82 (s, 1H), 4.99 (m, 1H), 3.06 (s, 3H), 3.01 (m, 1H), 2.96 (s, 3H), 2.45 (s, 3H), 2.43 (m, 1H), 2.05-1.98 (m, 1H), 1.98-1.82 (m, 1H), 1.74-1.65 (m, 2H), 1.58 (m, 1H), 1.23 (s, 3H), 0.63-0.51 (m, 2H), 0.55-0.42 (m, 2H).

### Example 7A: (1R,3S)-3-(5-((2-methyl-6-(methylcarbamoyl)pyridin-3-yl)amino)-1H-pyrazol-3-yl)cyclopentyl (1-methylcyclopropyl)carbamate

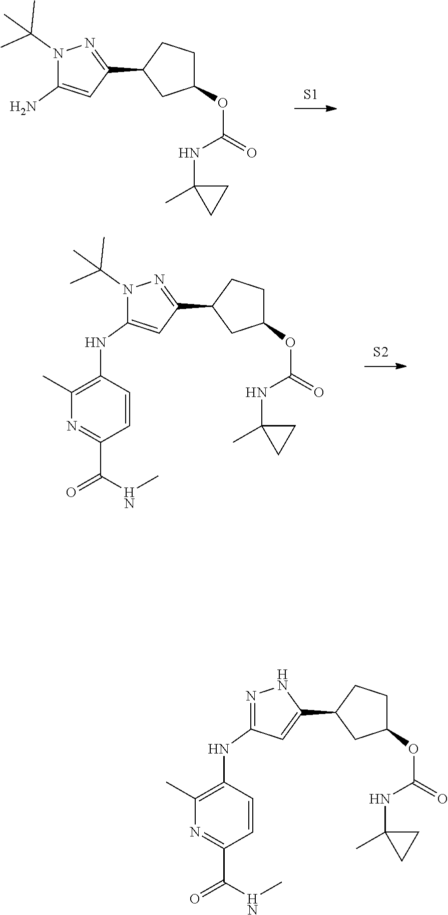 [SMILES: CC1(NC(=O)O[C@@H]2CC[C@H](c3cc(N)n(C(C)(C)C)n3)C2)CC1 ; "S1" ; CNC(=O)c1ccc(Nc2cc([C@H]3CC[C@@H](OC(=O)NC4(C)CC4)C3)nn2C(C)(C)C)c(C)n1 ; "S2" ; CNC(=O)c1ccc(Nc2cc([C@H]3CC[C@@H](OC(=O)NC4(C)CC4)C3)[nH]n2)c(C)n1]

Step 1: (1R,3S)-3-(1-(tert-butyl)-5-((2-methyl-6-(methylcarbamoyl)pyridin-3-yl)amino)-1H-pyrazol-3-yl)cyclopentyl (1-methylcyclopropyl)carbamate. A mixture of (1R,3S)-3-(5-amino-1-(tert-butyl)-1H-pyrazol-3-yl)cyclopentyl (1-methylcyclopropyl)carbamate (52 mg, 0.16 mmol), 5-bromo-N,6-dimethylpicolinamide (44 mg, 0.19 mmol), cesium carbonate (157 mg, 0.48 mmol), and [(4,5-bis(diphenylphosphino)-9,9-dimethylxanthene)-2-(2′-amino-1,1′-biphenyl)]palladium(II) methanesulfonate (32 mg, 0.032 mmol) in 2-methyl-2-butanol (860 μL) was stirred at 100° C. for 16 h under nitrogen atmosphere and concentrated under reduced pressure. The residue was purified by column chromatography (silica gel, 100-200 mesh, 0-100% isopropylacetate:heptane) to afford (1R,3S)-3-(1-(tert-butyl)-5-((2-methyl-6-(methylcarbamoyl)pyridin-3-yl)amino)-1H-pyrazol-3-yl)cyclopentyl (1-methylcyclopropyl)carbamate (40.4 mg, 54%). LCMS (ES-API, m/z): 469.2 [M+H]⁺.

Step 2: (1R,3S)-3-(5-((2-methyl-6-(methylcarbamoyl)pyridin-3-yl)amino)-1H-pyrazol-3-yl)cyclopentyl (1-methylcyclopropyl)carbamate. A solution of (1R,3S)-3-(1-(tert-butyl)-5-((2-methyl-6-(methylcarbamoyl)pyridin-3-yl)amino)-1H-pyrazol-3-yl)cyclopentyl (1-methylcyclopropyl)carbamate (40 mg, 0.086 mmol) in formic acid (1 mL) was stirred at 90° C. for 2 h and concentrated under reduced pressure. The residue was purified by Purification Procedure H with 0.1% formic acid in water-acetonitrile to afford (1R,3S)-3-(5-((2-methyl-6-(methylcarbamoyl)pyridin-3-yl)amino)-1H-pyrazol-3-yl)cyclopentyl (1-methylcyclopropyl)carbamate (16 mg, 44%). LCMS (ES-API, m/z): 413.1 [M+H]; ¹H NMR (400 MHz, DMSO-d₆) δ 11.95 (s, 1H), 8.26 (q, J=4.8 Hz, 1H), 8.23-8.16 (m, 1H), 7.86 (s, 1H), 7.72 (d, J=8.5 Hz, 1H), 7.33 (s, 1H), 5.85 (s, 1H), 5.10-4.92 (m, 1H), 3.11-3.01 (m, 1H), 2.79 (d, J=4.9 Hz, 3H), 2.50 (s, 3H), 2.49-2.40 (m, 1H), 2.06-1.97 (s, 1H), 1.98-1.82 (m, 1H), 1.77-1.64 (m, 2H), 1.63-1.52 (s, 1H), 1.23 (s, 3H), 0.59 (q, J=4.4 Hz, 2H), 0.51-0.42 (m, 2H).

Other compounds were synthesized according to General Procedure and Example 7, as set forth in the below Table L1.

TABLE L1
General Procedure 7 and Example 7 Compounds

| | | Starting | | MS |
|---|---|---|---|---|
| Ex | Structure/Name | Material(s) | ¹H NMR | (m/z) |
| 7A | 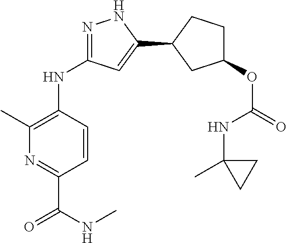 [SMILES: CNC(=O)c1ccc(Nc2cc([C@H]3CC[C@@H](OC(=O)NC4(C)CC4)C3)[nH]n2)c(C)n1] | 1-methylcyclo- propanamine hydrochloride and 5-bromo-N,6- dimethyl- picolinamide | ¹H NMR (400 MHz, DMSO- d₆) δ 11.95 (br, s, 1H), 8.26 (q, J = 4.8 Hz, 1H), 8.23- 8.16 (m, 1H), 7.86 (s, 1H), 7.72 (d, J = 8.5 Hz, 1H), 7.33 (s, 1H), 5.85 (s, 1H), 4.99 (m, 1H), 3.11-3.01 (m, 1H), 2.79 (d, J = 4.9 Hz, 3H), 2.49 (s, 3H), 2.47 (m, 1H), 2.03 (m, 1H), 1.98-1.82 (m, 1H), 1.70 (m, 2H), 1.58 (m, 1H), 1.23 (s, 3H), 0.59 (m, 2H), 0.51-0.42 (m, 2H). | 413.1 [M + H]⁺ |
| | (1R,3S)-3-(3-((2-methyl-6- | | | |
| | (methylcarbamoyl)pyridin-3- | | | |
| | \|yl)amino)-1H-pyrazol-5- | | | |
| | yl)cyclopentyl (1- | | | |
| | methylcyclopropyl)carbamate | | | |
| 7B | 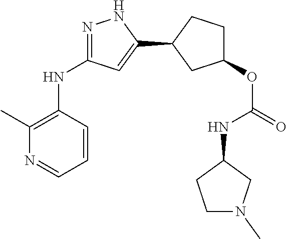 [SMILES: Cc1ncccc1Nc1cc([C@H]2CC[C@@H](OC(=O)N[C@@H]3CCN(C)C3)C2)[nH]n1] | (R)-1-methyl- pyrrolidin-3- amine and 3-bromo-2- methyl-pyridine | ¹H NMR (400 MHz, DMSO- d6) δ 11.79 (br s, 1H), 8.08 (br s, 1H), 7.81 (d, J = 4.0 Hz, 1H), 7.44 (s, 1H), 7.19 (d, J = 7.2 Hz, 1H), 7.06-7.03 (m, 1H), 5.77 (s, 1H), 4.98 (br s, 1H), 3.92 (br s, 1H), 3.08- 3.02 (m, 1H), 2.63-2.59 (m, 1H), 2.47-2.42 (m, 5H), 2.33-2.31 (m, 1H), 2.23- 2.11 (m, 4H), 2.07-1.98 (m, 2H), 1.93-1.83 (m, 1H), 1.76-1.66 (m, 2H), 1.63- 1.49 (m, 2H). | 385.3 [M + H]⁺ |
| | (1R,3S)-3-(3-((2-methylpyridin-3- | | | |
| | yl)amino)-1H-pyrazol-5- | | | |
| | yl)cyclopentyl ((R)-1- | | | |
| | methylpyrrolidin-3-yl)carbamate | | | |
| 7C | 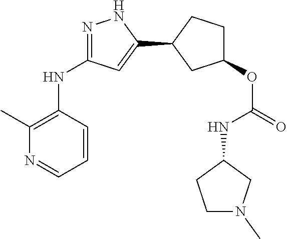 [SMILES: Cc1ncccc1Nc1cc([C@H]2CC[C@@H](OC(=O)N[C@H]3CCN(C)C3)C2)[nH]n1] | (S)-1-methyl- pyrrolidin-3- amine and 3-bromo-2- methylpyridine | ¹H NMR (400 MHz, DMSO- d6) δ 11.79 (br s, 1H), 8.10- 8.08 (m, 1H), 7.82-7.81 (m, 1H), 7.44 (s, 1H), 7.22-7.20 (m, 1H), 7.06-7.02 (m, 1H), 5.77 (s, 1H), 4.99 (br s, 1H), 3.92 (br s, 1H), 3.07-3.05 (m, 1H), 3.32 (s, 8H), 2.64- 2.55 (m, 1H), 2.47-2.40 (m, 5H), 2.37-2.31 (m, 1H), 2.23-2.13 (m, 4H), 2.08- 1.83 (m, 3H), 1.78-1.08 (m, 5H). | 385.2 [M + H]⁺ |
| | (1R,3S)-3-(3-((2-methylpyridin-3- | | | |
| | yl)amino)-1H-pyrazol-5- | | | |
| | yl)cyclopentyl ((S)-1- | | | |
| | methylpyrrolidin-3-yl)carbamate | | | |
| 7D | 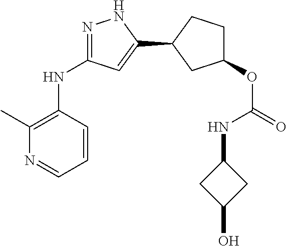 | (1S,3S)-3-((tert- butyldimethyl- silyl)oxy)- cyclobutanamine and 3-bromo-2- methylpyridine | ¹H NMR (400 MHz, DMSO- d6) δ 11.80 (br.s, 1H), 8.06 (br.s, 1H), 7.82-7.81 (m, 1H), 7.45 (s, 1H), 7.30-7.28 (m, 1H), 7.06-7.03 (m, 1H), 5.77 (s, 1H), 5.03-4.98 (m, 1H), 3.73 (br s, 1H), 3.50- 3.48 (m, 1H), 3.03-2.99 (m, 1H), 2.42 (s, 6H), 2.02-2.00 (m, 1H),1.93-1.82 (m, 1H), 1.74-1.58 (m, 5H). | 372.1 [M + H]⁺ |
| | (1R,3S)-3-(3-((2-methylpyridin-3- | | | |
| | yl)amino)-1H-pyrazol-5- | | | |
| | yl)cyclopentyl ((1s,3s)-3- | | | |
| | hydroxycyclobutyl)carbamate | | | |

### General Procedure 8

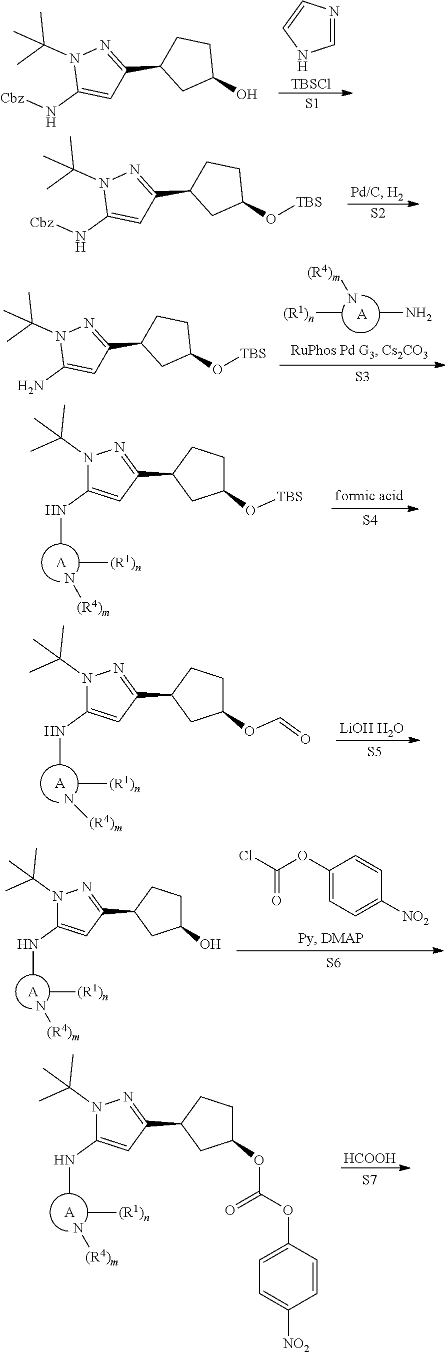

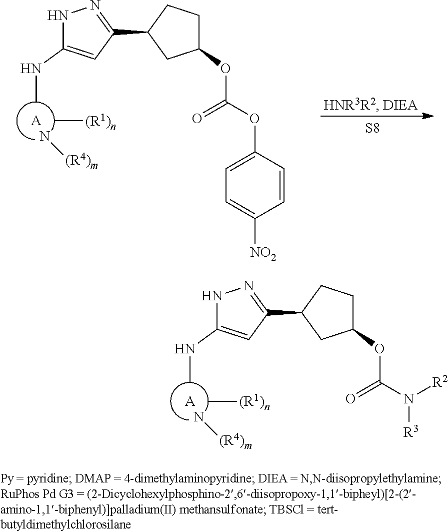

### Example 8. (1R,3S)-3-(3-((6-(dimethylcarbamoyl)-2-methylpyridin-3-yl)amino)-1H-pyrazol-5-yl)cyclopentyl (1-methylcyclopropyl)carbamate

The title compound was prepared according to General Procedure 8 as described below.

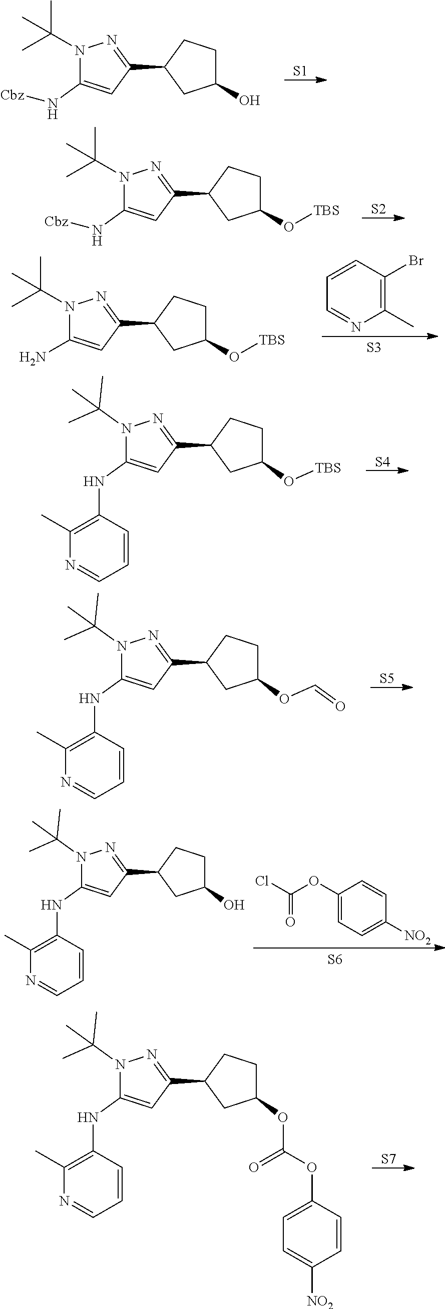

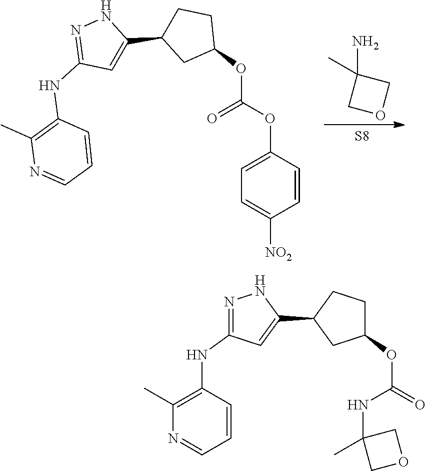

Step 1: benzyl (1-(tert-butyl)-3-((1S,3R)-3-((tert-butyldimethylsilyl)oxy)cyclopentyl)-1H-pyrazol-5-yl)carbamate. To solution of benzyl N-[2-tert-butyl-5-[(1S,3R)-3-hydroxycyclopentyl]pyrazol-3-yl]carbamate (Intermediate C) (5.0 g, 13.99 mmol) and imidazole (3.81 g, 55.95 mmol) in dichloromethane (80 mL) at 0° C., then tert-butyldimethylchlorosilane (3.16 g, 20.98 mmol) was added. The mixture was stirred at 25° C. for 16 hours. The TLC (30% ethyl acetate in petroleum ether, Rf=0.5) showed the reaction was completed. The resulting solution was diluted with water (50 mL) and extracted with ethyl acetate (2×40 mL). The combined organic layers were dried over anhydrous sodium sulfate and concentrated to give the crude which was purified by column chromatography (silica gel, 100-200 mesh, 5% ethyl acetate in petroleum ether) to give benzyl (1-(tert-butyl)-3-((1S,3R)-3-((tert-butyldimethylsilyl)oxy)cyclopentyl)-1H-pyrazol-5-yl)carbamate (5.7 g, 86.4%). LCMS (ES-API, M/Z): 472.2 [M+H]⁺.

Step 2: 1-(tert-butyl)-3-((1S,3R)-3-((tert-butyldimethylsilyl)oxy)cyclopentyl)-1H-pyrazol-5-amine. To a solution of benzyl (1-(tert-butyl)-3-((1S,3R)-3-((tert-butyldimethylsilyl)oxy)cyclopentyl)-1H-pyrazol-5-yl)carbamate (5.7 g, 12.08 mmol) in ethyl acetate (40 mL) and tetrahydrofuran (20 mL) was added palladium (10% on carbon, 2.57 g). The mixture was stirred at 20° C. for 16 hours under hydrogen atmosphere (15 psi) and filtered. The filtrate was concentrated under reduced pressure to give 1-(tert-butyl)-3-((1S,3R)-3-((tert-butyldimethylsilyl)oxy)cyclopentyl)-1H-pyrazol-5-amine (3.6 g, 88.3%). LCMS (ES-API, M/Z): 338.4 [M+H]⁺.

Step 3: N-(1-(tert-butyl)-3-((1S,3R)-3-((tert-butyldimethylsilyl)oxy)cyclopentyl)-1H-pyrazol-5-yl)-2-methylpyridin-3-amine. To a solution of 1-(tert-butyl)-3-((1S,3R)-3-((tert-butyldimethylsilyl)oxy)cyclopentyl)-1H-pyrazol-5-amine (500 mg, 1.48 mmol) in 1,4-dioxane (10 mL) was added 3-bromo-2-methylpyridine (509 mg, 2.96 mmol), cesium carbonate (1447.8 mg, 4.44 mmol) and (2-dicyclohexylphosphino-2′,6′-diisopropoxy-1,1′-biphenyl)[2-(2′-amino-1,1′-biphenyl)]palladium(ii) methanesulfonate (123.9 mg, 0.15 mmol). The mixture was stirred at 100° C. for 16 hours under N₂. and concentrated. The residue was purified by column chromatography (silica gel, 100-200 mesh, 20% ethyl acetate in petroleum ether) to give N-(1-(tert-butyl)-3-((1S,3R)-3-((tert-butyldimethylsilyl)oxy)cyclopentyl)-1H-pyrazol-5-yl)-2-methylpyridin-3-amine (600 mg, 94.5%). LCMS (ES-API, M/Z): 429.3 [M+H]⁺.

Step 4: (1R,3S)-3-(1-(tert-butyl)-5-((2-methylpyridin-3-yl)amino)-1H-pyrazol-3-yl)cyclopentyl formate. A mixture of N-(1-(tert-butyl)-3-((1S,3R)-3-((tert-butyldimethylsilyl)oxy)cyclopentyl)-1H-pyrazol-5-yl)-2-methylpyridin-3-amine (300 mg, 0.70 mmol) in formic acid (8.0 mL, 212.04 mmol) was stirred at 25° C. for 16 hours and concentrated. The residue was concentrated to give (1R,3S)-3-(1-(tert-butyl)-5-((2-methylpyridin-3-yl)amino)-1H-pyrazol-3-yl)cyclopentyl formate (220 mg, 91.8%). LCMS (ES-API, M/Z): 343.3 [M+H]⁺.

Step 5: (1R,3S)-3-(1-(tert-butyl)-5-((2-methylpyridin-3-yl)amino)-1H-pyrazol-3-yl)cyclopentanol. To a solution of (1R,3S)-3-(1-(tert-butyl)-5-((2-methylpyridin-3-yl)amino)-1H-pyrazol-3-yl)cyclopentyl formate (220.0 mg, 0.64 mmol) in methanol (10 mL) and water (5 mL) was added lithium hydroxide hydrate (81.0 mg, 1.93 mmol). The reaction mixture was stirred at 25° C. for 1 hour and concentrated. The residue was purified by column chromatography (silica gel, 100-200 mesh, 80% ethyl acetate in petroleum ether) to afford (1R,3S)-3-(1-(tert-butyl)-5-((2-methylpyridin-3-yl)amino)-1H-pyrazol-3-yl)cyclopentanol (195 mg, 96.5%). LCMS (ES-API, M/Z): 315.0 [M+H]⁺.

Step 6: (1R,3S)-3-(1-(tert-butyl)-5-((2-methylpyridin-3-yl)amino)-1H-pyrazol-3-yl)cyclopentyl (4-nitrophenyl) carbonate. To a solution of (1R,3S)-3-(1-(tert-butyl)-5-((2-methylpyridin-3-yl)amino)-1H-pyrazol-3-yl)cyclopentanol (85.0 mg, 0.27 mmol) and 4-nitrophenylchloroformate (109.0 mg, 0.54 mmol) in dichloromethane (10 mL) was added pyridine (0.07 mL, 0.81 mmol) and 4-dimethylaminopyridine (3.3 mg, 0.03 mmol). The mixture was stirred at 25° C. for 3 hours and concentrated. The residue was purified by column chromatography(silica gel, 100-200 mesh, 10% methanol in dichloromethane) to afford (1R,3S)-3-(1-(tert-butyl)-5-((2-methylpyridin-3-yl)amino)-1H-pyrazol-3-yl)cyclopentyl (4-nitrophenyl) carbonate (70.0 mg, 54%). LCMS (ES-API, M/Z): 480.2 [M+H]⁺.

Step 7: (1R,3S)-3-(3-((2-methylpyridin-3-yl)amino)-1H-pyrazol-5-yl)cyclopentyl (4-nitrophenyl) carbonate. To a solution of (1R,3S)-3-(1-(tert-butyl)-5-((2-methylpyridin-3-yl)amino)-1H-pyrazol-3-yl)cyclopentyl (4-nitrophenyl) carbonate (200.0 mg, 0.42 mmol) in formic acid (5.0 mL, 132.52 mmol) was stirred at 75° C. for 16 hours and concentrated to give crude (1R,3S)-3-(3-((2-methylpyridin-3-yl)amino)-1H-pyrazol-5-yl)cyclopentyl (4-nitrophenyl) carbonate (150 mg, 85%). LCMS (ES-API, M/Z): 424.0 [M+H]⁺.

Step 8: (1R,3S)-3-(3-((2-methylpyridin-3-yl)amino)-1H-pyrazol-5-yl)cyclopentyl (3-methyloxetan-3-yl)carbamate. To a solution of 3-methyloxetan-3-amine hydrochloride (65.7 mg, 0.53 mmol) in tetrahydrofuran (5 mL) was added (1R,3S)-3-(3-((2-methylpyridin-3-yl)amino)-1H-pyrazol-5-yl)cyclopentyl (4-nitrophenyl) carbonate (150.0 mg, 0.35 mmol) and N,N-Diisopropylethylamine (0.19 mL, 1.06 mmol). The mixture was stirred at 25° C. for 16 hours and concentrated. The residue was purified with Purification Procedure J with 0.225% formic acid in water-acetonitrile (5-35%) to give (1R,3S)-3-(3-((2-methylpyridin-3-yl)amino)-1H-pyrazol-5-yl)cyclopentyl (3-methyloxetan-3-yl)carbamate (16.2 mg, 12.2%). ¹H NMR (400 MHz, DMSO-d6) δ 11.90 (br, 1H), 8.15 (s, 1H), 8.05 (d, J=4.0 Hz, 1H), 7.82 (d, J=7.2 Hz, 1H), 7.62 (br s, 1H), 7.45 (s, 1H), 7.08-7.03 (m, 1H), 5.77 (s, 1H), 5.05 (br s, 1H), 4.63 (br s, 2H), 4.25-4.18 (m, 2H), 3.07-3.01 (m, 1H), 2.46 (s, 1H), 2.43 (s, 3H), 2.05-2.01 (m, 1H), 1.96-1.90 (m, 1H), 1.79-1.57 (m, 3H), 1.47 (s, 3H). LCMS (ES-API, M/Z): 372.1 [M+H]⁺.

### Example 8B: (1R,3S)-3-(3-((3-methylpyrazin-2-yl)amino)-1H-pyrazol-5-yl)cyclopentyl (3-methyloxetan-3-yl)carbamate

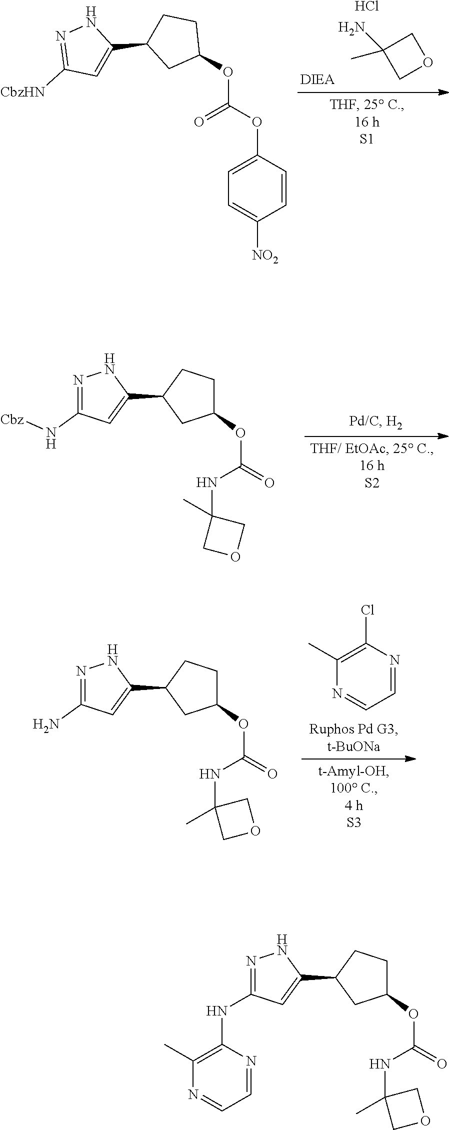

Step 1: (1R,3S)-3-(3-(((benzyloxy)carbonyl)amino)-1H-pyrazol-5-yl)cyclopentyl (3-methyloxetan-3-yl)carbamate. To a solution of benzyl (5-((1S,3R)-3-(((4-nitrophenoxy)carbonyl)oxy)cyclopentyl)-1H-pyrazol-3-yl)carbamate (4.0 g, 8.58 mmol), 3-methyloxetan-3-amine hydrochloride (1.6 g, 12.86 mmol) in tetrahydrofuran (40 mL) was added N,N-diisopropylethylamine (4.48 mL, 25.73 mmol). The mixture was stirred at 25° C. for 16 hours. The reaction was diluted with 10% aqueous sodium hydroxide (20 mL), then extracted with ethyl acetate (3×30 mL). The organic layer was dried over anhydrous sodium sulfate, filtered and concentrated under reduced pressure. The crude was purified by column chromatography (silica gel, 100-200 mesh, 0 to 55% ethyl acetate in petroleum ether) to afford (1R,3S)-3-(3-(((benzyloxy)carbonyl)amino)-1H-pyrazol-5-yl)cyclopentyl (3-methyloxetan-3-yl)carbamate (2.1 g, 58%). LCMS (ES-API, m/z): 415.4 [M+H]⁺.

Step 2: (1R,3S)-3-(3-amino-1H-pyrazol-5-yl)cyclopentyl (3-methyloxetan-3-yl)carbamate. To a solution of (1R,3S)-3-(3-(((benzyloxy)carbonyl)amino)-1H-pyrazol-5-yl)cyclopentyl (3-methyloxetan-3-yl)carbamate (1.0 g, 2.41 mmol) in tetrahydrofuran (10 mL) and ethyl acetate (20 mL) was added palladium (10% on carbon, 0.5 g). The mixture was stirred at 25° C. for 16 h under hydrogen atmosphere (15 psi). The mixture was filtered and the filtrate was concentrated under reduced pressure. The crude was purified by column chromatography (silica gel, 100-200 mesh, 0-100% ethyl acetate in petroleum ether) to give (1R,3S)-3-(3-amino-1H-pyrazol-5-yl)cyclopentyl (3-methyloxetan-3-yl)carbamate (1.0 g, 99%). LCMS (ES-API, M/Z): 281.2 [M+H]⁺.

Step 3: (1R,3S)-3-(3-((3-methylpyrazin-2-yl)amino)-1H-pyrazol-5-yl)cyclopentyl (3-methyloxetan-3-yl)carbamate. A mixture of (1R,3S)-3-(3-(((benzyloxy)carbonyl)amino)-1H-pyrazol-5-yl)cyclopentyl (3-methyloxetan-3-yl)carbamate (100 mg, 0.36 mmol), 2-chloro-3-methylpyrazine (50 mg, 0.39 mmol), sodium tert-butoxide (69 mg, 0.71 mmol) and [9-[dicyclohexyl-[2-(2,6-diisopropoxyphenyl)phenyl]-5-phosphanyl]-8-aza-94-palladatricyclo[8.4.0.02,7]tetradeca-1(10),2(7),3,5,11,13-hexaen-9-yl] methanesulfonate (30 mg, 0.04 mmol) in 2-Methyl-2-propanol (5 mL) was stirred at 100° C. for 4 hours under nitrogen atmosphere. Filtered and the filtrate was concentrated under reduced pressure. Then the crude was purified by preparative TLC (100% ethyl acetate in petroleum ether) to get the crude (20 mg), which was further purified by Purification Procedure R with 0.225% formic acid in water-acetonitrile (30-60%) to afford (1R,3S)-3-(3-((3-methylpyrazin-2-yl)amino)-1H-pyrazol-5-yl)cyclopentyl (3-methyloxetan-3-yl)carbamate (18.8 mg, 15%). LCMS (ES-API, m/z): 373.3 [M+H]⁺; ¹H NMR (400 MHz, DMSO-d6) δ 12.17 (s, 1H), 8.62 (br s, 1H), 7.95 (d, J=2.8 Hz, 1H), 7.78 (d, J=2.8 Hz, 1H), 7.64 (br s, 1H), 6.34 (s, 1H), 5.02 (br s, 1H), 4.56-4.55 (m, 2H), 4.24 (d, J=6.4 Hz, 2H), 3.08-3.04 (m, 1H), 2.48-2.46 (m, 1H), 2.45 (s, 3H), 2.04-1.83 (m, 2H), 1.69-1.63 (m, 3H), 1.47 (s, 3H).

Other compounds were synthesized according to General Procedure and Example 8, as set forth in the below Table L2.

TABLE L2
General Procedure 8 and Example 8 Compounds

| | | Starting | | MS |
|---|---|---|---|---|
| Ex | Structure/Name | Material(s) | ¹H NMR | (m/z) |
| 8A | 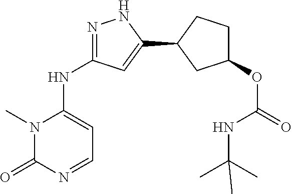 [SMILES: Cn1c(Nc2cc([C@H]3CC[C@@H](OC(=O)NC(C)(C)C)C3)[nH]n2)ccnc1=O] | 2,4- dichloro- pyrimidine and 2- methylpropan-2- amine | ¹H NMR (400 MHz, DMSO- d6) δ 11.91 (br s, 1H), 10.52 (s, 1H), 7.01 (s, 1H), 6.77 (s, 1H), 6.12-5.96 (m, 1H), 5.58 (s, 1H), 4.95 (s, 1H), 3.25 (s, 3H), 3.05-2.94 (m, 1H), 2.48-2.40 (m, 1H), 1.96-1.71 (m, 5H), 1.20 (m, 9H). | 375.3 [M + H]⁺ |
| | (1R,3S)-3-(3-((3-methyl-2-oxo- | | | |
| | 2,3-dihydropyrimidin-4-yl)amino)- | | | |
| | 1H-pyrazol-5-yl)cyclopentyl tert- | | | |
| | butylcarbamate | | | |
| 8B | 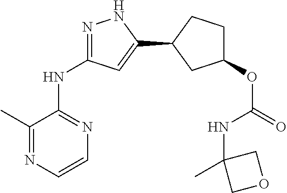 [SMILES: Cc1nccnc1Nc1cc([C@H]2CC[C@@H](OC(=O)NC3(C)COC3)C2)[nH]n1] | 2-chloro-3- methylpyrazine and 3- methyloxetan-3- amine hydrochloride | ¹H NMR (400 MHz, DMSO- d6) δ 12.17 (s, 1H), 8.62 (br s, 1H), 8.14 (s, 1H), 7.95 (s, 1H), 7.78 (s, 1H), 7.64 (s, 1H), 6.34 (s, 1H), 5.02 (s, 1H), 4.56 (s, 2H), 4.24 (s, 2H), 3.08-3.04 (m, 1H), 2.48-2.46 (m, 1H), 2.45 (s, 3H), 2.06-1.83 (m, 2H), 1.79 1.60 (m, 3H), 1.47 (s, 3H). | 373.0 [M + H]⁺ |
| | (1R,3S)-3-(3-((3-methylpyrazin-2- | | | |
| | yl)amino)-1H-pyrazol-5- | | | |
| | yl)cyclopentyl (3-methyloxetan-3- | | | |
| | yl)carbamate | | | |

### General Procedure 9

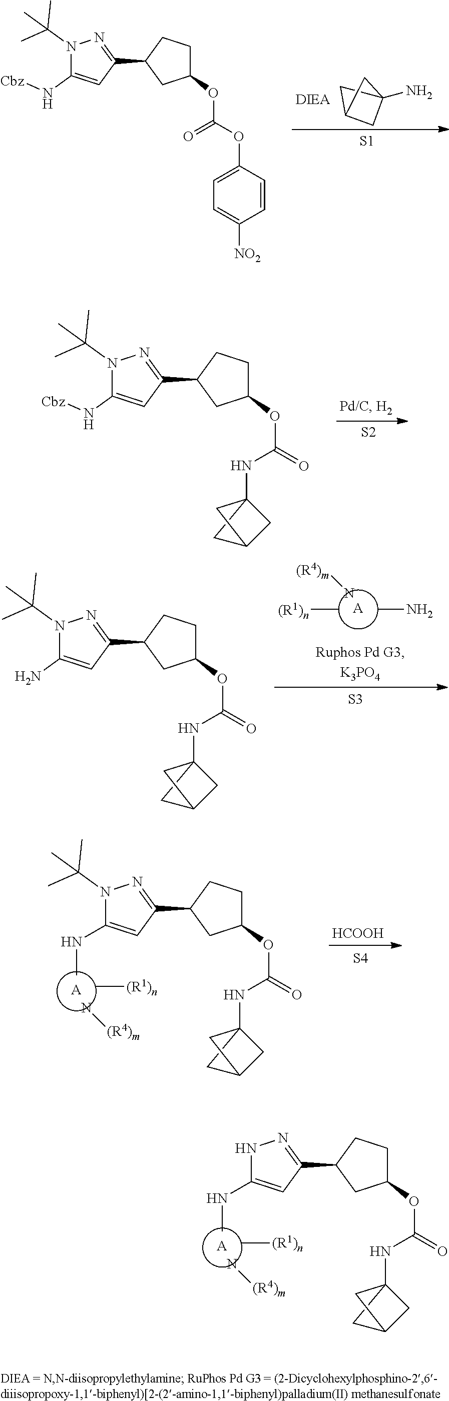

### Example 9. (1R,3S)-3-(3-((2-methylpyridin-4-yl)amino)-1H-pyrazol-5-yl)cyclopentyl bicyclo[1.1.1]pentan-1-ylcarbamate

The title compound was prepared according to General Procedure 9 as described below.

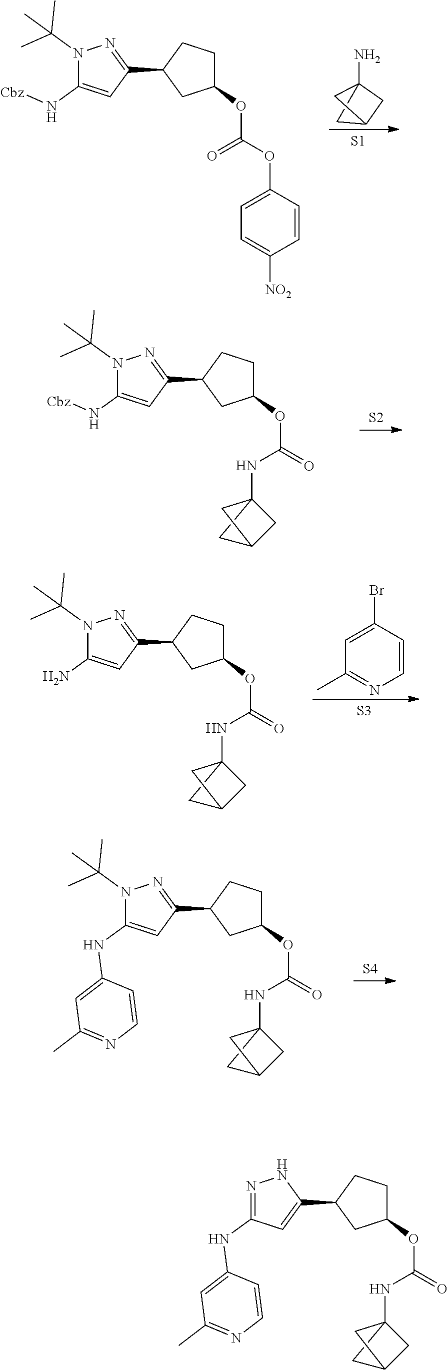

Step 1: (1R,3S)-3-(5-(((benzyloxy)carbonyl)amino)-1-(tert-butyl)-1H-pyrazol-3-yl)cyclopentyl bicyclo[1.1.1]pentan-1-ylcarbamate. To a solution of (4-nitrophenyl) [(1R,3S)-3-[5-(benzyloxycarbonylamino)-1-tert-butyl-pyrazol-3-yl]cyclopentyl] carbonate (8100 mg, 15.5 mmol) in tetrahydrofuran (80 mL) was added N,N-Diisopropylethylamine (8.1 mL, 46.5 mmol) and bicyclo[1.1.1]pentan-1-amine; hydrochloride (2781 mg, 23.2 mmol). The mixture was stirred at 25° C. for 16 hours and concentrated under reduced pressure. The residue was purified by column chromatography (silica gel, 100-200 mesh, 25-35% ethyl acetate in petroleum ether) to give (1R,3S)-3-(5-(((benzyloxy)carbonyl)amino)-1-(tert-butyl)-1H-pyrazol-3-yl)cyclopentyl bicyclo[1.1.1]pentan-1-ylcarbamate (5000 mg, 69.1%). LCMS (ES-API, M/Z): 467.2 [M+H]⁺.

Step 2: (1R,3S)-3-(5-amino-1-(tert-butyl)-1H-pyrazol-3-yl)cyclopentyl bicyclo[1.1.1]pentan-1-ylcarbamate. To a solution of (1R,3S)-3-(5-(((benzyloxy)carbonyl)amino)-1-(tert-butyl)-1H-pyrazol-3-yl)cyclopentyl bicyclo[1.1.1]pentan-1-ylcarbamate (4.0 g, 8.57 mmol) in ethyl acetate (40 mL) and tetrahydrofuran (20 mL) was added palladium (10% on carbon, 1.82 g). The mixture was stirred at 20° C. for 16 hours under hydrogen atmosphere (15 psi) and filtered. The filtrate was concentrated under reduced pressure to give crude (1R,3S)-3-(5-amino-1-(tert-butyl)-1H-pyrazol-3-yl)cyclopentyl bicyclo[1.1.1]pentan-1-ylcarbamate (2.3 g, 80.7%). ¹H NMR (400 MHz, CDCl₃) δ 5.48 (s, 1H), 5.13 (br s, 2H), 3.79 (br, 1H), 3.13 (br s, 1H), 2.53-2.48 (m, 1H), 2.41 (s, 1H), 2.03 (br s, 6H), 1.91-1.86 (m, 3H), 1.77-1.71 (m, 2H), 1.65 (s, 9H). LCMS (ES-API, M/Z): 333.3 [M+H]⁺.

Step 3: (1R,3S)-3-(1-(tert-butyl)-5-((2-methylpyridin-4-yl)amino)-1H-pyrazol-3-yl)cyclopentyl bicyclo[1.1.1]pentan-1-ylcarbamate. A mixture of (1R,3S)-3-(5-amino-1-(tert-butyl)-1H-pyrazol-3-yl)cyclopentyl bicyclo[1.1.1]pentan-1-ylcarbamate (50.0 mg, 0.15 mmol), 4-bromo-2-methylpyridine (0.02 mL, 0.1800 mmol), potassium phosphate (96 mg, 0.45 mmol) and (2-dicyclohexylphosphino-2′,6′-diisopropoxy-1,1′-biphenyl)[2-(2′-amino-1,1′-biphenyl)]palladium(ii) methanesulfonate (12.6 mg, 0.02 mmol) in 2-methyl-2-butanol (4 mL) was stirred at 100° C. for 16 hours under N₂. and concentrated. The residue was purified by column chromatography (silica gel, 100-200 mesh, 30% ethyl acetate in petroleum ether) to give (1R,3S)-3-(1-(tert-butyl)-5-((2-methylpyridin-4-yl)amino)-1H-pyrazol-3-yl)cyclopentyl bicyclo[1.1.1 ]pentan-1-ylcarbamate (55 mg, 86.3%). LCMS (ES-API, M/Z): 424.3 [M+H]⁺.

Step 4: (1R,3S)-3-(3-((2-methylpyridin-4-yl)amino)-1H-pyrazol-5-yl)cyclopentyl bicyclo[1.1.1]pentan-1-ylcarbamate. To a solution of (1R,3S)-3-(1-(tert-butyl)-5-((3-methylpyridin-4-yl)amino)-1H-pyrazol-3-yl)cyclopentyl bicyclo[1.1.1 ]pentan-1-ylcarbamate (40.0 mg, 0.09 mmol) in formic acid (1.0 mL, 26.5 mmol) was stirred at 60° C. for 16 hours and concentrated. The residue was purified with Purification Procedure K with 0.225% formic acid in water-acetonitrile (20-50%) to give (1R,3S)-3-(3-((2-methylpyridin-4-yl)amino)-1H-pyrazol-5-yl)cyclopentyl bicyclo[1.1.1]pentan-1-ylcarbamate (4.5 mg, 12.3%). ¹H NMR (400 MHz, DMSO-d6) δ 9.08 (s, 1H), 8.23 (br s, 1H), 8.04 (d, J=6.0 Hz, 1H), 7.77 (br s, 1H), 7.11 (s, 1H), 7.05 (d, J=5.6 Hz, 1H), 5.72 (s, 1H), 4.99 (br s, 1H), 3.08-3.03 (m, 1H), 2.48-2.44 (m, 1H), 2.33 (s, 3H), 2.08-2.03 (m, 1H), 2.03-1.97 (m, 1H), 1.89 (br s, 6H), 1.71-1.60 (m, 3H). LCMS (ES-API, M/Z): 368.1 [M+H]⁺.

### Example 9A: (1R,3S)-3-(3-((1-methyl-6-oxo-1,6-dihydropyrimidin-2-yl)amino)-1H-pyrazol-5-yl)cyclopentyl bicyclo[1.1.1]pentan-1-ylcarbamate

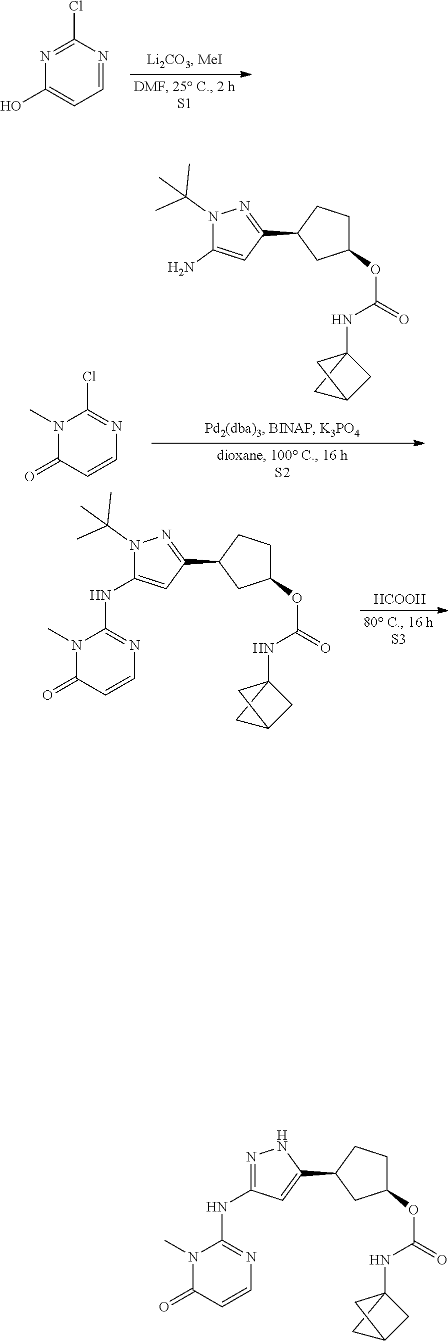

Step 1: 2-chloro-3-methyl-pyrimidin-4-one. To a solution of 2-chloropyrimidin-4-ol (500 mg, 3.83 mmol) in N,N-dimethylformamide (10 mL) was added lithium carbonate (570 mg, 7.66 mmol) and iodomethane (0.48 mL, 7.66 mmol). The mixture was stirred at 25° C. for 2 hours. The mixture was extracted with ethyl acetate (3×20 mL) and water (10 mL), washed with brine (3×30 mL). The organic layer was dried over anhydrous sodium sulfate, filtered and concentrated under reduced pressure. The crude was purified by column chromatography (silica gel, 100-200 mesh, 0 to 20% ethyl acetate in petroleum ether) to give 2-chloro-3-methyl-pyrimidin-4-one (300 mg, 54%). ¹H NMR (400 MHz, CDCl₃) δ 7.74 (d, J=6.4 Hz, 1H), 6.42 (d, J=6.4 Hz, 1H), 3.68 (s, 3H).

Step 2: (1R,3S)-3-(1-(tert-butyl)-5-((1-methyl-6-oxo-1,6-dihydropyrimidin-2-yl)amino)-1H-pyrazol-3-yl)cyclopentyl bicyclo[1.1.1]pentan-1-ylcarbamate. A mixture of (1R,3S)-3-(5-amino-1-(tert-butyl)-1H-pyrazol-3-yl)cyclopentyl bicyclo[1.1.1]pentan-1-ylcarbamate (300 mg, 0.90 mmol), 2-chloro-3-methyl-pyrimidin-4-one (196 mg, 1.35 mmol), potassium phosphate (575 mg, 2.71 mmol), (R)-(+)-2,2′-bis(diphenylphosphino)-1,1′-binaphthyl (56 mg, 0.09 mmol) and tris(dibenzylideneacetone)dipalladium (0) (50 mg, 0.05 mmol) in 1,4-dioxane (10 mL) was stirred at 100° C. for 16 hours under nitrogen atmosphere. To the reaction was added methanol (10 mL) and the mixture was filtered. The filtrate was concentrated under reduced pressure and the residue was purified by column chromatography (silica gel, 100-200 mesh, 0 to 50% ethyl acetate in petroleum ether) to give (1R,3S)-3-(1-(tert-butyl)-5-((1-methyl-6-oxo-1,6-dihydropyrimidin-2-yl)amino)-1H-pyrazol-3-yl)cyclopentyl bicyclo[1.1.1]pentan-1-ylcarbamate (113 mg, 28%). LCMS (ES-API, m/z): 441.2 [M+H]⁺.

Step 3: (1R,3S)-3-(3-((1-methyl-6-oxo-1,6-dihydropyrimidin-2-yl)amino)-1H-pyrazol-5-yl)cyclopentyl bicyclo[1.1.1]pentan-1-ylcarbamate. A mixture of (1R,3S)-3-(1-(tert-butyl)-5-((1-methyl-6-oxo-1,6-dihydropyrimidin-2-yl)amino)-1H-pyrazol-3-yl)cyclopentyl bicyclo[1.1.1]pentan-1-ylcarbamate (113 mg, 0.26 mmol) in formic acid (5 mL) was stirred at 80° C. for 16 hours. The mixture was concentrated under reduced pressure and the residue was purified by Purification Procedure N with 0.05% ammonium hydroxide and 10 mM ammonium bicarbonate in water-acetonitrile (30-60%) to obtain (1R,3S)-3-(3-((1-methyl-6-oxo-1,6-dihydropyrimidin-2-yl)amino)-1H-pyrazol-5-yl)cyclopentyl bicyclo[1.1.1]pentan-1-ylcarbamate (38 mg, 38%). LCMS (ES-API, m/z): 385.1 [M+H]⁺; ¹H NMR (400 MHz, DMSO-d6) δ 12.2 (br s, 1H), 9.14 (br s, 1H), 7.77 (s, 1H), 7.60 (s, 1H), 5.78 (s, 1H), 5.52 (s, 1H), 4.79 (s, 1H), 3.26 (s, 3H), 3.08-3.04 (m, 1H), 2.50-2.34 (m, 2H), 2.07-1.55 (m, 11H).

Other compounds were synthesized according to General Procedure and Example 9, as set forth in the below Table M.

TABLE M
General Procedure 9 and Example 9 Compounds

| | | Starting | | |
|---|---|---|---|---|
| Ex | Structure/Name | Material | ¹H NMR | MS (m/z) |
| 9A | 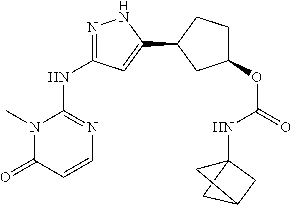 [SMILES: Cn1c(Nc2cc([C@H]3CC[C@@H](OC(=O)NC45CC(C4)C5)C3)[nH]n2)nccc1=O] | 2-chloro-3- methyl- pyrimidin- 4(3H)-one | ¹H NMR (400 MHz, DMSO- d6) δ 12.20-12.00 (m, 1H), 11.73 (s, 0.5H), 9.14 (s, 0.5H), 7.77 (s, 1H), 7.60 (s, 1H), 6.26 (s, 0.5H), 5.78 (s, 1H), 5.52 (s, 0.5H), 4.99 (s, 1H), 3.41 (s, 1.5H), 3.26 (s, 1.5H), 3.13-2.98 (m, 1H), 2.48-2.40 (m, 1H), 2.34 (s, 1H), 2.11-1.97 (m, 1H), 1.90-1.55 (m, 10H). | 385.1 [M + H]⁺ |
| | (1R,3S)-3-(3-((1-methyl-6-oxo-1,6- | | | |
| | dihydropyrimidin-2-yl)amino)-1H- | | | |
| | pyrazol-5-yl)cyclopentyl | | | |
| | bicyclo[1.1.1]pentan-1-ylcarbamate | | | |
| 9B | 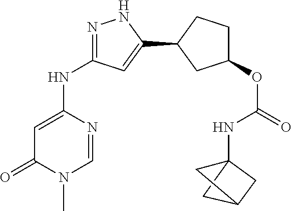 [SMILES: Cn1cnc(Nc2cc([C@H]3CC[C@@H](OC(=O)NC45CC(C4)C5)C3)[nH]n2)cc1=O] | 6-chloro-3- methyl- pyrimidin- 4(3H)-one | ¹H NMR (400 MHz, DMSO- d6) δ 12.01 (br s, 1H), 9.24 (s, 1H), 8.18 (s, 1H), 7.76 (br s, 1H), 6.36 (br s, 1H), 5.78 (s, 1H), 4.98 (br s, 1H), 3.28 (s, 3H), 3.08-2.98 (m, 1H), 2.47-2.40 (m, 1H), 2.34 (s, 1H), 2.01-1.99 (m, 1H), 1.89-1.57 (m, 10H). | 385.1 [M + H]⁺ |
| | (1R,3S)-3-(3-((1-methyl-6-oxo-1,6- | | | |
| | dihydropyrimidin-4-yl)amino)-1H- | | | |
| | pyrazol-5-yl)cyclopentyl | | | |
| | bicyclo[1.1.1]pentan-1-ylcarbamate | | | |
| 9C | 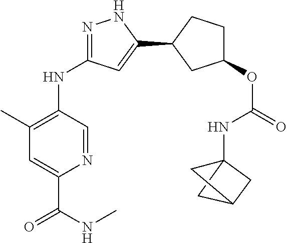 [SMILES: CNC(=O)c1cc(C)c(Nc2cc([C@H]3CC[C@@H](OC(=O)NC45CC(C4)C5)C3)[nH]n2)cn1] | 5-bromo- N,4- dimethyl- pyridine-2- carboxamide | ¹H NMR (400 MHz, DMSO- d6) δ 11.98 (br s, 1H), 9.04 (br s, 1H), 8.40-8.39 (m, 1H), 7.83 (s, 1H), 7.73 (s, 1H), 5.84 (s, 1H), 4.99 (br s, 1H), 3.12-3.00 (m, 1H), 2.78 (d, J = 4.8 Hz, 3H), 2.67 (s, 1H), 2.35-2.33 (m, 2H), 2.29 (s, 3H), 2.03-1.99 (m, 1H), 1.90-1.60 (m, 10H). | 425.4 [M + 1] |
| | (1R,3S)-3-(3-((4-methyl-6- | | | |
| | (methylcarbamoyl)pyridin-3- | | | |
| | yl)amino)-1H-pyrazol-5- | | | |
| | yl)cyclopentyl bicyclo[1.1.1]pentan- | | | |
| | 1-ylcarbamate | | | |
| 9D | 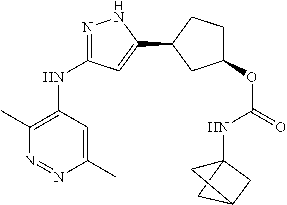 [SMILES: Cc1cc(Nc2cc([C@H]3CC[C@@H](OC(=O)NC45CC(C4)C5)C3)[nH]n2)c(C)nn1] | 4-chloro-3,6- dimethyl- pyridazine | ¹H NMR (400 MHz, DMSO- d6) δ 12.63 (br s, 1H), 9.83 (br s, 1H), 8.06 (s, 1H), 7.75 (br s, 1H), 6.12 (s, 1H), 5.00 (s, 1H), 3.18-3.11 (m, 1H), 2.58 (s, 3H), 2.55 (s, 3H), 2.53-2.51 (m, 1H), 2.38- 2.27 (m, 1H), 2.11-2.01 (m, 1H), 2.00-1.78 (m, 8H), 1.72-1.62 (m, 2H). | 383.1 [M + H]⁺ |
| | (1R,3S)-3-(3-((3,6- | | | |
| | dimethylpyridazin-4-yl)amino)-1H- | | | |
| | pyrazol-5-yl)cyclopentyl | | | |
| | bicyclo[1.1.1]pentan-1-ylcarbamate | | | |
| 9E | 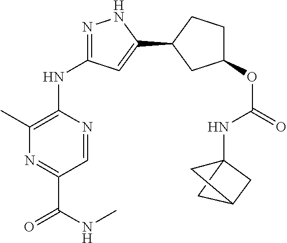 [SMILES: CNC(=O)c1cnc(Nc2cc([C@H]3CC[C@@H](OC(=O)NC45CC(C4)C5)C3)[nH]n2)c(C)n1] | 5-chloro- N,6- dimethyl- pyrazine-2- carboxamide | ¹H NMR (400 MHz, DMSO- d6) δ 12.16 (s, 1H), 9.09 (s, 1H), 8.50 (s, 1H), 8.29-8.28 (m, 1H), 7.77 (s, 1H), 6.46 (s, 1H), 5.00 (s, 1H), 3.08 (s, 1H), 2.79 (s, 3H), 2.52 (s, 3H), 2.34 (s, 1H), 2.03 (s, 1H), 1.90 (s, 7H), 1.73-1.64 (m, 4H). | 448.1 [M + Na]⁺ |
| | (1R,3S)-3-(3-((3-methyl-5- | | | |
| | (methylcarbamoyl)pyrazin-2- | | | |
| | yl)amino)-1H-pyrazol-5- | | | |
| | yl)cyclopentyl bicyclo[1.1.1]pentan- | | | |
| | 1-ylcarbamate | | | |
| 9F | 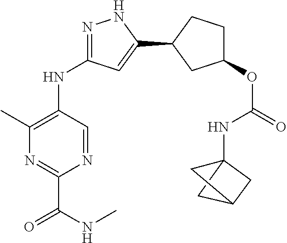 [SMILES: CNC(=O)c1ncc(Nc2cc([C@H]3CC[C@@H](OC(=O)NC45CC(C4)C5)C3)[nH]n2)c(C)n1] | 5-bromo-4- methyl- pyrimidine- 2- carboxylate | ¹H NMR (400 MHz, DMSO- d6) δ 12.06 (s, 1H), 9.32 (s, 1H), 8.52 (s, 1H), 8.18 (s, 1H), 7.76 (s, 1H), 5.86 (s, 1H), 3.09-3.04 (m, 1H), 2.77 (s, 3H), 2.51 (s, 3H), 2.50-2.49 (m, 1H), 2.34- 2.32 (m, 1H), 2.07-2.02 (m, 1H), 1.89-1.59 (m, 10H). | 426.1 [M + H] |
| | (1R,3S)-3-(3-((4-methyl-2- | | | |
| | (methylcarbamoyl)pyrimidin-5- | | | |
| | yl)amino)-1H-pyrazol-5- | | | |
| | yl)cyclopentyl bicyclo[1.1.1]pentan- | | | |
| | 1-ylcarbamate | | | |
| 9G | 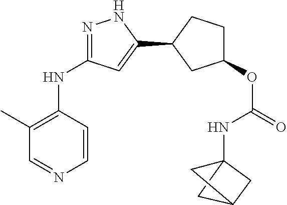 [SMILES: Cc1cnccc1Nc1cc([C@H]2CC[C@@H](OC(=O)NC34CC(C3)C4)C2)[nH]n1] | 4-bromo-3- methyl- pyridine hydro- chloride | ¹H NMR (400 MHz, DMSO- d6) δ 12.55 (s, 1H), 8.25 (s, 1H), 8.09-7.99 (m, 2H), 7.86 (s, 1H), 7.80-7.70 (m, 1H), 7.64-7.54 (m, 1H), 5.86 (s, 1H), 4.99 (s, 1H), 3.09-3.05 (m, 1H), 2.34- 2.30 (m, 1H), 2.16 (s, 3H), 2.05-1.97 (m, 2H), 1.89 (br s, 6H), 1.77-1.60 (m, 3H). | 368.1 [M + H] |
| | (1R,3S)-3-(3-((3-methylpyridin-4- | | | |
| | yl)amino)-1H-pyrazol-5- | | | |
| | yl)cyclopentyl bicyclo[1.1.1]pentan- | | | |
| | 1-ylcarbamate | | | |
| 9H | 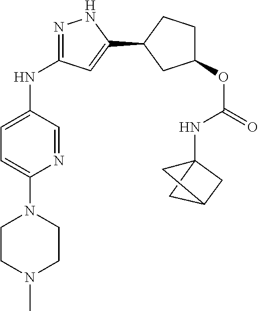 [SMILES: CN1CCN(c2ccc(Nc3cc([C@H]4CC[C@@H](OC(=O)NC56CC(C5)C6)C4)[nH]n3)cn2)CC1] | 1-(5-bromo- pyridin-2- yl)-4-methyl- piperazine | ¹H NMR (400 MHz, DMSO- d6) δ 11.59 (br s, 1H), 8.20 (br s, 1H), 8.03 (s, 1H), 7.76- 7.67 (m, 2H), 6.78 (d, J = 9.2 Hz, 1H), 5.50 (s, 1H), 4.96 (br s, 1H), 3.01-2.97 (m, 1H), 2.96-2.81 (m, 3H), 2.47-2.41 (m, 8H), 2.38- 2.28 (m, 2H), 2.08-1.97 (m, 1H), 1.88-1.67 (m, 10H). | 452.3 [M + H]⁺ |
| | (1R,3S)-3-(3-((6-(4- | | | |
| | methylpiperazin-1-yl)pyridin-3- | | | |
| | yl)amino)-1H-pyrazol-5- | | | |
| | yl)cyclopentyl bicyclo[1.1.1]pentan- | | | |
| | 1-ylcarbamate | | | |
| 9I | 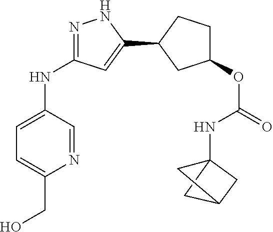 [SMILES: O=C(NC12CC(C1)C2)O[C@@H]1CC[C@H](c2cc(Nc3ccc(CO)nc3)n[nH]2)C1] | (5-bromo-2- pyridyl)- methanol | ¹H NMR (400 MHz, Methanol-d4) δ 8.40 (s, 1H), 8.28 (br s, 1H), 7.77-7.74 (m, 1H), 7.38 (d, J = 8.8 Hz, 1H), 5.77 (s, 1H), 5.08 (br s, 1H), 4.61 (s, 2H), 3.19-3.10 (m, 1H), 2.55-2.52 (m, 1H), 2.36 (s, 1H), 2.16-2.08 (m, 1H), 1.99-1.76 (m, 10H). | 384.3 [M + H]⁺ |
| | (1R,3S)-3-(3-((6- | | | |
| | (hydroxymethyl)pyridin-3- | | | |
| | yl)amino)-1H-pyrazol-5- | | | |
| | yl)cyclopentyl bicyclo[1.1.1]pentan- | | | |
| | 1-ylcarbamate | | | |
| 9J | 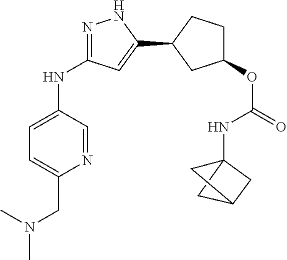 [SMILES: CN(C)Cc1ccc(Nc2cc([C@H]3CC[C@@H](OC(=O)NC45CC(C4)C5)C3)[nH]n2)cn1] | 1-(5-bromo- pyridin-2- yl)-N,N- dimethyl- methanamine | ¹H NMR (400 MHz, DMSO- d6) δ 11.78 (br s, 1H), 8.48 (s, 1H), 8.42 (s, 1H), 7.77- 7.76 (m, 1H), 7.52 (s, 1H), 7.18 (d, J = 8.0 Hz, 1H), 5.61 (br s, 1H), 4.98 (br s, 1H), 3.03 (br s, 1H), 2.34 (br s, 2H), 2.13 (s, 6H), 2.13-2.00 (m, 3H), 1.89-1.59 (m, 10H). | 411.1 [M + H]⁺ |
| | (1R,3S)-3-(3-((6- | | | |
| | ((dimethylamino)methyl)pyridin-3- | | | |
| | yl)amino)-1H-pyrazol-5- | | | |
| | yl)cyclopentyl bicyclo[1.1.1]pentan- | | | |
| | 1-ylcarbamate | | | |
| 9K | 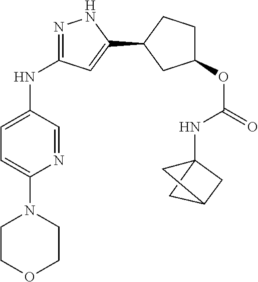 [SMILES: O=C(NC12CC(C1)C2)O[C@@H]1CC[C@H](c2cc(Nc3ccc(N4CCOCC4)nc3)n[nH]2)C1] | 5-bromo-2- fluoro- pyridine | ¹H NMR (400 MHz, DMSO- d6) δ 11.59 (br s, 1H), 8.27- 8.17 (m, 1H), 7.99 (s, 1H), 7.75 (br s, 1H), 7.68-7.65 (m, 1H), 6.74 (d, J = 8.8 Hz, 1H), 5.52 (s, 1H), 4.98 (br s, 1H), 3.71-3.67 (m, 4H), 3.27-3.23 (m, 4H), 3.01 (t, J = 8.4 Hz, 1H), 2.47-2.34 (m, 2H), 2.01-1.98 (m, 1H), 1.89-1.58 (m, 10H). | 439.2 [M + H]⁺ |
| | (1R,3S)-3-(3-((6- | | | |
| | morpholinopyridin-3-yl)amino)-1H- | | | |
| | pyrazol-5-yl)cyclopentyl | | | |
| | bicyclo[1.1.1]pentan-1-ylcarbamate | | | |

### General Procedure 10

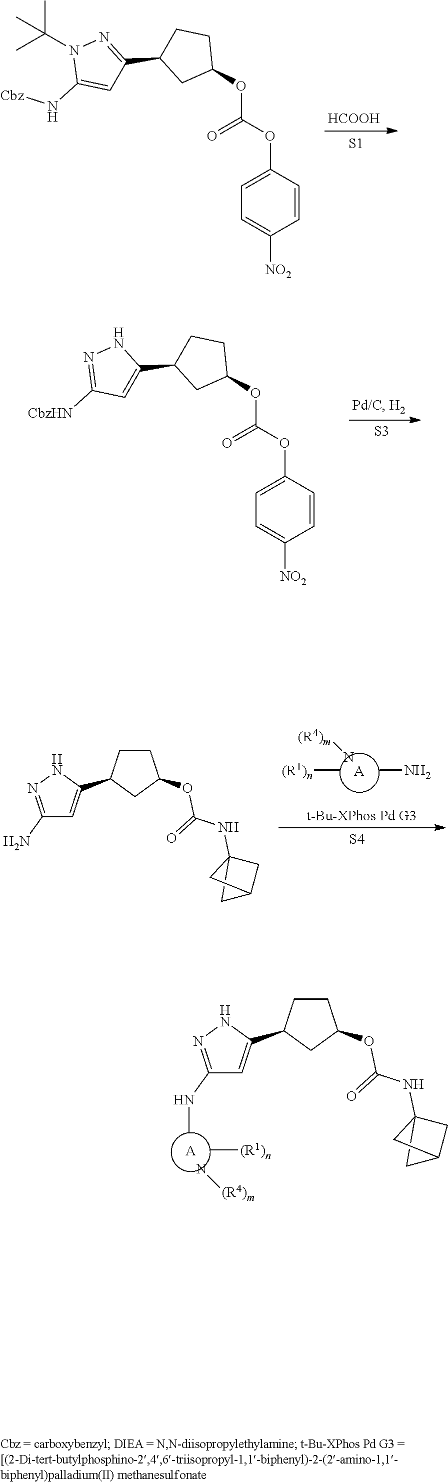

### Example 10. (1R,3S)-3-(3-((4-methoxy-1-methyl-6-oxo-1,6-dihydropyridin-3-yl)amino)-1H-pyrazol-5-yl)cyclopentyl bicyclo[1.1.1]pentan-1-ylcarbamate

The title compound was prepared according to General Procedure 10 as described below.

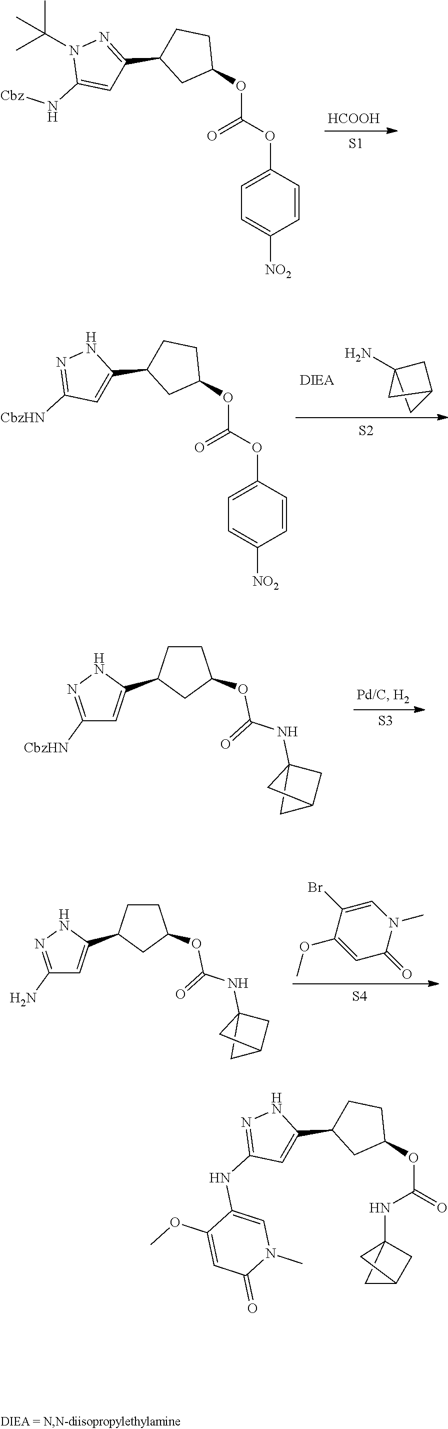

Step 1: benzyl (5-((1S,3R)-3-(((4-nitrophenoxy)carbonyl)oxy)cyclopentyl)-1H-pyrazol-3-yl)carbamate. A solution of benzyl (1-(tert-butyl)-3-((1S,3R)-3-(((4-nitrophenoxy)carbonyl)oxy)cyclopentyl)-1H-pyrazol-5-yl)carbamate (4.0 g, 7.65 mmol) in formic acid (30 mL, 795.13 mmol) was stirred at 75° C. for 16 hours and concentrated to give crude benzyl (5-((1S,3R)-3-(((4-nitrophenoxy)carbonyl)oxy)cyclopentyl)-1H-pyrazol-3-yl)carbamate (3.0 g, 84%). LCMS (ES-API, M/Z): 467.0 [M+H]⁺.

Step 2: benzyl (3-((1S,3R)-3-((bicyclo[1.1.1]pentan-1-ylcarbamoyl)oxy)cyclopentyl)-1H-pyrazol-5-yl)carbamate. To a solution of benzyl (5-((1S,3R)-3-(((4-nitrophenoxy)carbonyl)oxy)cyclopentyl)-1H-pyrazol-3-yl)carbamate (3.0 g, 6.43 mmol) in tetrahydrofuran (20 mL) was added bicyclo[1.1.1]pentan-1-amine; hydrochloride (1.5 g, 12.86 mmol) and N,N-diisopropylethylamine (3.4 mL, 19.03 mmol). The mixture was stirred at 25° C. for 16 hours and concentrated under reduced pressure. The residue was purified by column chromatography (silica gel, 100-200 mesh, 55% ethyl acetate in petroleum ether) to give benzyl (3-((1S,3R)-3-((bicyclo[1.1.1]pentan-1-ylcarbamoyl)oxy)cyclopentyl)-1H-pyrazol-5-yl)carbamate (2.3 g, 87.1%). LCMS (ES-API, M/Z): 411.3 [M+H]⁺.

Step 3: (1R,3S)-3-(3-amino-1H-pyrazol-5-yl)cyclopentyl bicyclo[1.1.1]pentan-1-ylcarbamate. To a solution of benzyl (3-((1S,3R)-3-((bicyclo[1.1.1]pentan-1-ylcarbamoyl)oxy)cyclopentyl)-1H-pyrazol-5-yl)carbamate (1.15 g, 2.8 mmol) in ethyl acetate (15 mL) and tetrahydrofuran (8 mL) was added palladium (10% on carbon, 0.60 g). The mixture was stirred at 20° C. for 16 hours under hydrogen atmosphere (15 psi) and filtered. The filtrate was concentrated to give (1R,3S)-3-(3-amino-1H-pyrazol-5-yl)cyclopentyl bicyclo[1.1.1]pentan-1-ylcarbamate (1.10 g). ¹H NMR (400 MHz, DMSO-d6) δ 11.08 (br s, 1H), 7.75 (m, 1H), 5.18 (s, 1H), 4.95 (br s, 1H), 4.45 (br s, 1H), 2.91-2.84 (m, 1H), 2.42-2.34 (m, 2H), 1.95-1.73 (m, 9H), 1.69-1.47 (m, 3H). LCMS (ES-API, M/Z): 277.2 [M+H]⁺.

Step 4: (1R,3S)-3-(3-((4-methoxy-1-methyl-6-oxo-1,6-dihydropyridin-3-yl)amino)-1H-pyrazol-5-yl)cyclopentyl bicyclo[1.1.1]pentan-1-ylcarbamate. A mixture of (1R,3S)-3-(3-amino-1H-pyrazol-5-yl)cyclopentyl bicyclo[1.1.1]pentan-1-ylcarbamate (150.0 mg, 0.54 mmol), 5-bromo-4-methoxy-1-methyl-pyridin-2-one (142.0 mg, 0.65 mmol), cesium carbonate (530.6 mg, 1.63 mmol) and t-BuXPhos Phos palladium(II) biphenyl-2-amine mesylate (43.0 mg, 0.05 mmol) in 2-Methyl-2-propanol (10 mL) was stirred at 80° C. under nitrogen for 16 hours and concentrated. The residue was purified first by column chromatography (silica gel, 100-200 mesh, 0-10% methanol in dichloromethane), and then with Purification Procedure L 0.05% ammonium hydroxide-10 mM ammonium bicarbonate in water-acetonitrile (20-50%) to give (1R,3S)-3-(3-((4-methoxy-1-methyl-6-oxo-1,6-dihydropyridin-3-yl)amino)-1H-pyrazol-5-yl)cyclopentyl bicyclo[1.1.1]pentan-1-ylcarbamate (11.6 mg, 5%). ¹H NMR (400 MHz, DMSO-d6) δ 11.58 (br s, 1H), 8.16 (s, 1H), 7.76 (br s, 1H), 7.16 (s, 1H), 5.86 (s, 1H), 5.65 (s, 1H), 4.98 (m, 1H), 3.82 (s, 3H), 3.34 (s, 3H), 3.01-2.97 (m, 1H), 2.44-2.42 (m, 1H), 2.38-2.35 (m, 1H), 2.03-1.98 (m, 1H), 1.89-1.81 (m, 7H), 1.68-1.65 (m, 3H). LCMS (ES-API, M/Z): 414.1 [M+H]⁺.

### Example 10D: (1R,3S)-3-(3-((6-cyano-2-methylpyridin-3-yl)amino)-1H-pyrazol-5-yl)cyclopentyl tert-butylcarbamate

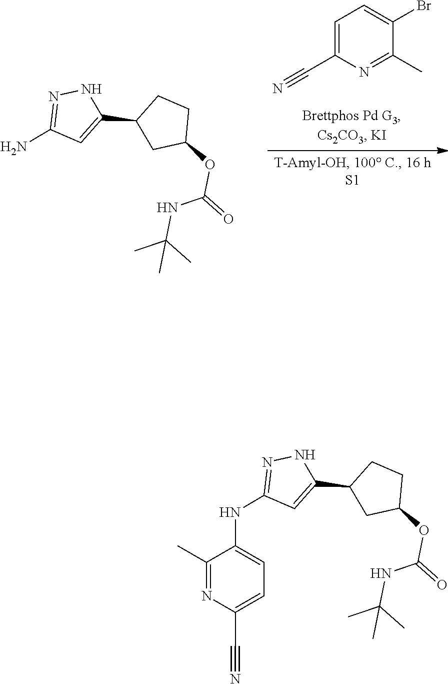

To a mixture of 5-bromo-6-methyl-pyridine-2-carbonitrile (266 mg, 1.35 mmol), (1R,3S)-3-(3-amino-1H-pyrazol-5-yl)cyclopentyl tert-butylcarbamate (300 mg, 1.13 mmol), cesiumcarbonate (1101 mg, 3.38 mmol) and potassium iodide (224 mg, 1.35 mmol) in 2-methyl-2-butanol (6 mL) was added [2-(2-aminophenyl)phenyl] methylsulfonyloxy-palladium dicyclohexyl-[3,6-dimethoxy-2-(2,4,6-triisopropylphenyl)phenyl]phosphane (102 mg, 0.11 mmol). The reaction was stirred at 100° C. for 16 hours under nitrogen atmosphere. The mixture was filtered and the filtrate was concentrated under reduced pressure. The residue was purified by column chromatography (silica gel, 100-200 mesh, 0 to 3% methyl alcohol in dichloromethane) to give (1R,3S)-3-(3-((6-cyano-2-methylpyridin-3-yl)amino)-1H-pyrazol-5-yl)cyclopentyl tert-butylcarbamate (110 mg, 25%). LCMS (ES-API, m/z): 383.3 [M+H]⁺; ¹H NMR (400 MHz, DMSO-d6) δ 12.10 (br s, 1H), 8.27-8.24 (m, 2H), 7.67 (d, J=8.4 Hz, 1H), 6.77 (br s, 1H), 5.91 (d, J=2.0 Hz, 1H), 4.98 (br s, 1H), 3.09-3.05 (m, 1H), 2.48 (s, 1H), 2.47-2.45 (m, 1H), 2.04-1.90 (m, 2H), 1.75-1.95 (m, 3H), 1.75-1.58 (m, 3H), 1.20 (s, 9H).

### General Procedure 11

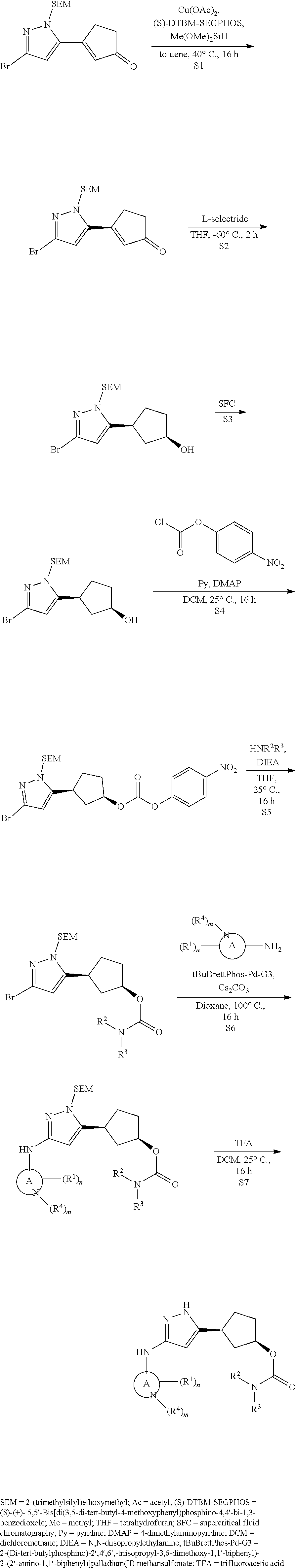

### Example 11: (1R,3S)-3-(3-((3-cyclopropylpyridazin-4-yl)amino)-1H-pyrazol-5-yl)cyclopentyl bicyclo[1.1.1]pentan-1-ylcarbamate

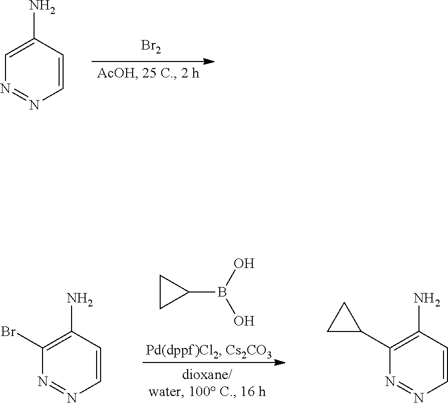 [SMILES: Nc1ccnnc1 ; "Br2" ; Nc1ccnnc1Br ; OB(O)C1CC1 ; Nc1ccnnc1C1CC1]

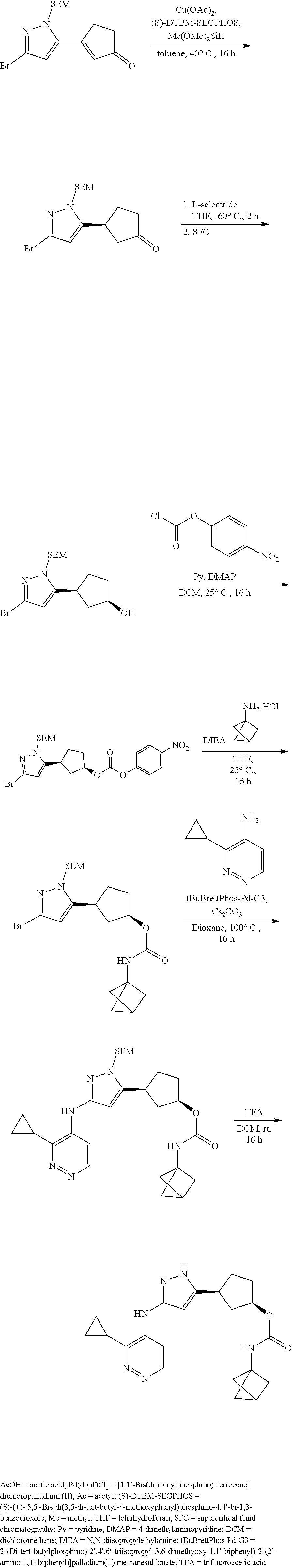

Step 1: 3-bromopyridazin-4-amine. To a solution of pyridazin-4-amine (5.0 g, 52.58 mmol) in acetic acid (50 mL) was added bromine (2.42 mL, 47.32 mmol). The reaction was stirred at 25° C. for 2 h. The mixture was adjusted to pH=10 with aqueous sodium hydroxide (2 M), and then extracted with dichloromethane (3×50 mL). The combined organic layers were dried over anhydrous sodium sulfate and concentrated under reduced pressure. The residue was purified by column chromatography (silica gel, 100-200 mesh, 0-80% ethyl acetate in petroleum ether) to obtain 3-bromopyridazin-4-amine (1.0 g, 11%). ¹H NMR (400 MHz, DMSO-d6) δ 8.49 (d, J=5.6 Hz, 1H), 6.73 (d, J=5.6 Hz, 1H), 6.65 (br s, 2H).

Step 2: 3-cyclopropylpyridazin-4-amine. A mixture of 3-bromopyridazin-4-amine (900 mg, 5.17 mmol), cesium carbonate (6.7 g, 20.69 mmol), cyclopropylboronic acid (4.0 g, 46.55 mmol) and [1,1′-Bis(diphenylphosphino)ferrocene]dichloropalladium(II) (380 mg, 0.52 mmol) in 1,4-dioxane (20 mL) and water (4 mL) was stirred at 100° C. under nitrogen atmosphere for 16 h. The reaction mixture was diluted with ethyl acetate (50 mL) and filtered. The filtrate was concentrated under reduced pressure and the crude was purified by column chromatography (silica gel, 100-200 mesh, 0-10% methanol in ethyl acetate) to obtain 3-cyclopropylpyridazin-4-amine (400 mg, 57%). ¹H NMR (400 MHz, DMSO-d6) δ 8.32 (d, J=5.6 Hz, 1H), 6.54 (d, J=5.6 Hz, 1H), 6.33 (br s, 2H), 2.13-2.09 (m, 1H), 0.97-0.91 (m, 4H).

Step 3: (S)-3-(3-bromo-1-((2-(trimethylsilyl)ethoxy)methyl)-1H-pyrazol-5-yl)cyclopentanone. To a solution of 3-(3-bromo-1-((2-(trimethylsilyl)ethoxy)methyl)-1H-pyrazol-5-yl)cyclopent-2-enone (3.0 g, 8.40 mmol), [4-[5-bis(3,5-ditert-butyl-4-methoxy-phenyl)phosphanyl-1,3-benzodioxol-4-yl]-1,3-benzodioxol-5-yl]-bis(3,5-ditert-butyl-4-methoxy-phenyl)phosphane (1.0 g, 0.84 mmol) in toluene (80 mL) was added copper(II) acetate (150 mg, 0.84 mmol) and dimethoxy(methyl)silane (2.18 mL, 16.79 mmol). The reaction was stirred at 40° C. for 16 h under nitrogen atmosphere. To the mixture was added water (20 mL), followed by tetrabutylammonium fluoride (5 mL, 1 M in tetrahydrofuran). The reaction mixture was stirred for 30 mins, and then extracted with ethyl acetate (2×50 mL). The organic layers were dried over anhydrous sodium sulfate and concentrated under reduced pressure to afford (S)-3-(3-bromo-1-((2-(trimethylsilyl)ethoxy)methyl)-1H-pyrazol-5-yl)cyclopentanone (3.0 g, 99%). The crude product was used for next step directly.

Step 4: (1R,3S)-3-(3-bromo-1-((2-(trimethylsilyl)ethoxy)methyl)-1H-pyrazol-5-yl)cyclopentanol. To above solution of (S)-3-(3-bromo-1-((2-(trimethylsilyl)ethoxy)methyl)-1H-pyrazol-5-yl)cyclopentanone (3.0 g, 8.35 mmol) in tetrahydrofuran (50 mL) was added tri-sec-butylborohydride (10.02 mL, 10.02 mmol) (1 M in tetrahydrofuran) dropwise at −60° C. under nitrogen atmosphere. The mixture was stirred at −60° C. for 2 h under nitrogen atmosphere. The mixture was poured into water (100 mL) and stirred for 30 mins. The aqueous layer was extracted with ethyl acetate (2×100 mL). The combined organic layers were dried over anhydrous sodium sulfate and filtered. The filtrate was concentrated under reduced pressure. The crude product was purified by column chromatography (silica gel, 100-200 mesh, 0-15% ethyl acetate in petroleum ether) to afford (1R,3S)-3-(3-bromo-1-((2-(trimethylsilyl)ethoxy)methyl)-1H-pyrazol-5-yl)cyclopentanol (1.9 g, 64%), which was further separated by Purification Procedure M (Chiral SFC) with 0.1% ammonium hydroxide-25% ethanol-carbon dioxide to afford (1R,3S)-3-(3-bromo-1-((2-(trimethylsilyl)ethoxy)methyl)-1H-pyrazol-5-yl)cyclopentanol (816 mg, 43%, retention time=4.056 min). LCMS (ES-API, M/Z): 361.1 [M+H]⁺. ¹H NMR (400 MHz, CDCl₃) δ 6.23 (s, 1H), 5.38 (s, 2H), 4.45 (br s, 1H), 3.61-3.47 (m, 2H), 3.28-3.17 (m, 1H), 2.52-2.42 (m, 1H), 2.18-2.06 (m, 1H), 1.96-1.85 (m, 2H), 1.73-1.63 (m, 1H), 1.63-1.59 (m, 1H), 1.29-1.16 (m, 1H), 0.91-0.85 (m, 2H), 0.00 (s, 9H).

Step 5: (1R,3S)-3-(3-bromo-1-((2-(trimethylsilyl)ethoxy)methyl)-1H-pyrazol-5-yl)cyclopentyl (4-nitrophenyl) carbonate. To a solution of (1R,3S)-3-(3-bromo-1-((2-(trimethylsilyl)ethoxy)methyl)-1H-pyrazol-5-yl)cyclopentanol (3.0 g, 8.3 mmol) in dichloromethane (30 mL) was added N,N-dimethylpyridin-4-amine (101 mg, 0.83 mmol), pyridine (2.0 g, 24.91 mmol) and 4-nitrophenyl carbonchloridate (2.5 g, 12.45 mmol). The mixture was stirred at 25° C. for 16 h. The reaction was quenched by the addition of saturated aqueous ammonium chloride (200 mL), and then extracted with ethyl acetate (2×100 mL). The combined organic layers were dried over sodium sulfate, filtered and concentrated under reduced pressure. The residue was purified by column chromatography (silica gel, 100-200 mesh, 0-15% ethyl acetate in petroleum ether) to afford (1R,3S)-3-(3-bromo-1-((2-(trimethylsilyl)ethoxy)methyl)-1H-pyrazol-5-yl)cyclopentyl (4-nitrophenyl) carbonate (4.2 g, 96%). LCMS (ES-API, M/Z): 526.1 [M+H]⁺.

Step 6: (1R,3S)-3-(3-bromo-1-((2-(trimethylsilyl)ethoxy)methyl)-1H-pyrazol-5-yl)cyclopentyl bicyclo[1.1.1]pentan-1-ylcarbamate. To a solution of (1R,3S)-3-(3-bromo-1-((2-(trimethylsilyl)ethoxy)methyl)-1H-pyrazol-5-yl)cyclopentyl (4-nitrophenyl) carbonate (1000 mg, 1.90 mmol) in tetrahydrofuran (5 mL) was added bicyclo[1.1.1]pentan-1-amine hydrochloride (341 mg, 2.85 mmol) and N,N-diisopropylethylamine (0.99 mL, 5.70 mmol). The reaction was stirred at 25° C. for 16 h. The mixture was concentrated under reduced pressure. The residue was diluted with ethyl acetate (200 mL), washed with 1 M aqueous sodium hydroxide (2×50 mL) and brine (2×50 mL). The organic layer was dried over sodium sulfate, filtered and concentrated under reduced pressure. The residue was purified by column chromatography (silica gel, 100-200 mesh, 0-35% ethyl acetate in petroleum ether) to give (1R,3S)-3-(3-bromo-1-((2-(trimethylsilyl)ethoxy)methyl)-1H-pyrazol-5-yl)cyclopentyl bicyclo[1.1.1]pentan-1-ylcarbamate (600 mg, 67%). LCMS (ES-API, M/Z): 470.0 [M+H]⁺.

Step 7: (1R,3S)-3-(3-((3-cyclopropylpyridazin-4-yl)amino)-1-((2-(trimethylsilyl)ethoxy)methyl)-1H-pyrazol-5-yl)cyclopentyl bicyclo[1.1.1]pentan-1-ylcarbamate. A mixture of (1R,3S)-3-(3-bromo-1-((2-(trimethylsilyl)ethoxy)methyl)-1H-pyrazol-5-yl)cyclopentyl bicyclo[1.1.1]pentan-1-ylcarbamate (80 mg, 0.17 mmol), 3-cyclopropylpyridazin-4-amine (34 mg, 0.26 mmol), cesium carbonate (222 mg, 0.68 mmol) and [2-(2-aminophenyl)phenyl]methyl methanesulfonate ditert-butyl-[3,6-dimethoxy-2-(2,4,6-triisopropylphenyl) phenyl] phosphane (13 mg, 0.02 mmol) in 1,4-dioxane (5 mL) was stirred at 100° C. for 16 h under nitrogen atmosphere. The reaction mixture was diluted with ethyl acetate (40 mL), washed with brine (30 mL). The organic layer was dried over sodium sulfate, filtered and concentrated under reduced pressure. The residue was purified by preparative TLC (10% methanol in dichloromethane) to give (1R,3S)-3-(3-((3-cyclopropylpyridazin-4-yl)amino)-1-((2-(trimethylsilyl)ethoxy)methyl)-1H-pyrazol-5-yl)cyclopentyl bicyclo[1.1.1 ]pentan-1-ylcarbamate (15 mg, 17%). LCMS (ES-API, M/Z): 525.3 [M+H]⁺.

Step 8: (1R,3S)-3-(3-((3-cyclopropylpyridazin-4-yl)amino)-1H-pyrazol-5-yl)cyclopentyl bicyclo[1.1.1]pentan-1-ylcarbamate. A solution of [(1R,3S)-3-[5-[(3-cyclopropylpyridazin-4-yl)amino]-2-(2-trimethylsilylethoxymethyl)pyrazol-3-yl]cyclopentyl] N-(1-bicyclo[1.1.1]pentanyl)carbamate (15 mg, 0.03 mmol) in dichloromethane (1 mL) was added trifluoroacetic acid (0.1 mL). The reaction stirred at 25° C. for 16 h. The reaction mixture was adjusted to pH=7 by the addition of aqueous ammonia (37%, 3 mL). After filtration, the filtrate was concentrated under reduced pressure. The residue was purified by Purification Procedure G with 0.05% ammonium hydroxide-10 mM ammonium bicarbonate in water-acetonitrile (30-60%) to give (1R,3S)-3-(3-((3-cyclopropylpyridazin-4-yl)amino)-1H-pyrazol-5-yl)cyclopentyl bicyclo[1.1.1]pentan-1-ylcarbamate (1.1 mg, 9.4%). LCMS (ES-API, M/Z): 395.1 [M+H]⁺. ¹H NMR (400 MHz, Methanol-d4) δ 8.05 (br s, 1H), 7.59 (br s, 1H), 6.05 (s, 1H), 5.19-5.14 (m, 1H), 3.23-3.19 (m, 1H), 2.58-2.37 (m, 1H), 2.21-2.19 (m, 2H), 2.18-2.0 (m, 10H), 1.14-1.10 (m, 4H).

### Example 11A: (1R,3S)-3-(3-((3-methylpyridazin-4-yl)amino)-1H-pyrazol-5-yl)cyclopentyl tert-butylcarbamate

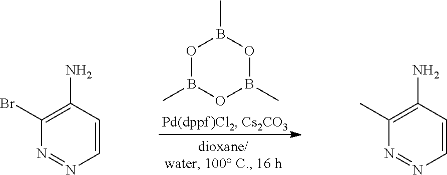

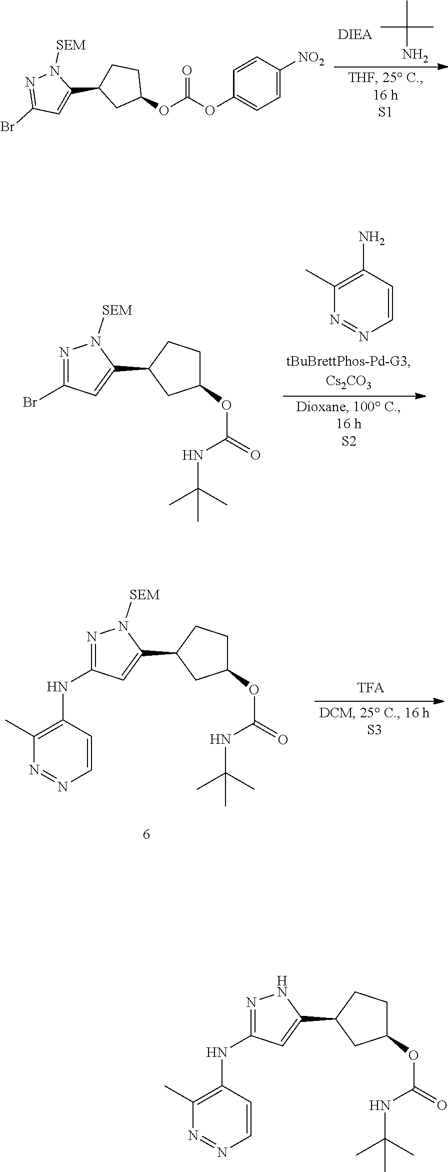

Step 1: 3-methylpyridazin-4-amine. A solution of 2,4,6-trimethyl-1,3,5,2,4,6-trioxatriborinane (8.03 mL, 28.74 mmol), 3-bromopyridazin-4-amine (2.0 g, 11.49 mmol), cesium carbonate (14.9 g, 45.98 mmol) and dichloro [1,1′-bis(diphenylphosphino)ferrocene] palladium (II) (841. mg, 1.15 mmol) in 1,4-dioxane (30 mL) and water (6 mL) was stirred at 100° C. for 16 hours under nitrogen atmosphere. Ethyl acetate (50 mL) and methanol (50 mL) were added and the mixture was filtered. The filtrate was concentrated under reduced pressure and the crude was purified by column chromatography (silica gel, 100-200 mesh, 0 to 10% methanol in ethyl acetate) to obtain 3-methylpyridazin-4-amine (700 mg, 56%). ¹H NMR (400 MHz, DMSO-d6) δ 8.38 (d, J=6.0 Hz, 1H), 6.55 (d, J=6.0 Hz, 1H), 6.14 (br s, 2H), 2.36 (s, 3H).

Step 2: (1R,3S)-3-(3-bromo-1-((2-(trimethylsilyl)ethoxy)methyl)-1H-pyrazol-5-yl)cyclopentyl tert-butylcarbamate. To a solution of 3-(3-bromo-1-((2-(trimethylsilyl)ethoxy)methyl)-1H-pyrazol-5-yl)cyclopentyl (4-nitrophenyl) carbonate (800 mg, 1.52 mmol) in tetrahydrofuran (15 mL) was added N,N-diisopropylethylamine (0.79 mL, 4.56 mmol) and tert-butylamine (0.48 mL, 4.56 mmol). The reaction was stirred at 25° C. for 16 hours. Ethyl acetate (50 mL) was added and the organic layer was washed with aqueous sodium hydroxide (15 mL, 1 M), brine (20 mL), dried over anhydrous sodium sulfate and filtered. The filtrate was concentrated under reduced pressure. The crude was purified by column chromatography (silica gel, 100-200 mesh, 0 to 30% ethyl acetate in petroleum ether) to obtain (1R,3S)-3-(3-bromo-1-((2-(trimethylsilyl)ethoxy)methyl)-1H-pyrazol-5-yl)cyclopentyl tert-butylcarbamate (270 mg, 39%). LCMS (ES-API, M/Z): 460.2 [M+H]⁺.

Step 3: (1R,3S)-3-(3-((3-methylpyridazin-4-yl)amino)-1-((2-(trimethylsilyl)ethoxy)methyl)-1H-pyrazol-5-yl)cyclopentyl tert-butylcarbamate. A solution of (1R,3S)-3-(3-bromo-1-((2-(trimethylsilyl)ethoxy)methyl)-1H-pyrazol-5-yl)cyclopentyl tert-butylcarbamate (400 mg, 0.87 mmol), 3-methylpyridazin-4-amine (237 mg, 2.17 mmol), methanesulfonato 2-di-t-butylphosphino-3,6-dimethoxy-2′-4′-6′-tri-i-propyl-1,1′-bipheny) (2′-amino-1,1′-biphenyl-2-yl) palladium (II) (74 mg, 0.09 mmol) and cesium carbonate (1.1 g, 3.47 mmol) in 1,4-dioxane (15 mL) was stirred at 100° C. for 16 hours under nitrogen atmosphere. Ethyl acetate (100 mL) was added and the mixture was filtered. The filtrate was concentrated under reduced pressure and the crude was purified by column chromatography (silica gel, 100-200 mesh, 0 to 5% methanol in ethyl acetate) to obtain (1R,3S)-3-(3-((3-methylpyridazin-4-yl)amino)-1-((2-(trimethylsilyl)ethoxy)methyl)-1H-pyrazol-5-yl)cyclopentyl tert-butylcarbamate (210 mg, 50%). LCMS (ES-API, M/Z): 489.3 [M+H]⁺.

Step 4: (1R,3S)-3-(3-((3-methylpyridazin-4-yl)amino)-1H-pyrazol-5-yl)cyclopentyl tert-butylcarbamate. A solution of (1R,3S)-3-(3-((3-methylpyridazin-4-yl)amino)-1-((2-(trimethylsilyl)ethoxy)methyl)-1H-pyrazol-5-yl)cyclopentyl tert-butylcarbamate (210 mg, 0.43 mmol) in dichloromethane (20 mL) was added trifluoroacetic acid (2 mL). The reaction stirred at 25° C. for 16 h. The reaction mixture was adjusted to pH=7 by the addition of aqueous ammonia (37%, 3 mL). After filtration, the filtrate was concentrated under reduced pressure. The residue was purified by column chromatography (silica gel, 100-200 mesh, 0 to 10% methanol in dichloromethane) to give the crude (150 mg), which was further purified by Purification Procedure P (SFC separation) with 0.1% ammonium hydroxide-40% ethyl alcohol-carbon dioxide to afford (1R,3S)-3-(3-((3-methylpyridazin-4-yl)amino)-1H-pyrazol-5-yl)cyclopentyl tert-butylcarbamate (137 mg, 87%). LCMS (ES-API, M/Z): 359.0 [M+H]⁺; ¹H NMR (400 MHz, DMSO-d6) δ 12.15 (br s, 1H), 8.61 (d, J=5.6 Hz, 1H), 8.31 (s, 1H), 7.83 (d, J=6.0 Hz, 1H), 6.76 (br s, 1H), 5.92 (s, 1H). 4.98 (br s, 1H), 3.09-3.05 (m, 1H), 2.56 (s, 3H), 2.48-2.46 (m, 1H), 2.04-1.90 (m, 2H), 1.76-1.59 (m, 3H), 1.20 (s, 9H).

Other compounds may be synthesized according to General Procedure and Example 10, or General Procedure and Example 11, as set forth in the below Table N.

TABLE N
General Procedure 10 & 11 and Example 10 & 11 Compounds

| | | | | MS |
|---|---|---|---|---|
| Ex | Structure/Name | Procedure | ¹H NMR | (m/z) |
| 10A | 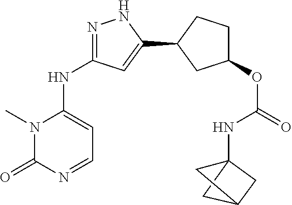 [SMILES: Cn1c(Nc2cc([C@H]3CC[C@@H](OC(=O)NC45CC(C4)C5)C3)[nH]n2)ccnc1=O] | May be prepared by General Procedure 10 from 6-bromo- 1-methylpyrimidin-2(1H)- one | | |
| | (1R,3S)-3-(3-((3-methyl-2-oxo- | | | |
| | 2,3-dihydropyrimidin-4- | | | |
| | yl)amino)-1H-pyrazol-5- | | | |
| | yl)cyclopentyl | | | |
| | bicyclo[1.1.1]pentan-1- | | | |
| | ylcarbamate | | | |
| 11 | 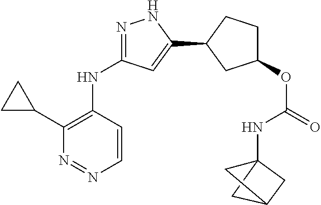 [SMILES: O=C(NC12CC(C1)C2)O[C@@H]1CC[C@H](c2cc(Nc3ccnnc3C3CC3)n[nH]2)C1] | Prepared according to Example 11 procedure May also be prepared by General Procedure 10 from 4-chloro-3- cyclopropylpyridazine | ¹H NMR (400 MHz, Methanol-d4) δ 8.05 (br s, 1H), 7.59 (br s, 1H), 6.05 (s, 1H), 5.19-5.14 (m, 1H), 3.23-3.19 (m, 1H), 2.58-2.37 (m, 1H), 2.21-2.19 (m, 2H), 2.18-2.0 (m, 10H), 1.14-1.10 (m, 4H). | 395.1 [M + H]⁺ |
| | (1R,3S)-3-(3-((3- | | | |
| | cyclopropylpyridazin-4- | | | |
| | yl)amino)-1H-pyrazol-5- | | | |
| | yl)cyclopentyl | | | |
| | bicyclo[1.1.1]pentan-1- | | | |
| | ylcarbamate | | | |
| 10C | 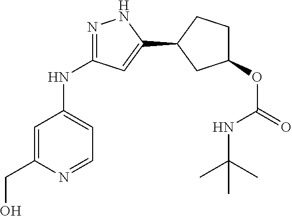 [SMILES: CC(C)(C)NC(=O)O[C@@H]1CC[C@H](c2cc(Nc3ccnc(CO)c3)n[nH]2)C1] | 2-methylpropan-2-amine and (4-bromopyridin-2- yl)methanol | ¹H NMR (400 MHz, DMSO-d6) δ 11.95 (br s, 1H), 8.92 (s, 1H), 8.06-8.04 (m, 1H), 7.31 (s, 1H), 7.10-7.09 (m, 1H), 6.80 (s, 1H), 5.69 (s, 1H), 5.25-5.22 (m, 1H), 4.97 (br s, 1H), 4.45-4.40 (m, 2H), 3.06-3.02 (m, 1H), 2.46-2.44 (m, 1H), 2.02-1.87 (m, 2H), 1.75-1.58 (m, 3H), 1.20 (s, 9H). | 374.1 [M + H]⁺ |
| | (1R,3S)-3-(3-((2- | | | |
| | (hydroxymethyl)pyridin-4- | | | |
| | yl)amino)-1H-pyrazol-5- | | | |
| | yl)cyclopentyl tert- | | | |
| | butylcarbamate | | | |
| 10D | 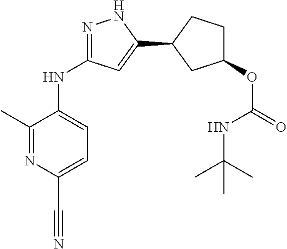 [SMILES: Cc1nc(C#N)ccc1Nc1cc([C@H]2CC[C@@H](OC(=O)NC(C)(C)C)C2)[nH]n1] | 2-methylpropan-2-amine and (5-bromo-6- methylpicolinonitrile | ¹H NMR (400 MHz, DMSO-d6) δ 12.10 (br s, 1H), 8.26-8.25 (m, 1H), 7.72-7.66 (m, 1H), 6.77 (br s, 1H), 5.91 (s, 1H), 4.97 (br s, 1H), 3.09- 3.05 (m, 1H), 2.50 (s, 3H), 2.48-2.45 (m, 1H), 2.04-2.02 (m, 1H), 1.92-1.90 (m, 1H), 1.76-1.55 (m, 3H), 1.20 (s, 9H). | 383.2 [M + H]⁺ |
| | (1R,3S)-3-(3-((6-cyano-2- | | | |
| | methylpyridin-3-yl)amino)-1H- | | | |
| | pyrazol-5-yl)cyclopentyl tert- | | | |
| | butylcarbamate | | | |
| 10E | 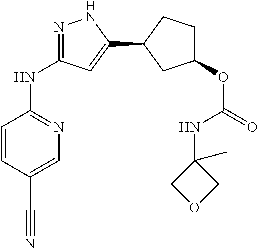 [SMILES: CC1(NC(=O)O[C@@H]2CC[C@H](c3cc(Nc4ccc(C#N)cn4)n[nH]3)C2)COC1] | 3-methyloxetan-3-amine hydrochloride and 6- bromonicotinonitrile | ¹H NMR (400 MHz, DMSO-d6) δ 12.10 (br s, 1H), 10.01 (br s, 1H), 8.52 (s, 1H), 7.89-7.87 (m, 1H), 7.65 (br s, 1H), 7.28 (br s, 1H), 6.21 (br s, 1H), 5.01 (br s, 1H), 4.55 (br s, 2H), 4.24- 4.23 (m, 2H), 3.09- 3.07 (m, 1H), 2.52- 2.50 (m, 1H), 2.07- 2.02 (m, 1H), 1.95- 1.89 (m, 1H), 1.78- 1.62 (m, 3H), 1.47 (s, 3H). | 383.2 [M + H]⁺ |
| | (1R,3S)-3-(3-((5-cyanopyridin- | | | |
| | 2-yl)amino)-1H-pyrazol-5- | | | |
| | yl)cyclopentyl (3-methyloxetan- | | | |
| | 3-yl)carbamate | | | |
| 11A | 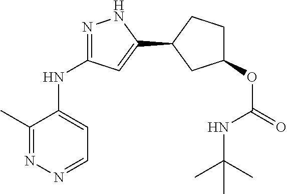 [SMILES: Cc1nnccc1Nc1cc([C@H]2CC[C@@H](OC(=O)NC(C)(C)C)C2)[nH]n1] | Prepared by General Procedure 11 from 2- methylpropan-2-amine and 3-methylpyridazin-4-amine | ¹H NMR (400 MHz, DMSO-d6) δ 12.15 (br s, 1H), 8.61-8.59 (m, 1H), 8.31 (s, 1H), 7.84-7.83 (m, 1H), 6.76 (br s, 1H), 5.92 (s, 1H), 4.98 (br s, 1H), 3.09-3.05 (m, 1H), 2.56 (s, 3H), 2.50-2.46 (m, 1H), 2.04-2.02 (m, 1H), 1.92-1.90 (m, 1H), 1.76-1.58 (m, 3H), 1.20 (s, 9H). | 359.0 [M + H]⁺ |
| | (1R,3S)-3-(3-((3- | | | |
| | methylpyridazin-4-yl)amino)- | | | |
| | 1H-pyrazol-5-yl)cyclopentyl | | | |
| | tert-butylcarbamate | | | |

### Assay Methods

### Enzymatic Assays

Cyclin Dependent Kinase 1/Cyclin A2 (CDK1-cycA2), Cyclin Dependent Kinase 2/Cyclin E1 (CDK2-cycE1), Cyclin Dependent Kinase 4/Cyclin D3 (CDK4-cycD3) and Cyclin Dependent Kinase 6/Cyclin D3 (CDK6-cycD3) enzyme assays were carried out as described below. K, values were then determined using the Morrison tight binding model (Morrison, J. F., Biochim. Biophys. Acta. 185:269-296 (1969); William, J. W. and Morrison, J. F., Meth. Enzymol., 63:437-467 (1979)) modified for ATP-competitive inhibition K=K app (1+[ATP]/K m pp).

### (1) CDK1-cycA2 TR-FRET (Lance®)

An Echo acoustic dispenser is used to create dose responses of compound dissolved in DMSO into a 384-well microplate. A 5 μL volume of enzyme mix is added to the well, and pre-incubated with compound for 30 minutes. A 5 μL volume of substrate mix is added to the enzyme and compound, and allowed to react at room temperature for 90 min. Final concentrations are: 0.625 nM CDK1, 50 nM Ulight-4E-BP1 substrate, and 20 μM ATP (Km=16 μM). Buffer conditions are 50 mM HEPES, pH 7.5, 10 mM MgCl₂, 2 mM TCEP, 0.05% BGG and 0.01% Brij 35. The reaction is quenched with 5 μL of EDTA in detection buffer (15 mM [EDTA final]). Finally, a 5 μL TR-FRET detection mix is added to the quenched reaction, which includes Europium labeled anti-phospho-4E-BP1. After a 60 min incubation, the 20 uL total reaction is read on an Envision plate reader (ex320, dual em615/665). Signal is a ratio of the Acceptor and Donor fluorescence. Inhibition constants are calculated via the Morrison equation. Enzyme source of CDK1-cycA2 is ProQinase, catalog #: 0134-0054-1.

### (ii) CDK2-cycE1 TR-FRET (LANCE®)

An Echo acoustic dispenser is used to create dose responses of compound dissolved in DMSO into a 384-well microplate. A 5 μL volume of enzyme mix is added to the well, and pre-incubated with compound for 30 minutes. A 5 μL volume of substrate mix is added to the enzyme and compound, and allowed to react at room temperature for 90 min. Final concentrations are: 0.625 nM CDK2, 50 nM Ulight-MBP substrate, and 100 μM ATP (Kₘ=103 μM). Buffer conditions are 50 mM HEPES, pH 7.5, 10 mM MgCl₂, 2 mM TCEP, 0.05% BGG and 0.01% Brij 35. The reaction is quenched with 5 μL of EDTA in detection buffer (15 mM [EDTA final]). Finally, a 5 μL TR-FRET detection mix is added to the quenched reaction, which includes Europium labeled anti-phospho-MBP. After a 60 min incubation, the 20 μL total reaction is read on an Envision plate reader (ex320, dual em615/665). Signal is a ratio of the Acceptor and Donor fluorescence. Inhibition constants are calculated via the Morrison equation. Enzyme source of CDK2-cycE1 is ProQinase, catalog #: 0050-0055-1.

(iii) CDK4-cycD3 TR-FRET (LANCE®)

An Echo acoustic dispenser is used to create dose responses of compound dissolved in DMSO into a 384-well microplate. A 5 μL volume of enzyme mix is added to the well, and pre-incubated with compound for 30 minutes. A 5 μL volume of substrate mix is added to the enzyme and compound, and allowed to react at room temp for 90 min. Final concentrations are: 3 nM CDK4, 50 nM Ulight-4E-BP1 substrate, and 500 μM ATP (Km=699 μM). Buffer conditions are 50 mM HEPES, pH 7.5, 10 mM MgCl₂, 2 mM TCEP, 0.05% BGG and 0.01% Brij 35. The reaction is quenched with 5 μL of EDTA in detection buffer (15 mM [EDTA final]). Finally, a 5 μL TR-FRET detection mix is added to the quenched reaction, which includes Europium labeled anti-phospho-4E-BP1. After a 60 min incubation, the 20 μL total reaction is read on an Envision plate reader (ex320, dual em615/665). Signal is a ratio of the Acceptor and Donor fluorescence. Inhibition constants are calculated via the Morrison equation. Enzyme source of CDK4-cycD3 is Carna Biosciences, catalog #: 04-105.

### (iv) CDK6-cycD3 TR-FRET (LANCE®)

An Echo acoustic dispenser is used to create dose responses of compound dissolved in DMSO into a 384-well microplate. A 5 μL volume of enzyme mix is added to the well, and pre-incubated with compound for 30 minutes. A 5 μL volume of substrate mix is added to the enzyme and compound, and allowed to react at room temp for 60 min. Final concentrations are: 0.625 nM CDK6, 50 nM Ulight-4E-BP1 substrate, and 1000 μM ATP (Km=959 μM). Buffer conditions are 50 mM HEPES, pH 7.5, 10 mM MgCl2, 2 mM TCEP, 0.05% BGG and 0.01% Brij 35. The reaction is quenched with 5 μL of EDTA in detection buffer (15 mM [EDTA final]). Finally, a 5 uL TR-FRET detection mix is added to the quenched reaction, which includes Europium labeled anti-phospho-4E-BP1. After a 60 min incubation, the 20 uL total reaction is read on an Envision plate reader (ex320, dual em615/665). Signal is a ratio of the Acceptor and Donor fluorescence. Inhibition constants are calculated via the Morrison equation. Enzyme source of CDK4-cycD3 is Carna Biosciences, catalog #: 04-107.

### Cell Proliferation Assays

### (i) OVISE and SKOV-3 EdU Incorporation Cell Proliferation Assays

Ovarian cancer cell proliferation is measured by incorporation of EdU (5-ethynyl-2′-deoxyuridine) into newly synthesized DNA. CDK2-dependent proliferation is determined using OVISE (JCRB1043, CCNE1-amplified) while CDK4/6-dependent effect is determined with SKOV-3 (ATCC HTB-77) which is not CCNE1-amplified.

Cells are plated in 384-well plates in RPMI medium supplemented with 10% FBS. Compounds are added at desired concentrations with a final DMSO content of 0.5%, and cells incubated at 37° C./5% CO₂ for 16 hours. EdU (Life Technologies) is added to a final concentration of 0.5 μM and incubation continued for 8 hours, followed by fixation with 4% paraformaldehyde. EdU incorporated into chromosomal DNA is labeled with Alexa Flour 488 dye using the Click-It kit reagents according to manufacturer's instructions (Invitrogen-C10351), and total nuclear DNA is stained with HCS NuclearMask Blue (Invitrogen). Images are acquired using Yokogawa CQ1 imaging cytometer with a 10×objective. Images are analyzed to count the total number of nuclei (NuclearMask Blue, ex405 nm/em447 nm) and number of EdU-positive nuclei (ex488 nm/em525 nm). The IC₅₀ for inhibition of cell division is determined by fitting the fraction of EdU-positive cells as a function of compound dose using a 4-parameter logistic model.

(ii) p-H3 HCT-116 Mitotic Release Cell Proliferation Assay

Inhibition of cellular CDK1 determined by release of cells from nocodazole-induced mitotic arrest, as measured by decrease in phospho-Histone H3. Histone H3 (Ser 10) level is detected in cell lysates by producing a Fluorescence Resonance Energy Transfer (FRET) signal between two antibodies, Phospho-Histone H3 Cryptate (acting as the donor) and Phospho-Histone H3 d2 (acting as the acceptor).

HCT116 cells are seeded at 20,000 cells/well in Falcon 96-well plates in RPMI 1640 medium supplemented with 10% FBS and 1% L-glutamine. Plates are allowed to rest overnight at 37° C./5% CO₂. The following morning, nocodazole is diluted in complete medium, and 5 μL is added to each test well and positive control well for a final concentration of 500 nM. Plates are then incubated for 16 hours at 37° C./5% CO₂. An Echo acoustic dispenser is used to create a dose response of compound dissolved in DMSO into a 96-well plate, these compounds are diluted to 10-fold the desired assay concentration using assay medium (RPMI 1640 medium supplemented with 10% FBS and 1% L-glutamine). 10 μL of diluted compound is then transferred to the cell plate (final DMSO concentration 0.1%). Cell plates are then incubated for 4 hours in 37° C. 5% CO₂. A cell lysis buffer is prepared on ice using reagents from the Phospho-Histone H3 (Ser10) cellular kit (Cisbio-64HH3PEG). Supernatant is discarded from the cell plate and 80 μL of the supplemented cell lysis buffer (1:99 dilution of the Blocking Reagent and the 1× cell lysis buffer provided in the kit) is added to each of the experimental wells. The cell lysis reaction is allowed to proceed at room temperature while shaking vigorously for 30 minutes. The 384-well HTRF (Homogeneous Time Resolved Fluorescence) plate is prepared by adding 2 μL of each diluted antibody (Phospho-Histone H3 Cryptate antibody and Phospho-Histone H3 d2 antibody diluted 19:1 in a detection buffer) and 12 μL of the supplemented cell lysis buffer to each well in the HTRF plate. 4 μL of the cell lysate is added to the prepared HTRF plate which is then sealed and the reaction is allowed to proceed for a minimum of 2 hours at room temperature. Fluorescence is read on a Perkin Elmer Envision (wavelengths at 665 nm and 620 nm).

### Compound Activity Data

Compounds as described herein were tested for activity against the above-described assays. As can be seen from the below Table O, all of the compounds tested had less than 2 micromolar activity against CDK2, with the majority showing ≤10 nM activity, and of those many showing ≤1 nM nanomolar activity. See, e.g., compounds 2, 2E, 2F, 2H, 2I, 2L-2O, 2R, 2T, 2V, 2CC, 4, 4B, 4D, 4K, 6, 6A-6C, and 7, showing ≤1 nM activity. Furthermore, all of the compounds tested demonstrated selectivity for CDK2 over CDK1 and CDK4, see, e.g., fold selectivity (FS) for CDK1/CDK2 and CDK4/CDK2 enzymatic assay data, and fold selectivity (FS) for SKOV-3/OVISE cellular assay data (measuring CDK4/6 over CDK2), with many compounds demonstrating ≥30-fold selectivity for CDK2 over CDK1 (see, e.g., fold selectivity enzymatic data for compounds 1, 2, 2A-2C, 2E, 2G, 2T-2V, 2Y, 2Z, 2CC-2EE, 4, 4A, 4B, 4D, 4E, 4G, 4H, 4J, 4K, 6, 6A, 6C, 7) and >100-fold selectivity for CDK2 over CDK 4 (see, e.g., fold selectivity enzymatic data for compounds 2, 2B, 2F, 2H, 2K-2M, 2O, 2R, 2T-2V, 2Z, 2AA, 2CC, 4, 4A-4D, 4K, 6-6C).

The compounds described herein comprise an amine (—NH—) linker. The activity of compounds comprising an acetamide linker (—NH—C(═O)—CH₂—; see Comparator A) and amide linker (—NH—C(═O)—; see Comparator B) were compared to an exemplary Compound 4K comprising an amine (—HN—) linker. See Table P. While the CDK2 potency of the amine Compound 4K shared nanomolar activity with the acetamide Comparator A (compare 0.45 nM to 0.17 nM), and both were more CDK2 potent than the amide Comparator B (1.0 nM), Compound 4K was at least three times more CDK2 selective over CDK4/6, as can be seen from the SKOV-3/OVISE fold-selectivity cellular assay data. Compound 4K was also was at least 3 times more CDK2 selective over CDK1, as can be seen from the pH3/OVISE cellular assay data (measuring CDK 1 over CDK2): compare Compound 4K (2.1-fold selective) versus Comparator A (0.78-fold selective) and Comparator B (0.31-fold selective). The (+) sign in Table P signifies desirable increased fold-selectivity of the exemplary Compound 4K over both Comparators.

In addition to the amine (—NH—) linker, the compounds described herein comprise a 6-membered (monocyclic) heteroaryl Ring A comprising at least one N heteroatom, and optionally 1 or 2 additional N heteroatoms.

As can be seen from Table Q, Comparator C, with a 5-membered pyrazole Ring A, has >10 nanomolar CDK2 activity (32 nM in the enzymatic assay), and <10-fold selectivity against CDK4 and CDK4/6 (9.1-fold against CDK4 in the enzymatic assay, and >3.6-fold selectivity against CDK4/6 in the cellular assay). In contrast, many of the comparable exemplary compounds comprising a 6-membered heteroaryl Ring A and isopropyl R² group, as provided in Table Q, show between a 1.9-fold to over 300-fold improvement in potency against CDK2 (see CDK2 enzymatic assay data, comparing Comparator C's CDK2 activity of 32 nM to the test compounds CDK2 activity) and an increased fold selectivity for CDK2 versus CDK4, as measured by the enzymatic assay. Similar improvements in potency and selectivity are observed in the cellular assays. The (+) sign in Table Q signifies desirable increased fold-selectivity of the exemplary compounds over Comparator C.

As can be seen from Table R, Comparator D, with a bicyclic 6,7-dihydro-5H-cyclopenta[b]pyridinyl Ring A, is more CDK2 potent (2.7 nM in the enzymatic assay) than the 5-membered pyrazole Comparator C (32 nM in the enzymatic assay), but is still less CDK2 cell potent and less selective for CDK2 over both CDK1 or CDK4 when compared to the large majority of exemplary compounds comprising a 6-membered heteroaryl Ring A and tert-butyl R² group. The (+) sign in Table R signifies desirable increased fold-selectivity of the exemplary compounds over Comparator D.

As can be seen from Table S, Comparator E, with a phenyl Ring A, is more CDK2 potent (0.52 nM in the enzymatic assay) than 5-membered Ring A Comparator C (32 nM in the enzymatic assay) and bicyclic Ring A Comparator D (2.7 nM in the enzymatic assay). Including an additional N ring heteroatom in the ring system, particularly at the meta or para position relative to the point of attachment, such as shown in exemplary Compounds 1, 2A, 4, 4A, 4C, 4D, 4E, 4H, 4I, 4K, and 6C, generally leads to an increase in selectivity for CDK2 over CDK4, as can be seen from the SKOV-3/OVISE fold-selectivity cellular assay data. Furthermore, many of the compounds following this trend are also shown to be more potent CDK2 cell inhibitors compared to Comparator E, as can be seen from the CDK2 OVISE cellular assay data for compounds 2A, 4, 4D, 4E, 4H, 4K, and 6C. The (+) sign in Table S signifies desirable increased fold-selectivity of the exemplary compounds over Comparator E.

The data provided herein demonstrates having a shorter amine linker is preferred over longer linkers, leading to increased potency and selectivity. The data further demonstrates having a 6-membered monocyclic heteroaryl ring leads to more potent and selective CDK2 inhibitors compared to compounds comprising a smaller (5-membered) or larger (bicyclic) heteroaryl Ring A. Finally, the data demonstrates improvements in potency and/or selectivity with inclusion of a nitrogen (N) ring heteroatom in the aromatic monocyclic ring system of Ring A, particularly where at least one of the N ring heteroatoms is at the meta or para position relative to the point of attachment.

TABLE O
Compound Activity Data

| | | | | | | Cellular assays | | | | |
|---|---|---|---|---|---|---|---|---|---|---|
| | Enzymatic assays | | | | | CDK2 | CDK1 | CDK4/6 | | FS |
| | | | CDK4 | FS | FS | OVISE | p-H3 | SKOV-3 | FS | SKOV- |
| | CDK2 | CDK1 | Kᵢ | CDK1/ | CDK4/ | IC₅₀ | IC₅₀ | IC₅₀ | p-H3/ | 3/ |
| Ex | Kᵢ [μM] | Kᵢ [μM] | [μM] | CDK2 | CDK2 | [μM] | [μM] | [μM] | OVISE | OVISE |
| 1 | 0.0076 | 0.27 | 0.70 | 36 | 92 | 0.22 | 0.10 | >20 | 0.45 | >91 |
| 2 | 0.00044 | 0.016 | 0.24 | 36 | 550 | 0.093 | 0.19 | 11 | 1.9 | 120 |
| 2A | 0.0017 | 0.071 | 0.075 | 42 | 44 | 0.11 | 0.094 | 2.2 | 0.66 | 20 |
| 2B | 0.0034 | 0.16 | 0.39 | 47 | 120 | 0.23 | 2.9 | >20 | 1.1 | >70 |
| 2C | 0.0011 | 0.043 | 0.095 | 39 | 86 | 0.11 | 0.052 | 4.1 | 0.47 | 37 |
| 2D | 0.014 | 0.35 | 0.48 | 25 | 34 | 0.70 | — | 14 | — | 20 |
| 2E | 0.00013 | 0.0084 | 0.0074 | 65 | 57 | 0.016 | 0.014 | 0.13 | 0.88 | 8.1 |
| 2F | 0.00041 | 0.011 | 0.19 | 21 | 270 | 0.065 | 0.076 | 3.5 | 1.3 | 58 |
| 2G | 0.0015 | 0.088 | 0.15 | 59 | 100 | 0.28 | 0.28 | 10 | 1.0 | 36 |
| 2H | 0.00028 | 0.0075 | 0.018 | 27 | 64 | 0.067 | 0.032 | 1.1 | 0.48 | 16 |
| 2I | 0.00031 | 0.0060 | 0.027 | 19 | 87 | 0.067 | 0.0094 | 1.0 | 0.14 | 15 |
| 2J | 0.010 | 0.20 | 0.23 | 20 | 23 | 0.44 | — | 11 | — | 25 |
| 2K | 0.0032 | 0.059 | 0.34 | 18 | 110 | 0.19 | 0.087 | 7.7 | 0.46 | 41 |
| 2L | 0.00017 | 0.0029 | 0.11 | 17 | 650 | 0.012 | 0.0018 | 0.64 | 0.15 | 53 |
| 2M | 0.00026 | 0.0063 | 0.028 | 24 | 110 | 0.020 | 0.0070 | 0.58 | 0.35 | 29 |
| 2N | 0.00049 | 0.0037 | 0.026 | 7.6 | 53 | 0.029 | 0.011 | 1.2 | 0.38 | 41 |
| 2O | 0.00087 | 0.024 | 0.25 | 28 | 290 | 0.070 | 0.022 | 3.9 | 0.31 | 56 |
| 2P | 0.0042 | 0.033 | 0.18 | 7.9 | 43 | 0.37 | — | 5.3 | — | 14 |
| 2Q | 0.041 | 0.67 | 1.5 | 16 | 37 | 2.4 | — | >20 | — | >8.3 |
| 2R | <0.00010 | 0.00040 | 0.0090 | 6.3 | 140 | 0.0056 | 0.0035 | 0.17 | 0.63 | 30 |
| 2S | 0.89 | 7.4 | 12 | 8.3 | 13 | >20 | — | >20 | — | 1.0 |
| 2T | 0.00086 | 0.064 | 0.15 | 74 | 170 | 0.052 | 0.12 | 1.5 | 2.3 | 29 |
| 2U | 0.015 | 0.56 | 1.8 | 37 | 120 | 0.40 | — | >20 | — | >50 |
| 2V | 0.00092 | 0.036 | 0.26 | 39 | 280 | 0.11 | 0.097 | 2.5 | 0.88 | 23 |
| 2W | 0.042 | 1.1 | 3.1 | 26 | 74 | 2.7 | — | >20 | — | >7.4 |
| 2X | 0.059 | 1.2 | 3.8 | 20 | 64 | 2.8 | — | >20 | — | >7.1 |
| 2Y | 0.21 | 7.5 | 49 | 36 | 230 | 16 | — | >20 | — | >1.3 |
| 2Z | 0.0050 | 0.55 | 1.3 | 110 | 260 | 0.25 | 0.41 | 18 | 1.6 | 72 |
| 2AA | 0.017 | 0.44 | 0.45 | 26 | 26 | 4.1 | — | >20 | — | >49 |
| 2BB | 1.1 | 32 | 6.9 | 29 | 6.3 | >20 | — | >20 | — | 1.0 |
| 2CC | 0.00033 | 0.034 | 0.13 | 100 | 390 | 2.0 | — | >20 | — | >10 |
| 2DD | 0.013 | 0.68 | 0.35 | 52 | 27 | 0.61 | — | >20 | — | 23 |
| 2EE | 0.0032 | 0.29 | 0.19 | 91 | 59 | 0.34 | 0.9 | 9.7 | 2.6 | 29 |
| 2FF | 0.014 | 0.32 | 0.95 | 23 | 68 | 1.2 | — | >20 | — | >17 |
| 2GG | 0.48 | 8.8 | 15 | 18 | 31 | >20 | — | >20 | — | 1 |
| 2HH | 0.00043 | 0.0069 | 0.21 | 16 | 490 | 0.06 | 0.032 | 4.6 | 0.53 | 77 |
| 2II | 0.0096 | 0.11 | 0.95 | 11 | 99 | 2.6 | — | >20 | — | >7.7 |
| 2JJ | 0.077 | 1.2 | 1.2 | 16 | 16 | 4.1 | — | 0.0032 | — | <1 |
| 3 | 0.0018 | 0.047 | 0.11 | 26 | 61 | 0.15 | 0.073 | 2.3 | 0.49 | 15 |
| 3A | 0.0025 | 0.072 | 0.15 | 29 | 60 | 0.13 | 0.16 | 2.7 | 1.2 | 21 |
| 3B | 0.0085 | 0.20 | 0.29 | 24 | 34 | 0.57 | — | 10 | — | 18 |
| 3C | 0.018 | 0.50 | 0.43 | 28 | 24 | 0.39 | — | 12 | — | 31 |
| 3D | 0.061 | 1.4 | 2.3 | 23 | 38 | 7.9 | — | >20 | — | >2.5 |
| 3E | 0.010 | 0.28 | 0.72 | 28 | 72 | 0.42 | 1.4 | 13 | 3.3 | 31 |
| 3F | 0.0040 | 0.11 | 0.024 | 27 | 6 | 0.32 | 0.35 | 1.3 | 1.1 | 4.1 |
| 3G | 0.0083 | 0.24 | 0.73 | 29 | 88 | 0.75 | — | >20 | — | >27 |
| 4 | 0.00022 | 0.025 | 0.034 | 110 | 160 | 0.020 | 0.030 | 0.92 | 1.5 | 46 |
| 4A | 0.0026 | 0.21 | 0.41 | 78 | 170 | 0.18 | 0.093 | 9.9 | 0.72 | 71 |
| 4B | 0.00052 | 0.020 | 0.019 | 38 | 37 | 0.033 | 0.023 | 0.39 | 0.70 | 12 |
| 4C | 0.0021 | 0.065 | 0.58 | 31 | 280 | 0.60 | — | 19 | — | 32 |
| 4D | <0.00010 | 0.0037 | 0.039 | >59 | >620 | 0.026 | 0.0092 | 1.5 | 0.35 | 58 |
| 4E | 0.0016 | 0.075 | 0.084 | 47 | 53 | 0.096 | 0.10 | 2.6 | 1.1 | 27 |
| 4F | 0.0025 | 0.052 | 0.037 | 21 | 15 | 0.19 | 0.066 | 1.7 | 0.35 | 8.9 |
| 4G | 0.0017 | 0.075 | 0.027 | 44 | 16 | 0.24 | 0.24 | 1.5 | 1.0 | 6.3 |
| 4H | 0.0020 | 0.18 | 0.11 | 90 | 55 | 0.038 | 0.054 | 0.95 | 1.4 | 25 |
| 4I | 0.0025 | 0.045 | 0.033 | 18 | 13 | 0.24 | 0.15 | 4.9 | 0.63 | 20 |
| 4J | 0.0025 | 0.17 | 0.011 | 68 | 4.4 | 0.32 | 0.36 | 1.2 | 1.1 | 3.8 |
| 4K | 0.00045 | 0.027 | 0.052 | 54 | 104 | 0.078 | 0.16 | 5.5 | 2.1 | 71 |
| 4L | 0.0029 | 0.095 | 0.47 | 33 | 162 | 0.31 | 0.31 | 16 | 1 | 52 |
| 5 | 0.011 | 0.27 | 0.28 | 25 | 25 | 0.46 | — | 5.8 | — | 13 |
| 5A | 0.063 | 1.7 | 2.1 | 27 | 33 | 2.2 | — | >20 | — | >9 |
| 5B | 0.026 | 0.31 | 0.51 | 12 | 20 | 2.9 | — | >20 | — | >6.9 |
| 5C | 0.0003 | 0.0094 | 0.13 | 31 | 433 | 0.063 | 0.15 | 5.3 | 2.4 | 84 |
| 6 | <0.00010 | 0.021 | 0.020 | >33 | >320 | 0.016 | 0.023 | 1.3 | 1.4 | 81 |
| 6A | <0.00010 | 0.0054 | 0.048 | >62 | >550 | 0.048 | 0.011 | 5.6 | 0.23 | 120 |
| 6B | <0.00010 | 0.00078 | 0.0074 | >12 | >120 | 0.027 | 0.0087 | 0.39 | 0.32 | 14 |
| 6C | <0.00010 | 0.0036 | 0.045 | >47 | >584 | 0.03 | 0.026 | 1.5 | 0.087 | 50 |
| 6D | 0.00021 | 0.0036 | 0.061 | 17 | 290 | 0.077 | 0.072 | 4.3 | 0.94 | 56 |
| 6E | 0.0029 | 0.048 | 0.018 | 17 | 6.2 | 0.32 | 0.29 | 15 | 0.91 | 47 |
| 7 | 0.00036 | 0.023 | 0.038 | 64 | 110 | 0.058 | 0.050 | 3.8 | 0.86 | 66 |
| 7A | 0.014 | 0.55 | 0.59 | 39 | 42 | 1.4 | — | >20 | — | >14 |
| 7B | 0.3 | 3.3 | 6.5 | 11 | 22 | 17 | — | >20 | — | >1.2 |
| 7C | 0.26 | 3.5 | 4.2 | 13 | 16 | 8 | — | >20 | — | >2.5 |
| 7D | 0.023 | 0.37 | 1.2 | 16 | 52 | 1.6 | — | >20 | — | >13 |
| 8 | 0.0031 | 0.16 | 0.66 | 52 | 213 | 0.32 | 0.56 | 15 | 1.8 | 47 |
| 8A | 0.27 | 11 | 50 | 41 | 185 | 20 | — | >20 | — | >1 |
| 8B | 0.00066 | 0.052 | 0.14 | 79 | 212 | 0.087 | 0.14 | 3.0 | 1.6 | 34 |
| 9 | 0.0019 | 0.061 | 0.15 | 32 | 79 | 0.17 | 0.21 | 3.3 | 1.2 | 19 |
| 9A | 0.00049 | 0.074 | 0.15 | 150 | 310 | 0.041 | 0.076 | 3.0 | 1.9 | 73 |
| 9B | 0.00035 | 0.012 | 0.021 | 34 | 60 | 0.033 | 0.046 | 0.47 | 1.4 | 14 |
| 9C | 0.0020 | 0.19 | 0.40 | 95 | 200 | 0.18 | 0.18 | 9.1 | 1.0 | 51 |
| 9D | 0.012 | 0.34 | 6 | 28 | 500 | 5 | — | >20 | — | >4 |
| 9E | 0.0021 | 0.069 | 0.19 | 33 | 90 | 0.20 | 0.22 | 1.0 | 1.0 | 5 |
| 9F | 0.0050 | 0.080 | 0.46 | 16 | 92 | 1.2 | — | >20 | — | >17 |
| 9G | 0.012 | 0.77 | 1.3 | 64 | 110 | 0.34 | — | 15 | — | 44 |
| 9H | 0.0047 | 0.03 | 0.0095 | 6.4 | 2 | 0.15 | 0.039 | 0.15 | 0.26 | 1 |
| 9I | 0.00038 | 0.0089 | 0.037 | 23 | 97 | 0.03 | 0.0069 | 0.36 | 0.23 | 12 |
| 9J | 0.0019 | 0.064 | 0.017 | 34 | 8.9 | 0.089 | 0.039 | 0.28 | 0.44 | 3.1 |
| 9K | 0.0056 | 0.041 | 0.067 | 7.3 | 12 | 0.19 | 0.049 | 0.71 | 0.26 | 3.7 |
| 10 | 0.13 | 5.1 | >50 | 39 | 385 | 16 | — | >20 | — | >1.3 |
| 10A | — | — | — | — | — | — | — | — | — | — |
| 11 | 0.012 | 0.89 | 1.1 | 74 | 92 | 2.6 | — | >20 | — | >7.7 |
| 10C | 0.0018 | 0.058 | 0.051 | 32 | 28 | 0.13 | 0.18 | 5.7 | 1.4 | 44 |
| 10D | 0.00044 | 0.059 | 0.086 | 134 | 195 | 0.046 | 0.097 | 2.8 | 2.1 | 61 |
| 10E | 0.00078 | 0.025 | 0.064 | 32 | 82 | 0.11 | 0.12 | 2.2 | 1.1 | 20 |
| 11A | 0.00063 | 0.083 | 0.28 | 132 | 444 | 0.12 | 0.23 | 12 | 1.9 | 100 |

TABLE P
Linker Comparison

| | | Enzymatic Assays | | | | | Cellular Assays | | | | |
|---|---|---|---|---|---|---|---|---|---|---|---|
| | | | | | FS | FS | CDK2 | CDK1 | CDK4/6 | FS | FS |
| | | CDK2 | CDK1 | CDK4 | CDK1/ | CDK4/ | OVISE | p-H3 | SKOV-3 | p-H3/ | SKOV-3/ |
| Ex | Compound | Kᵢ [μM] | Kᵢ [μM] | Kᵢ [μM] | CDK2 | CDK2 | IC₅₀ [μM] | IC₅₀ [μM] | IC₅₀ [μM] | OVISE | OVISE |
| Comparator A | 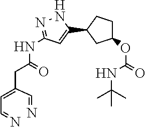 [SMILES: CC(C)(C)NC(=O)O[C@@H]1CC[C@H](c2cc(NC(=O)Cc3ccnnc3)n[nH]2)C1] | 0.00017 | 0.0091 | 0.038 | 54 | 224 | 0.040 | 0.031 | 1.7 | 0.78 | 43 |
| Comparator B | 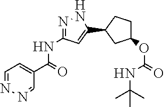 [SMILES: CC(C)(C)NC(=O)O[C@@H]1CC[C@H](c2cc(NC(=O)c3ccnnc3)n[nH]2)C1] | 0.0010 | 0.032 | 0.25 | 32 | 250 | 0.14 | 0.043 | 6.7 | 0.31 | 48 |
| 4K | 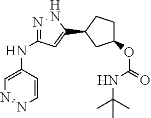 [SMILES: CC(C)(C)NC(=O)O[C@@H]1CC[C@H](c2cc(Nc3ccnnc3)n[nH]2)C1] | 0.00045 (0.4-fold) (2-fold) | 0.027 | 0.052 | 54 | 104 | 0.078 (0.5-fold) (1.8-fold) | 0.16 | 5.5 | 2.1 (+) (+) | 71 (+) (+) |

TABLE Q
5-membered ring versus 6-membered Ring A Comparison

| | | | | | | | Cellular Assays | | |
|---|---|---|---|---|---|---|---|---|---|
| | | Enzymatic Assays | | | | | CDK2 | CDK4/6 | FS |
| | | | | | FS | FS | OVISE | SKOV-3 | SKOV- |
| | | CDK2 | CDK1 | CDK4 | CDK1/ | CDK4/ | IC₅₀ | IC₅₀ | 3/ |
| Ex | Compound | Kᵢ [μM] | Kᵢ [μM] | Kᵢ [μM] | CDK2 | CDK2 | [μM] | [μM] | OVISE |
| Com- par- ator C | 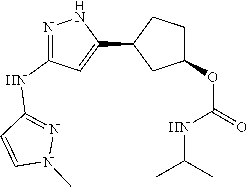 [SMILES: CC(C)NC(=O)O[C@@H]1CC[C@H](c2cc(Nc3ccn(C)n3)n[nH]2)C1] | 0.032 | 1.2 | 0.29 | 38 | 9.1 | 5.5 | >20 | >3.6 |
| 2 | 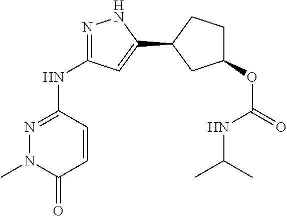 [SMILES: CC(C)NC(=O)O[C@@H]1CC[C@H](c2cc(Nc3ccc(=O)n(C)n3)n[nH]2)C1] | 0.00044 (73- fold) | 0.016 | 0.24 | 36 | 550 (+) | 0.093 (59- fold) | 11 | 120 (+) |
| 2A | 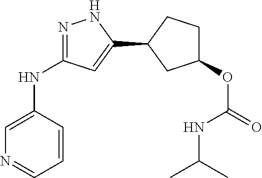 [SMILES: CC(C)NC(=O)O[C@@H]1CC[C@H](c2cc(Nc3cccnc3)n[nH]2)C1] | 0.0017 (19- fold) | 0.071 | 0.075 | 42 (+) | 44 (+) | 0.11 (50- fold) | 2.2 | 20 (+) |
| 2B | 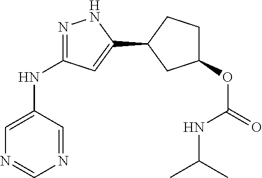 [SMILES: CC(C)NC(=O)O[C@@H]1CC[C@H](c2cc(Nc3cncnc3)n[nH]2)C1] | 0.0034 (9- fold) | 0.16 | 0.39 | 47 (+) | 120 (+) | 0.23 (24- fold) | >20 | >70 (+) |
| 2C | 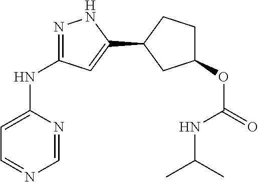 [SMILES: CC(C)NC(=O)O[C@@H]1CC[C@H](c2cc(Nc3ccncn3)n[nH]2)C1] | 0.0011 (32- fold) | 0.043 | 0.095 | 39 (+) | 86 (+) | 0.11 (50- fold) | 4.1 | 37 (+) |
| 2D | 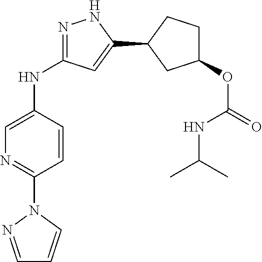 [SMILES: CC(C)NC(=O)O[C@@H]1CC[C@H](c2cc(Nc3ccc(-n4cccn4)nc3)n[nH]2)C1] | 0.014 (2.3- fold) | 0.35 | 0.48 | 25 | 34 (+) | 0.70 (7.9- fold) | 14 | 20 (+) |
| 2E | 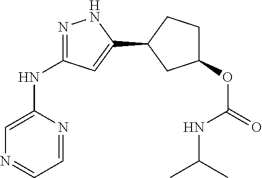 [SMILES: CC(C)NC(=O)O[C@@H]1CC[C@H](c2cc(Nc3cnccn3)n[nH]2)C1] | 0.00013 (246- fold) | 0.0084 | 0.0074 | 65 (+) | 57 (+) | 0.016 (344- fold) | 0.13 | 8.1 (+) |
| 2F | 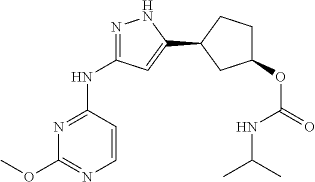 [SMILES: COc1nccc(Nc2cc([C@H]3CC[C@@H](OC(=O)NC(C)C)C3)[nH]n2)n1] | 0.00041 (78- fold) | 0.011 | 0.19 | 21 | 270 (+) | 0.065 (84.6- fold) | 3.5 | 58 (+) |
| 2G | 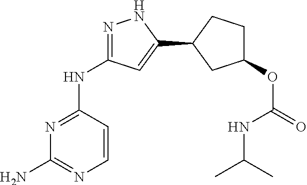 [SMILES: CC(C)NC(=O)O[C@@H]1CC[C@H](c2cc(Nc3ccnc(N)n3)n[nH]2)C1] | 0.0015 (21- fold) | 0.088 | 0.15 | 59 (+) | 100 (+) | 0.28 (19.6- fold) | 10 | 36 (+) |
| 2H | 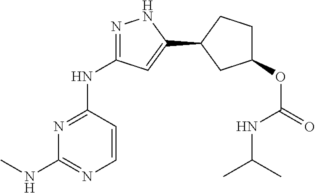 [SMILES: CNc1nccc(Nc2cc([C@H]3CC[C@@H](OC(=O)NC(C)C)C3)[nH]n2)n1] | 0.00028 (114- fold) | 0.0075 | 0.018 | 27 | 64 (+) | 0.067 (82- fold) | 1.1 | 16 (+) |
| 2I | 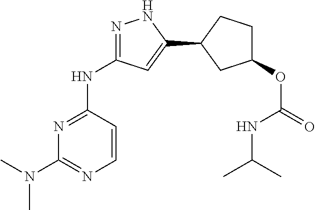 [SMILES: CC(C)NC(=O)O[C@@H]1CC[C@H](c2cc(Nc3ccnc(N(C)C)n3)n[nH]2)C1] | 0.00031 (103- fold) | 0.0060 | 0.027 | 19 | 87 (+) | 0.067 (82- fold) | 1.0 | 15 (+) |
| 2K | 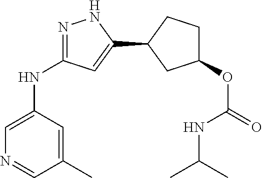 [SMILES: Cc1cncc(Nc2cc([C@H]3CC[C@@H](OC(=O)NC(C)C)C3)[nH]n2)c1] | 0.0032 (10- fold) | 0.059 | 0.34 | 18 | 110 (+) | 0.19 (28.9- fold) | 7.7 | 41 (+) |
| 2L | 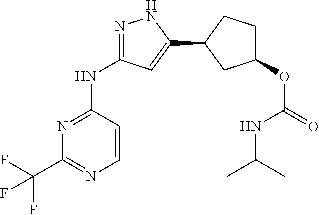 [SMILES: CC(C)NC(=O)O[C@@H]1CC[C@H](c2cc(Nc3ccnc(C(F)(F)F)n3)n[nH]2)C1] | 0.00017 (188- fold) | 0.0029 | 0.11 | 17 | 650 (+) | 0.012 (458- fold) | 0.64 | 53 (+) |
| 2M | 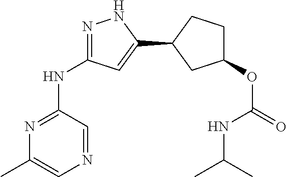 [SMILES: Cc1cncc(Nc2cc([C@H]3CC[C@@H](OC(=O)NC(C)C)C3)[nH]n2)n1] | 0.00026 (123- fold) | 0.0063 | 0.028 | 24 | 110 (+) | 0.020 (275- fold) | 0.58 | 29 (+) |
| 2N | 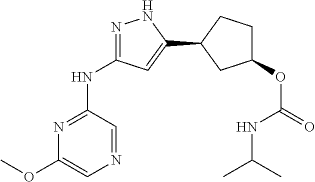 [SMILES: COc1cncc(Nc2cc([C@H]3CC[C@@H](OC(=O)NC(C)C)C3)[nH]n2)n1] | 0.00049 (65- fold) | 0.0037 | 0.026 | 7.6 | 53 (+) | 0.029 (190- fold) | 1.2 | 41 (+) |
| 2O | 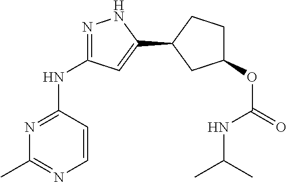 [SMILES: Cc1nccc(Nc2cc([C@H]3CC[C@@H](OC(=O)NC(C)C)C3)[nH]n2)n1] | 0.00087 (37- fold) | 0.024 | 0.25 | 28 | 290 (+) | 0.070 (78.6- fold) | 3.9 | 56 (+) |
| 2P | 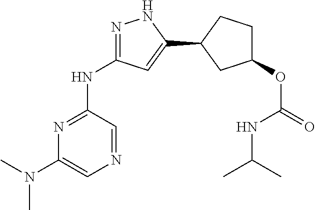 [SMILES: CC(C)NC(=O)O[C@@H]1CC[C@H](c2cc(Nc3cncc(N(C)C)n3)n[nH]2)C1] | 0.0042 (8- fold) | 0.033 | 0.18 | 7.9 | 43 (+) | 0.37 (14.9- fold) | 5.3 | 14 (+) |
| 2Q | 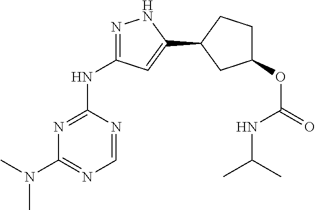 [SMILES: CC(C)NC(=O)O[C@@H]1CC[C@H](c2cc(Nc3ncnc(N(C)C)n3)n[nH]2)C1] | 0.041 (0.8- fold) | 0.67 | 1.5 | 16 | 37 (+) | 2.4 (2.3- fold) | >20 | >8.3 (+) |
| 2R | 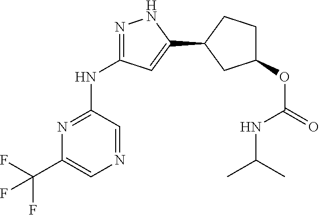 [SMILES: CC(C)NC(=O)O[C@@H]1CC[C@H](c2cc(Nc3cncc(C(F)(F)F)n3)n[nH]2)C1] | <0.00010 (320- fold) | 0.00040 | 0.0090 | 6.3 | 140 (+) | 0.0056 (982- fold) | 0.17 | 30 (+) |
| 2S | 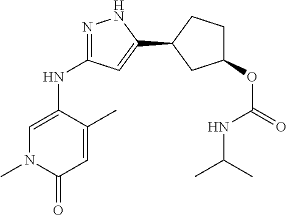 [SMILES: Cc1cc(=O)n(C)cc1Nc1cc([C@H]2CC[C@@H](OC(=O)NC(C)C)C2)[nH]n1] | 0.89 (0.4- fold) | 7.4 | 12 | 8.3 | 13 (+) | >20 (0.28- fold) | >20 | 1.0 |
| 2T | 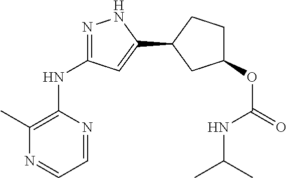 [SMILES: Cc1nccnc1Nc1cc([C@H]2CC[C@@H](OC(=O)NC(C)C)C2)[nH]n1] | 0.00086 (37- fold) | 0.064 | 0.15 | 74 (+) | 170 (+) | 0.052 (106- fold) | 1.5 | 29 (+) |
| 2U | 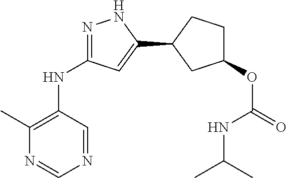 [SMILES: Cc1ncncc1Nc1cc([C@H]2CC[C@@H](OC(=O)NC(C)C)C2)[nH]n1] | 0.015 (2- fold) | 0.56 | 1.8 | 37 | 120 (+) | 0.40 (13.8- fold) | >20 | >50 (+) |
| 2V | 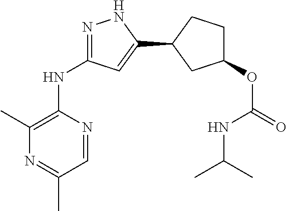 [SMILES: Cc1cnc(Nc2cc([C@H]3CC[C@@H](OC(=O)NC(C)C)C3)[nH]n2)c(C)n1] | 0.00092 (35- fold) | 0.036 | 0.26 | 39 (+) | 280 (+) | 0.11 (50- fold) | 2.5 | 23 (+) |
| 2W | 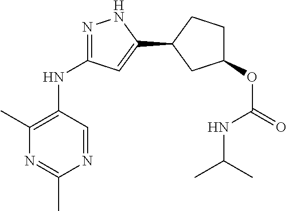 [SMILES: Cc1ncc(Nc2cc([C@H]3CC[C@@H](OC(=O)NC(C)C)C3)[nH]n2)c(C)n1] | 0.042 (0.8- fold) | 1.1 | 3.1 | 26 | 74 (+) | 2.7 (2- fold) | >20 | >7.4 (+) |
| 2X | 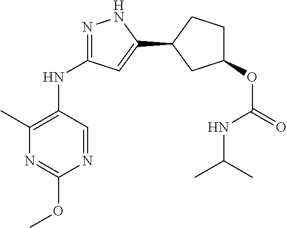 [SMILES: COc1ncc(Nc2cc([C@H]3CC[C@@H](OC(=O)NC(C)C)C3)[nH]n2)c(C)n1] | 0.059 (0.5- fold) | 1.2 | 3.8 | 20 | 64 (+) | 2.8 (2- fold) | >20 | >7.1 (+) |
| 2Y | 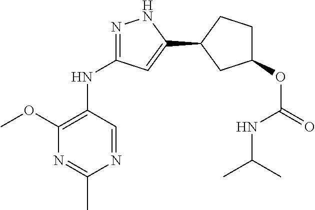 [SMILES: COc1nc(C)ncc1Nc1cc([C@H]2CC[C@@H](OC(=O)NC(C)C)C2)[nH]n1] | 0.21 (0.15- fold) | 7.5 | 49 | 36 | 230 (+) | 16 (0.18- fold) | >20 | >1.3 |
| 2Z | 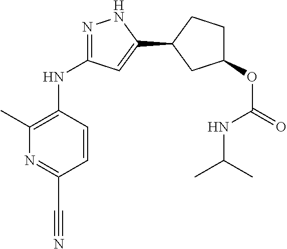 [SMILES: Cc1nc(C#N)ccc1Nc1cc([C@H]2CC[C@@H](OC(=O)NC(C)C)C2)[nH]n1] | 0.0050 (6.4- fold) | 0.55 | 1.3 | 110 (+) | 260 (+) | 0.25 (22- fold) | 18 | 72 (+) |
| 2AA | 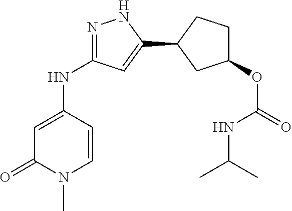 [SMILES: CC(C)NC(=O)O[C@@H]1CC[C@H](c2cc(Nc3ccn(C)c(=O)c3)n[nH]2)C1] | 0.017 (1.9- fold) | 0.44 | 0.45 | 26 | 26 (+) | 4.1 (1.3- fold) | >20 | >4.9 (+) |
| 2BB | 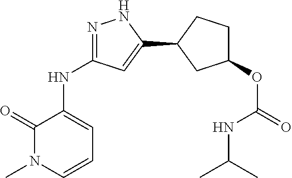 [SMILES: CC(C)NC(=O)O[C@@H]1CC[C@H](c2cc(Nc3cccn(C)c3=O)n[nH]2)C1] | 1.1 (0.3- fold) | 32 | 6.9 | 29 | 6.3 | >20 (0.28- fold) | >20 | 1.0 |
| 2CC | 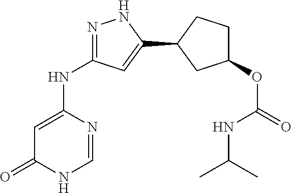 [SMILES: CC(C)NC(=O)O[C@@H]1CC[C@H](c2cc(Nc3cc(=O)[nH]cn3)n[nH]2)C1] | 0.00033 (97- fold) | 0.034 | 0.13 | 100 (+) | 390 (+) | 2.0 (2.8- fold) | >20 | >10 (+) |
| 2DD | 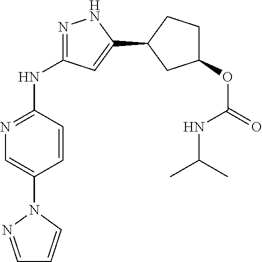 [SMILES: CC(C)NC(=O)O[C@@H]1CC[C@H](c2cc(Nc3ccc(-n4cccn4)cn3)n[nH]2)C1] | 0.013 (2.5- fold) | 0.68 | 0.35 | 52 (+) | 27 (+) | 0.61 (9- fold) | 14 | 23 (+) |
| 2EE | 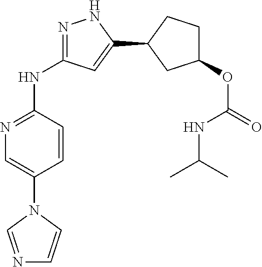 [SMILES: CC(C)NC(=O)O[C@@H]1CC[C@H](c2cc(Nc3ccc(-n4ccnc4)cn3)n[nH]2)C1] | 0.0032 (10- fold) | 0.29 | 0.19 | 91 (+) | 59 (+) | 0.34 (16- fold) | 9.7 | 29 (+) |

TABLE R
Bicyclic versus Monocyclic Heteroaryl Ring A Comparison

| | | | | | | | Cellular Assays | | |
|---|---|---|---|---|---|---|---|---|---|
| | | Enzymatic Assays | | | | | CDK2 | CDK4/6 | FS |
| | | | | | FS | FS | OVISE | SKOV- | SKOV- |
| | | CDK2 | CDK1 | CDK4 | CDK1/ | CDK4/ | IC₅₀ | 3 | 3/ |
| Ex | Compound | Kᵢ [μM] | Kᵢ [μM] | Kᵢ [μM] | CDK2 | CDK2 | [μM] | IC₅₀ [μM] | OVISE |
| Comparator D | 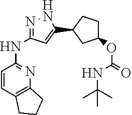 [SMILES: CC(C)(C)NC(=O)O[C@@H]1CC[C@H](c2cc(Nc3ccc4c(n3)CCC4)n[nH]2)C1] | 0.0027 | 0.054 | 0.0052 | 20 | 1.9 | 3.8 | 1.8 | 0.47 |
| | (1R,3S)-3-(3- | | | | | | | | |
| | ((6,7-dihydro-5H- | | | | | | | | |
| | cyclopenta[b]pyridin-2- | | | | | | | | |
| | yl)amino)-1H-pyrazol-5- | | | | | | | | |
| | yl)cyclopentyl tert- | | | | | | | | |
| | butylcarbamate | | | | | | | | |
| 4G | 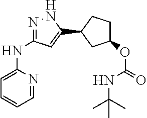 [SMILES: CC(C)(C)NC(=O)O[C@@H]1CC[C@H](c2cc(Nc3ccccn3)n[nH]2)C1] | 0.0017 (1.6-fold) | 0.075 | 0.027 | 44 (+) | 16 (+) | 0.24 (16-fold) | 1.5 | 6.3 (+) |
| | (1R,3S)-3-(3-(pyridin-2- | | | | | | | | |
| | ylamino)-1H-pyrazol-5- | | | | | | | | |
| | yl)cyclopentyl tert- | | | | | | | | |
| | butylcarbamate | | | | | | | | |
| 2A | 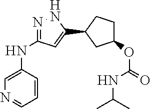 [SMILES: CC(C)NC(=O)O[C@@H]1CC[C@H](c2cc(Nc3cccnc3)n[nH]2)C1] | 0.0017 (1.6-fold) | 0.071 | 0.075 | 42 (+) | 44 (+) | 0.11 (35-fold) | 2.2 | 20 (+) |
| | (1R,3S)-3-(3-(pyridin-3- | | | | | | | | |
| | ylamino)-1H-pyrazol-5- | | | | | | | | |
| | yl)cyclopentyl | | | | | | | | |
| | isopropylcarbamate | | | | | | | | |
| 4 | 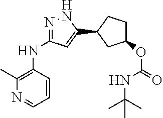 [SMILES: Cc1ncccc1Nc1cc([C@H]2CC[C@@H](OC(=O)NC(C)(C)C)C2)[nH]n1] | 0.00022 (12.3-fold) | 0.025 | 0.034 | 110 (+) | 160 (+) | 0.020 (190-fold) | 0.92 | 46 (+) |
| | (1R,3S)-3-(3-((2- | | | | | | | | |
| | methylpyridin-3-yl)amino)- | | | | | | | | |
| | 1H-pyrazol-5-yl) | | | | | | | | |
| | cyclopentyl | | | | | | | | |
| | tert-butylcarbamate | | | | | | | | |
| 4A | 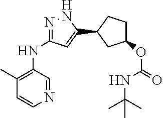 [SMILES: Cc1ccncc1Nc1cc([C@H]2CC[C@@H](OC(=O)NC(C)(C)C)C2)[nH]n1] | 0.0026 (1-fold) | 0.21 | 0.41 | 78 (+) | 170 (+) | 0.18 (21-fold) | 9.9 | 71 (+) |
| | (1R,3S)-3-(3-((4- | | | | | | | | |
| | methylpyridin-3-yl) | | | | | | | | |
| | amino)-1H-pyrazol-5- | | | | | | | | |
| | yl)cyclopentyl | | | | | | | | |
| | tert-butylcarbamate | | | | | | | | |
| 4B | 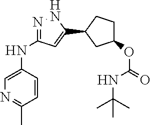 [SMILES: Cc1ccc(Nc2cc([C@H]3CC[C@@H](OC(=O)NC(C)(C)C)C3)[nH]n2)cn1] | 0.00052 (5.2-fold) | 0.020 | 0.019 | 38 (+) | 37 (+) | 0.033 (115-fold) | 0.39 | 12 (+) |
| | (1R,3S)-3-(3-((6- | | | | | | | | |
| | methylpyridin-3-yl)amino)- | | | | | | | | |
| | 1H-pyrazol-5- | | | | | | | | |
| | yl)cyclopentyl | | | | | | | | |
| | tert-butylcarbamate | | | | | | | | |
| 4H | 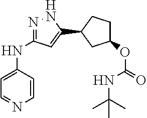 [SMILES: CC(C)(C)NC(=O)O[C@@H]1CC[C@H](c2cc(Nc3ccncc3)n[nH]2)C1] | 0.0020 (1.4-fold) | 0.18 | 0.11 | 90 (+) | 55 (+) | 0.038 (100-fold) | 0.95 | 25 (+) |
| | (1R,3S)-3-(3-(pyridin-4- | | | | | | | | |
| | ylamino)-1H-pyrazol-5- | | | | | | | | |
| | yl)cyclopentyl tert- | | | | | | | | |
| | butylcarbamate | | | | | | | | |
| 4E | 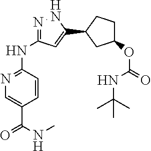 [SMILES: CNC(=O)c1ccc(Nc2cc([C@H]3CC[C@@H](OC(=O)NC(C)(C)C)C3)[nH]n2)nc1] | 0.0016 (1.7-fold) | 0.075 | 0.084 | 47 (+) | 53 (+) | 0.096 (40-fold) | 2.6 | 27 (+) |
| | (1R,3S)-3-(3-((5- | | | | | | | | |
| | (methylcarbamoyl)pyridin- | | | | | | | | |
| | 2-yl)amino)-1H-pyrazol-5- | | | | | | | | |
| | yl)cyclopentyl tert- | | | | | | | | |
| | butylcarbamate | | | | | | | | |
| 4F | 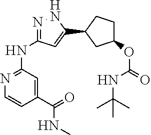 [SMILES: CNC(=O)c1ccnc(Nc2cc([C@H]3CC[C@@H](OC(=O)NC(C)(C)C)C3)[nH]n2)c1] | 0.0025 (1.1-fold) | 0.052 | 0.037 | 21 (+) | 15 (+) | 0.19 (20-fold) | 1.7 | 8.9 (+) |
| | (1R,3S)-3-(3-((4- | | | | | | | | |
| | (methylcarbamoyl)pyridin- | | | | | | | | |
| | 2-yl)amino)-1H-pyrazol-5- | | | | | | | | |
| | yl)cyclopentyl tert- | | | | | | | | |
| | butylcarbamate | | | | | | | | |
| 1 | 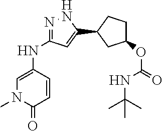 [SMILES: Cn1cc(Nc2cc([C@H]3CC[C@@H](OC(=O)NC(C)(C)C)C3)[nH]n2)ccc1=O] | 0.0076 (0.4-fold) | 0.27 | 0.70 | 36 (+) | 92 (+) | 0.22 (17-fold) | >20 | >91 (+) |
| | (1R,3S)-3-(3-((1-methyl-6- | | | | | | | | |
| | oxo-1,6-dihydropyridin-3- | | | | | | | | |
| | yl)amino)-1H-pyrazol-5- | | | | | | | | |
| | yl)cyclopentyl tert- | | | | | | | | |
| | butylcarbamate | | | | | | | | |
| 4D | 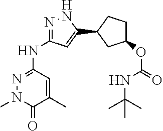 [SMILES: Cc1cc(Nc2cc([C@H]3CC[C@@H](OC(=O)NC(C)(C)C)C3)[nH]n2)nn(C)c1=O] | <0.00010 (27-fold) | 0.0037 | 0.039 | >59 (+) | >620 (+) | 0.026 (146-fold) | 1.5 | 58 (+) |
| | (1R,3S)-3-(3- | | | | | | | | |
| | ((1,5-dimethyl-6-oxo- | | | | | | | | |
| | 1,6-dihydropyridazin- | | | | | | | | |
| | 3-yl)amino)-1H-pyrazol-5- | | | | | | | | |
| | yl)cyclopentyl tert- | | | | | | | | |
| | butylcarbamate | | | | | | | | |
| 4C | 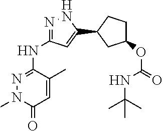 [SMILES: Cc1cc(=O)n(C)nc1Nc1cc([C@H]2CC[C@@H](OC(=O)NC(C)(C)C)C2)[nH]n1] | 0.0021 (1.3-fold) | 0.065 | 0.58 | 31 (+) | 280 (+) | 0.60 (6-fold) | 19 | 32 (+) |
| | (1R,3S)-3-(3-((1,4- | | | | | | | | |
| | dimethyl-6-oxo-1,6- | | | | | | | | |
| | dihydropyridazin- | | | | | | | | |
| | 3-yl)amino)-1H-pyrazol-5- | | | | | | | | |
| | yl)cyclopentyl tert- | | | | | | | | |
| | butylcarbamate | | | | | | | | |
| 6C | 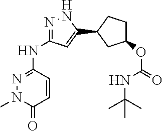 [SMILES: Cn1nc(Nc2cc([C@H]3CC[C@@H](OC(=O)NC(C)(C)C)C3)[nH]n2)ccc1=O] | <0.00010 (27-fold) | 0.0036 | 0.045 | >47 (+) | >584 (+) | 0.03 (127-fold) | 1.5 | 50 (+) |
| | (1R,3S)-3-(3-((1-methyl-6- | | | | | | | | |
| | oxo-1,6-dihydropyridazin- | | | | | | | | |
| | 3-yl)amino)-1H-pyrazol-5- | | | | | | | | |
| | yl)cyclopentyl tert- | | | | | | | | |
| | butylcarbamate | | | | | | | | |
| 4J | 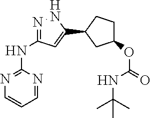 [SMILES: CC(C)(C)NC(=O)O[C@@H]1CC[C@H](c2cc(Nc3ncccn3)n[nH]2)C1] | 0.0025 (1.1-fold) | 0.17 | 0.011 | 68 (+) | 4.4 (+) | 0.32 (12-fold) | 1.2 | 3.8 (+) |
| | (1R,3S)-3-(3-(pyrimidin-2- | | | | | | | | |
| | ylamino)-1H-pyrazol-5- | | | | | | | | |
| | yl)cyclopentyl tert- | | | | | | | | |
| | butylcarbamate | | | | | | | | |
| 4I | 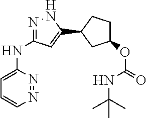 [SMILES: CC(C)(C)NC(=O)O[C@@H]1CC[C@H](c2cc(Nc3cccnn3)n[nH]2)C1] | 0.0025 (1.1-fold) | 0.045 | 0.033 | 18 | 13 (+) | 0.24 (16-fold) | 4.9 | 20 (+) |
| | (1R,3S)-3-(3-(pyridazin-3- | | | | | | | | |
| | ylamino)-1H-pyrazol-5- | | | | | | | | |
| | yl)cyclopentyl tert- | | | | | | | | |
| | butylcarbamate | | | | | | | | |
| 4K | 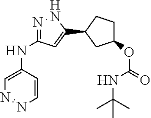 [SMILES: CC(C)(C)NC(=O)O[C@@H]1CC[C@H](c2cc(Nc3ccnnc3)n[nH]2)C1] | 0.00045 (6-fold) | 0.027 | 0.052 | 54 (+) | 104 (+) | 0.078 (49-fold) | 5.5 | 71 (+) |
| | (1R,3S)-3-(3-(pyridazin-4- | | | | | | | | |
| | ylamino)-1H-pyrazol-5- | | | | | | | | |
| | yl)cyclopentyl tert- | | | | | | | | |
| | butylcarbamate | | | | | | | | |

TABLE S
Aryl versus Monocyclic Heteroaryl Ring A Comparison

| | | | | | | | Cellular Assays | | |
|---|---|---|---|---|---|---|---|---|---|
| | | Enzymatic Assays | | | | | CDK2 | CDK4/6 | FS |
| | | | | | FS | FS | OVISE | SKOV-3 | SKOV- |
| | | CDK2 | CDK1 | CDK4 | CDK1/ | CDK4/ | IC₅₀ | IC₅₀ | 3/ |
| Ex | Compound | Kᵢ [μM] | Kᵢ [μM] | Kᵢ [μM] | CDK2 | CDK2 | [μM] | [μM] | OVISE |
| Comparator E | 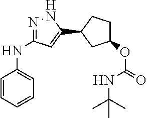 [SMILES: CC(C)(C)NC(=O)O[C@@H]1CC[C@H](c2cc(Nc3ccccc3)n[nH]2)C1] | 0.00052 | 0.033 | 0.023 | 63 | 44 | 0.17 | 2.7 | 16 |
| | (1R,3S)-3-(3-(phenylamino)-1H- | | | | | | | | |
| | pyrazol-5-yl)cyclopentyl tert- | | | | | | | | |
| | butylcarbamate | | | | | | | | |
| 4G | 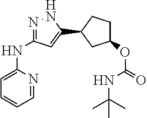 [SMILES: CC(C)(C)NC(=O)O[C@@H]1CC[C@H](c2cc(Nc3ccccn3)n[nH]2)C1] | 0.0017 (0.3-fold) | 0.075 | 0.027 | 44 | 16 | 0.24 (0.7-fold) | 1.5 | 6.3 |
| | (1R,3S)-3-(3-(pyridin-2- | | | | | | | | |
| | ylamino)-1H-pyrazol-5- | | | | | | | | |
| | yl)cyclopentyl tert- | | | | | | | | |
| | butylcarbamate | | | | | | | | |
| 2A | 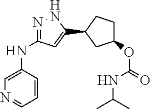 [SMILES: CC(C)NC(=O)O[C@@H]1CC[C@H](c2cc(Nc3cccnc3)n[nH]2)C1] | 0.0017 (0.3-fold) | 0.071 | 0.075 | 42 | 44 | 0.11 (1.5-fold) | 2.2 | 20 (+) |
| | (1R,3S)-3-(3-(pyridin-3- | | | | | | | | |
| | ylamino)-1H-pyrazol-5- | | | | | | | | |
| | yl)cyclopentyl | | | | | | | | |
| | isopropylcarbamate | | | | | | | | |
| 4 | 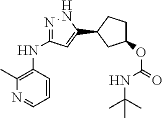 [SMILES: Cc1ncccc1Nc1cc([C@H]2CC[C@@H](OC(=O)NC(C)(C)C)C2)[nH]n1] | 0.00022 (2.4-fold) | 0.025 | 0.034 | 110 (+) | 160 (+) | 0.020 (8.5-fold) | 0.92 | 46 (+) |
| | (1R,3S)-3-(3-((2- | | | | | | | | |
| | methylpyridin-3-yl)amino)-1H- | | | | | | | | |
| | pyrazol-5-yl)cyclopentyl tert- | | | | | | | | |
| | butylcarbamate | | | | | | | | |
| 4A | 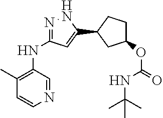 [SMILES: Cc1ccncc1Nc1cc([C@H]2CC[C@@H](OC(=O)NC(C)(C)C)C2)[nH]n1] | 0.0026 (0.2-fold) | 0.21 | 0.41 | 78 (+) | 170 (+) | 0.18 (0.9-fold) | 9.9 | 71 (+) |
| | (1R,3S)-3-(3-((4- | | | | | | | | |
| | methylpyridin-3-yl)amino)-1H- | | | | | | | | |
| | pyrazol-5-yl)cyclopentyl tert- | | | | | | | | |
| | butylcarbamate | | | | | | | | |
| 4B | 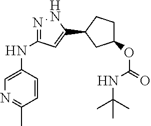 [SMILES: Cc1ccc(Nc2cc([C@H]3CC[C@@H](OC(=O)NC(C)(C)C)C3)[nH]n2)cn1] | 0.00052 (1-fold) | 0.020 | 0.019 | 38 | 37 | 0.033 (5.2-fold) | 0.39 | 12 |
| | (1R,3S)-3-(3-((6- | | | | | | | | |
| | methylpyridin-3-yl)amino)-1H- | | | | | | | | |
| | pyrazol-5-yl)cyclopentyl tert- | | | | | | | | |
| | butylcarbamate | | | | | | | | |
| 4H | 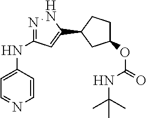 [SMILES: CC(C)(C)NC(=O)O[C@@H]1CC[C@H](c2cc(Nc3ccncc3)n[nH]2)C1] | 0.0020 (0.3-fold) | 0.18 | 0.11 | 90 (+) | 55 (+) | 0.038 (4.4-fold) | 0.95 | 25 (+) |
| | (1R,3S)-3-(3-(pyridin-4- | | | | | | | | |
| | ylamino)-1H-pyrazol-5- | | | | | | | | |
| | yl)cyclopentyl tert- | | | | | | | | |
| | butylcarbamate | | | | | | | | |
| 4E | 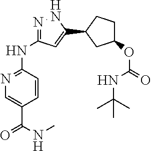 [SMILES: CNC(=O)c1ccc(Nc2cc([C@H]3CC[C@@H](OC(=O)NC(C)(C)C)C3)[nH]n2)nc1] | 0.0016 (0.3-fold) | 0.075 | 0.084 | 47 | 53 (+) | 0.096 (1.8-fold) | 2.6 | 27 (+) |
| | (1R,3S)-3-(3-((5- | | | | | | | | |
| | (methylcarbamoyl)pyridin-2- | | | | | | | | |
| | yl)amino)-1H-pyrazol-5- | | | | | | | | |
| | yl)cyclopentyl tert- | | | | | | | | |
| | butylcarbamate | | | | | | | | |
| 4F | 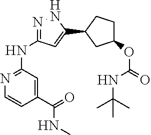 [SMILES: CNC(=O)c1ccnc(Nc2cc([C@H]3CC[C@@H](OC(=O)NC(C)(C)C)C3)[nH]n2)c1] | 0.0025 (0.2-fold) | 0.052 | 0.037 | 21 | 15 | 0.19 (0.9-fold) | 1.7 | 8.9 |
| | (1R,3S)-3-(3-((4- | | | | | | | | |
| | (methylcarbamoyl)pyridin-2- | | | | | | | | |
| | yl)amino)-1H-pyrazol-5- | | | | | | | | |
| | yl)cyclopentyl tert- | | | | | | | | |
| | butylcarbamate | | | | | | | | |
| 1 | 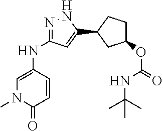 [SMILES: Cn1cc(Nc2cc([C@H]3CC[C@@H](OC(=O)NC(C)(C)C)C3)[nH]n2)ccc1=O] | 0.0076 (0.1-fold) | 0.27 | 0.70 | 36 | 92 (+) | 0.22 (0.8-fold) | >20 | >91 (+) |
| | (1R,3S)-3-(3-((1-methyl-6-oxo- | | | | | | | | |
| | 1,6-dihydropyridin-3- | | | | | | | | |
| | yl)amino)-1H-pyrazol-5- | | | | | | | | |
| | yl)cyclopentyl tert- | | | | | | | | |
| | butylcarbamate | | | | | | | | |
| 4D | 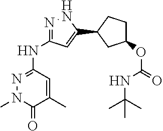 [SMILES: Cc1cc(Nc2cc([C@H]3CC[C@@H](OC(=O)NC(C)(C)C)C3)[nH]n2)nn(C)c1=O] | <0.00010 (5.2-fold) | 0.0037 | 0.039 | >59 | >620 (+) | 0.026 (6.5-fold) | 1.5 | 58 (+) |
| | (1R,3S)-3-(3-((1,5-dimethyl-6- | | | | | | | | |
| | oxo-1,6-dihydropyridazin-3- | | | | | | | | |
| | yl)amino)-1H-pyrazol-5- | | | | | | | | |
| | yl)cyclopentyl tert- | | | | | | | | |
| | butylcarbamate | | | | | | | | |
| 4C | 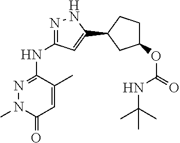 [SMILES: Cc1cc(=O)n(C)nc1Nc1cc([C@H]2CC[C@@H](OC(=O)NC(C)(C)C)C2)[nH]n1] | 0.0021 (0.3-fold) | 0.065 | 0.58 | 31 | 280 (+) | 0.60 (0.3-fold) | 19 | 32 (+) |
| | (1R,3S)-3-(3-((1,4-dimethyl-6- | | | | | | | | |
| | oxo-1,6-dihydropyridazin-3- | | | | | | | | |
| | yl)amino)-1H-pyrazol-5- | | | | | | | | |
| | yl)cyclopentyl tert- | | | | | | | | |
| | butylcarbamate | | | | | | | | |
| 6C | 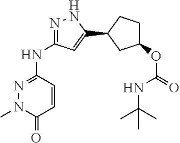 [SMILES: Cn1nc(Nc2cc([C@H]3CC[C@@H](OC(=O)NC(C)(C)C)C3)[nH]n2)ccc1=O] | <0.00010 (5.2-fold) | 0.0036 | 0.045 | >47 (+) | >584 (+) | 0.03 (5.7-fold) | 1.5 | 50 (+) |
| | (1R,3S)-3-(3-((1-methyl-6-oxo- | | | | | | | | |
| | 1,6-dihydropyridazin-3- | | | | | | | | |
| | yl)amino)-1H-pyrazol-5- | | | | | | | | |
| | yl)cyclopentyl tert- | | | | | | | | |
| | butylcarbamate | | | | | | | | |
| 4J | 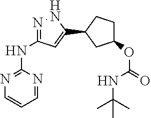 [SMILES: CC(C)(C)NC(=O)O[C@@H]1CC[C@H](c2cc(Nc3ncccn3)n[nH]2)C1] | 0.0025 (0.2-fold) | 0.17 | 0.011 | 68 (+) | 4.4 | 0.32 (0.5-fold) | 1.2 | 3.8 |
| | (1R,3S)-3-(3-(pyrimidin-2- | | | | | | | | |
| | ylamino)-1H-pyrazol-5- | | | | | | | | |
| | yl)cyclopentyl tert- | | | | | | | | |
| | butylcarbamate | | | | | | | | |
| 4I | 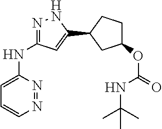 [SMILES: CC(C)(C)NC(=O)O[C@@H]1CC[C@H](c2cc(Nc3cccnn3)n[nH]2)C1] | 0.0025 (0.2-fold) | 0.045 | 0.033 | 18 | 13 | 0.24 (0.7-fold) | 4.9 | 20 (+) |
| | (1R,3S)-3-(3-(pyridazin-3- | | | | | | | | |
| | ylamino)-1H-pyrazol-5- | | | | | | | | |
| | yl)cyclopentyl tert- | | | | | | | | |
| | butylcarbamate | | | | | | | | |
| 4K | 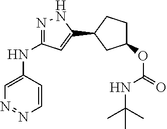 [SMILES: CC(C)(C)NC(=O)O[C@@H]1CC[C@H](c2cc(Nc3ccnnc3)n[nH]2)C1] | 0.00045 (1.2-fold) | 0.027 | 0.052 | 54 | 104 (+) | 0.078 (2.2-fold) | 5.5 | 71 (+) |
| | (1R,3S)-3-(3-(pyridazin-4- | | | | | | | | |
| | ylamino)-1H-pyrazol-5- | | | | | | | | |
| | yl)cyclopentyl tert- | | | | | | | | |
| | butylcarbamate | | | | | | | | |

### OTHER EMBODIMENTS

Although the foregoing disclosure has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula (I): 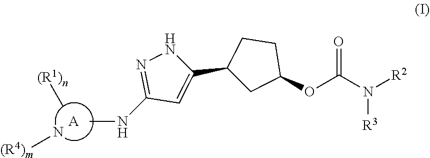
or a pharmaceutically acceptable salt thereof, wherein:
Ring A is a 6-membered heteroaryl having at least 1 N-ring heteroatom, and having 0 to 2 additional N-ring heteroatoms;
each R¹ is independently C₁₋₆ alkyl, C₁₋₆ alkoxy, C₁₋₆ hydroxyalkyl, C₂₋₆ alkoxyalkyl, halogen, C₁₋₆ haloalkyl, C₁₋₆ haloalkoxy, —CN, —N(R^{1a})(R^{1b}), —C₁₋₆ alkyl-N(R^{1a})(R^{1b}), —C(O)N(R^{1a})(R^{1b}), —O—R^{1c}, —S(O)₂R^{1a}, C₃₋₈ cycloalkyl, a 3 to 6 membered heterocycloalkyl having 1 or 2 heteroatoms each independently N or O, or a 5 membered heteroaryl having 1 or 2 heteroatoms each independently N or O, wherein each heterocycloalkyl and heteroaryl is substituted with 0, 1, 2, or 3 R^{1d};
each R^{1a} and R^{1b} a is independently hydrogen, C₁₋₆ alkyl, or C₁₋₆ haloalkyl;
R^{1c} is a 3 to 6 membered heterocycloalkyl having 1 to 2 heteroatoms each independently N and O;
R² is independently C₁₋₆ alkyl, C₁₋₆ haloalkyl, C₃₋₈ cycloalkyl, a 3- to 6-membered heterocycloalkyl having 1 or 2 heteroatoms each independently N or O, or C₁₋₆ alkylaryl, wherein the cycloalkyl, the heterocycloalkyl, and the aryl are each independently substituted with 0, 1, 2, or 3 R^{2a} groups;
each R^{1d} and R^{2a} is independently hydrogen, C₁₋₆ alkyl, halogen, C₁₋₆ haloalkyl or hydroxy;
R³ is hydrogen, C₁₋₆ alkyl, or C₁₋₆ haloalkyl;
R⁴ is hydrogen, C₁₋₄ alkyl, C₂₋₄ alkoxyalkyl or C₁₋₃ haloalkyl;
subscript n is an integer 0, 1, 2, 3, or 4; and
subscript m is an integer 0 or 1.

2.-3. (canceled)

4. The compound of claim 1, or a pharmaceutically acceptable salt thereof, wherein Ring A is

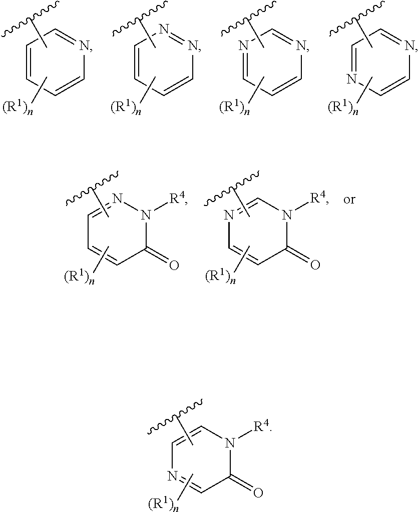

5.-7. (canceled)

8. The compound of claim 1, or a pharmaceutically acceptable salt thereof, wherein Ring A is

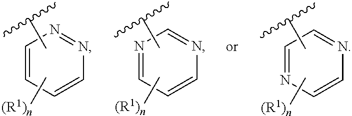

9.-18. (canceled)

19. The compound of claim 1, or a pharmaceutically acceptable salt thereof, wherein Ring A is pyridine, pyridazine, pyrimidine, pyrazine, triazine, pyridine-2-one, pyridazine-3-one, pyrimidine-2-one, or pyrazine-2-one.

20.-29. (canceled)

30. The compound of claim 1, or a pharmaceutically acceptable salt thereof, wherein
each R¹ is independently C₁₋₄ alkyl, C₁₋₃ alkoxy, C₁₋₃ hydroxyalkyl, halogen, C₁₋₃ haloalkyl, —CN, —N(R^{1a})(R^{1b}), —C₁₋₃ alkyl-N(R^{1a})(R^{1b}), —C(O)N(R^{1a})(R^{1b}), —S(O)₂R^{1a}, C₃₋₅ cycloalkyl, a 6 membered heterocycloalkyl having 2 heteroatoms each independently N or O, or a 5 membered heteroaryl having 2 heteroatoms each independently N, wherein each heterocycloalkyl and heteroaryl is substituted with 0 or 1 R^{1d};
each R^{1a} and R^{1b} is independently hydrogen, C₁₋₃ alkyl, or C₁₋₃ haloalkyl; and
R^{1d} is hydrogen, C₁₋₆ alkyl, halogen, C₁₋₆ haloalkyl or hydroxy.

31. (canceled)

32. The compound of claim 1, or a pharmaceutically acceptable salt thereof, wherein R¹ is independently Me, —OCH₃, —CH₂OH, —CF₃, —CN, —NH₂, —NHCH₃, —N(CH₃)₂, —CH₂N(CH₃)₂, —C(O)NHCH₃, —C(O)N(CH₃)₂, —S(O)₂CH₃,

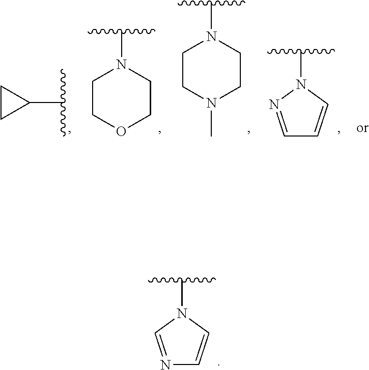

33. The compound of claim 1, or a pharmaceutically acceptable salt thereof, wherein R⁴ is hydrogen, or C₁₋₄ alkyl.

34.-36. (canceled)

37. The compound of claim 1, or a pharmaceutically acceptable salt thereof, wherein
R² is independently C₁₋₄ alkyl, C₁₋₄ haloalkyl, C₃₋₆ cycloalkyl, a 4, 5, or 6 membered heterocycloalkyl having 1 heteroatom of N or O, or C₁₋₄ alkylaryl, wherein the cycloalkyl, the heterocycloalkyl, and aryl are each independently substituted with 0, 1, 2, or 3 R^{2a} groups; and
each R^{2a} is independently C₁₋₃ alkyl, C₁₋₃ haloalkyl or hydroxy.

38. (canceled)

39. The compound of claim 1, or a pharmaceutically acceptable salt thereof, wherein
R² is independently C₁₋₄ alkyl, C₁₋₄ haloalkyl, cyclopropyl, cyclobutyl, bicyclo[1.1.1]pentyl, azetidine, pyrrolidine, piperidine, oxetane, tetrahydrofuran, tetrahydropyran, or phenylethyl, wherein the cyclopropyl and cyclobutyl are each independently substituted with 0 or 1 R^{2a} group, wherein each R^{2a} is Me, CF₃ or —OH, and wherein the azetidine, pyrrolidine, piperidine, oxetane, tetrahydrofuran, or tetrahydropyran are each independently substituted with 0 or 1 Me group.

40. The compound of claim 1, or a pharmaceutically acceptable salt thereof, wherein R² is independently C₃₋₄ alkyl, cyclopropyl, cyclobutyl, or bicyclo[1.1.1]pentyl, wherein the cyclopropyl and cyclobutyl are each independently substituted with 0 or 1 Me group.

41.-42. (canceled)

43. The compound of claim 1, or a pharmaceutically acceptable salt thereof, wherein R³ is hydrogen or C₁₋₃ alkyl.

44.-47. (canceled)

48. The compound of claim 1 selected from the group consisting of: 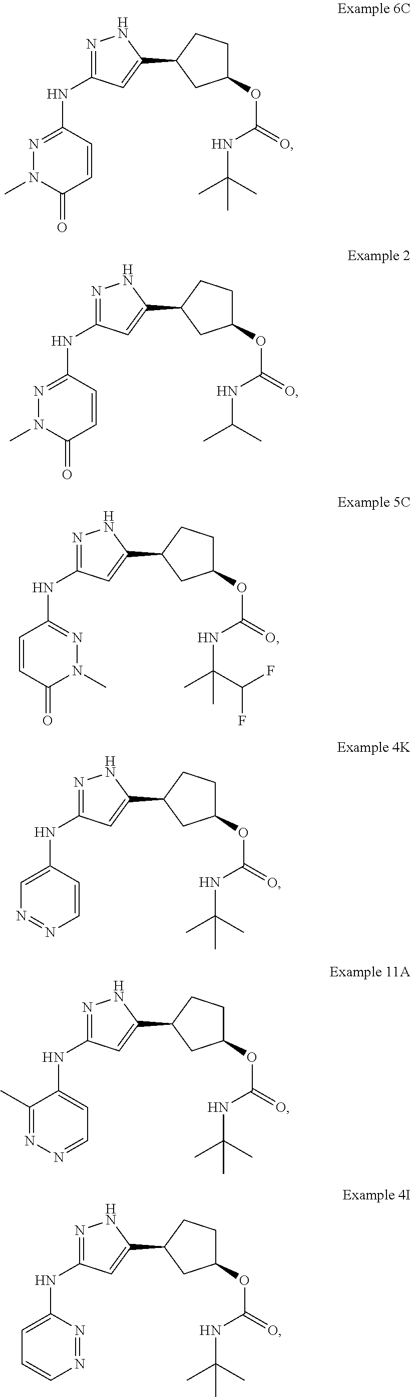 [SMILES: Cn1nc(Nc2cc([C@H]3CC[C@@H](OC(=O)NC(C)(C)C)C3)[nH]n2)ccc1=O ; CC(C)NC(=O)O[C@@H]1CC[C@H](c2cc(Nc3ccc(=O)n(C)n3)n[nH]2)C1 ; Cn1nc(Nc2cc([C@H]3CC[C@@H](OC(=O)NC(C)(C)C(F)F)C3)[nH]n2)ccc1=O ; CC(C)(C)NC(=O)O[C@@H]1CC[C@H](c2cc(Nc3ccnnc3)n[nH]2)C1 ; Cc1nnccc1Nc1cc([C@H]2CC[C@@H](OC(=O)NC(C)(C)C)C2)[nH]n1 ; CC(C)(C)NC(=O)O[C@@H]1CC[C@H](c2cc(Nc3cccnn3)n[nH]2)C1] 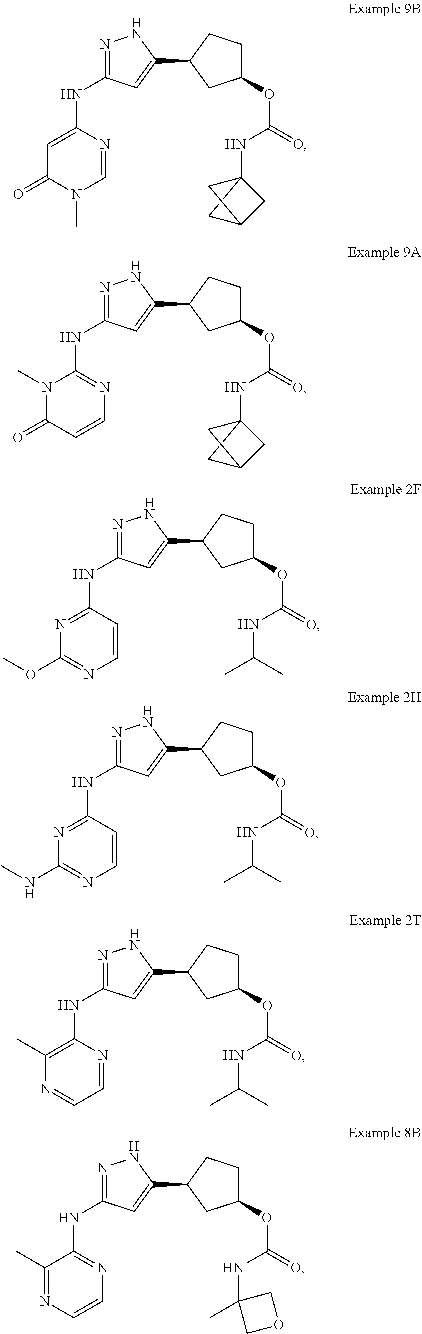 [SMILES: Cn1cnc(Nc2cc([C@H]3CC[C@@H](OC(=O)NC45CC(C4)C5)C3)[nH]n2)cc1=O ; Cn1c(Nc2cc([C@H]3CC[C@@H](OC(=O)NC45CC(C4)C5)C3)[nH]n2)nccc1=O ; COc1nccc(Nc2cc([C@H]3CC[C@@H](OC(=O)NC(C)C)C3)[nH]n2)n1 ; CNc1nccc(Nc2cc([C@H]3CC[C@@H](OC(=O)NC(C)C)C3)[nH]n2)n1 ; Cc1nccnc1Nc1cc([C@H]2CC[C@@H](OC(=O)NC(C)C)C2)[nH]n1 ; Cc1nccnc1Nc1cc([C@H]2CC[C@@H](OC(=O)NC3(C)COC3)C2)[nH]n1] 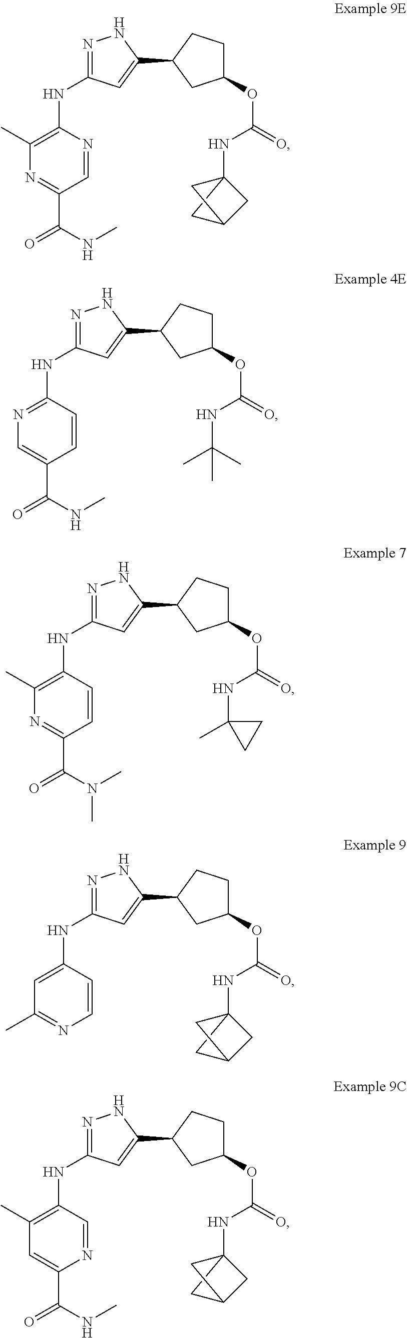 [SMILES: CNC(=O)c1cnc(Nc2cc([C@H]3CC[C@@H](OC(=O)NC45CC(C4)C5)C3)[nH]n2)c(C)n1 ; CNC(=O)c1ccc(Nc2cc([C@H]3CC[C@@H](OC(=O)NC(C)(C)C)C3)[nH]n2)nc1 ; Cc1nc(C(=O)N(C)C)ccc1Nc1cc([C@H]2CC[C@@H](OC(=O)NC3(C)CC3)C2)[nH]n1 ; Cc1cc(Nc2cc([C@H]3CC[C@@H](OC(=O)NC45CC(C4)C5)C3)[nH]n2)ccn1 ; CNC(=O)c1cc(C)c(Nc2cc([C@H]3CC[C@@H](OC(=O)NC45CC(C4)C5)C3)[nH]n2)cn1] 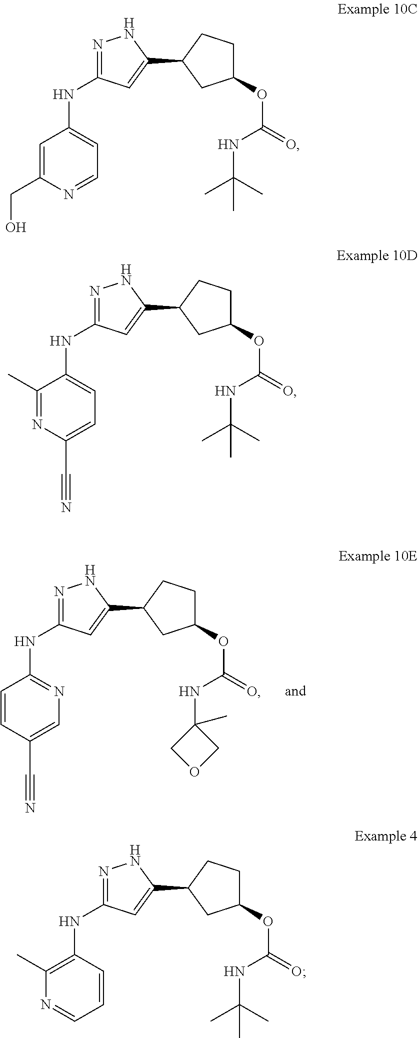 [SMILES: CC(C)(C)NC(=O)O[C@@H]1CC[C@H](c2cc(Nc3ccnc(CO)c3)n[nH]2)C1 ; Cc1nc(C#N)ccc1Nc1cc([C@H]2CC[C@@H](OC(=O)NC(C)(C)C)C2)[nH]n1 ; CC1(NC(=O)O[C@@H]2CC[C@H](c3cc(Nc4ccc(C#N)cn4)n[nH]3)C2)COC1 ; Cc1ncccc1Nc1cc([C@H]2CC[C@@H](OC(=O)NC(C)(C)C)C2)[nH]n1]
or pharmaceutically acceptable salts thereof.

49. The compound of claim 1 of the formula: 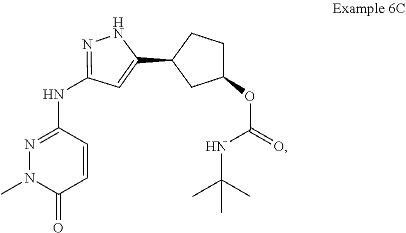 [SMILES: Cn1nc(Nc2cc([C@H]3CC[C@@H](OC(=O)NC(C)(C)C)C3)[nH]n2)ccc1=O]
or a pharmaceutically acceptable salt thereof.

50. The compound of claim 1 of the formula: 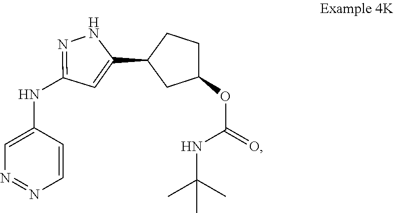 [SMILES: CC(C)(C)NC(=O)O[C@@H]1CC[C@H](c2cc(Nc3ccnnc3)n[nH]2)C1]
or a pharmaceutically acceptable salt thereof.

51. The compound of claim 1 of the formula: 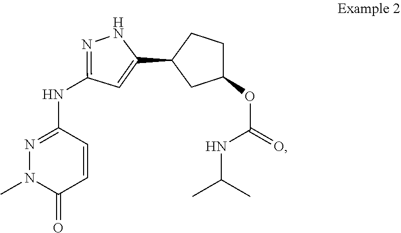 [SMILES: CC(C)NC(=O)O[C@@H]1CC[C@H](c2cc(Nc3ccc(=O)n(C)n3)n[nH]2)C1]
or a pharmaceutically acceptable salt thereof.

52.-53. (canceled)

54. The compound of claim 1 of the formula: 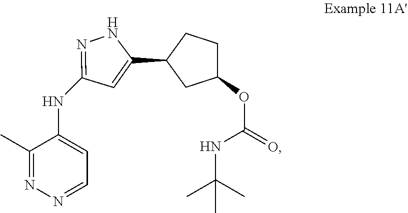 [SMILES: Cc1nnccc1Nc1cc([C@H]2CC[C@@H](OC(=O)NC(C)(C)C)C2)[nH]n1]
or a pharmaceutically acceptable salt thereof.

55.-69. (canceled)

70. A pharmaceutical composition comprising a compound of claim 1, or a pharmaceutically acceptable salt thereof, and a pharmaceutically acceptable excipient.

71. A method of treating a CDK2-mediated disorder in a subject in need thereof, the method comprising administering to the subject a therapeutically effective amount of a compound of claim 1, or a pharmaceutically acceptable salt thereof.

72. The method of claim 71, wherein the disorder is cancer.

73. The method of claim 72, wherein the cancer is acute myelocytic leukemia, bladder cancer, brain cancer, breast cancer, cervical cancer, colon cancer, rectal cancer, endometrial cancer, esophageal cancer, stomach adenocarcinoma, renal cell carcinoma, hepatocellular cancer, non-small cell lung cancer,
small cell lung cancer, neuroblastoma, serous ovarian cancer, prostate cancer, melanoma, thyroid cancer, or uterine carcinosarcoma.

74.-76. (canceled)
